(19) United States
(12) Patent Application Publication
(10) Pub. No.: US 2004/0122096 A1
(43) Pub. Date: Jun. 24, 2004

(54) Use of inhibitors of the cellular Na+/H+ exchanger (NHE) for preparing a medicament for normalizing serum lipids

(75) Inventors: Hans Jochen Lang, Hofheim (DE); Hans-Willi Jansen, Niederhausen (DE); Jan-Robert Schwark, Kelkheim (DE); Heinz-Werner Kleemann, Bischofsheim (DE); Oliver Jung, Lahnau (DE); Hans-Ludwig Schafer, Hochheim (DE); Wolfgang Linz, Mainz (DE); Werner Kramer, Mainz-Laubenheim (DE); Bernward Scholkens, Kelkheim (DE); Eugen Falk, Frankfurt (DE)

Correspondence Address: FINNEGAN, HENDERSON, FARABOW, GARRETT & DUNNER, LLP, 1300 I STREET, NW, WASHINGTON, DC, 20005, US

(73) Assignee: Hoechst Aktiengesellschaft

(21) Appl. No.: 10/680,275

(22) Filed: Oct. 8, 2003

Related U.S. Application Data
(63) Continuation of application No. 09/689,692, filed on Oct. 13, 2000, now abandoned.
Application No. 09/689,692 is a continuation of application No. 09/194,749, filed on Dec. 3, 1998, now abandoned.
Application No. 09/194,749 is a 371 of international application No. PCT/EP97/02548, filed on May 20, 1997.

(30) Foreign Application Priority Data
Jun. 3, 1996 (DE) 196 22 222.2
Mar. 26, 1997 (DE) 197 12 636.7

(51) Int. Cl. A61K 31/20

(52) U.S. Cl. 514/560

(57) ABSTRACT

Use of inhibitors of cellular Na⁺/H⁺ exchanger (NHE) for the production of a medicament for the normalization of serum lipids

The active compounds identified as inhibitors of the cellular Na⁺/H⁺ exchanger (NHE are used for the production of a medicament for the normalization of serum lipids.

They are used for the production of a medicament for lowering the blood lipid level and illnesses caused thereby, as well as of endothelial dysfunction syndrome and illnesses caused thereby.

Use of inhibitors of the cellular Na⁺/H⁺ exchanger (NHE) for the production of a medicament for the normalization of serum lipids

The invention relates to the use of NHE inhibitors for the production of a medicament for the normalization of serum lipids. This applies to all substances which show NHE inhibitor action.

The active compounds which are known and identified as NHE inhibitors are guanidine derivatives, preferably acylguanidines, inter alia such as described in the following publications and patent disclosures: Edward J. Cragoe, Jr., “DIURETICS, Chemistry, Pharmacology and Medicine”, J. WILEY & Sons (1983), 303-341, additionally compounds of the following formulae:

1. (HOE 89/F 288-U.S. Pat. No. 5,292,755)

a) benzoylguanidines of the formula I

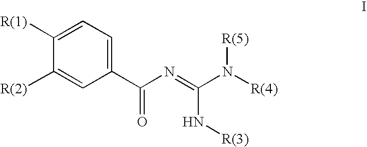

in which:

R(1) or R(2)

is R(6)—S(O)ₙ— or R(7)R(8)N—O₂S—;

and the other substituent R(1) or R(2) in each case

is H, F, Cl, Br, (C₁-C₄)-alkyl, (C₁-C₄)-alkoxy or phenoxy,

which is unsubstituted or substituted by 1-3 substituents selected from the group consisting of fluorine, chlorine, methyl and methoxy;

or the other substituent R(1) or R(2) in each case

is R(6)—S(O)ₙ or R(7)R(8)N—;

n is zero, 1 or 2;

R(6) is (C₁-C₆)-alkyl, (C₅-C₇)-cycloalkyl, cyclopentylmethyl, cyclohexylmethyl or phenyl,

which is unsubstituted or substituted by 1-3 substituents selected from the group consisting of fluorine, chlorine, methyl and methoxy;

R(7) and R(8)

identically or differently are H or (C₁-C₆)-alkyl;

or

R(7) is phenyl-(CH₂)ₘ;

m is 1-4;

or

R(7) is phenyl,

which is unsubstituted or substituted by 1-2 substituents selected from the group consisting of fluorine, chlorine, methyl and methoxy;

or

R(7) and R(8)

together are a straight-chain or branched (C₄-C₇)-chain, where the chain can additionally be interrupted by O, S or NR(9);

R(9) is H or methyl;

or

R(7) and R(8)

together with the nitrogen atom to which they are bonded, are a dihydroindole, tetrahydroquinoline or tetrahydroisoquinoline system;

R(3), R(4) and R(5)

independently of one another are H or (C₁-C₂)-alkyl,

or

R(3) and R(4)

together are a (C₂-C₄)-alkylene chain;

or

R(4) and R(5)

together are a (C₄-C₇)-alkylene chain;

and their pharmaceutically tolerable salts;

(HOE 92/F 34-U.S. Pat. No. 5,373,924)

b) benzoylguanidines of the formula I

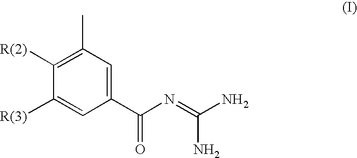

in which:

R(1) is R(4)—SOₘ or R(5)R(6)N—SO₂—;

m is zero, 1 or 2;

R(4) and R(5)

are C₁-C₈-alkyl, C₃-C₆-alkenyl or —CₙH₂ₙ—R(7);

n is zero, 1, 2, 3 or 4;

R(7) is C₅-C₇-cycloalkyl or phenyl,

which is unsubstituted or substituted by 1-3 substituents selected from the group consisting of F, Cl, CF₃, methyl, methoxy and NR(8)R(9);

R(8) and R(9) are H or C₁-C₄-alkyl;

or

R(5) is H;

R(6) is H or C₁-C₄-alkyl,

or

R(5) and R(6)

together are 4 or 5 methylene groups, of which one CH₂ group can be replaced by an O, S, NH, NCH₃ or N-benzyl;

R(2) is hydrogen, F, Cl, Br, (C₁-C₄)-alkyl-, O—(CH₂)ₘCₚF₂ₚ₊₁ or —X—R(10);

m is zero or 1;

p is 1, 2 or 3;

x is O, S or NR(11);

R(10) is H, C₁-C₆-alkyl, C₅-C₇-Cycloalkyl, cyclohexylmethyl, cyclopentylmethyl or —CₙH₂ₙ—R(12);

n is zero, 1, 2, 3 or 4;

R(12) is phenyl,

which is unsubstituted or substituted by 1-3 substituents selected from the group consisting of F, Cl, CF₃, methyl, methoxy und NR(8)R(9);

R(8) and R(9) are H or C₁-C₄-alkyl;

R(1 1) is hydrogen or C₁-C₃-alkyl;

or

R(10) and R(11)

together are 4 or 5 methylene groups, of which one CH₂ group can be replaced by O, S, NH, N—CH₃ or N-benzyl;

R(3) is defined as R(1), or is C₁-C₆-alkyl, nitro, cyano, trifluoromethyl, F, Cl, Br, I or —X—R(10);

x is O, S or NR(11);

R(10) is H, C₁-C₆-alkyl, C₅-C₇-cycloalkyl, cyclohexylmethyl, cyclopentylmethyl or —CₙH₂ₙ—R(12);

n is zero to 4;

R(12) is phenyl,

which is unsubstituted or substituted by 1-3 substituents selected from the group consisting of F, Cl, CF₃, methyl, methoxy und NR(8)R(9);

R(8) and R(9) are H or C₁-C₄-alkyl;

R(1 1) is C₁-C₃-alkyl,

or

R(10) and R(1 1)

together are 4 or 5 methylene groups, of which one CH₂ group can be replaced by O, S, NH, N—CH₃ or N-benzyl;

and their pharmaceutically tolerable salts;

(HOE 92/F 035 EP-Offenlegungsschrift 556 673)

c) ortho-substituted benzoylguanidines of the formula I

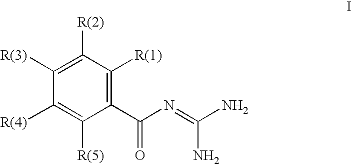

in which:

R(1) is F, Cl, Br, I, C₁-C₆-alkyl or —X—R(6);

X is O, S, NR(7) or Y-ZO;

Y is O or NR(7);

Z is C or SO;

R(6) is H, C₁-C₆-alkyl, C₅-C₇-cycloalkyl, cyclohexylmethyl, cyclopentylmethyl, —(CH₂)ₘCₚF₂ₚ₊₁ or —CₙH₂ₙ—R(8);

m is zero or 1;

p is 1-3;

n is zero to 4;

R(8) is phenyl,

which is unsubstituted or substituted by 1-3 substituents selected from the group consisting of the groups F, Cl, CF₃, methyl, methoxy and NR(9)R(10);

R(9) and R(10) are H or C₁-C₄-alkyl;

R(7) is H or C₁-C₃-alkyl;

or

R(6) and R(7)

together are 4 or 5 methylene groups, of which one CH₂ group can be replaced by O, S, NH, N—CH₃ or N-benzyl;

R(3) is H or —X—R(6);

X is O, S, NR(7) or Y-ZO;

R(7) is H or C₁-C₃-alkyl;

Y is O or NR(7);

where Y is bonded to the phenyl radical of the formula 1,

z is C or SO;

R(6) is H, C₁-C₆-alkyl, C₅-C₇-cycloalkyl, cyclohexylmethyl, cyclopentylmethyl, —(CH₂)ₘCₚF₂ₚ₊₁ or —CₙH₂ₙ—R(8);

m is zero or 1;

p is 1-3;

n is zero to 4;

R(8) is phenyl,

which is unsubstituted or substituted by 1-3 substituents selected from the group consisting of F, Cl, CF₃, methyl, methoxy and NR(9)R(10);

R(9) and R(10) are H or C₁-C₄-alkyl;

or

R(6) and R(7)

together are 4 or 5 methylene groups, of which one CH₂ group can be replaced by O, S, NH, N—CH₃ or N-benzyl;

R(2) and R(4)

identically or differently are R(11)—SO_{q}— or R(12)R(13)N—SO₂—;

q is zero −2;

R(11) is C₁-C₄-alkyl,

which is unsubstituted or carries phenyl as a substituent, where phenyl is unsubstituted or substituted by 1-3 substituents selected from the group consisting of F, Cl, CF₃, methyl, methoxy and NR(9)R(10);

R(9) and R(10)

are H or C₁-C₄-alkyl;

R(12) and R(13)

are defined as R(6) and R(7);

or one of the two radicals R(2) or R(4)

is hydrogen or is defined as R(1);

R(5) is H, methyl, F, Cl or methoxy,

and their pharmaceutically tolerable salts;

(HOE 92/F 036-U.S. Pat. No. 5,364,868)

d) benzoylguanidines of the formula I

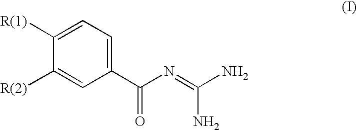

in which:

R(1) or R(2)

is an amino group —NR(3)R(4);

R(3) and R(4)

identically or differently are H, C₁-C₆-alkyl or C₃-C₇₋cycloalkyl;

or

R(3) is phenyl-(CH₂)ₚ—;

p is 0, 1, 2, 3 or 4;

or

R(3) is phenyl,

where the phenyl in each case is unsubstituted or carries one to two substituents selected from the group consisting of fluorine, chlorine, methyl and methoxy;

or

R(3) and R(4)

together can be a straight-chain or branched C₄-C₇-methylene chain, where one —CH₂— member of the methylene chain can be replaced by oxygen, S or NR(5);

R(5) is H or lower alkyl;

the other substituent R(1) or R(2) in each case

is H, F, Cl, C₁-C₄-alkyl, C₁-C₄-alkoxy, CF₃, CₘF₂ₘ₊₁—CH₂—, benzyl or phenoxy,

where the respective phenyl radical is unsubstituted or carries one to two substituents selected from the group consisting of methyl, methoxy, fluorine and chlorine;

m is 1, 2 or 3;

and their pharmaceutically tolerable salts;

(92/F 197 K-NZ 248 013)

e) benzoylguanidines of the formula I

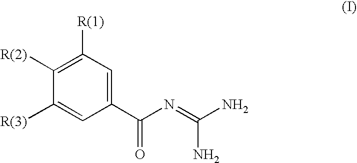

in which:

R(1) is R(4)—SOₘ or R(5)R(6)N—SO₂—;

m is zero, 1 or 2;

R(4) and R(5)

are C₁-C₈-alkyl, C₃-C₆-alkenyl or —CₙH₂ₙ—R(7);

n is zero, 1, 2, 3 or 4;

R(7) is C₅C₇-cycloalkyl or phenyl,

which is unsubstituted or substituted by 1-3 substituents selected from the group consisting of F, Cl, CF₃, methyl, methoxy and NR(8)R(9);

R(8) and R(9) are H or C₁-C₄-alkyl;

or

R(5) is H;

R(6) is H or C₁-C₄-alkyl;

or

R(5) and R(6)

together are 4 or 5 methylene groups, of which one CH₂ group can be replaced by an O, S, NH, N—CH₃ or N-benzyl;

R(2) is hydrogen, straight-chain or branched (C₅-C₈)-alkyl, —CR(13)═CHR(12) or —C≡CR(12);

R(12) is phenyl,

which is unsubstituted or substituted by 1-3 substituents selected from the group consisting of F, Cl, CF₃, methyl, methoxy and NR(14)R(15);

R(14) and R(15)

are H or (C₁-C₄)-alkyl;

or

R(12) is (C₁-C₉)-heteroaryl,

which is unsubstituted or substituted as phenyl,

or

R(12) is (C₁-C₆)-alkyl,

which is unsubstituted or substituted by 1-3 OH,

or

R(12) is (C₃-C₈)-cycloalkyl;

R(13) is hydrogen or methyl,

or

R(12) is (C₃-C₈)-cycloalkyl, (C₃-C₈)-cycloalkyl-(C₁-C₄)alkyl, phenyl, C₆H₅—(C₁-C₄)-alkyl, naphthyl, biphenylyl, 1,1-diphenyl-(C₁-C₄)-alkyl, cyclopentadienyl, pyridyl, pyrrolyl, furanyl, thienyl, thiazolyl, oxazolyl, indenyl, quinolyl, indolyl, benzofuranyl, benzothienyl, benzothiazolyl, benzoxazolyl, imidazolyl, pyrazolyl, triazolyl, tetrazolyl, isoxazolyl, isothiazolyl, pyrazinyl, pyrimidinyl, pyndazinyl, indazolyl, isoquinolyl, phthalazinyl, quinoxalinyl, quinazolinyl or cinnolinyl;

R(3) is defined as R(2);

and where the aromatic substituents R(2) and R(3) are unsubstituted or substituted by 1-3 substituents from the groups F, Cl, CF₃, (C₁-C₄)-alkyl or -alkoxy, or NR(10)R(11) with R(10) and R(11) being H or (C₁-C₄)-alkyl;

and their pharmaceutically tolerable salts;

(HOE 92JF 303 K-EP-Offenlegungsschrift 589 336, NZ 248 703)

f) benzoylguanidines of the formula I

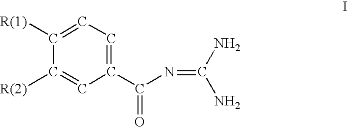

in which:

R(1) or R(2)

is R(3)—S(O)ₙ— or

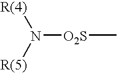

the other substituent R(1) or R(2) in each case

is H, OH, F, Cl, Br, I, C₁-C₄-alkyl, C₁-C₄-alkoxy, benzyloxy or phenoxy,

which is unsubstituted or carries one to three substituents selected from the group consisting of fluorine, chlorine, methyl, methoxy, hydroxyl and benzyloxy,

R(3)—S(O)ₙ, —NR(4)R(5) or 3,4-dehydropiperidine

R(3) is C₁-C₆-alkyl, C₅-C₇-cycloalkyl, cyclopentylmethyl, cyclohexylmethyl or phenyl,

which is unsubstituted or substituted by one to three substituents selected from the group consisting of fluorine, chlorine, methyl and methoxy;

R(4) and R(5)

identically or differently, are H or C₁-C₆-alkyl;

or

R(4) is phenyl-(CH₂)ₘ—;

m is 1, 2, 3 or 4;

or

R(4) is phenyl,

which is unsubstituted or carries one to two substituents selected from the group consisting of fluorine, chlorine, methyl and methoxy;

or

R(4) and R(5)

together are a straight-chain or branched C₄-C₇-chain, where the chain can additionally be interrupted by O, S or NR(6),

R(6) is H or methyl;

or

R(4) and R(5)

together with the nitrogen atom to which they are bonded, are a dihydroindole, tetrahydroquinoline or tetrahydroisoquinoline system;

n is zero, 1 or 2;

and their pharmaceutically tolerable salts;

(92/F 304-U.S. Pat. No. 5,416,094)

g) isoquinolines of the formula I

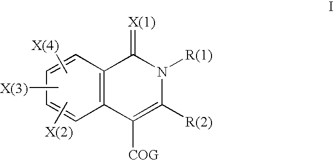

in which:

R(1) is hydrogen, alkyl, cycloalkyl, arylalkyl, alkenyl, substituted aminoalkyl or an aryl or heteroaryl ring;

where the rings are unsubstituted or substituted by 1-3 groups selected from the group consisting of halogen, nitro, amino, mono(lower alkyl)amino, di(lower alkyl)amino, lower alkyl, lower alkoxy, benzyloxy, phenoxy, hydroxyl, trifluoromethyl,

R(2) is hydrogen, halogen, alkyl or aryl;

which is unsubstituted or substituted by 1-3 groups selected from the group consisting of halogen, nitro, amino, mono(lower alkyl)amino, di(lower alkyl)amino, lower alkyl, lower alkoxy, benzyloxy, phenoxy, hydroxyl,

G is

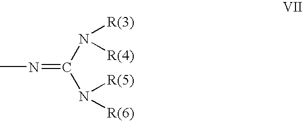

X(2), X(3) and X(4)

independently of one another are hydrogen, halogen, nitro, amino, alkyl, sulfonamide, mono(lower alkyl)amino, di(lower alkyl)amino, lower alkyl, benzyloxy, hydroxyl;

X(1) is hydrogen, oxygen, sulfur or NR(7);

R(7) is hydrogen, alkyl, cycloalkyl, arylalkyl, alkenyl, substituted

aminoalkyl or an aryl or a heteroaryl ring;

which rings are unsubstituted or substituted by 1-3 groups selected from the group consisting of halogen, nitro, amino, mono(lower alkyl)amino, di(lower alkyl)amino, lower alkyl, lower alkoxy, benzyloxy, phenoxy, hydroxyl and trifluoromethyl;

in which substituents each alkyl chain or alkenyl chain can be interrupted by oxygen, sulfur or NR(8);

R(8) is hydrogen, alkyl, cycloalkyl, arylalkyl, alkenyl, substituted aminoalkyl or an aryl or heteroaryl ring,

which rings are unsubstituted or substituted by 1-3 groups selected from the group consisting of halogen, nitro, amino, mono(lower alkyl)amino, di(lower alkyl)amino, lower alkyl, lower alkoxy, benzyloxy, phenoxy, hydroxyl and trifluoromethyl;

and their pharmaceutically acceptable salts;

(92/F 404-EP 602 522, NZ 250 438)

h) compounds of the formula I

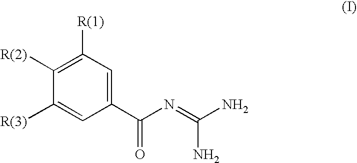

in which:

R(1) is hydrogen, F, Cl, Br, I, —NO₂, —C≡N, —CF₃, R(4)—SOₘ or

R(5)R(6)N—SO₂—;

m is zero, 1 or 2;

R(4) and R(5)

are (C₁-C₈)-alkyl, (C₃-C₆)-alkenyl, —CₙH₂ₙ—R(7) or CF₃;

n is zero, 1, 2, 3 or 4;

R(7) is (C₃-C₇)-cycloalkyl or phenyl,

which is not substituted or is substituted by 1-3 substituents selected from the group consisting of F, Cl, CF₃, methyl, methoxy and NR(8)R(9);

R(8) and R(9) are H or C₁-C₄-alkyl;

or

R(5) is H;

R(6) is H or (C₁-C₄)-alkyl;

or

R(5) and R(6)

together are 4 or 5 methylene groups, of which one CH₂ group can be replaced by oxygen, S, NH, N—CH₃ or N-benzyl;

R(2) is —SR(10), —OR(10), —NHR(10), —NR(10)R(11), —CHR(10)R(12), —[CR(12)R(13)OR(13′)], —{C—[CH₂—OR(13′)]R(12) (R(13)} or —[CR(18)R(17)]ₚ—(CO)—[CR(19)R(20)]_{q}—R(14);

R(10), R(11)

identically or differently

are —[CHR(16)]ₛ—(CH₂)ₚ—(CHOH)_{q}—(CH₂)ᵣ—(CHOH)ₜ—R(21) or —(CH₂)ₚ—O—(CH₂—CH₂O)_{q}—R(21),

R(21) is hydrogen, methyl,

p, q, r identically or differently

are zero, 1, 2, 3 or 4;

s is zero or 1;

t is 1, 2, 3 or 4;

R(12) and R(13)

identically or differently are hydrogen, (C₁-C₆)-alkyl or, together with the carbon atom carrying them, are a (C₃-C₈)-cycloalkyl,

R(13′) is hydrogen or (C₁-C₄)-alkyl;

R(14) is H, (C₁-C₆)-alkyl, (C₃-C₈)-cycloalkyl or —CₐH₂ₐ—R(15);

a is zero, 1, 2, 3 or 4;

R(15) is phenyl,

which is unsubstituted or substituted by 1-3 substituents selected from the group consisting of F, Cl, CF₃, methyl, methoxy and NR(8)R(9);

R(8) and R(9) are H or (C₁-C₄)-alkyl;

or

R(15) is (C₁-C₉)-heteroaryl,

which is unsubstituted or substituted as phenyl,

or

R(15) is (C₁-C₆)-alkyl,

which is unsubstituted or substituted by 1-3 OH;

R(16), R(17), R(18), R(19) and R(20)

are hydrogen or (C₁-C₃)-alkyl;

R(3) is defined as R(1),

or

R(3) is (C₁-C₆)-alkyl or —X—R(22);

X is oxygen, S or NR(16);

R(16) is H or (C₁-C₃)-alkyl;

or

R(22) and R(16)

together are 4 or 5 methylene groups, of which one CH₂ group can be replaced by oxygen, S, NH, N—CH₃ or N-benzyl;

R(22) is defined as R(14);

and their pharmaceutically tolerable salts;

(HOE 921F 405-EP 602 523, NZ 250 437)

i) benzoylguanidines of the formula I

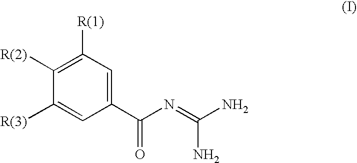

in which:

R(1) is hydrogen, F, Cl, Br, I, —NO₂, —C≡N, R(16)-CₚH₂ₚ—O_{q}, R(4)-SOₘ or

R(5)R(6)N—SO₂—;

m is zero, 1 or 2;

p is zero or 1;

q is zero, 1, 2 or 3;

R(16) is CᵣF₂ᵣ₊₁;

r is 1, 2 or 3;

R(4) and R(5)

are (C₁-C₈)-alkyl, (C₃-C₆)-alkenyl, —CₙH₂ₙ—R(7) or CF₃;

n is zero, 1, 2, 3 or 4;

R(7) is (C₃-C₇)-cycloalkyl or phenyl,

which is not substituted or is substituted by 1-3 substituents selected from the group consisting of F, Cl, CF₃, methyl, methoxy and NR(8)R(9);

R(8) and R(9) are H or C₁-C₄-alkyl;

or

R(5) is H;

R(6) is H or (C₁-C₄)-alkyl;

or

R(5) and R(6)

together are 4 or 5 methylene groups, of which one CH₂ group can be replaced by oxygen, S, NH₁, N—CH₃ or N-benzyl,

R(2) is (C₁-C₉)-heteroaryl,

which is linked via C or N and which is unsubstituted or substituted by 1-3 substituents selected from the group consisting of F, Cl, CF₃, CH₃, methoxy, hydroxyl, amino, methylamino and dimethylamino;

or

R(2) is —SR(10), —OR(10), —NR(10)R(11), —CR(10)R(11)R(12);

R(10) is —CₐH₂ₐ—(C₁-C₉)-heteroaryl,

which is unsubstituted or substituted by 1-3 substituents selected from the group consisting of F, Cl, CF₃, CH₃, methoxy, hydroxyl, amino, methylamino and dimethylamino;

a is zero, 1 or 2;

R(11) and R(12)

independently of one another are defined as R(10) or are hydrogen or (C₁-C₄)-alkyl;

R(3) is defined as R(1), or is (C₁-C₆)-alkyl or —X—R(13);

X is oxygen, S, or NR(14);

R(14) is H or (C₁-C₃)-alkyl;

R(13) is H, (C₁-C₆)-alkyl, (C₃-C₈)-cycloalkyl or —C_{b}H_{2b}—R(15);

b is zero, 1, 2, 3 or 4;

or

R(13) and R(14)

together are 4 or 5 methylene groups, of which one CH₂ group can be replaced by oxygen, S, NH, N—CH₃ or N-benzyl;

R(15) is phenyl,

which is unsubstituted or substituted by 1-3 substituents selected from the group consisting of F, Cl, CF₃, methyl, methoxy and NR(8)R(9);

R(8) and R(9) are H or (C₁-C₄)-alkyl;

and their pharmaceutically tolerable salts;

(HOE 92/F 411-NZ 250 450, EP 603 650)

k) benzoylguanidines of the formula I

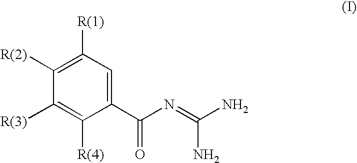

in which:

one of the substituents R(1), R(2), R(3) or R(4)

is an amino group

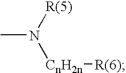

R(5) is hydrogen or C₍₁₋₆₎-alkyl;

n is zero, 1, 2, 3 or 4;

R(6) is H or C₍₁₋₄₎-alkyl;

in which one CH₂ group can be replaced by 1 sulfur atom or a group

NR(7);

R(7) is hydrogen, methyl or ethyl;

or

R(6) is C₍₃₋₈₎-cycloalkyl or phenyl,

which is unsubstituted or carries 1, 2 or 3 substituents selected from the group consisting of F, Cl, Br, methyl, methoxy, —NR(8)R(9);

R(8) and R(9)

are H, methyl or ethyl;

or

R(5) and R(6)

together with the nitrogen atom are a 5-, 6- or 7-membered ring, in which 1 carbon atom can be replaced by oxygen, S or NR(10);

R(10) is H, C₍₁₋₃₎-alkyl or benzyl;

and the other substituents R(1), R(2), R(3), R(4) in each case are:

hydrogen, F, Cl, Br, I, CN, CF₃, NO₂, CF₃—O—, CₘF₂ₘ₊₁—CH₂—O— or R(11)-C_{q}H_{2q}—Xₚ—;

m is 1, 2 or 3;

q is zero, 1, 2, 3 or 4;

p is zero or 1;

X is oxygen or NR(12);

R(12) is H or C₍₁₋₃₎-alkyl;

R(11) is hydrogen, C₍₁₋₆₎-alkyl, C₍₃₋₈₎-cycloalkyl or phenyl,

which is unsubstituted or substituted by 1, 2 or 3 substituents selected from the group consisting of F, Cl, CH₃, CH₃—O— and NR(13)R(14);

R(13), R(14)

are H, methyl or ethyl;

and their pharmaceutically tolerable salts;

(HOE 92/F 422-EP 604 852)

l) benzoylguanidines of the formula I

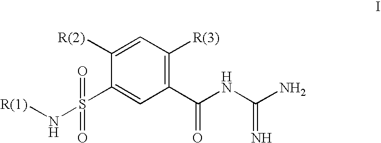

in which:

R(1) is R(4)R(5)N—C(X)—;

X is oxygen, S or N—R(6);

R(4) and R(5)

identically or differently, are H, (C₁-C₈)-alkyl, (C₃-C₆)-alkenyl or —CₙH₂ₙ—R(7);

n is zero, 1, 2, 3 or 4;

R(7) is (C₅-C₇)-cycloalkyl or phenyl,

which is unsubstituted or substituted by 1-3 substituents selected from the group consisting of F, Cl, CF₃, methoxy and (C₁-C₄)-alkyl;

or

R(4) and R(5)

together are 4 or 5 methylene groups, of which one CH₂ group can be replaced by oxygen, S, NH, N—CH₃ or N-benzyl;

R(6) is defined as R(4) or is amidine:

R(2) is H, F, Cl, Br, I, (C₁-C₈)-alkyl, 1-alkenyl or 1-alkynyl, (C₃-C₈)-cycloalkyl, (C₃-C₈)cycloalkyl-(C₁-C₄)-alkyl, phenyl, C₆H₅—(C₁-C₄)-alkyl, naphthyl, biphenylyl, 1,1-diphenyl-(C₁-C₄)-alkyl, cyclopentadienyl, pyridyl, thiopyridyl, pyrrolyl, furanyl, thienyl, thiazolyl, oxazolyl, indenyl, quinolyl, indolyl, benzofuranyl, benzothienyl, benzothiazolyl, benzoxazolyl or —W—R(8);

W is oxygen, S or NR(9);

R(8) is H, (C₁-C₆)-alkyl, (C₅-C₇)-cycloalkyl, cyclohexylmethyl, cyclopentylmethyl, —(CH₂)ₘCₚF₂ₚ₊₁ or —C_{q}H_{2q}—R(10);

m is zero or 1;

p is 1, 2 or 3;

q is zero, 1, 2, 3 or 4;

R(10) is phenyl,

which is unsubstituted or substituted by 1-3 substituents selected from the group consisting of F, Cl, CF₃, methyl, methoxy and NR(11)R(12);

R(11) and R(12) are H or (C₁-C₄)-alkyl;

R(9) is H or (C₁-C₃)-alkyl;

or

R(8) and R(9)

together are 4 or 5 methylene groups, of which one CH₂ group can be replaced by oxygen, S, NH, N—CH₃ or N-benzyl;

R(3) is H, F, Cl, Br, I, (C₁-C₆)-alkyl or —W—R(8) as defined for R(2),

and their pharmaceutically acceptable salts;

(93/F 054-NZ 250 919, EP-Offenlegungsschrift 612 723)

m) benzoylguanidines of the formula I

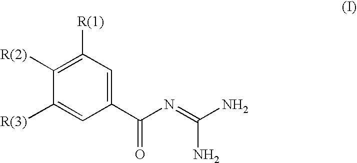

in which:

R(1), R(2), R(3)

are hydrogen, F, Cl, Br, I or (C₁-C₁₂)-alkyl;

one of the substituents R(1), R(2) or R(3)

is N₃, CN, OH or (C₁-C₁₀)-alkyloxy, if at least one of the remaining substituents R(1), R(2) or R(3) is a sufficiently lipophilic alkyl radical having 3 to 12 carbon atoms;

or

one of the substituents R(1), R(2) and R(3)

is R(4)-CₙH₂ₙ—Oₘ—;

m is zero or 1;

n is zero, 1, 2 or 3;

R(4) is CₚF₂ₚ₊₁;

p is 1, 2 or 3, if n is zero or 1;

or

R(4) is (C₃-C₁₂)-cycloalkyl, phenyl, pyridyl, quinolyl or isoquinolyl,

where the aromatic and heteroaromatic ring systems are unsubstituted or substituted by a substituent selected from the group consisting of F, Cl, CF₃, methyl, methoxy and NR(5)R(6);

R(5) and R(6)

are hydrogen or (C₁-C₄)-alkyl;

or one of the substituents R(1), R(2) and R(3)

is —C≡CR(5) or —C[R(6))═CR(5);

R(5) is phenyl,

which is unsubstituted or substituted by 1-3 substituents selected from the group consisting of F, Cl, CF₃, methyl, methoxy, hydroxyl, amino, methylamino and dimethylamino, (C₁-C₉)-heteroaryl,

which is unsubstituted or substituted as phenyl,

or

R(5) is (C₁-C₆)-alkyl,

which is unsubstituted or substituted by 1-3 OH;

or

R(5) is (C₃-C₈)-cycloalkyl,

R(6) is hydrogen or methyl;

and their pharmacologically acceptable salts;

(93/F 153-EP-Offenlegungsschrift 627 413, NZ 260 660)

o) benzoylguanidines of the formula I

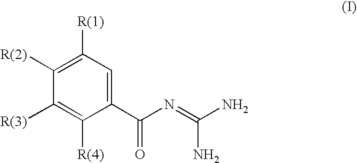

in which:

R(1) is hydrogen, F, Cl, Br, I, —NO₂, —C≡N, Xₒ—(CH₂)ₚ—(CF₂)_{q}—CF₃, R(5)-SOₘ, R(6)-CO— or R(6)R(7)N—SO₂—, where

X is oxygen, S or NR(14);

m is zero, 1 or 2;

o is zero or 1;

p is zero, 1 or 2;

q is zero, 1, 2, 3, 4, 5 or 6;

R(5) and R(6)

are (C₁-C₈)-alkyl, (C₃-C₆)-alkenyl, —CₙH₂ₙ—R(8) or CF₃;

n is zero, 1, 2, 3 or 4;

R(8) is (C₃-C₇)-cycloalkyl or phenyl,

which is not substituted or is substituted by 1-3 substituents selected from the group consisting of F, Cl, CF₃, methyl, methoxy and NR(9)R(10);

R(9) and R(10) are H or C₁-C₄-alkyl;

or

R(6) is H;

R(7) is H or (C₁-C₄)-alkyl;

or

R(6) and R(7)

together are 4 or 5 methylene groups, of which one CH₂ group can be replaced by oxygen, S, NH, N—CH₃ or N-benzyl;

R(2)is

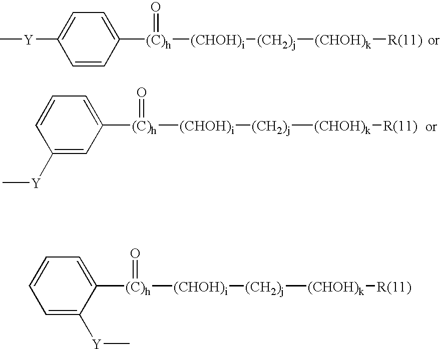

Y is oxygen, —S— or —NR(12)-;

R(11) and R(12)

are hydrogen or (C₁-C₃)-alkyl;

h is zero or 1;

i, j and k

independently are zero, 1, 2, 3 or 4;

but where h, i and k are not simultaneously zero,

R(3) is defined as R(1), or is (C₁-C₆)-alkyl or —X—R(13);

X is oxygen, S or NR(14);

R(14) is H or (C₁-C₃)-alkyl;

R(13) is H, (C₁-C₆)-alkyl, (C₃-C₈)-cycloalkyl or —C_{b}H_{2b}—R(15);

b is zero, 1, 2, 3 or 4;

or

R(13) and R(14)

together are 4 or 5 methylene groups, where one CH₂ group can be replaced by oxygen, S, NH, N—CH₃ or N-benzyl;

R(15) is phenyl,

which is unsubstituted or substituted by 1-3 substituents selected from the group consisting of F, Cl, CF₃, methyl, methoxy and NR(9)R(10);

R(9) and R(10) are H or (C₁-C₄)-alkyl;

R(4) is hydrogen, —OR(16) or —NR(16)R(17);

R(16) and R(17)

independently are hydrogen or (C₁-C₃)-alkyl;

and their pharmaceutically tolerable salts;

(HOE 93/F 154-EP-Offenlegungsschrift 628 543, NZ 260 681)

p) benzoylguanidines of the formula I

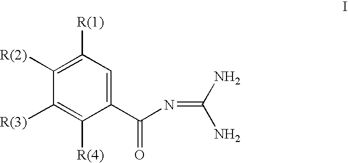

in which:

R(1) is R(6)-CO or R(7)R(8)N—CO;

R(6) is (C₁-C₈)-alkyl, (C₁-C₈)-perfluoroalkyl, (C₃-C₈)-alkenyl or —CₙH₂ₙ—R(9);

n is zero, 1, 2, 3 or 4;

R(9) is (C₃-C₈)-cycloalkyl, phenyl, biphenylyl or naphthyl,

where the aromatics are not substituted or are substituted by 1-3 substituents selected from the group consisting of F, Cl, CF₃, methyl, methoxy and NR(10)R(11);

R(10) and R(11) are H, (C₁-C₄)-alkyl or (C₁-C₄)-perfluoroalkyl

R(7) is H, (C₁-C₈)-alkyl, (C₁-C₈)-perfluoroalkyl, (C₃-C₈)-alkenyl or —CₙH₂ₙ—R(12);

n is zero, 1, 2, 3 or 4;

R(12) is (C₃-C₈)-cycloalkyl, phenyl, biphenylyl or naphthyl,

where the aromatics are not substituted or are substituted by 1-3 substituents selected from the group consisting of F, Cl, CF₃, methyl, methoxy and NR(13)R(14);

R(13) and R(14) are H, (C₁-C₄)-alkyl or (C₁-C₄)-perfluoroalkyl

R(8) is H, (C₁-C₄)-alkyl or (C₁-C₄)-perfluoroalkyl;

or

R(7) and R(8)

together are 4 or 5 methylene groups, of which one CH₂ group can be replaced by oxygen, S, NH, N—CH₃ or N-benzyl;

R(2) is defined as R(1), or is H, F, Cl, Br, I, CN, NO₂, (C₁-C₈)-alkyl, (C₁-C₈)-perfluoroalkyl, (C₃-C₈)-alkenyl or —CₙH₂ₙR(15);

n is zero 1, 2, 3, 4;

R(15) is (C₃-C₈)-cycloalkyl, phenyl, biphenylyl or naphthyl,

where the aromatics are not substituted or are substituted by 1-3 substituents selected from the group consisting of F, Cl, CF₃, methyl, methoxy and NR(16)R(17);

R(16) and R(17) are H, (C₁-C₄)-alkyl or (C₁-C₄)-perfluoroalkyl

or

R(2) is (C₁-C₉)-heteroaryl,

which is linked via C or N and which is unsubstituted or substituted by 1-3 substituents selected from the group consisting of F, Cl, CF₃₁ CH₃, methoxy, hydroxyl, amino, methylamino and dimethylamino;

or

R(2) is SR(18), —OR(18), —NR(18)R(19), —CR(18)R(19)R(20);

R(18) is —CₐH₂ₐ—(C₁-C₉)-heteroaryl,

which is unsubstituted or substituted by 1-3 substituents selected from the group consisting of F, Cl, CF₃, CH₃, methoxy, hydroxyl, amino, methylamino and dimethylamino;

a is zero, 1 or 2;

R(19) and R(20)

independently of one another are defined as R(18) or are hydrogen, (C₁-C₄)-alkyl or (C₁-C₄)-perfluoroalkyl;

or

R(2) is R(21)-SOₘ or R(22)R(23)N—SO₂—;

m is 1 or 2;

R(21) is (C₁-C₈)-alkyl, (C₁-C₈)-perfluoroalkyl, (C₃-C₈)-alkenyl, —CₙH₂ₙ—R(24),

n is zero, 1, 2, 3 or 4;

R(24) is (C₃-C₈)-cycloalkyl, phenyl, biphenylyl or naphthyl,

where the aromatics are not substituted or are substituted by 1-3 substituents selected from the group consisting of F, Cl, CF₃, methyl, methoxy and NR(27)R(28);

R(27) and R(28) are H, (C₁-C₄)-alkyl or (C₁-C₄)-perfluoroalkyl

R(22) is H, (C₁-C₈)-alkyl, (C₁-C₈)-perfluoroalkyl, (C₃-C₈)-alkenyl, —CₙH₂ₙ—R(29);

n is zero, 1, 2, 3 or 4;

R(29) is (C₃-C₈)-cycloalkyl, phenyl, biphenylyl or naphthyl,

where the aromatics are not substituted or are substituted by 1-3 substituents selected from the group consisting of F, Cl, CF₃, methyl, methoxy and NR(30)R(31);

R(30) and R(31) are H, (C₁-C₄)-alkyl or (C₁-C₄)-perfluoroalkyl;

R(23) is H, (C₁-C₄)-alkyl or (C₁-C₄)-perfluoroalkyl;

or

R(22) and R(23)

together are 4 or 5 methylene groups, of which one CH₂ group can be replaced by oxygen, S, NH, N—CH₃ or N-benzyl;

or

R(2) is R(33)X—;

X is oxygen, S, NR(34), (D=O)A-, NR(34)C=MN(*)R(35)-;

M is oxygen or S;

A is oxygen or NR(34);

D is C or SO;

R(33) is (C₁-C₈)-alkyl, (C₃-C₈)-alkenyl, (CH₂)_{b}C_{d}F_{2d+1}, —CₙH₂ₙ—R(36),

b is zero or 1;

d is 1, 2, 3, 4, 5, 6 or 7;

n is zero, 1, 2, 3 or 4;

R(36) is (C₃-C₈)-cycloalkyl, phenyl, biphenylyl or naphthyl,

where the aromatics are not substituted or are substituted by 1-3 substituents selected from the group consisting of F, Cl, CF₃, methyl, methoxy and NR(37)R(38);

R(37) and R(38) are H, (C₁-C₄)-alkyl or (C₁-C₄)-perfluoroalkyl;

R(34) is H, (C₁-C₄)-alkyl or (C₁-C₄)-perfluoroalkyl;

R(35) is defined as R(33);

or

R(33) and R(34)

together are 4 or 5 methylene groups, of which one CH₂ group can be replaced by oxygen, S, NH, N—CH₃ or N-benzyl;

where A and N^{(*)} are bonded to the phenyl nucleus of the benzoylguanidine parent structure;

or

R(2) is —SR(40), —OR(40), —NHR(40), —NR(40)R(41), —CHR(40)R(42), —C(R(42)R(43)OH], —C≡CR(45), —CR(46)═CHR(45), —[CR(47)R(48)]ᵤ—(CO)—[CR49)R(50)]ᵥ—R(44);

R(40), R(41)

identically or differently are —(CH₂)ₚ—(CHOH)_{q}—(CH₂)ᵣ—(CHOH)ₜ—R(51) or —(CH₂)ₚ—O—(CH₂—CH₂O)_{q}—R(51);

R(51) is hydrogen or methyl;

u is 1, 2, 3 or 4;

v is zero, 1, 2, 3 or 4;

p, q, r

identically or differently are zero, 1, 2, 3 or 4;

t is 1, 2, 3 or 4;

R(42) and R(43)

identically or differently are hydrogen or (C₁-C₆)-alkyl;

or

R(42) and R(43)

together with the carbon atom carrying them form a (C₃-C₈)-cycloalkyl;

R(44) is H, (C₁-C₆)-alkyl, (C₃-C₈)-cycloalkyl or —CₑH₂ₑ—R(45);

e is zero, 1, 2, 3 or 4;

R(45) is phenyl,

which is unsubstituted or substituted by 1-3 substituents from the group consisting of F, Cl, CF₃, methyl, methoxy and NR(52)R(53) where

R(52) and R(53) are H or (C₁-C₄)-alkyl, or

R(45) is (C₁-C₉)-heteroaryl,

which is unsubstituted or substituted as phenyl;

or

R(45) is (C₁-C₆)-alkyl,

which is unsubstituted or substituted by 1-3 OH;

R(46), R(47), R(48), R(49) and R(50)

are hydrogen or methyl;

or

R(2) is R(55)-NH—SO₂—;

R(55) is R(56)R(57)N—(C═Y)—;

Y is oxygen, S or N—R(58);

R(56) and R(57)

identically or differently are H, (C₁-C₈)-alkyl, (C₃-C₆)-alkenyl or —C_{f}H_{2f}—R(59);

f is zero, 1, 2, 3 or 4;

R(59) is (C₅-C₇)-cycloalkyl or phenyl,

which is unsubstituted or substituted by 1-3 substituents selected from the group consisting of F, Cl, CF₃, methoxy and (C₁-C₄)-alkyl;

or

R(56) and R(57)

together are 4 or 5 methylene groups, of which one CH₂ group can be replaced by oxygen, S, NH, N—CH₃ or N-benzyl;

R(58) is defined as R(56) or is amidine;

R(3), R(4) and R(5)

independently of one another are defined as R(1) or R(2);

and their pharmaceutically tolerable salts;

(HOE 93/F 220-EP-Offenlegungsschrift 640 593, NZ 264 117)

q) benzoylguanidines of the formula I

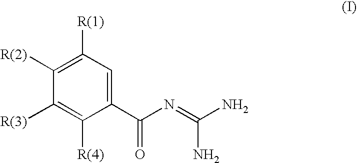

in which:

R(1) is hydrogen, F, Cl, Br, I, —NO₂, —C≡N, —Xₒ—(CH₂)ₚ—(CF₂)_{q}—CF₃,

R(5)-SOₘ—, R(6)-CO—, R(6)R(7)N—CO— or R(6)R(7)N—SO₂—;

X is oxygen, —S— or NR(14);

m is zero, 1 or 2;

o is zero or 1;

p is zero, 1 or 2;

q is zero, 1, 2, 3, 4, 5 or 6;

R(5) and R(6)

are (C₁-C₈)-alkyl (C₃-C₆)-alkenyl, −CₙH₂ₙ—R(8) or CF₃;

n is zero, 1, 2, 3 or 4;

R(8) is (C₃-C₇)cycloalkyl, phenyl,

which is not substituted or is substituted by 1 to 3 substituents selected from the group consisting of F, Cl, CF₃, methyl, methoxy and NR(9)R(10);

R(9) and R(10) are H or (C₁-C₄)-alkyl;

or

R(6) is hydrogen;

R(7) is hydrogen or (C₁-C₄)-alkyl;

or

R(6) and R(7)

together are 4 or 5 methylene groups, of which one CH₂ group can be replaced by oxygen, S, NH, N—CH₃ or N-benzyl;

R(2) is

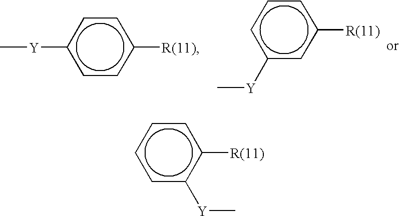

R(11) is (C₁-C₉)-heteroaryl,

which is linked via C or N and which is unsubstituted or substituted by 1 to 3 substituents selected from the group consisting of F, Cl, CF₃, CH₃, methoxy, hydroxyl, amino, methylamino, dimethylamino and benzyl;

Y is oxygen, —S— or NR(12);

R(12) is H or (C₁-C₄)-alkyl;

R(3) is defined as R(1);

or

R(3) is (C₁-C₆)-alkyl or —X—R(13);

X is oxygen, —S— or NR(14);

R(14) is H or (C₁-C₃)-alkyl;

R(13) is H, (C₁-C₆)-alkyl, (C₃-C₈)-cycloalkyl or —C_{b}H_{2b}—R(15);

b is zero, 1, 2, 3 or 4;

or

R(13) and R(14)

together are 4 or 5 methylene groups, of which one CH₂ group can be replaced by oxygen, S, NH, N—CH₃ or N-benzyl;

R(15) is phenyl,

which is unsubstituted or substituted by 1-3 substituents selected from the group consisting of F, Cl, CF₃, methyl, methoxy and NR(9)R(10);

R(9) and R(10) are H or (C₁-C₄)-alkyl;

R(4) is hydrogen, —OR(16), —NR(16)R(17) or CᵣF₂ᵣ₊₁;

R(16) and R(17)

independently are hydrogen or (C₁-C₃)-alkyl;

r is 1, 2, 3 or 4;

and their pharmaceutically tolerable salts;

(HOE 93/F 223 K-EP 639 573, NZ 264 130)

r) benzo-fused 5-membered ring heterocycles of the formula I

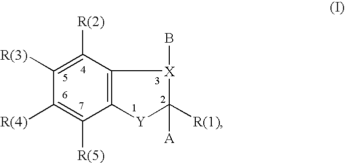

in which:

X is N or CR(6);

Y is oxygen, S or NR(7);

A, B together are a bond

or

A, B are both hydrogen, if X is simultaneously CR(6) and Y is NR(7);

one of the substituents R(1) to R(6) is a —CO—N═C(NH₂)₂ group;

the other substituents R(1) to R(6) in each case

are hydrogen, F, Cl, Br, I or (C₁-C₆)-alkyl;

up to two of the other substituents R(1) to R(6)

are CN, NO₂, N₃, (C₁-C₄)-alkyloxy or CF₃;

up to one of the other substituents

is R(8)-CₙH₂ₙ-Z-;

n is zero to 10;

where the alkylene chain —CₙH₂ₙ— is straight-chain or branched and where one carbon atom can be replaced by an oxygen or sulfur atom or by a nitrogen atom;

R(8) is hydrogen, (C₂-C₆)-alkenyl or (C₃-C₁₀)-cycloalkyl,

which is unsubstituted or substituted by 1 to 4 methyl groups or an OH group, or can contain an ethylene group —CH═CH—, and in which one methylene group can be replaced by an oxygen or sulfur atom or by a nitrogen atom;

or

R(8) is phenyl,

which is unsubstituted or substituted by 1 to 3 substituents selected from the group consisting of F, Cl, Br, I, CF₃, CH₃—S(O)ₛ— or R(9)W_{y}—;

s is zero, 1 or 2;

R(9) is H, methyl, ethyl,

W is oxygen or NR(10);

R(10) is H or methyl;

y is zero or 1;

or

R(8) is CₘF₂ₘ₊₁;

m is 1 to 3;

or

R(8) is 1- or 2-naphthyl, pyridyl, quinolyl or isoquinolyl;

Z is —CO—, —CH₂— or —[CR(11)(OH)]_{q}—;

q is 1, 2 or 3;

R(11) is H or methyl;

or

Z is oxygen or —NR(12)-;

R(12) is H or methyl;

or

Z is —S(O)ₛ—;

s is zero, 1 or 2;

or

Z is —SO₂—NR(13)-;

R(13) is H or (C₁-C₄)-alkyl;

R(7) is hydrogen, (C₁-C₁₀)-alkyl, (C₂-C₁₀)-alkenyl or R(8)-CₙH₂ₙ—;

and their pharmaceutically tolerable salts;

(HOE 93/F 236-EP-Offenlegungsschrift 638 548, NZ 264 216)

s) benzoylguanidines of the formula I

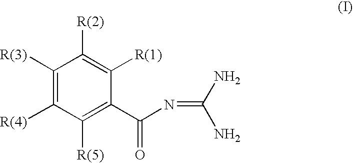

in which:

R(1), R(3) or R(4)

is —NR(6) C═X NR(7)R(8);

X is oxygen or S;

R(6) is hydrogen, (C₁-C₈)-alkyl, (C₁-C₈)-perfluoroalkyl, (C₃-C₈)-alkenyl or —CₙH₂ₙ—R(9);

n is zero, 1, 2, 3 or 4;

R(9) is (C₃-C₈)-cycloalkyl, phenyl, biphenylyl or naphthyl,

where the aromatics are not substituted or are substituted by 1-3 substituents selected from the group consisting of F, Cl, CF₃, methyl, methoxy and NR(10)R(11);

R(10) and R(11) are H, (C₁-C₄)-alkyl or (C₁-C₄)-perfluoroalkyl;

R(7) is hydrogen, (C₁-C₈)-alkyl, (C₁-C₈)-perfluoroalkyl, (C₃-C₈)-alkenyl or —CₒH₂ₒ—R(12);

o is zero, 1, 2, 3 or 4;

R(12) is (C₃-C₈)-cycloalkyl, phenyl, biphenylyl or naphthyl,

where the aromatics are not substituted or are substituted by 1-3 substituents selected from the group consisting of F, Cl, CF₃, methyl, methoxy and NR(1 3)R(1 4);

R(13) and R(14) are H, (C₁-C₄)-alkyl or (C₁-C₄)-perfluoroalkyl;

R(8) is defined as R(7);

or

R(7) and R(8)

together are 4 or 5 methylene groups, of which one CH₂ group can be replaced by oxygen, S, NH, N—CH₃ or N-benzyl;

the remaining substituents R(2), R(3), R(4), R(5) or R(1), R(2), R(4), R(5) or R(1), R(2), R(3), R(5) in each case

independently of one another are hydrogen, F, Cl, Br, I, —Oₜₐ(C₁-C₈)-alkyl, —O_{tb}(C₃-C₈)-alkenyl,

—O_{tc}(CH₂)_{b}C_{d}F_{2d+1}, —O_{td}CₚH₂ₚR(18),

or up to 2 groups CN, NO₂, NR(16)R(17),

b is zero or 1;

d is 1, 2, 3, 4, 5, 6 or 7;

ta is zero or 1;

tb is zero or 1;

tc is zero or 1;

td is zero or 1;

p is zero, 1, 2, 3 or 4;

R(18) is (C₃-C₈)-cycloalkyl, phenyl, biphenylyl or naphthyl,

where the aromatics are not substituted or are substituted by 1-3 substituents selected from the group consisting of F, Cl, CF₃, methyl, methoxy and NR(19)R(20);

R(19) and R(20)

are hydrogen or (C₁-C₄)-alkyl or (C₁-C₄)-perfluoroalkyl;

R(16) is hydrogen, (C₁-C₈)-alkyl, (C₁-C₈)-perfluoroalkyl (C₃-C₈)-alkenyl, —C_{q}H_{2q}—R(21),

q is zero, 1, 2, 3 or 4;

R(21) is (C₃-C₈)-cycloalkyl, phenyl, biphenylyl or naphthyl,

where the aromatics are not substituted or are substituted by 1-3 substituents from the group F, Cl, CF₃, methyl, methoxy or NR(22)R(23), R(22) and R(23) are hydrogen, (C₁-C₄)-alkyl or (C₁-C₄)-perfluoroalkyl;

R(17) is hydrogen, (C₁-C₈)-alkyl, (C₁-C₈)-perfluoroalkyl, (C₃-C₈)-alkenyl, —CᵣH₂ᵣ—R(24);

r is zero, 1, 2, 3 or 4;

R(24) is (C₃-C₈)-cycloalkyl, phenyl, biphenylyl or naphthyl,

where the aromatics are not substituted or are substituted by 1-3 substituents selected from the group consisting of F, Cl, CF₃, methyl, methoxy and NR(25)R(26);

R(25) and R(26) are hydrogen, (C₁-C₄)-alkyl or (C₁-C₄)-perfluoroalkyl;

or

R(16) and R(17)

together are 4 or 5 methylene groups, of which one CH₂ group can be replaced by oxygen, S, NH, N—CH₃ or N-benzyl;

and their pharmaceutically tolerable salts;

(HOE 93/F 249-EP-Offenlegungsschrift 640 587, NZ 264 282)

t) diacyl-substituted guanidines of the formula I

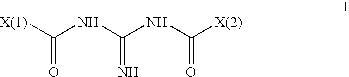

in which:

X(1) and X(2) are

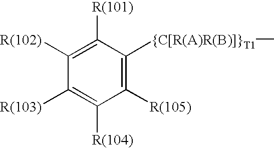

T1 is zero, 1, 2, 3 or 4;

R(A) and R(B)

independently of one another are hydrogen, F, Cl, Br, I, CN, OR(106), (C₁-C₈)-alkyl, (C₃-C₈)-cycloalkyl, O_{zk}(CH₂)_{zl}C_{zm}F_{2zm+1}, NR(107)R(108), phenyl or benzyl,

where the aromatics are not substituted or are substituted by 1-3 substituents selected from the group consisting of F, Cl, CF₃, methyl, methoxy and NR(109)R(110);

R(109) and R(110)

are hydrogen, (C₁-C₄)-alkyl or (C₁-C₄)-perfluoroalkyl;

zl is zero,1, 2, 3 or4;

zk is zero or 1;

zm is 1, 2, 3, 4, 5, 6, 7 or 8;

R(106)

is hydrogen, (C₁-C₈)-alkyl, (C₁-C₈)-perfluoroalkyl, (C₃-C₈)-alkenyl, (C₃-C₈)-cycloalkyl, phenyl or benzyl,

where the aromatics are not substituted or are substituted by 1-3 substituents selected from the group consisting of F, Cl, CF₃, methyl, methoxy and NR(111)R(112);

R(111) and R(112) are hydrogen, (C₁-C₄)-alkyl or (C₁-C₄)-perfluoroalkyl;

R(107) and R(108)

independently of one another are defined as R(106),

or

R(107) and R(108)

together are 4 or 5 methylene groups, of which one CH₂ group can be replaced by oxygen, S, NH, N—CH₃ or N-benzyl;

or

X(1) and X(2) are

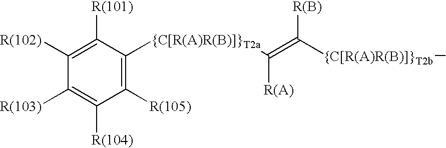

T2a and T2b

independently of one another are zero, 1 or 2; where the double bond can have the (E)- or (Z)-configuration;

or

X(1) and X(2) are

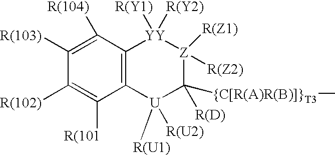

T3 is zero, 1 or 2;

U, YY and Z

independently of one another are C or N, where U, YY, Z can carry the following number of substituents:

| U, YY | Bonded in the ring to | Number of permitted |
|---|---|---|
| or Z | a double bond | substituents |
| C | yes | 1 |
| C | no | 2 |
| N | yes | 0 |
| N | no | 1 |

R(D) is hydrogen, (C₁-C₈)-alkyl or (C₁-C₈)-perfluoroalkyl,

R(U1), R(U2), R(Y1), R(Y2), R(Z1), R(Z2)

independently of one another are hydrogen, F, Cl, Br, I, CN,

OR(114), (C₁-C₈)-alkyl, (C₃-C₈)-cycloalkyl,

O_{zka}(CH₂)_{zla}C_{zma}F_{2zma+1}, NR(115)R(116), phenyl or benzyl,

where the aromatics are not substituted or are substituted by 1-3 substituents selected from the group consisting of F, Cl, CF₃, methyl, methoxy, NR(117)R(118),

R(117) and R(118)

are hydrogen, (C₁-C₄)-alkyl or (C₁-C₄)-perfluoroalkyl,

zka is zero or 1;

zla is zero, 1, 2, 3 or 4;

zma is 1, 2, 3, 4, 5, 6, 7 or 8;

R(114)

is hydrogen, (C₁-C₈)-alkyl, (C₁-C₈)-perfluoroalkyl, (C₃-C₈)-alkenyl, (C₃-C₈)-cycloalkyl, phenyl or benzyl,

where the aromatics are not substituted or are substituted by 1-3 substituents selected from the group consisting of F, Cl, CF₃, methyl, methoxy and NR(119)R(120);

R(119) and R(120) are hydrogen, (C₁-C₄)-alkyl or (C₁-C₄)-perfluoroalkyl;

R(115) and R(116) independently of one another are defined as R(114);

or

R(115) and R(116)

together are 4 or 5 methylene groups, of which one CH₂ group can be replaced by oxygen, S, NH, N—CH₃ or N-benzyl;

but where the constitution of U is nitrogen (N), YY is nitrogen (N) and Z is carbon (C) is excluded,

R(101), R(102), R(103), R(104) and R(105)

independently of one another are hydrogen, F, Cl, Br, I, —C≡N,

X_{zoa}—(CH₂)_{zpa}—(C_{zqa}F_{2zqa+1}), R(110a)—SO_{zbm}, R(110b)R(110c)N—CO,

R(111a)—CO— or R(112a)R(113a)N—SO₂—,

where the perfluoroalkyl group is straight-chain or branched,

X is oxygen, S or NR(114a);

R(114a)

is H or (C₁-C₃)-alkyl;

zoa is zero or 1;

zbm is zero, 1 or2;

zpa is zero, 1, 2, 3 or 4;

zqa is 1, 2, 3, 4, 5, 6, 7 or 8;

R(110a), R(110b), R(111a) and R(112a)

independently of one another are (C₁-C₈)-alkyl, (C₃-C₈)-alkenyl, —C_{zn}H_{2zn}—R(115a) or (C₁-C₈)-perfluoroalkyl;

zn is zero, 1, 2, 3 or 4;

R(115a)

is (C₃-C₈)-cycloalkyl, phenyl, biphenylyl or naphthyl, where the aromatics are not substituted or are substituted by 1-3 substituents selected from the group consisting of F, Cl, CF₃, methyl, methoxy and NR(116a)R(117a);

R(116a) and R(117a) are hydrogen, (C₁-C₄)-perfluoroalkyl or (C₁-C₄)-alkyl;

or

R(110b), R(111a) and R(112a)

are hydrogen;

R(110c) and R(113a)

independently are hydrogen, (C₁-C₄)-perfluoroalkyl or (C₁-C₄)-alkyl;

or

R(110b) and R(110c) and R(112a) and R(113a)

together are 4 or 5 methylene groups, of which one CH₂ group can be replaced by oxygen, sulfur, NH, N—CH₃ or N-benzyl;

or

R(101), R(102), R(103), R(104), R(105)

independently of one another are (C₁-C₈)-alkyl, —C_{zal}H_{2zal}R(118a) or (C₃-C₈)-alkenyl,

zal is zero, 1, 2, 3 or 4;

R(118a)

is (C₃-C₈)-cycloalkyl, phenyl, biphenylyl or naphthyl,

where the aromatics are not substituted or are substituted by 1-3 substituents from the group consisting of F, Cl, CF₃, methyl, methoxy or NR(119a)R(119b);

R(119a) and R(119b) are hydrogen, (C₁-C₄)-alkyl or (C₁-C₄)-perfluoroalkyl;

or

R(101), R(102), R(103), R(104), R(105)

independently of one another are (C₁-C₉)-heteroaryl,

which is linked via C or N and which is unsubstituted or substituted by 1-3 substituents from the group consisting of F, Cl, CF₃, CH₃, methoxy, hydroxyl, amino, methylamino and dimethylamino;

or

R(101), R(102), R(103), R(104), R(105)

independently of one another are —C≡C—R(193);

R(193)

is phenyl which is not substituted or is substituted by 1-3 substituents from the group consisting of F, Cl, CF₃, methyl, methoxy or NR(194)R(195);

R(194) and R(195)

are hydrogen or CH₃;

or

R(101), R(102), R(103), R(104), R(105)

independently of one another are

—Y-para-C₆H₄—(CO)_{zh}—(CHOH)_{zi}—(CH₂)_{zj}—(CHOH)_{zk}—R(123),

—Y-meta-C₆H₄—(CO)_{zad}—(CHOH)_{zae}—(CH₂)_{zaf}—(CHOH)_{zag}—R(124)

or

—Y-ortho-C₆H₄—(CO)_{zah}—(CHOH)_{zao}—(CH₂)_{zap}—(CHOH)_{zak}—R(125);

Y is oxygen, —S— or —NR(122d)-;

zh, zad, zah

independently are zero or 1;

zi, zj, zk, zae, zaf, zag, zao, zap and zak

independently are zero, 1, 2, 3 or 4;

but where in each case

zh, zi and zk are not simultaneously zero,

zad, zae and zag are not simultaneously zero, and

zah, zao and zak are not simultaneously zero,

R(123), R(124) R(125) and R(122d)

independently are hydrogen or (C₁-C₃)-alkyl;

or

R(101), R(102), R(103), R(104) and R(105)

independently of one another are SR(129), —OR(130), —NR(131)R(132) or —CR(133)R(134)R(135);

R(129), R(130), R(131) and R(133)

independently are —C_{zab}H_{2zab}—(C₁-C₉)-heteroaryl,

which is unsubstituted or substituted by 1-3 substituents from the group consisting of F, Cl, CF₃, CH₃, methoxy, hydroxyl, amino, methylamino and dimethylamino;

zab is zero, 1 or 2;

R(132), R(134) and R(135)

independently are defined as R(1 29) or are hydrogen, (C₁-C₄)-alkyl or (C₁-C₄)-perfluoroalkyl;

or

R(101), R(102), R(103), R(104) and R(105)

independently of one another are —W-para-(C₆H₄)—R(196), —W-meta-(C₆H₄)—R(197) or —W-ortho-(C₆H₄)—R(198);

R(196), R(197) and R(198)

independently of one another are (C₁-C₉)-heteroaryl,

which is linked via C or N and which is unsubstituted or substituted by 1 to 3 substituents from the group consisting of F, Cl, CF₃, CH₃, methoxy, hydroxyl, amino, methylamino, dimethylamino and benzyl;

W is oxygen, S or NR(136)-;

R(136)

is hydrogen or (C₁-C₄)-alkyl;

or

R(101), R(102), R(103), R(104) and R(105)

independently of one another are R(146)X(1a)-;

X(1a)

is oxygen, S, NR(147), (D=O)A-, NR(148)C=MN^{(*)}R(149)-;

M is oxygen or sulfur;

A is oxygen or NR(150),

D is C or SO;

R(146)

is (C₁-C₈)-alkyl, (C₃-C₈)-alkenyl, (CH₂)_{zbz}C_{zdz}F_{2zdz+1} or —C_{zxa}H_{2zxa}—R(151);

zbz is zero or 1;

zdz is 1, 2, 3, 4, 5, 6 or 7;

zxa is zero, 1, 2, 3 or 4;

R(151)

is (C₃-C₈)-cycloalkyl, phenyl, biphenylyl or naphthyl, where the aromatics are not substituted or are substituted by 1-3 substituents from the group consisting of F, Cl, CF₃, methyl, methoxy and NR(152)R(153);

R(152) and R(153) are hydrogen, (C₁-C₄)-alkyl or (C₁-C₄)-perfluoroalkyl;

R(147), R(148) and R(150)

independently are hydrogen, (C₁-C₄)-alkyl, (C₁-C₄)-perfluoroalkyl;

R(149) is defined as R(146),

or

R(146) and R(147), or R(146) and R(148)

together are 4 or 5 methylene groups, of which one CH₂ group can be replaced by oxygen, sulfur, NH, N—CH₃ or N-benzyl;

where A and N^{(*)} are bonded to the phenyl nucleus of the alkanoyl parent structure;

or

R(101), R(102), R(103), R(104) and R(105)

independently of one another are —SR(164), —OR(165), —NHR(166), —NR(167)R(168), —CHR(169)R(170), CR(154)R(155)OH, —C≡CR(156), —CR(158)═CR(157) or —[CR(159)R(160)]_{zu}—(C═O)—[CR(161)R(162)]_{zv}—R(163);

R(164), R(165), R(166), R(167), R(169)

identically or differently are —(CH₂)_{zy}—(CHOH)_{zz}—(CH₂)_{zaa}—(CHOH)_{zl}—R(171) or —(CH₂)_{zab}—O—(CH₂—CH₂O)_{zac}—R(172);

R(171) and R(172)

are hydrogen or methyl;

zu is 1, 2, 3 or 4;

zv is zero, 1, 2, 3 or 4;

zy, zz, zaa, zab, zac

identically or differently are zero, 1, 2, 3 or 4;

zt is 1, 2, 3 or 4;

R(168), R(170), R(154), R(155)

identically or differently are hydrogen or (C₁-C₆)-alkyl,

or

R(169) and R(170), or R(154) and R(155)

together with the carbon atom carrying them are a (C₃-C₈)-cycloalkyl;

R(163)

is hydrogen, (C₁-C₆)-alkyl, (C₃-C₈)-cycloalkyl or —C_{zeb}H_{2zeb}—R(173);

zeb is zero, 1, 2, 3 or 4;

R(156), R(157) and R(173)

independently are phenyl which is unsubstituted or is substituted by 1-3 substituents from the group consisting of F, Cl, CF₃, methyl, methoxy and NR(174)R(175);

R(174) and R(175) are hydrogen or (C₁-C₄)-alkyl;

or

R(166), R(157) and R(173)

independently are (C₁-C₉)-heteroaryl,

which is unsubstituted or substituted as phenyl;

R(158), R(159), R(160), R(161) and R(162)

are hydrogen or methyl,

or

R(101), R(102), R(103), R(104), R(105)

independently of one another are R(176)-NH—SO₂—;

R(176)

is R(177)R(178)N—(C═Y′)—;

Y′ is oxygen, S or N—R(179);

R(177) and R(178)

identically or differently are hydrogen, (C₁-C₈)-alkyl, (C₃-C₆)-alkenyl or —C_{zfa}H_{zfa}—R(180);

zfa is zero, 1, 2, 3 or 4;

R(180)

is (C₅-C₇)-cycloalkyl or phenyl,

which is unsubstituted or substituted by 1-3 substituents selected from the group consisting of F, Cl, CF₃, methoxy or (C₁-C₄)-alkyl;

or

R(177) and R(178)

together are 4 or 5 methylene groups, of which one CH₂ group can be replaced by oxygen, sulfur, NH, N—CH₃ or N-benzyl;

R(179)

is defined as R(177) or is amidine,

or

R(101), R(102), R(103), R(104), R(105)

independently of one another are NR(184a)R(185), OR(184b), SR(184c) or —C_{znx}H_{2znx}—R(184d);

znx is zero, 1, 2, 3 or 4;

R(184d)

is (C₃-C₇)-cycloalkyl or phenyl,

which is not substituted or substituted by 1-3 substituents from the group consisting of F, Cl, CF₃2 methyl, methoxy and NR(116k)R(117k);

R(116k) and R(117k) are hydrogen or C₁-C₄-alkyl;

R(184a), R(184b), R(184c), R(185)

independently of one another are hydrogen, (C₁-C₈)-alkyl, (C₁-C₈)-perfluoroalkyl or (CH₂)_{zao}—R(184g);

zao is zero, 1, 2, 3 or 4;

R(184g)

is (C₃-C₇)-cycloalkyl or phenyl,

which is not substituted or substituted by 1-3 substituents from the group consisting of F, Cl, CF₃, methyl, methoxy and NR(184u)R(184v);

R(184u) and R(184v) are hydrogen or C₁-C₄-alkyl;

or

R(184a) and R(185)

together are 4 or 5 methylene groups, of which one CH₂ group can be replaced by oxygen, sulfur, NH, N—CH₃ or N-benzyl;

and their pharmaceutically tolerable salts;

(HOE 93/F 254-EP-Offenlegungsschrift 640 588, NZ 264 307)

u) benzoylguanidines of the formula I

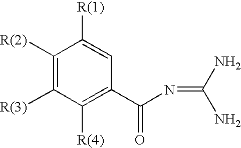

in which:

R(1) is H, F, Cl, Br, I, CN, NO₂, (C₁-C₈)-alkyl, (C₃-C₈)-cycloalkyl or

Xₐ—(CH₂)_{b}—(CF₂)_{c}—CF₃;

X is oxygen, S or NR(5);

a is zero or 1;

b is zero, 1 or 2;

c is zero, 1, 2 or 3;

R(5) is H, (C₁-C₄)-alkyl or —C_{d}H_{2d}R(⁶);

d is zero, 1, 2, 3 or 4;

R(6) is (C₃-C₈)-cycloalkyl, phenyl, biphenylyl or naphthyl,

where the aromatics are not substituted or are substituted by 1 to 3 substituents selected from the group consisting of F, Cl, CF₃, methyl, methoxy and NR(7)R(8);

R(7) and R(8) independently are H or (C₁-C₄)-alkyl;

or

R(1) is —SR(10), —OR(10) or —CR(10)R(11)R(12);

R(10) is —C_{f}H_{2f}—(C₃-C₈)-cycloalkyl, —(C₁-C₉)-heteroaryl or phenyl,

where the aromatic systems are unsubstituted or substituted by one to 3 substituents selected from the group consisting of F, Cl, CF₃, CH₃, methoxy, hydroxyl, amino, methylamino and dimethylamino;

f is zero, 1 or 2;

R(11) and R(12)

independently of one another are defined as R(10) or are hydrogen or (C₁-C₄)-alkyl;

or

R(1) is phenyl, naphthyl, biphenylyl or (C₁-C₉)-heteroaryl,

the latter linked via C or N,

and which are unsubstituted or substituted by 1 to 3 substituents selected from the group consisting of F, Cl, CF₃, CH₃, methoxy, hydroxyl, amino, methylamino and dimethylamino;

or

R(1) is —SR(13), —OR(13), —NHR(13), —NR(13)R(14), —CHR(13)R(15), —C[R(15)R(16)]OH, —C≡CR(18), —C[R(19)]═CR(18), —[CR(20)R(21)]ₖ—(CO)—[CR(22)R(23)R(24)];

R(13) and R(14)

identically or differently are —(CH₂)_{g}—(CHOH)ₕ—(CH₂)ᵢ—(CHOH)ⱼ—R(17),

R(17) is hydrogen or methyl;

—(CH₂)_{g}—O—(CH₂—CH₂O)ₕ—R(24),

g, h, i

identically or differently are zero, 1, 2, 3 or 4;

j is 1, 2, 3 or 4;

R(15) and R(16)

identically or differently are hydrogen, (C₁-C₆)-alkyl or together with the carbon atom carrying them are a (C₃-C₈)-cycloalkyl;

R(18) is phenyl,

which is unsubstituted or substituted by 1 to 3 substituents selected from the group consisting of F, Cl, CF₃, methyl, methoxy and NR(25)R(26);

R(25) and R(26)

are H or (C₁-C₄)-alkyl;

or

R(18) is (C₁-C₉)-heteroaryl,

which is unsubstituted or substituted as phenyl;

or

R(18) is (C₁-C₆)-alkyl,

which is unsubstituted or substituted by 1 to 3 OH;

or

R(18) is (C₃-C₈)-cycloalkyl;

R(19), R(20), R(21), R(22) and R(23)

are hydrogen or methyl;

k is zero, 1, 2, 3 or 4;

l is zero, 1, 2, 3 or 4;

R(24) is H, (C₁-C₆)-alkyl, (C₃-C₈)-cycloalkyl or —CₘH₂ₘ—R(18);

m is 1, 2, 3 or 4;

R(2) and R(3)

independently of one another are defined as R(1);

R(4) is (C₁-C₃)-alkyl, F, Cl, Br, 1, CN or —(CH₂)ₙ—(CF₂)ₒ—CF₃;

n is zero or 1;

o is zero, 1 or 2;

and their pharmaceutically tolerable salts;

(HOE 93/F 436-EP-Offenlegungsschrift 659 748), NZ 270 264)

v) acylguanidines of the formula I

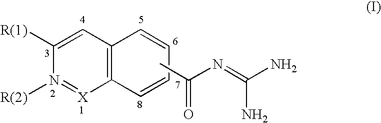

in which:

X is carbonyl, sulfonyl,

R(1) is H, (C₁-C₈)-alkyl,

unsubstituted or substituted by hydroxyl,

(C₃-C₈)-cycloalkyl, phenyl,

which is unsubstituted or substituted by 1-3 substituents from the group F, Cl, CF₃, CH₃, methoxy, hydroxyl, amino, methylamino or dimethylamino,

R(2) is H. (C₁-C₄)-alkyl,

or a pharmaceutically tolerable salt thereof;

(HOE 94/F 014 K-EP-OS 666 252, NZ 270 370)

w) a phenyl-substituted alkycarboxylic acid guanidide, carrying perfluoroalkyl groups, of the formula

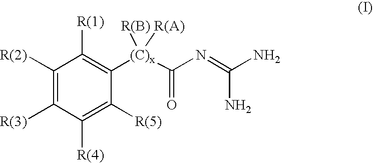

in which:

R(A) is hydrogen, F, Cl, Br, I, CN, OR(6), (C₁-C₈)-alkyl, (C₃-C₈)-cycloalkyl, Oᵣ(CH₂)ₐC_{b}F_{2b+1} or NR(7)R(8);

r is zero or 1;

a is zero, 1, 2, 3 or 4;

b is 1, 2, 3, 4, 5, 6, 7 or 8;

R(6) is hydrogen, (C₁-C₈)-alkyl, (C₃-C₈)-alkenyl, (C₃-C₈)-cycloalkyl, phenyl or benzyl,

where the aromatics are not substituted or are substituted by 1-3 substituents selected from the group consisting of F, Cl, CF₃, methyl, methoxy and NR(9)R(10);

R(9) and R(10)

are H, (C₁-C₄)-alkyl or (C₁-C₄)-perfluoroalkyl;

R(7) and R(8)

independently of one another are defined as R(6);

R(B) independently is defined as R(A);

X is 1, 2 or 3;

R(1) is hydrogen, (C₁-C₈)-alkyl, (C₃-C₈)-cycloalkyl, —Oₜ(CH₂)_{d}CₑF₂ₑ₊₁, F, Cl, Br, I or CN;

t is zero or 1;

d is zero, 1, 2, 3 or 4;

e is 1, 2, 3, 4, 5, 6, 7 or 8;

R(2), R(3), R(4) and R(5)

independently of one another are defined as R(1);

but with the condition

that at least one of the substituents R(1), R(2), R(3), R(4), R(5), R(A) and R(B) is an —Oₜ(CH₂)_{d}CₑF₂ₑ₊₁ or an Oᵣ(CH₂)ₐC_{b}F_{2b+1} group,

and their pharmaceutically tolerable salts;

(HOE 94/F 094-EP-Offenlegungsschrift 676 395, NZ 270 894)

x) heteroaroylguanidines of the formua I

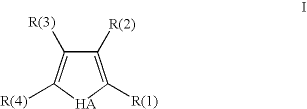

in which:

HA is SOₘ, O or NR(5);

m is zero, 1 or 2;

R(5) is hydrogen, (C₁-C₈)-alkyl or —CₐₘH₂ₐₘR(⁸¹);

am is zero, 1 or 2;

R(81) is (C₃-C₈)-cycloalkyl or phenyl,

which is not substituted or is substituted by 1-3 substituents selected from the group consisting of F, Cl, CF₃, methyl, methoxy and NR(82)R(83);

R(82) and R(83) is H or CH₃;

or

R(81) is (C₁-C₉)-heteroaryl,

which is linked via C or N and which is unsubstituted or substituted by 1-3 substituents selected from the group consisting of F, Cl, CF₃, CH₃, methoxy, hydroxyl, amino, methylamino and dimethylamino;

one of the two substituents R(1) and R(2)

is —CO—N═C(NH₂)₂;

and the other in each case

is hydrogen, F, Cl, Br, I, (C₁-C₃)-alkyl, —OR(6), CᵣF₂ᵣ₊₁, —CO—N═C(NH₂)₂ or —NR(6)R(7);

R(6) and R(7)

independently are hydrogen or (C₁-C₃)-alkyl;

r is 1, 2, 3 or 4;

R(3) and R(4)

independently of one another are hydrogen, F, Cl, Br, I, —C≡N, X—(CH₂)ₚ—(C_{q}—F_{2q+1}), R(⁸)—SO_{bm}, R(9)R(10)N—CO, R(11)-CO— or R(12)R(13)N—SO₂—,

where the perfluoroalkyl group is straight-chain or branched,

X is oxygen, S or NR(14);

R(14) is H or (C₁-C₃)-alkyl;

bm is zero, 1 or 2;

p is zero, 1 or 2;

q is zero, 1, 2, 3, 4, 5 or 6;

R(8), R(9), R(11) and R(12)

independently are (C₁-C₈)-alkyl, (C₃-C₆)-alkenyl, —CₙH₂ₙ—R(15), CF₃;

n is zero, 1, 2, 3 or 4;

R(15) is (C₃-C₇)-cycloalkyl or phenyl;

which is not substituted or is substituted by 1-3 substituents selected from the group consisting of F, Cl, CF₃, methyl, methoxy or NR(16)R(17);

R(16) and R(17) are H or C₁-C₄-alkyl;

or

R(9), R(11) and R(12)

are H;

R(10) and R(13)

independently are H or (C₁-C₄)-alkyl;

or

R(9) and R(10), and R(12) and R(13)

together are 4 or 5 methylene groups, of which one CH₂ group can be replaced by oxygen, S, NH, N—CH₃ or N-benzyl,

or

R(3) and R(4)

independently of one another are (C₁-C₈)-alkyl or —CₐₗH₂ₐₗR(¹⁸);

al is zero, 1 or 2;

R(18) is (C₃-C₈)-cycloalkyl or phenyl;

which is not substituted or is substituted by 1-3 substituents selected from the group consisting of F, Cl, CF₃, methyl, methoxy and NR(19)R(20);

R(19) and R(20) are H or CH₃;

or

R(3) and R(4)

independently of one another are (C₁-C₉)-heteroaryl,

which is linked via C or N and which is unsubstituted or substituted by 1-3 substituents selected from the group consisting of F, Cl, CF₃, CH₃, methoxy, hydroxyl, amino, methylamino and dimethylamino;

or

R(3) and R(4)

independently of one another are

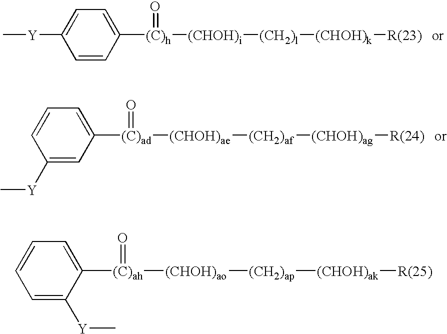

Y is oxygen, —S— or —NR(22)-;

h, ad, ah independently are zero or 1;

i, j, k, ae, af, ag, ao, ap and ak independently are zero, 1, 2, 3, 4, but where in each case

h, i and k are not simultaneously zero,

ad, ae and ag are not simultaneously zero,

ah, ao and ak are not simultaneously zero,

R(23), R(24) R(25) and R(22)

independently are hydrogen or (C₁-C₃)-alkyl;

or

R(3) and R(4)

independently are hydrogen, F, Cl, Br, I, CN, (C₁-C₈)-alkyl, (C₁-C₈)-perfluoroalkyl, (C₃-C₈)-alkenyl or —C_{g}H_{2g}R(26);

g is zero, 1, 2, 3 or 4;

R(26) is (C₃-C₈)-cycloalkyl, phenyl, biphenylyl or naphthyl,

where the aromatics are not substituted or are substituted by 1-3 substituents selected from the group consisting of F, Cl, CF₃, methyl, methoxy and NR(27)R(28);

R(27) and R(28)

are H, (C₁-C₄)-alkyl or (C₁-C₄)-perfluoroalkyl;

or

R(3) and R(4)

independently of one another are SR(29), —OR(30), —NR(31)R(32) or —CR(33)R(34)R(35);

R(29), R(30), R(31) and R(33)

independently of one another are —CₐH₂ₐ—(C₁-C₉)-heteroaryl,

which is unsubstituted or substituted by 1-3 substituents selected from the group consisting of F, Cl, CF₃, CH₃, methoxy, hydroxyl, amino, methylamino and dimethylamino;

a is zero, 1 or 2;

R(32), R(34) and R(35)

independently of one another are defined as R(29) or are hydrogen, (C₁-C₄)-alkyl or (C₁-C₄)-perfluoroalkyl;

or

R(3) and R(4)

independently of one another are

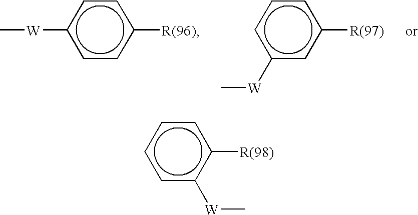

R(96), R(97) and R(98)

independently are (C₁-C₉)-heteroaryl,

which is linked via C or N and which is unsubstituted or substituted by 1 to 3 substituents selected from the group consisting of F, Cl, CF₃, CH₃, methoxy, hydroxyl, amino, methylamino, dimethylamino or benzyl;

W is oxygen, S or NR(36)-;

R(36) is H or (C₁-C₄)-alkyl;

or

R(3) and R(4)

independently of one another are R(³⁷)-SO_{cm} or R(38)R(39)N—SO₂—;

cm is 1 or 2;

R(37) is (C₁-C₈)-alkyl, (C₁-C₈)-perfluoroalkyl, (C₃-C₈)-alkenyl or —CₛH₂ₛR(40);

s is zero, 1, 2, 3 or 4;

R(40) is (C₃-C₈)-cycloalkyl, phenyl, biphenylyl or naphthyl,

where the aromatics are not substituted or are substituted by 1-3 substituents selected from the group consisting of F, Cl, CF₃, methyl, methoxy and NR(41)R(42);

R(41) and R(42) are H, (C₁-C₄)-alkyl or (C₁-C₄)-perfluoroalkyl;

R(38) is H, (C₁-C₈)-alkyl, (C₁-C₈)-perfluoroalkyl, (C₃-C₈)-alkenyl or —C_{w}H_{2w}—R(43);

w is zero, 1, 2, 3 or 4;

R(43) is (C₃-C₈)-cycloalkyl, phenyl, biphenylyl or naphthyl,

where the aromatics are not substituted or are substituted by 1-3 substituents selected from the group consisting of F, Cl, CF₃, methyl, methoxy and NR(44)R(45);

R(44) and R(45) are H, (C₁-C₄)-alkyl or (C₁-C₄)-perfluoroalkyl;

R(39) is H, (C₁-C₄)-alkyl or (C₁-C₄)-perfluoroalkyl;

or

R(38) and R(39)

together are 4 or 5 methylene groups, of which one CH₂ group can be replaced by oxygen, S, NH, N—CH₃ or N-benzyl;

or

R(3) and R(4)

independently of one another are R(46)X(1)-;

X(1) is oxygen, S, NR(47), (D=O)A-, NR(48)C=MN^{(*)}R(49)-,

M is oxygen or S;

A is oxygen or NR(50);

D is C or SO;

R(46) is (C₁-C₈)-alkyl, (C₃-C₈)-alkenyl, (CH₂)_{b}C_{d}F_{2d+1} or —CₓH₂ₓ—R(51);

b is zero or 1;

d is 1, 2, 3, 4, 5, 6 or 7;

x is zero, 1, 2, 3 or 4;

R(51) is (C₃-C₈)-cycloalkyl, phenyl, biphenylyl or naphthyl,

where the aromatics are not substituted or are substituted by 1-3 substituents selected from the group consisting of F, Cl, CF₃, methyl, methoxy and NR(52)R(53);

R(52) and R(53) are H, (C₁-C₄)-alkyl or (C₁-C₄)-perfluoroalkyl;

R(47), R(48) and R(50)

independently are hydrogen, (C₁-C₄)-alkyl or (C₁-C₄)-perfluoroalkyl;

R(49) is defined as R(46);

or

R(46) and R(47), or R(46) and R(48)

together are 4 or 5 methylene groups, of which one CH₂ group can be replaced by oxygen, S, NH, N—CH₃ or N-benzyl,

where A and N^{(*)} are bonded to the phenyl nucleus of the benzoylguanidine parent structure;

or

R(3) and R(4)

independently of one another are —SR(64), —OR(65), —NHR(66), —NR(67)R(68), —CHR(69)R(70), —C(OH)R(54)R(55), —C≡CR(56), —CR(58)=CHR(57), —[CR(59)R(60)]ᵤ—(CO)—[CR(61)R(62)]ᵥ—R(C₆₃);

R(64), R(65), R(66), R(67) and R(69)

identically or differently are —(CH₂)_{y}—(CHOH)_{z}—(CH₂)ₐₐ—(CH₂OH)ₜ—R(71) or —(CH₂)_{ab}—O—(CH₂—CH₂O)_{ac}—R(72),

R(71) and R(72)

are hydrogen or methyl;

u is 1, 2, 3 or 4;

v is zero, 1, 2, 3 or 4;

y, z, aa

identically or differently are zero, 1, 2, 3 or 4;

t is 1, 2, 3 or 4;

R(68), R(70), R(54) and R(55)

identically or differently are hydrogen, (C₁-C₆)-alkyl;

or

R(69) and R(70), or R(54) and R(55)

together with the carbon atom carrying them are a (C₃-C₈)-cycloalkyl;

R(63)

is H, (C₁-C₆)-alkyl, (C₃-C₈)-cycloalkyl or —CₑH₂ₑ—R(73);

e is zero, 1, 2, 3 or 4;

R(56), R(57) and R(73)

independently are phenyl,

which are unsubstituted or substituted by 1-3 substituents selected from the group consisting of F. Cl, CF₃, methyl, methoxy and NR(74)R(75);

R(74) and R(75) are H or (C₁-C₄)-alkyl;

or

R(56), R(57) and R(73)

independently are (C₁-C₉)-heteroaryl,

which is unsubstituted or substituted as phenyl;

R(58), R(59), R(60), R(61) and R(62)

are hydrogen or methyl,

or

R(3) and R(4)

independently of one another are R(76)-NH—SO₂—;

R(76) is R(77)R(78)N—(C═Y′)—;

Y′ is oxygen, S or N—R(79);

R(77) and R(78)

identically or differently are H, (C₁-C₈)-alkyl, (C₃-C₆)-alkenyl, —C_{f}H_{2f}—R(80);

f is zero, 1, 2, 3 or 4;

R(80) is (C₅-C₇)-cycloalkyl or phenyl,

which is unsubstituted or substituted by 1-3 substituents selected from the group consisting of F, Cl, CF₃, methoxy and (C₁-C₄)-alkyl;

or

R(77) and R(78)

together are 4 or 5 methylene groups, of which one CH₂ group can be replaced by oxygen, S, NH, N—CH₃ or N-benzyl, R(79)

is defined as R(77) or is amidine;

or

R(3) and R(4)

independently of one another are NR(84)R(85);

R(84) and R(85)

independently of one another are H, (C₁-C₄)-alkyl, or together are 4 or 5 methylene groups,

of which one CH₂ group can be replaced by oxygen, S, NH, N—CH₃ or N-benzyl; or of which one or two CH₂ groups can be replaced by CH—C_{dm}H_{2dm+1},

and their pharmaceutically tolerable salts;

(HOE 94/F 123-EP-Offenlegungsschrift 682 017, NZ 272 058)

y) bicyclic heteroaroylguanidines of the formula I

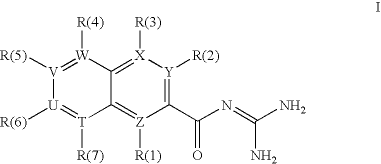

in which:

T, U, V, W, X, Y and Z

independently of one another are nitrogen or carbon;

but with the restriction

that X and Z are not simultaneously nitrogen,

and that T, U, V, W, X, Y and Z carry no substituents if they are nitrogen,

and that no more than four of them are simultaneously nitrogen,

R(1) and R(2)

independently of one another are hydrogen, F, Cl, Br, I, (C₁-C₃)-alkyl, (C₁-C₃)-perfluoroalkyl, OR(8), NR(8)R(9) or C(═O)N═C(NH₂)₂;

R(8) and R(9)

independently of one another are hydrogen or (C₁-C₃)-alkyl,

or

R(8) and R(9)

together are 4 or 5 methylene groups, of which one CH₂ group can be replaced by oxygen, S, NH, N—CH₃ or N-benzyl;

R(3), R(4), R(5), R(6) and R(7)

independently of one another are hydrogen, F, Cl, Br, I, —C≡N, Xₖ—(CH₂)ₚ—(C_{q}F_{2q+1}), R(10a)—SO_{bm}, R(10b)R(10c)N—CO, R(11)-CO— or

R(12)R(13)N—SO₂—,

where the perfluoroalkyl group is straight-chain or branched;

X is oxygen, S or NR(14);

R(14) is H or (C₁-C₃)-alkyl;

bm is zero, 1 or 2;

p is zero, 1 or 2;

k is zero or 1;

q 1, 2, 3, 4, 5 or 6;

R(10a), R(10b), R(11) and R(12)

independently of one another are (C₁-C₈)-alkyl, (C₃-C₆)-alkenyl, —CₙH₂ₙ—R(15) or (C₁-C₈)-perfluoroalkyl;

n is zero, 1, 2, 3 or 4;

R(15) is (C₃-C₇)-cycloalkyl or phenyl, which is not substituted or is substituted by 1-3 substituents selected from the group consisting of F, Cl, CF₃, methyl, methoxy and NR(16)R(17);

R(16) and R(17) are H or C₁-C₄-alkyl;

or

R(10b), R(11) and R(12)

are hydrogen;

R(10c) and R(13)

independently are hydrogen or (C₁-C₄)-alkyl;

or

R(10b) and R(10c) and R(12) and R(13)

together are 4 or 5 methylene groups, of which one CH₂ group can be replaced by oxygen, sulfur, NH, N—CH₃ or N-benzyl;

or

R(3), R(4), R(5), R(6) and R(7)

independently of one another are (C₁-C₈)-alkyl, —CₐₗH₂ₐₗR(18) or (C₃-C₈)-alkenyl;

al is zero, 1 or 2;

R(18) is (C₃-C₈)-cycloalkyl, phenyl, biphenylyl or naphthyl, where the aromatics are not substituted or are substituted by 1-3 substituents selected from the group consisting of F, Cl, CF₃, methyl, methoxy and NR(19a)R(19b);

R(19a) and R(19b)

are hydrogen, (C₁-C₄)-alkyl or (C₁-C₄)-perfluoroalkyl;

or

R(3), R(4), R(5), R(6) and R(7)

independently of one another are (C₁-C₉)-heteroaryl, which is linked via C or N and which is unsubstituted or substituted by 1-3 substituents from the group consisting of F, Cl, CF₃, CH₃, methoxy, hydroxyl, amino, methylamino or dimethylamino;

or

R(3), R(4), R(5), R(6) and R(7)

independently of one another are

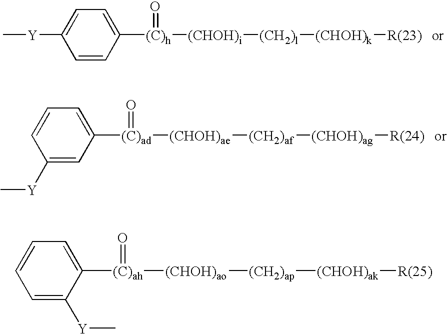

Y is oxygen, —S— or —NR(22)-;

h, ad, ah

independently of one another are zero or 1;

i,j, k, ae, af, ag, ao, ap and ak

independently of one another are zero, 1, 2, 3 or 4;

but where in each case

h, i and k are not simultaneously zero,

ad, ae and ag are not simultaneously zero, and

ah, ao and ak are not simultaneously zero,

R(23), R(24) R(25) and R(22)

independently of one another are hydrogen or (C₁-C₃)-alkyl;

or

R(3), R(4), R(5), R(6) and R(7)

independently of one another are SR(29), —OR(30), —NR(31)R(32) or —CR(33)R(34)R(35);

R(29), R(30), R(31) and R(33)

independently of one another are —CₐH₂ₐ—(C₁-C₉)-heteroaryl, which is unsubstituted or substituted by 1-3 substituents selected from the group consisting of F, Cl, CF₃, CH₃, methoxy, hydroxyl, amino, methylamino and dimethylamino;

a is zero, 1 or 2;

R(32), R(34) and R(35)

independently of one another are defined as R(29) or are hydrogen, (C₁-C₄)-alkyl or (C₁-C₄)-perfluoroalkyl;

or

R(3), R(4), R(5), R(6) and R(7)

independently of one another are

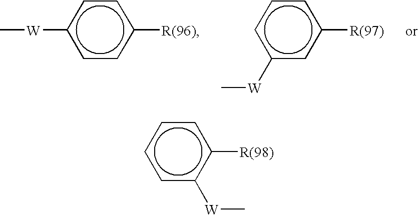

R(96), R(97) and R(98)

independently of one another are (C₁-C₉)-heteroaryl, which is linked via C or N and which is unsubstitued or substituted by 1 to 3 substituents from the group consisting of F, Cl, CF₃, CH₃, methoxy, hydroxyl, amino, methylamino, dimethylamino or benzyl;

W is oxygen, S or NR(36)-;

R(36) is H or (C₁-C₄)-alkyl;

or

R(3), R(4), R(5), R(6) and R(7)

independently of one another are R(46)X(1)-;

X(1) is oxygen, S, NR(47), (D=O)A- or NR(48)C=MN^{(*)}R(49)-;

M is oxygen or sulfur;

A is oxygen or NR(50);

D is C or SO;

R(46) is (C₁-C₈)-alkyl, (C₃-C₈)-alkenyl, (CH₂)_{b}C_{d}F_{2d+1} or —CₓH₂ₓ—R(51);

b is zero or 1;

d is 1, 2, 3, 4, 5, 6 or 7;

x is zero, 1, 2, 3 or 4;

R(51) is (C₃-C₈)-cycloalkyl, phenyl, biphenylyl or naphthyl,

where the aromatics are not substituted or are substituted by 1-3 substituents selected from the group consisting of F, Cl, CF₃, methyl, methoxy and NR(52)R(53);

R(52) and R(53) are hydrogen, (C₁-C₄)-alkyl or (C₁-C₄)-perfluoroalkyl;

R(47), R(48) and R(50) independently

are hydrogen, (C₁-C₄)-alkyl or (C₁-C₄)-perfluoroalkyl;

R(49) is defined as R(46);

or

R(46) and R(47), or R(46) and R(48)

together are 4 or 5 methylene groups, of which one CH₂ groups can be replaced by oxygen, sulfur, NH, N—CH₃ or N-benzyl;

where A and N^{(*)} are bonded to the phenyl nucleus of the heteroaroylguanidine parent structure;

or

R(3), R(4), R(5), R(6) and R(7)

independently of one another are —SR(64), —OR(65), —NHR(66), —NR(67)R(68), —CHR(69)R(70) or —CR(54)R(55)OH, —C≡CR(56), —CR(58)=CR(57) or —[CR(59)R(60)]ᵤ—CO—[CR(61)R(62)]ᵥ—R(63);

R(64), R(65), R(66), R(67) and R(69)

identically or differently are

—(CH₂)_{y}—(CHOH)_{z}—(CH₂)ₐₐ—(CHOH)ₜ—R(71) or —(CH₂)_{ab}—O—(CH₂—CH₂O)_{ac}—R(⁷²);

R(71) and R(72)

independently of one another are hydrogen or methyl;

u is 1, 2, 3 or 4;

v is zero, 1, 2, 3 or 4;

y, z, aa identically or differently

are zero, 1, 2, 3 or 4;

t is 1, 2, 3 or 4;

R(68), R(70), R(54) and R(55)

identically or differently are hydrogen or (C₁-C₆)-alkyl;

or

R(69) and R(70), or R(54) and R(55)

together with the carbon atom carrying them are (C₃-C₈)-cycloalkyl;

R(63)

is hydrogen, (C₁-C₆)-alkyl, (C₃-C₈)-cycloalkyl or —CₑH₂ₑ—R(73);

e is zero, 1, 2, 3 or 4;

R(56), R(57) and R(73) independently

are phenyl, which is unsubstituted or substituted by 1-3 substituents selected from the group consisting of F, Cl, CF₃, methyl, methoxy and NR(74)R(75);

R(74) and R(75)

are hydrogen or (C₁-C₄)-alkyl;

or

R(56), R(57) and R(73) independently

are (C₁-C₉)-heteroaryl, which is unsubstituted or substituted as phenyl;

R(58), R(59), R(60), R(61) and R(62)

are hydrogen or methyl;

or

R(3), R(4), R(5), R(6) and R(7)

independently of one another are R(76)-NH—SO₂—;

R(76) is R(77)R(78)N—(C═Y′)—;

Y′ is oxygen, S or N—R(79);

R(77) and R(78)

identically or differently are hydrogen, (C₁-C₈)-alkyl, (C₃-C₆)-alkenyl or —C_{f}H_{2f}—R(80);

f is zero, 1, 2, 3 or 4;

R(80) is (C₅-C₇)-cycloalkyl or phenyl, which is unsubstituted or substituted by 1-3 substituents selected from the group consisting of F, Cl, CF₃, methoxy and (C₁-C₄)-alkyl;

or

R(77) and R(78)

together are 4 or 5 methylene groups, of which one CH₂ group can be replaced by oxygen, sulfur, NH, N—CH₃ or N-benzyl;

R(79) is defined as R(77) or is amidine;

or

R(3), R(4), R(5), R(6) and R(7)

independently of one another are NR(84a)R(85), OR(84b), SR(84c) or —CₙH₂ₙ—R(84d);

n is zero, 1, 2, 3 or 4;

R(84d) is (C₃-C₇)-cycloalkyl or phenyl, which is not substituted or is substituted by 1-3 substituents from the group consisting of F, Cl, CF₃, methyl, methoxy and NR(16)R(17);

R(16) and R(17)

are hydrogen or C₁-C₄-alkyl;

R(84a), R(84b), R(84c) and R(85)

independently of one another are hydrogen, (C₁-C₈)-alkyl, (C₁-C₈)-perfluoroalkyl or (CH₂)ₐₓ—R(84g);

ax is zero, 1, 2, 3 or 4;

R(84g) is (C₃-C₇)-cycloalkyl or phenyl, which is not substituted or is substituted by 1-3 substituents selected from the group consisting of F, Cl, CF₃, methyl, methoxy and NR(84u)R(84v);

R(84u) and R(84v)

are hydrogen or C₁-C₄-alkyl;

or

R(84a) and R(85)

together are 4 or 5 methylene groups, of which one CH₂ group can be replaced by oxygen, sulfur, NH, N—CH₃ or N-benzyl,

and their pharmaceutically tolerable salts;

(HOE 94/F 134-EP-Offenlegungsschrift 686 627, NZ 272 103)

z) benzoylguanidines of the formula I

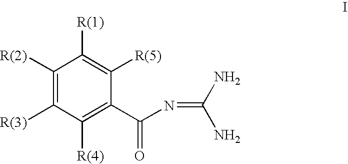

in which:

R(1) is R(⁶)-SOₘ;

m is zero, 1 or 2;

R(6) is perfluoroalkyl having 1, 2, 3, 4, 5 or 6 carbon atoms, which is straight-chain or branched;

R(2) and R(3)

independently of one another are hydrogen, F, Cl, Br, I, alkyl having 1, 2, 3 or 4 carbon atoms, alkoxy having 1, 2, 3 or 4 carbon atoms or phenoxy,

which is unsubstituted or substituted by 1-3 substituents selected from the group consisting of F, Cl, methyl and methoxy;

or

R(2) and R(3)

independently of one another are pyrrol-1-yl, pyrrol-2-yl or pyrrol-3-yl,

which is not substituted or is substituted by 1 to 4 substituents selected from the group consisting of F, Cl, Br, I, CN, alkanoyl having 2, 3, 4, 5, 6, 7 or 8 carbon atoms, alkoxycarbonyl having 2, 3, 4, 5, 6, 7 or 8 carbon atoms, formyl, carboxyl, CF₃, methyl and methoxy;

R(4) and R(5)

independently of one another are hydrogen, alkyl having 1, 2 or 3 carbon atoms, F, Cl, Br, I, CN, OR(7), NR(8)R(9) or —(CH₂)ₙ—(CF₂)ₒ—CF₃;

R(7), R(8) and R(9)

independently of one another are hydrogen or alkyl having 1, 2, 3 or 4 carbon atoms;

n is zero or 1;

o is zero, 1 or 2;

and their pharmacologically acceptable salts;

(HOE 94/F 168-EP-Offenlegungsschrift 690 048, NZ 272 373)

ab) Phenyl-substituted alkenylcarboxylic acid guanidides, carrying perfluoroalkyl groups, of the formula I

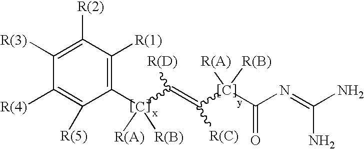

in which:

R(A) is hydrogen, F, Cl, Br, I, CN, OH, OR(6), (C₁-C₈)-alkyl, Oᵣ(CH₂)ₐC_{b}F_{2b+1}, (C₃-C₈)-cycloalkyl or NR(7)R(8);

r is zero or 1;

a is zero, 1, 2, 3 or 4;

b is 1, 2, 3, 4, 5, 6, 7 or 8;

R(6) is (C₁-C₈)-alkyl, (C₁-C₄)-perfluoroalkyl, (C₃-C₈)-alkenyl, (C₃-C₈)-cycloalkyl, phenyl or benzyl;

where the aromatics are not substituted or are substituted by 1-3 substituents from the group consisting of F, Cl, CF₃, methyl, methoxy and NR(9)R(10);

R(9) and R(10)

are H, (C₁-C₄)-alkyl or (C₁-C₄)-perfluoroalkyl;

R(7) and R(8)

independently of one another are defined as R(6);

or

R(7) and R(8)

together are 4 or 5 methylene groups, of which one CH₂ group can be replaced by oxygen, sulfur, NH, N—CH₃ or N-benzyl;

R(B) independently is defined as R(A);

x is zero, 1 or 2;

y is zero, 1 or 2;

R(C) is hydrogen, F, Cl, Br, I, CN, OR(12), (C₁-C₈)-alkyl, Oₚ(CH₂)_{f}C_{g}F_{2g+1} or (C₃-C₈)-cycloalkyl;

p is zero or 1;

f is zero, 1, 2, 3 or 4;

g is 1, 2, 3, 4, 5, 6, 7 or 8;

R(12)

is (C₁-C₈)-alkyl, (C₁-C₄)-perfluoroalkyl, (C₃-C₈)-alkenyl, (C₃-C₈)-cycloalkyl, phenyl or benzyl;

where the aromatics phenyl or benzyl are not substituted or are substituted by 1-3 substituents from the group consisting of F, Cl, CF₃, methyl, methoxy and NR(13)R(14);

R(13) and R(14) independently of one another are H, (C₁-C₄)-alkyl or (C₁-C₄)-perfluoroalkyl;

R(D) independently is defined as R(C),

R(1) is hydrogen, (C₁-C₈)-alkyl, —Oₜ(CH₂)_{d}CₑF₂ₑ₊₁, (C₃-C₈)-cycloalkyl, F, Cl, Br, I or CN;

t is zero or 1;

d is zero, 1, 2, 3 or 4;

e is 1, 2, 3, 4, 5, 6, 7 or 8;

R(2), R(3), R(4) and R(5)

independently of one another are defined as R(1);

but with the condition

that at least one of the substituents R(A), R(B), R(C), R(D), R(1), R(2), R(4) or R(5) is a Oᵣ(CH₂)ₐC_{b}F_{2b+1}, Oₚ(CH₂)_{f}C_{g}F_{2g+1} or Oₜ(CH₂)_{d}CₑF₂ₑ₊₁ group and R(3) is not a Oₜ(CH₂)_{d}CₑF₂ₑ₊₁ group;

and their pharmaceutically tolerable salts;

(HOE 94/F 182-EP-Offenlegungsschrift 690 048, NZ 272 449)

ac) ortho-amino-substituted benzoylguanidines of the formula I

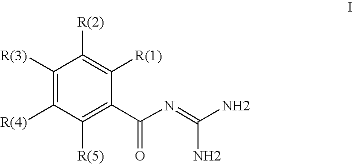

in which:

R(1) is NR(50)R(6),

R(50) and R(6)

independently of one another are hydrogen, (C₁-C₈)-alkyl or (C₁-C₈)-perfluoroalkyl;

R(2), R(3), R(4) and R(5)

independently of one another are R(10)-SOₐ—, R(11)R(12)N—CO—, R(13)-CO— or R(14)R(15)N—SO₂—;

a is zero, 1 or 2,

R(10), R(11), R(12), R(13), R(14) and R(15)

independently of one another are (C₁-C₈)-alkyl, (C₁-C₈)-perfluoroalkyl, (C₃-C₆)-alkenyl or C_{ab}H_{2ab}—R(16);

ab is zero, 1, 2, 3 or 4;

R(16) is (C₃-C₇)-cycloalkyl, phenyl,

which is not substituted or is substituted by 1-3 substituents selected from the group consisting of F, Cl, CF₃, methyl, methoxy and NR(17)R(18);

R(17) and R(18) independently of one another are H, CF₃ or (C₁-C₄)-alkyl;

or

R(11), R(12), and also R(14) and R(15)

together are 4 or 5 methylene groups, of which one CH₂ group can be replaced by oxygen, S, NH, N—CH₃ or N-benzyl;

or

R(11), R(12), R(14) and R(15)

independently of one another are hydrogen;

or

R(2), R(3), R(4) and R(5)

independently of one another are SR(21), —OR(22), —NR(23)R(24) or —CR(25)R(26)R(27);

R(21), R(22), R(23) and R(25)

independently of one another are —C_{b}H_{2b}—(C₁-C₉)-heteroaryl, which is unsubstituted or substituted by 1-3 substituents selected from the group consisting of F, Cl, CF₃, CH₃, methoxy, hydroxyl, amino, methylamino and dimethylamino;

b is zero, 1 or 2;

R(24), R(26) and R(27)

independently of one another are hydrogen, (C₁-C₄)-alkyl or (C₁-C₄)-perfluoroalkyl;

or

R(2), R(3), R(4) and R(5)

independently of one another are hydrogen, F, Cl, Br, I, CN, —(Xₐ)_{dg}—C_{da}H_{2da+1}, —(Xb)_{dh}—(CH₂)_{db}—C_{de}F_{2de+1}, (C₃-C₈)-alkenyl or —C_{df}H_{2df}R(30);

(Xa) is O, S or NR(33);

R(33)

is H, (C₁-C₄)-alkyl or (C₁-C₄)-perfluoroalkyl;

dg is zero or 1;

(Xb) is O, S or NR(34);

R(34)

is H, (C₁-C₄)-alkyl or (C₁-C₄)-perfluoroalkyl;

dh is zero or 1;

da is zero, 1, 2, 3, 4, 5, 6, 7, 8;

db is zero, 1, 2, 3, 4;

de is zero, 1, 2, 3, 4, 5, 6, 7;

di is zero, 1, 2, 3, 4;

R(30)

is (C₃-C₈)-cycloalkyl, phenyl, biphenylyl or naphthyl, where the aromatics phenyl, biphenylyl or naphthyl are not substituted or are substituted by 1-3 substituents selected from the group consisting of F, Cl, CF₃, methyl, methoxy and NR(31)R(32);

R(31) and R(32)

are H, (C₁-C₄)-alkyl or (C₁-C₄)-perfluoroalkyl;

or

R(2), R(3), R(4) and R(5)

independently of one another are NR(40)R(41) or —(Xe)—(CH₂)_{eb}R(⁴⁵);

R(40) and R(41)

independently of one another are hydrogen, (C₁-C₈)-alkyl, (C₁-C₈)-perfluoroalkyl or (CH₂)ₑ—R(42);

e is zero, 1, 2, 3 or 4;

R(42)

is (C₃-C₇)-cycloalkyl, phenyl, which is not substituted or is substituted by 1-3 substituents selected from the group consisting of F, Cl, CF₃, methyl, methoxy and NR(43)R(44);

R(43) and R(44) independently of one another are H, CF₃ or (C₁-C₄)-alkyl;

or

R(40) and R(41)

together are 4 or 5 methylene groups, of which one CH₂ group can be replaced by oxygen, sulfur, NH, N—CH₃ or N-benzyl;

(Xe) is O, S or NR(47);

R(47) is H, (C₁-C₄)-alkyl or (C₁-C₄)-perfluoroalkyl;

eb is zero, 1, 2, 3 or 4;

R(45) is (C₃-C₇)-cycloalkyl, phenyl, which is not substituted or is substituted by 1-3 substituents selected from the group consisting of F, Cl, CF₃, methyl, methoxy, NR(50)R(51) and —(Xfa)-(CH₂)_{ed}—(Xfb)R(46);

Xfa is CH₂, O, S or NR(48);

Xfb is O, S or NR(49);

ed is 1, 2, 3 or 4;

R(46)

is H, (C₁-C₄)-alkyl or (C₁-C₄)-perfluoroalkyl;

R(48), R(49), R(50) and R(51)

independently of one another are H or (C₁-C₄)-alkyl or (C₁-C₄)-perfluoroalkyl;

where R(3) and R(4), however, cannot be hydrogen,

and their pharmaceutically tolerable salts;

(HOE 94/F 265-NZ 272 946, EP-Offenlegungsschrift 700 904)

ad) benzoylguanidines of the formula I

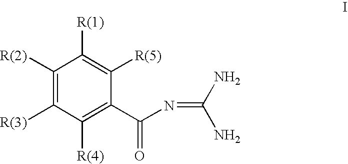

in which:

one of the three substituents R(1), R(2) and R(3)

is (C₁-C₉)-heteroaryl-N-oxide,

which is linked via C or N and which is unsubstituted or substituted by 1-3 substituents selected from the group consisting of F, Cl, CF₃, CH₃, methoxy, hydroxyl, amino, methylamino and dimethylamino;

or

one of the three substituents R(1), R(2) and R(3)

is —SR(10), —OR(10), —NR(10)R(11) or —CR(10)R(11)R(12);

R(10)

is —CₐH₂ₐ—(C₁-C₉)-heteroaryl-N-oxide,

which is unsubstituted or substituted by 1-3 substituents selected from the group consisting of F, Cl, CF₃, CH₃, methoxy, hydroxyl, amino, methylamino and dimethylamino;

a is zero, 1 or 2;

R(11) and R(12)

independently of one another are defined as R(10), are hydrogen or (C₁-C₄)-alkyl;

and the other substituents R(1), R(2) and R(3) in each case

independently of one another are (C₁-C₈)-alkyl, (C₂-C₈)-alkenyl or —CₘH₂ₘR(14);

m is zero, 1 or 2;

R(14) is (C₃-C₈)-cycloalkyl or phenyl,

which is not substituted or is substituted by 1-3 substituents selected from the group consisting of F, Cl, CF₃, methyl, methoxy and NR(15)R(16),

R(15) and R(16) are hydrogen or CH₃;

or

the other substituents R(1), R(2) and R(3) in each case

independently of one another are hydrogen, F, Cl, Br, I, —C≡N, X—(CH₂)ₚ—(C_{q}F_{2q+1}), R(22)-SOᵤ, R(23)R(24)N—CO, R(25)-CO— or R(26)R(27)N—SO₂,

where the perfluoroalkyl group is straight-chain or branched;

X is a bond, oxygen, S or NR(28);

u is zero, 1 or 2;

p is zero, 1 or 2;

q is zero, 1, 2, 3, 4, 5 or 6;

R(22), R(23), R(25) and R(26)

independently are (C₁-C₈)-alkyl, (C₂-C₆)-alkenyl, —CₙH₂ₙ—R(29) or CF₃;

n is zero, 1, 2, 3 or 4;

R(28) is hydrogen or (C₁-C₃)-alkyl;

R(29) is (C₃-C₇)-cycloalkyl or phenyl;

which is not substituted or is substituted by 1-3 substituents selected from the group consisting of F, Cl, CF₃, methyl, methoxy and NR(30)R(31);

R(30) and R(31) are hydrogen or C₁-C₄-alkyl,

or

R(23), R(25) and R(26)

are also hydrogen;

R(24) and R(27)

independently of one another are hydrogen or (C₁-C₄)-alkyl;

or

R(23) and R(24), and also R(26) and R(27) together are 4 or 5 methylene groups, of which one CH₂ group can be replaced by oxygen, S, NH, N—CH₃ or N-benzyl;

or

the other substituents R(1), R(2) and R(3) in each case

independently of one another are OR(35) or NR(35)R(36); R(35) and R(36)

independently of one another are hydrogen or (C₁-C₆)-alkyl;

or

R(35) and R(36)

together are 4-7 methylene groups, of which one CH₂ group can be replaced by oxygen, S, NH, N—CH₃ or N-benzyl,

R(4) and R(5)

independently of one another are hydrogen, (C₁-C₄)-alkyl, F, Cl, —OR(32), —NR(33)R(34) or CᵣF₂ᵣ₊₁;

R(32), R(33) and R(34)

independently of one another are hydrogen or (C₁-C₃)-alkyl;

r is 1, 2, 3 or 4;

and their pharmaceutically tolerable salts;

(HOE 94/F 266-EP-Offenlegungsschrift 702 001, NZ 272 948)

ae) benzoylguanidines of the formula I

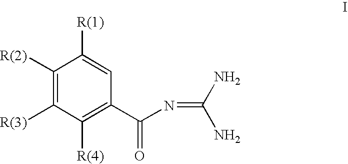

in which:

R(1) is hydrogen, F, Cl, Br, I, CN, NO₂, OH, (C₁-C₈)-alkyl, (C₃-C₈)-cycloalkyl, Oₐ—(CH₂)_{b}—(CF₂)_{c}—CF₃;

a is zero or 1;

b is zero, 1 or 2;

c is zero, 1, 2 or 3;

or

R(1) is R(5)—SOₘ or R(6)R(7)N—SO₂—;

m is zero, 1 or 2;

R(5) and R(6) independently of one another

are (C₁-C₈)-alkyl, (C₃-C₆)-alkenyl, CF₃ or —CₙH₂ₙ—R(8);

n is zero, 1, 2, 3 or 4;

R(7) is hydrogen or (C₁-C₄)-alkyl;

R(8) is (C₃-C₇)-Cycloalkyl or phenyl,

which is not substituted or is substituted by 1-3 substituents selected from the group consisting of F, Cl, CF₃, methyl, methoxy and NR(9)R(10);

R(9) and R(10) independently of one another

are hydrogen or (C₁-C₄)-alkyl;

or

R(6) is H;

or R(6) and R(7)

together are 4 or 5 methylene groups, of which one CH₂ group can be replaced by oxygen, S, NH, N—CH₃ or N-benzyl,

or

R(1) is —SR(11), —OR(11) or —CR(11)R(12)R(13);

R(11) is —CₚH₂ₚ—(C₃-C₈)-cycloalkyl, —(C₁-C₉)-heteroaryl or phenyl,

where the aromatic systems are unsubstituted or substituted by 1-3 substituents selected from the group consisting of F, Cl, CF₃, CH₃, methoxy, hydroxyl, amino, methylamino and dimethylamino;

R(12), R(13) independently of one another

are defined as R(11) or are hydrogen or (C₁-C₄)-alkyl;

p is zero, 1 or 2;

or

R(1) is phenyl, naphthyl, biphenylyl or (C₁-C₉)-heteroaryl, the latter linked via C or N,

which are unsubstituted or substituted by 1-3 substituents selected from the group consisting of F, Cl, CF₃₁ CH₃, methoxy, hydroxyl, amino, methylamino and dimethylamino;

R(2) is —CF₂R(14), —CF[R(15)][R(16)], CF[(CF₂)_{q}—CF₃)][R(15)], —C[(CF₂)ᵣ—CF₃]═CR(15)R(16);

R(14) is (C₁-C₄)-alkyl or (C₃-C₆)-cycloalkyl;

R(15) and R(16) independently of one another

are hydrogen or (C₁-C₄)-alkyl;

q is zero, 1 or 2;

r is zero, 1 or 2;

R(3) is defined as R(1);

R(4) is hydrogen, (C₁-C₃)-alkyl, F, Cl, Br, I, CN, —(CH₂)ₛ—(CF₂)ₜ—CF₃;

s is zero or 1;

t is zero, 1 or 2;

and their pharmaceutically tolerable salts;

(HOE 94/F 267-EP-Offenlegungsschrift 700 899, NZ 272 947)

af) benzoylguanidines of the formula I

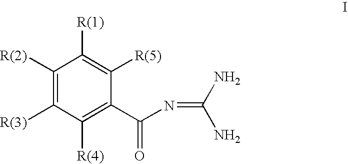

in which:

one of the three substituents R(1), R(2) and R(3)

is —Y-4-[(CH₂)ₖ—CHR(7)-(C═O)R(8)]-phenyl, —Y-3-(CH₂)ₖ—CHR(7)-(C═O)R(8)]-phenyl or —Y-2-[(CH₂)ₖ—CHR(7)-(C═O)R(8)]-phenyl,

where the phenyl in each case is unsubstituted or substituted by 1-2 substituents from the group F, Cl, -CF₃₁ methyl, hydroxyl, methoxy, or —NR(37)R(38);

R(37) and R(38)

independently of one another are hydrogen or —CH₃;

Y is a bond, oxygen, —S— or —NR(9);

R(9) is hydrogen or —(C₁-C₄)-alkyl;

R(7) is —OR(10) or —NR(10)R(11);

R(10) and R(11)

independently of one another are hydrogen, —(C₁-C₈)-alkyl, —(C₁-C₈)-alkanoyl, —(C₁-C₈)-alkoxycarbonyl, benzyl, benzyloxycarbonyl;

or

R(10) is trityl;

R(8) is —OR(12) or —NR(12)R(13);

R(12) and R(13)

independently of one another are hydrogen, —(C₁-C₈)-alkyl or benzyl;

k is zero, 1, 2, 3 or4;

and the other radicals R(1), R(2) and R(3) in each case

independently of one another are —(C₁C₈)-alkyl, —(C₂-C₈)-alkenyl or —(CH₂)ₘR(14);

m is zero, 1 or 2;

R(14) is —(C₃-C₈)-cycloalkyl or phenyl,

which is not substituted or is substituted by 1-3 substituents selected from the group consisting of F, Cl, —CF₃, methyl, methoxy and —NR(15)R(16);

R(15) and R(16)

are hydrogen or —CH₃;

or

the other radicals R(1), R(2) and R(3) in each case

independently of one another are R(18)R(19)N—(C═Y′)—NH—SO₂—;

Y′ is oxygen, —S— or —N—R(20);

R(18) and R(19)

independently of one another are hydrogen, —(C₁-C₈)-alkyl, —(C₃-C₆)-alkenyl or —(CH₂)ₜ—R(21);

t is zero, 1, 2, 3 or 4;

R(21) is —(C₅-C₇)-cycloalkyl or phenyl,

which is unsubstituted or substituted by 1-3 substituents selected from the group consisting of F. Cl, —CF₃, methoxy and —(C₁-C₄)-alkyl;

or

R(18) and R(19)

together are 4 or 5 methylene groups, of which one CH₂ group can be replaced by oxygen, —S—, —NH—, —N—CH₃ or —N-benzyl;

R(20)

is defined as R(18) or is amidine;

or

the other radicals R(1), R(2) and R(3) in each case

independently of one another are hydrogen, F, Cl, Br, I, —C≡N, X—(CH₂)ₚ—(C_{q}F_{2q+1}), R(22)-SOᵤ—, R(23)R(24)N—CO—, R(25)-CO— or R(26)R(27)N—SO₂—, where the perfluoroalkyl group is straight-chain or branched;

X is a bond, oxygen, —S— or —NR(28);

u is zero, 1 or 2;

p is zero, 1 or 2;

q is 1, 2, 3, 4, 5 or 6;

R(22), R(23), R(25) and R(26)

independently of one another are —(C₁-C₈)-alkyl, —(C₃-C₆)-alkenyl, —(CH₂)ₙ—R(29) or —CF₃;

n is zero, 1, 2, 3 or 4;

R(28) is hydrogen or —(C₁-C₃)-alkyl;

R(29) is —(C₃-C₇)-cycloalkyl or phenyl,

which is not substituted or is substituted by 1-3 substituents selected from the group consisting of F, Cl, —CF₃, methyl, methoxy and —NR(30)R(31);

R(30) and R(31) are hydrogen or (C₁-C₄)-alkyl;

or

R(23), R(25) and R(26)

are hydrogen;

R(24) and R(27)

independently of one another are hydrogen or —(C₁-C₄)-alkyl;

or

R(23) and R(24), and also R(26) and R(27)

together are 4 or 5 methylene groups, of which one CH₂ group can be replaced by oxygen, —S—, —NH—, —N—CH₃ or —N-benzyl;

or

the other radicals R(1), R(2) and R(3) in each case

independently of one another are —OR(35) or —NR(35)R(36);

R(35) and R(36)

independently of one another are hydrogen or —(C₁-C₆)-alkyl;

or

R(35) and R(36)

together are 4-7 methylene groups, of which one CH₂ group can be replaced by oxygen, —S—, —NH—, —N—CH₃ or —N-benzyl;

R(4) and R(5)

independently of one another are hydrogen, —(C₁-C₄)-alkyl, F, Cl, —OR(32), —NR(33)R(34) or —CᵣF₂ᵣ₊₁;

R(32), R(33) and R(34)

independently of one another are hydrogen or —(C₁-C₃)-alkyl;

r is 1, 2, 3 or 4;

and their pharmaceutically tolerable salts;

(HOE 94/F 352-EP-Offenlegungsschrift 713 684, NZ 280 517)

ag) benzoylguanidines of the formula I

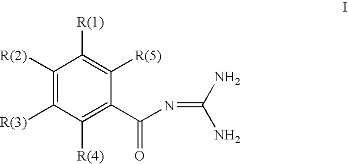

in which:

R(1) is R(6)-CO or R(7)R(8)N—CO;

R(6) is (C₁-C₈)-alkyl, (C₁-C₈)-perfluoroalkyl, (C₃-C₈)-alkenyl

or —CₙH₂ₙ—R(9),

n is zero, 1, 2, 3 or 4;

R(9) is (C₃-C₈)-cycloalkyl, phenyl, biphenylyl or naphthyl,

where the aromatics are not substituted or are substituted by 1-3 substituents selected from the group consisting of F, Cl, CF₃, methyl, methoxy and NR(1 O)R(11),

R(10) and R(11) are H, (C₁-C₄)-alkyl or (C₁-C₄)-perfluoroalkyl;

R(7) is H, (C₁-C₈)-alkyl, (C₁-C₈)-perfluoroalkyl, (C₃-C₈)-alkenyl or —CₙH₂ₙ—R(12);

n is zero, 1, 2, 3 or 4;

R(12) is (C₃-C₈)-cycloalkyl, phenyl, biphenylyl or naphthyl,

where the aromatics are not substituted or are substituted by 1-3 substituents selected from the group consisting of F, Cl, CF₃, methyl, methoxy and NR(13)R(14);

R(13) and R(14) are H, (C₁-C₄)-alkyl or (C₁-C₄)-perfluoroalkyl;

R(8) is H, (C₁-C₄)-alkyl or (C₁-C₄)-perfluoroalkyl;

or

R(7) and R(8) together

are 4 or 5 methylene groups, of which one CH₂ group can be replaced by oxygen, S, NH, N—CH₃ or N-benzyl;

R(2) is defined as R(1), or is H, OH, F, Cl, Br, I, CN, NO₂, (C₁-C₈)-alkyl, (C₁-C₈)-perfluoroalkyl, (C₃-C₈)-alkenyl or —CₙH₂ₙR(15);

n is zero, 1, 2, 3 or 4;

R(15) is (C₃-C₈)-cycloalkyl, phenyl, biphenylyl or naphthyl,

where the aromatics are not substituted or are substituted by 1-3 substituents selected from the group consisting of F, Cl, CF₃, methyl, methoxy and NR(16)R(17);

R(16) and R(17)

are H, (C₁-C₄)-alkyl or (C₁-C₄)-perfluoroalkyl;

or

R(2) is (C₁-C₉)-heteroaryl,

which is linked via C or N and which is unsubstituted or substituted by 1-3 substituents selected from the group consisting of F, Cl, CF₃, CH₃, methoxy, hydroxyl, amino, methylamino and dimethylamino;

or

R(2) is SR(18),-OR(18), -NR(18)R(19) or -CR(18)R(19)R(20);

R(18) is CₐH₂ₐ—(C₁-C₉)-heteroaryl,

which is unsubstituted or substituted by 1-3 substituents selected from the group consisting of F, Cl, CF₃, CH₃, methoxy, hydroxyl, amino, methylamino, dimethylamino;

a is zero, 1 or 2;

R(19) and R(20)

independently of one another are defined as R(18) or are hydrogen, (C₁-C₄)-alkyl of (C₁-C₄)-perfluoroalkyl;

or

R(2) is R(21)-SOₘ or R(22)R(23)N—SO₂—;

m is 1 or 2;

R(21) is (C₁-C₈)-alkyl, (C₁-C₈)-perfluoroalkyl, (C₃-C₈)-alkenyl or —CₙH₂ₙ—R(24);

n is zero, 1, 2, 3 or 4;

R(24) is (C₃-C₈)-cycloalkyl, phenyl, biphenylyl or naphthyl,

where the aromatics are not substituted or are substituted by 1-3 substituents selected from the group consisting of F, Cl, CF₃, methyl, methoxy and NR(27)R(28);

R(27) and R(28) are H, (C₁-C₄)-alkyl or (C₁-C₄)-perfluoroalkyl;

R(22) is H, (C₁-C₈)-alkyl, (C₁-C₈)-perfluoroalkyl, (C₃-C₈)-alkenyl or —CₙH₂ₙ—R(29);

n is zero, 1, 2, 3 or 4;

R(29) is (C₃-C₈)-cycloalkyl, phenyl, biphenylyl or naphthyl,

where the aromatics are not substituted or are substituted by 1-3 substituents selected from the group consisting of F, Cl, CF₃, methyl, methoxy and NR(30)R(31);

R(30) and R(31) are H, (C₁-C₄)-alkyl or (C₁-C₄)-perfluoroalkyl;

R(23) is hydrogen, (C₁-C₄)-alkyl or (C₁-C₄)-perfluoroalkyl;

or

R(22) and R(23)

together are 4 or 5 methylene groups, of which one CH₂ group can be replaced by oxygen, S, NH, N—CH₃ or N-benzyl;

or

R(2) is R(33)X-;

X is oxygen, S, NR(34), (D=O)A- or NR(34)C=MN^{(*)}R(35)-;

M is oxygen or S;

A is oxygen or NR(34);

D is C or SO;

R(33) is (C₁-C₈)-alkyl, (C₃-C₈)-alkenyl, (CH₂)_{b}C_{d}F_{2d+1} or —CₙH₂ₙ—R(36);

b is zero or 1;

d is 1, 2, 3, 4, 5, 6 or 7;

n is zero, 1, 2, 3, or 4;

R(36) is (C₃-C₈)-cycloalkyl, phenyl, biphenylyl or naphthyl,

where the aromatics are not substituted or are substituted by 1-3 substituents selected from the group consisting of F, Cl, CF₃, methyl, methoxy and NR(37)R(38);

R(37) and R(38) are H. (C₁-C₄)-alkyl or (C₁-C₄)-perfluoroalkyl;

R(34) is hydrogen, (C₁-C₄)-alkyl or (C₁-C₄)-perfluoroalkyl;

R(35) is defined as R(33);

or

R(33) and R(34)

together are 4 or 5 methylene groups, of which one CH₂ group can be replaced by oxygen, S, NH, N—CH₃ or N-benzyl;

where A and N^{(*)} are bonded to the phenyl nucleus of the benzoylguanidine parent structure;

or

R(2) is —SR(40), —OR(40), —NHR(40), —NR(40)R(41), —CHR(40)R(42), —CR(42)R(43)OH, —C≡CR(45), —CR(46)=CR(45) or —[CR(47)R(48)]ᵤ—C≡C(R49)R(50)]ᵥ—R(44);

R(40) and R(41)

independently of one another are —(CH₂)ₚ—(CHOH)_{q}—(CH₂)ᵣ—(CHOH)ₜ—R(51) or —(CH₂)ₚ—C(CH₂—CH₂O)_{q}—R(51);

R(51) is hydrogen or methyl;

u is 1, 2, 3 or 4;

v is zero, 1, 2, 3 or 4;

p, q and r

independently of one another are zero, 1, 2, 3 or 4;

t is 1, 2, 3 or 4;

R(42) and R(43)

independently of one another are hydrogen or (C₁-C₆)-alkyl;

or

R(42) and R(43)

together with the carbon atom carrying them are a (C₃-C₈)-cycloalkyl;

R(44) is hydrogen, (C₁-C₆)-alkyl, (C₃-C₈)-cycloalkyl, —CₑH₂ₑ—R(⁴⁵);

e is zero, 1, 2, 3 or 4;

R(45) is phenyl,

which is unsubstituted or substituted by 1-3 substituents selected from the group consisting of F, Cl, CF₃, methyl, methoxy and NR(52)R(53);

R(52) and R(53) are H or (C₁-C₄)-alkyl;

or

R(45) is (C₁-C₉)-heteroaryl,

which is unsubstituted or substituted as phenyl;

or

R(45) is (C₁-C₆)-alkyl,

which is unsubstituted or substituted by 1-3 OH;

R(46), R(47), R(48), R(49) and R(50)

independently of one another are hydrogen or methyl;

or

R(2) is R(55)-NH—SO₂—;

R(55) is R(56)R(57)N−(C═Y)—;

Y is oxygen, S or N—R(58);

R(56) and R(57)

independently of one another are hydrogen, (C₁-C₈)-alkyl, (C₃-C₆)-alkenyl or —C_{f}H_{2f}—R(59);

f is zero, 1, 2, 3 or4;

R(59) is (C₅-C₇)-cycloalkyl, phenyl,

which is unsubstituted or substituted by 1-3 substituents selected from the group consisting of F, Cl, CF₃, methoxy and (C₁-C₄)-alkyl;

or

R(56) and R(57)

together are 4 or 5 methylene groups, of which one CH₂ group can be replaced by oxygen, S, NH, N—CH₃ or N-benzyl;

R(58)

is defined as R(56) or is amidine;

R(3), R(4) and R(5) are independently of one another defined as R(1) or R(2), but where at least one of the substituents R(2), R(3), R(4) and R(5) must be OH;

and their pharmaceutically tolerable salts;

(HOE 95/F 007 K-EP-Offenlegungsschrift 723 956, NZ 280 887)

ah) benzoylguanidines of the formula I

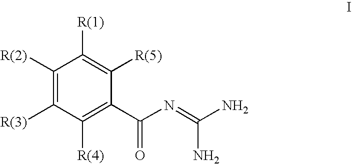

in which:

one of the three substituents R(1), R(2) and R(3)

is R(6)-A-B-D-;

R(6) is a basic protonatable radical, i.e. an amino group

—NR(7)R(8), an amidino group R(7)R(8)N—C[═N—R(9)]- or a guanidino group

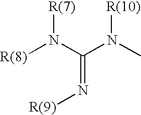

R(7), R(8), R(9) and R(10)

independently of one another are hydrogen or alkyl having 1, 2, 3 or 4 carbon atoms;

or

R(7) and R(8)

together are CₐH₂ₐ;

a is 4, 5, 6 or 7;

where if a=5, 6 or 7 a methylene group of the group CₐH₂ₐ can be replaced by a heteroatom group O, SOₘ or NR(11),

or

R(8) and R(9) or R(9) and R(10) or R(7) and R(10)

are a group CₐH₂ₐ;

a is 2, 3, 4 or 5;

where if a=3, 4 or 5 a methylene group of the group CₐH₂ₐ can be replaced by a heteroatom group O, SOₘ or NR(11);

m is zero, 1 or 2;

R(11) is hydrogen or methyl;

or

R(6) is a basic heteroaromatic ring system having 1-9 carbon atoms;

A is C_{b}H_{2b};

b is 1, 2, 3, 4, 5, 6, 7, 8, 9 or 10;

where in the group C_{b}H_{2b} one or two methylene groups can be replaced by one of the groupings selected from the group consisting of —O—, —CO—, —CH[OR(20)]-, —SOₘ—, —NR(20)-, —NR(20)-CO—, —NR(20)-CO—NH—, —NR(20)-CO—NH—SO₂—

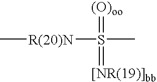

and —SOₐₐ[NR(19)]_{bb}-;

and where in the group C_{b}H_{2b} a methylene group can be replaced by —CH—R(99), where R(99) together with R(7) forms a pyrrolidine or piperidine ring;

aa is 1 or 2;

bb is 0 or 1;

aa+bb=2;

R(19) is hydrogen or alkyl having 1, 2, 3 or 4 carbon atoms;

R(20) is hydrogen or methyl;

B is a phenylene or naphthylene radical

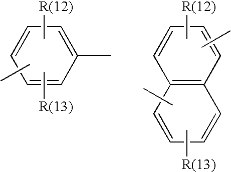

R(12) and R(13)

independently of one another are hydrogen, methyl, F, Cl, Br, 1, CF₃ or —SO_{w}—R(14);

R(14) is methyl or NR(15)R(16);

R(15) and R(16)

independently of one another are hydrogen or alkyl having 1, 2, 3 or 4 carbon atoms;

w is zero, 1 or2;

D is —C_{d}H_{2d}—X_{f}—;

d is zero, 1, 2, 3 or 4;

X is —O—, —CO—, —CH[OR(21)]-, —SOₘ, or —NR(21)-;

f is zero or 1;

R(21) is hydrogen or methyl;

m is zero, 1 or 2;

and the other substituents R(1) and R(2) and R(3) in each case

independently of one another are hydrogen, F, Cl, Br, I, —CN, —(C₁-C₈)-alkyl, —(C₂-C₈)-alkenyl, —NR(35)R(36) or R(17)-C_{g}H_{2g}-Zₕ-;

g is zero, 1, 2, 3 or 4;

h is zero or 1;

R(35) and R(36)

independently of one another are hydrogen or alkyl having 1, 2, 3, 4, 5 or 6 carbon atoms;

or

R(35) and R(36)

together are 4-7 methylene groups, of which one CH₂ group can be replaced by oxygen, —S—, —NH—, —NCH₃ or —N-benzyl;

Z is —O—, —CO—, —SOᵥ—, —NR(18)-, —NR(18)-CO—, —NR(18)-CO—NH—or —NR(18)-SO₂—;

R(18) is hydrogen or methyl;

v is zero, 1 or 2;

R(17) is hydrogen, cycloalkyl having 3, 5 or 6 carbon atoms or CₖF₂ₖ₊₁—;

k is 1, 2 or 3,

or

R(17) is pyrrol-1-yl, pyrrol-2-yl or pyrrol-3-yl,

which is not substituted or is substituted by 1-4 substituents selected from the group consisting of F, Cl, Br, I, —CN, (C₂-C₈)-alkanoyl, (C₂-C₈)-alkoxycarbonyl, formyl, carboxyl, —CF₃, methyl and methoxy;

or

R(17) is (C₃-C₈)-cycloalkyl or phenyl,

which is not substituted or is substituted by 1-3 substituents selected from the group consisting of F and Cl, —CF₃, methyl, hydroxyl, methoxy, —NR(37)R(38), CH₃SO₂— and H₂NO₂S—;

R(37) and R(38)

are hydrogen or —CH₃;

R(4) and R(5)

independently of one another are hydrogen, alkyl having 1, 2, 3 or 4 carbon atoms, F, Cl, —OR(32), —NR(33)R(34) or —CᵣF₂ᵣ₊₁;

R(32), R(33) and R(34)

independently of one another are hydrogen or alkyl having 1, 2 or 3 carbon atoms;

r is 1, 2, 3 or 4;

and their pharmacologically tolerable salts;

(HOE 95/F 072-EP-Offenlegungsschrift 738 712, NZ 286 380)

ai) indenoylguanidines of the formula I

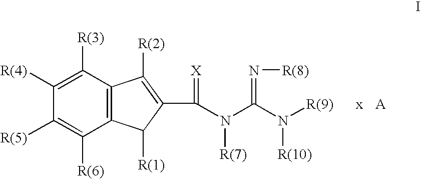

in which:

R(1) and R(2)

independently of one another are hydrogen, alkyl having 1, 2, 3, 4, 5, 6, 7, 8, 9 or 10 carbon atoms, cycloalkyl having 3, 4, 5 or 6 carbon atoms, O-alkyl having 1, 2, 3 or 4 carbon atoms, OC(═O)-alkyl having 1, 2, 3 or 4 carbon atoms or CₘH₂ₘ—NR(12)R(13);

R(12) and R(13)

independently of one another are hydrogen or alkyl having 1, 2, 3 or 4 carbon atoms;

m is zero, 1, 2, 3 or 4;

NH—C(═O)—NH₂, C(═O)—O-alkyl having 1, 2, 3 or 4 carbon atoms, C(═O)—NH₂, C(═O)—NH-alkyl having 1, 2, 3 or 4 carbon atoms, C(═O)—N(alkyl)₂ having 1, 2, 3 or 4 carbon atoms in each alkyl group, alkenyl having 2, 3, 4, 5, 6, 7, 8, 9 or 10 carbon atoms, alkynyl having 2, 3, 4, 5, 6, 7, 8, 9 or 10 carbon atoms, alkylaryl having 1, 2, 3 or 4 carbon atoms in the alkyl group, alkenylaryl having 2, 3, 4, 5, 6, 7, 8, 9 or 10 carbon atoms in the alkenyl group, alkynylaryl having 2, 3, 4, 5, 6, 7, 8, 9 or 10 carbon atoms in the alkynyl group, C₁-C₄-alkyl-substituted aryl, C₁-C₄-alkylheteroaryl, C₁-C₄-alkenylheteroaryl, aminoalkylaryl having 1, 2, 3 or 4 carbon atoms in the alkyl group, substituted aryl, heteroaryl and substituted heteroaryl;

R(3), R(4), R(5) and R(6)

independently of one another are hydrogen, alkyl having 1, 2, 3, 4, 5, 6, 7, 8, 9 or 10 carbon atoms, O-alkyl having 1, 2, 3, 4, 5, 6, 7, 8, 9 or 10 carbon atoms, halogen, (such as F, Cl, Br, I), OH, aryl, substituted aryl, heteroaryl, substituted heteroaryl, O-lower alkyl, O-aryl, O-lower alkylaryl, O-substituted aryl, O-lower alkyl-substituted aryl, O—C(=O)—C₁-C₄-alkylaryl, O—C(═O)—NH—C₁-C₄-alkyl, O—C(═O)—N(C₁-C₄-alkyl)₂, NO₂, CN, CF₃, NH₂, NH—C(═O)—C₁-C₄-alkyl, NH—C(═O)—NH₂, COOH, C(═O)—O—C₁-C₄-alkyl, C(═O)—NH₂, C(═O)—NH—C₁-C₄-alkyl, C(═O)—N(C₁-C₄-alkyl)₂, C₁-C₄—COOH, C₁-C₄-alkyl-C(═O)—O—C₁-C₄-alkyl, SO₃H, SO₂-alkyl, SO₂-alkylaryl, SO₂-N-(alkyl)₂, SO₂-N(alkyl)(alkylaryl), C(═O)—R(11), C₁-C₁₀-alkyl-C(═O)—R(11), C₂-C₁₀-alkenyl-C(═O)—R(11), C₂-C₁₀-alkynyl-C(═O)—R(11), NH—C(═O)—C₁-C₁₀-alkyl-C(═O)—R(11), O—C₁-C₁₁-alkyl-C(═O)—R(11);

R(11)is C₁-C₄-alkyl, C₁-C₄-alkynyl, aryl, substituted aryl, NH₂, NH—C₁-C₄-alkyl, N—(C₁-C₄-alkyl)₂, SO₃H, SO₂-alkyl, SO₂-alkylaryl, SO₂—N-(alkyl)₂, SO₂-N(alkyl)(alkylaryl);

X is O, S or NH;

R(7), R(8), R(9) and R(10)

independently of one another are hydrogen, alkyl, cycloalkyl, aryl, alkylaryl;

or

R(8) and R(9)

together are part of a 5, 6 or 7-membered heterocyclic ring;

A is absent or is a nontoxic organic or inorganic acid.

(HOE 95/F 109-EP 748 795, NZ 286 583)

ak) benzyloxycarbonylguanidines of the formula I

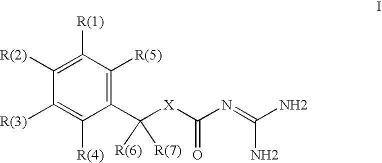

in which:

R(1), R(2) and R(3)

independently of one another are —Y-[4-R(8)-phenyl], —Y-[3-R(8)-phenyl] or —Y-[2-R(8)-phenyl],

where the phenyl is in each case unsubstituted or substituted by 1-2 substituents from the group consisting of F, Cl, —CF₃, methyl, hydroxyl, methoxy and -NR(96)R(97);

R(96) and R(97)

independently of one another are hydrogen or —CH₃;

Y is a bond, CH₂, oxygen, —S—or —NR(9);

R(9) is hydrogen or alkyl having 1, 2, 3 or 4 carbon atoms;

R(8) is SOₐ[NR(98)]_{b}NR(99)R(10);

a is 1 or 2;

b is 0 or 1;

a+b=2;

R(98), R(99) and R(10)

independently of one another are hydrogen, —(C₁-C₈)-alkyl, benzyl, —(C₂-C₈)-alkylene-NR(11)R(12), (C₂-C₈)-alkylene-NR(13)-(C₂-C₈)-alkylene-NR(37)R(38) or (C₀-C₈)-alkylene-CR(39)R(40)CR(41)R(42)(C₀-C₈)-alkylene-NR(43)R(44);

R(11), R(12), R(13), R(37), R(38), R(43) and R(44)

independently of one another are hydrogen, —(C₁-C₈)-alkyl or benzyl;

R(39), R(40), R(41) and R(42)

independently of one another are hydrogen, —(C₁-C₈)-alkyl or —(CO—C₃)-alkylenephenyl,

where the phenyl is not substituted or is substituted by 1-3 substituents selected from the group consisting of F, Cl, —CF₃, methyl and methoxy;

or

R(99) and R(10)

together are 4-6 methylene groups, of which one CH₂ group can be replaced by oxygen, —S—, —NH—, —N—CH₃ or —N-benzyl;

or

R(8) is SOₐ[NR(98)]_{b}NR(95)-C[═N—R(94)]-NR(93)R(92);

R(92), R(93), R(94) and R(95)

independently of one another are hydrogen or alkyl having 1, 2, 3 or 4 carbon atoms;

or

R(1), R(2) and R(3)

independently of one another are pyrrol-1-yl, pyrrol-2-yl or pyrrol-3-yl,

which is not substituted or is substituted by 1-4 substituents selected from the group consisting of F, Cl, Br, I, —CN, (C₂-C₈)-alkanoyl, (C₂-C₈)-alkoxycarbonyl, formyl, carboxyl, —CF₃, methyl, methoxy;

or

R(1), R(2) and R(3)

independently of one another are hydrogen, (C₁-C₈)-alkyl, —(C₂-C₈)-alkenyl or —(CH₂)ₘR(14);

m is zero, 1 or 2;

R(14) is —(C₃-C₈)-cycloalkyl or phenyl,

which is not substituted or is substituted by 1-3 substituents selected from the group consisting of F and —Cl, —CF₃, methyl, methoxy and —NR(15)R(16);

R(15) and R(16)

are hydrogen or —CH₃;

or

R(1), R(2) and R(3)

independently of one another are -Q-4-[(CH₂)ₖ—CHR(17)-(C═O)R(20)]-phenyl, —O-3-(CH₂)ₖ—CHR(17)-(C═O)R(20)]-phenyl or -Q-2-[(CH₂)ₖ—CHR(17)-(C═O)R(20)]-phenyl,

where the phenyl in each case is unsubstituted or substituted by 1-2 substituents from the group F, Cl, —CF₃₁ methyl, hydroxyl, methoxy and -NR(35)R(36);

R(35) and R(36)

independently of one another are hydrogen or —CH₃;

Q is a bond, oxygen, —S—or —NR(18);

R(18) is hydrogen or —(C₁-C₄)-alkyl;

R(17) is —OR(21) or —NR(21)R(22);

R(21) and R(22)

independently of one another are hydrogen, —(C₁-C₈)-alkyl, —(C₁-C₈)-alkanoyl, —(C₁-C₈)-alkoxycarbonyl, benzyl, benzyloxycarbonyl;

or

R(21) is trityl;

R(20) is —OR(23) or —NR(23)R(24);

R(23), R(24) independently of one another are hydrogen, —(C₁-C₈)-alkyl or benzyl;

k is zero, 1, 2, 3 or 4;

or

R(1), R(2) and R(3)

independently of one another are (C₁-C₉)-heteroaryl,

which is linked via C or N and which is unsubstituted or substituted by 1-3 substituents from the group F, Cl, CF₃, CH₃, methoxy, hydroxyl, amino, methylamino and dimethylamino;

or 4

R(1), R(2) and R(3)

are —SR(25), —OR(25), —NR(25)R(26), —CR(25)R(26)R(27);

R(25) is —C_{f}H_{2f}—(C₁-C₉)-heteroaryl,

which is unsubstituted or substituted by 1-3 substituents from the group F, Cl, CF₃, CH₃, methoxy, hydroxyl, amino, methylamino and dimethylamino;

f is zero, 1 or 2;

R(26) and R(27)

independently of one another are defined as R(25) or are hydrogen or (C₁-C₄)-alkyl,

or

R(1), R(2) and R(3)

independently of one another are (C₁-C₉)-heteroaryl-N-oxide,

which is linked via C or N and which is unsubstituted or substituted by 1-3 substituents selected from the group consisting of F, Cl, CF₃, CH₃, methoxy, hydroxyl, amino, methylamino and dimethylamino;

or

R(1), R(2) and R(3)

independently of one another are —SR(28), —OR(28), —NR(28)R(29) or —CR(28)R(29)R(30);

R(28) is —C_{g}H_{2g}—(C₁-C₉)-heteroaryl-N-oxide,

which is unsubstituted or substituted by 1-3 substituents selected from the group consisting of F, Cl, CF₃, CH₃, methoxy, hydroxyl, amino, methylamino and dimethylamino;

g is zero, 1 or 2;

R(29), R(30)

independently of one another are defined as R(28), hydrogen or (C₁-C₄)-alkyl;

or

R(1), R(2) and R(3)

independently of one another are hydrogen, F, Cl, Br, I, —C≡N, T-(CH₂)ₕ—(CᵢF₂ᵢ₊₁), R(31)SOₗ—, R(32)R(33)N—CO—, R(34)-CO— or R(45)R(46)N—SO₂, where the perfluoroalkyl group is straight-chain or branched;

T is a bond, oxygen, —S— or —NR(47);

l is zero, 1 or 2;

h is zero, 1 or 2;

i is 1, 2, 3, 4, 5 or 6;

R(31), R(32), R(34) and R(45)

independently of one another are —(C₁-C₈)-alkyl, —(C₃-C₆)-alkenyl, (CH₂) R(48) or —CF₃;

n is zero, 1, 2, 3 or 4;

R(47) is hydrogen or alkyl with 1, 2 or 3 carbon atoms;

R(48) is —(C₃-C₇)-cycloalkyl or phenyl,

which is not substituted or is substituted by 1-3 substituents selected from the group consisting of F, Cl, —CF₃, methyl, methoxy and —NR(49)R(50);

R(49) and R(S0)

are hydrogen or alkyl having 1, 2, 3 or 4 carbon atoms;

or

R(32), R(34) and R(45)

are hydrogen;

R(33) and R(46)

independently of one another are hydrogen or alkyl having 1, 2, 3 or 4 carbon atoms;

or

R(32) and R(33), and R(45) and R(46)

together are 5 or 6 methylene groups, of which one CH₂ group can be replaced by oxygen, —S—, —NH—, —NCH₃ or —N-benzyl;

or

R(1), R(2) and R(3)

independently of one another are R(51)-A-G-D-;

R(51) is a basic protonatable radical, i.e. an amino group

—NR(52)R(53), an amidino group R(52)R(53)N—C[═N—R(54)]- or a guanidino group R(52)R(53)N—C[═N—R(54)]-NR(55)-;

R(52), R(53), R(54) and R(55)

independently of one another are hydrogen or alkyl having 1, 2, 3 or 4 carbon atoms;

or

R(52) and R(53) are

a group C_{α}H_{2α};

α is 4, 5, 6 or 7;

where if α=5, 6 or 7 a carbon atom of the group

C_{α}H_{2α} can be replaced by a heteroatom group O, SO_{d} or NR(56),

or

R(53) and R(54) or R(54) and R(55) or R(52) and R(55) are

a group C_{γ}H_{2γ};

γ is 2, 3, 4 or 5;

where if γ=3, 4 or 5 a carbon atom of the group C_{γ}H_{2γ} can be replaced by a heteroatom group O, SO_{d} or NR(56);

d is zero, 1 or 2;

R(56) is hydrogen or methyl;

or

R(51) is a basic heteroaromatic ring system having 1-9 carbon atoms;

A is a group CₑH₂ₑ;

e is zero, 1, 2, 3, 4, 5, 6, 7, 8, 9 or 10;

where in the group CₑH₂ₑ a carbon atom can be replaced by one of the groupings —O—, —CO—, —CH[OR(57)]-, —SOᵣ—, —NR(57)-, —NR(57)-CO—, —NR(57)-CO—NH—, —NR(57)-CO—NH—SO₂— or —NR(57)-SO₂—;

r is zero, 1 or 2;

G is a phenylene radical

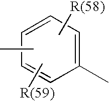

R(58) and R(59)

independently of one another are hydrogen, methyl, methoxy, F, Cl, Br, I, CF₃ or —SOₛ—R(60);

R(60) is methyl or NR(61)R(62);

R(61) and R(62)

independently of one another are hydrogen or alkyl having 1, 2, 3 or 4 carbon atoms;

D is —CᵥH₂ᵥ-E_{w}-;

v is zero, 1, 2, 3 or 4;

E is —O—, —CO—, —CH[OR(63)]-, —SOₐₐ— or —NR(63)-;

w is zero or 1;

aa is zero, 1 or 2

R(63) is hydrogen or methyl,

or

R(1), R(2) and R(3)

independently of one another are —CF₂R(64), —CF[R(65)][R(66)], —CF[(CF₂)_{q}—CF₃[R(65)), —C[(CF₂)ₚ—CF₃]=CR(65)R(66);

R(64) is alkyl having 1, 2, 3 or 4 carbon atoms or cycloalkyl having 3, 4, 5, 6 or 7 carbon atoms;

R(65) and R(66) Independently of one another are hydrogen or alkyl having 1, 2, 3 or 4 carbon atoms;

q is zero, 1 or 2;

p is zero, 1 or 2;

or

R(1), R(2) and R(3)

independently of one another are —OR(67) or —NR(67)R(68);

R(67) and R(68)

independently of one another are hydrogen or alkyl having 1, 2, 3, 4, 5 or 6 carbon atoms;

or

R(67) and R(68)

together are 4, 5, 6 or 7 methylene groups, of which one CH₂ group can be replaced by oxygen, —S—, SO₂, —NH—, —NCH₃ or —N-benzyl;

R(4) and R(5)

independently of one another are hydrogen, alkyl having 1, 2, 3 or 4 carbon atoms, F, Cl, —OR(69), —NR(70)R(71) or —C_{z}F_{2z+1};

R(69), R(70) and R(71)

independently of one another are hydrogen or alkyl having 1, 2 or 3 carbon atoms;

z is 1, 2, 3 or 4;

R(6) and R(7)

independently of one another are hydrogen or alkyl having 1, 2, 3 or 4 carbon atoms;

X is oxygen or NR(72);

R(72) is hydrogen or alkyl having 1, 2, 3 or 4 carbon atoms;

and their pharmaceutically tolerable salts;

(HOE 95/F 115-EP 744 397, NZ 286 622)

al) alkenylcarboxylic acid guanidides, carrying fluorophenyl groups, of the formula I

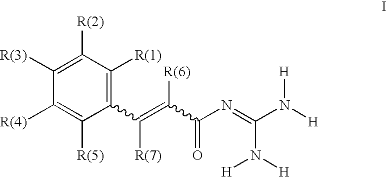

in which:

R(6) is hydrogen, (C₁-C₈)-alkyl, (C₃-C₈)-cycloalkyl or phenyl,

where the phenyl group is not substituted or is substituted by 1-3 substituents selected from the group consisting of F, Cl, CF₃, methyl, methoxy and NR(9)R(10);

R(9) and R(10)

are hydrogen, (C₁-C₄)-alkyl or (C₁-C₄)-perfluoroalkyl;

R(7) independently is defined as R(6);

R(1), R(2), R(3), R(4) and R(5)

independently of one another are hydrogen or F;

where, however, at least one of the radicals R(1), R(2), R(3), R(4) and R(5) must be fluorine;

and their pharmaceutically tolerable salts;

(HOE 95/F 167-NZ 299 015)

ak) benzoylguanidines of the formula I

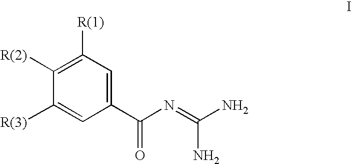

in which:

R(1) is R(4)-SOₘ

or R(5)R(6)N—SO₂—;

m is 1 or 2;

R(4) and R(5)

independently of one another are alkyl having 1, 2, 3, 4, 5, 6, 7 or 8 carbon atoms, alkenyl having 3, 4, 5 or 6 carbon atoms, CF₃ or —CₙH₂ₙ—R(7);

n is zero, 1, 2, 3 or 4;

R(6) is H or alkyl having 1, 2, 3 or 4 carbon atoms;

R(7) is cycloalkyl having 3, 4, 5, 6, 7 or 8 carbon atoms or phenyl,

which is not substituted or is substituted by 1-3 substituents selected from the group consisting of F, Cl, CF₃, methyl, methoxy and NR(8)R(9);

R(8) and R(9) are H or alkyl having 1, 2, 3 or 4 carbon atoms;

or

R(5) is also hydrogen;

or

R(5) and R(6)

together are 4 or 5 methylene groups, of which a CH₂ group can be replaced by oxygen, S, NH, N—CH₃ or N-benzyl;

or

R(1) is —Oₚ—(CH₂)_{q}—(CF₂)ᵣ—CF₃;

p is zero or 1;

q is zero, 1 or 2;

r is zero, 1, 2 or 3;

or

R(1) is —SR(10), —OR(10) or —CR(10)R(11)R(12);

R(10), R(11) and R(12)

independently of one another are hydrogen, alkyl having 1, 2, 3, 4, 5, 6, 7 or 8 carbon atoms, —CₛH₂ₛ—(C₃-C₈)-cycloalkyl or an aromatic system selected from the group consisting of pyridyl, pyrrolyl, quinolyl, isoquinolyl, imidazolyl or phenyl;

s is zero, 1 or 2;

where the aromatic systems pyridyl, pyrrolyl, quinolyl, isoquinolyl, imidazolyl and phenyl are unsubstituted or substituted by 1-3 substituents selected from the group consisting of F, Cl, CF₃, CH₃, methoxy, hydroxyl, amino, methylamino and dimethylamino;

R(2) is —(CH₂)ᵤ—(CF₂)ₜ—CF₃;

t is zero, 1, 2 or 3;

u is zero or 1;

R(3) is hydrogen or independently is defined as R(1);

and their pharmaceutically tolerable salts;

(HOE 95/F 173-NZ 299 052)

an) substituted cinnamic acid guanidides of the formula I

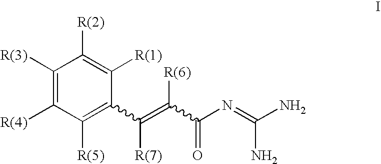

in which:

at least one of the substituents R(1), R(2), R(3), R(4) and R(5) is

—Xₐ—Y_{b}-Lₙ-U;

X is CR(16)R(17), O, S or NR(18);

R(16), R(17) and R(18)

independently of one another are H, alkyl having 1, 2, 3 or 4 carbon atoms or perfluoroalkyl having 1, 2, 3 or 4 carbon atoms;

a is zero or 1;

Y is alkylene having 1, 2, 3, 4, 5, 6, 7 or 8 carbon atoms, alkylene-T having 1, 2, 3, 4, 5, 6, 7 or 8 carbon atoms in the alkylene group, T, T-alkylene having 1, 2, 3, 4, 5, 6, 7 or 8 carbon atoms in the alkylene group;

T is NR(20), O, S or phenylene,

where the phenylene is not substituted or is substituted by 1-3 substituents selected from the group consisting of F, Cl, CF₃, methyl, methoxy and NR(21)R(22);

R(20), R(21) and R(22)

independently of one another are H, alkyl having 1, 2, 3 or 4 carbon atoms or perfluoroalkyl having 1, 2, 3 or 4 carbon atoms;

b is zero or 1;

L is O, S, NR(23) or CₖH₂ₖ;

k is 1, 2, 3, 4, 5, 6, 7, 8;

n is zero or 1;

U is NR(24)R(25) or an N-containing heterocycle having 1, 2, 3, 4, 5, 6, 7, 8 or 9 carbon atoms;

R(24) and R(25)

independently of one another are hydrogen, alkyl having 1, 2, 3, 4, 5, 6, 7 or 8 carbon atoms or perfluoroalkyl having 1, 2, 3, 4, 5, 6, 7 or 8 carbon atoms;

or

R(24) and R(25)

together are 4 or 5 methylene groups, of which one CH₂ group can be replaced by oxygen, S, NH, N—CH₃ or N-benzyl;

where the N-containing heterocycles are N- or C-bridged and are not substituted or are substituted by 1-3 substituents selected from the group consisting of F, Cl, CF₃, methyl, methoxy and NR(27)R(28);

R(23), R(27) and R(28)

independently of one another are H, alkyl having 1, 2, 3 or 4 carbon atoms or perfluoroalkyl having 1, 2, 3 or 4 carbon atoms;

and the other substituents R(1), R(2), R(3), R(4) and R(5) in each case

independently of one another are H, F, Cl, Br, I, CN, —Oₙ—CₘH₂ₘ₊₁, —Oₚ—(CH₂)ₛ—C_{q}F_{2q+1} or —CᵣH₂ᵣR(¹⁰);

n is zero or 1;

m is zero 1, 2, 3, 4, 5, 6, 7 or 8;

p is zero or 1;

q is 1, 2, 3, 4, 5, 6, 7 or 8;

s is zero, 1, 2, 3 or 4;

r is zero, 1, 2, 3 or 4;

R(10)

is cycloalkyl having 3, 4, 5, 6, 7 or 8 carbon atoms, or phenyl,

where the phenyl is not substituted or is substituted by 1-3 substituents selected from the group consisting of F, Cl, CF₃, methyl, methoxy and NR(11)R(12);

R(11) and R(12)

independently of one another are H, alkyl having 1, 2, 3 or 4 carbon atoms or perfluoroalkyl having 1, 2, 3 or 4 carbon atoms;

R(6) and R(7)

independently of one another are hydrogen, F, Cl, Br, I, CN, alkyl having 1, 2, 3, 4, 5, 6, 7 or 8 carbon atoms, perfluoroalkyl having 1, 2, 3, 4, 5, 6, 7 or 8 carbon atoms, cycloalkyl having 3, 4, 5, 6, 7 or 8 carbon atoms, or phenyl,

which is not substituted or is substituted by 1-3 substituents selected from the group consisting of F, Cl, CF₃, methyl, methoxy and NR(14)R(15);

R(14) and R(15)

independently of one another are H, alkyl having 1, 2, 3 or 4 carbon atoms or perfluoroalkyl having 1, 2, 3 or 4 carbon atoms;

and their pharmaceutically tolerable salts;

(HOE 95/F 220—NZ 299 052)

ao) benzoylguanidines of the formula I

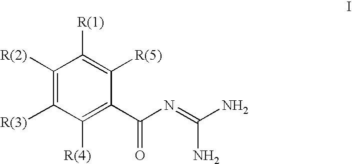

in which:

at least one of the substituents R(1), R(2) and R(3)

is R(6)-C(OH)₂—;

R(6) is perfluoroalkyl having 1, 2 or 3 carbon atoms, which is straight-chain or branched;

and the other substituents R(1), R(2) and R(3)

independently of one another are hydrogen, OH, F, Cl, Br, I, alkyl having 1, 2, 3, 4, 5 or 6 carbon atoms, cycloalkyl with 3, 4, 5 or 6 carbon atoms, alkoxy having 1, 2, 3 or 4 carbon atoms or phenoxy,

which is unsubstituted or is substituted by 1-3 substituents selected from the group consisting of F, Cl, methyl and methoxy;

or

the other substituents R(1), R(2) and R(3)

independently of one another are alkyl-SOₓ, —CR(7)=CR(8)R(9) or —C≡CR(9);

x is zero, 1 or 2;

R(7) is hydrogen or methyl;

R(8) and R(9)

independently of one another are hydrogen, alkyl having 1, 2, 3 or 4 carbon atoms, cycloalkyl having 3, 4, 5, 6, 7 or 8 carbon atoms or phenyl,

which is unsubstituted or is substituted by 1-3 substituents selected from the group consisting of F, Cl, CF₃, methyl and methoxy;

or

the other substituents R(1), R(2) and R(3)

independently of one another are phenyl, C₆H₅—(C₁-C₄)-alkyl, naphthyl, biphenylyl, quinolinyl, isoquinolinyl or imidazolyl,

where quinolinyl, isoquinolinyl or imidazolyl are bonded via C or N and where phenyl, C₆H₅—(C₁-C₄)-alkyl, naphthyl, biphenylyl, quinolinyl, isoquinolinyl and imidazolyl are unsubstituted or are substituted by 1-3 substituents selected from the group consisting of F, Cl, CF₃, CH₃, methoxy, hydroxyl, amino, methylamino and dimethylamino;

or

the other substituents R(1), R(2) and R(3)

independently of one another are SR(10), —OR(10), —CR(10)R(11 )R(12);

R(10)

is —C_{f}H_{2f}—(C₃-C₈)-cycloalkyl, quinolinyl, isoquinolinyl, pyridinyl, imidazolyl or phenyl,

where the aromatic systems quinolinyl, isoquinolinyl, pyridinyl, imidazolyl and phenyl are unsubstituted or are substituted by 1-3 substituents selected from the group consisting of F, Cl, CF₃, CH₃, methoxy, hydroxyl, amino, methylamino and dimethylamino;

f is zero, 1 or 2;

R(11) and R(12)

independently of one another are defined as R(10), hydrogen or alkyl having 1, 2, 3 or 4 carbon atoms;

R(4) and R(5)

independently of one another are hydrogen, alkyl having 1, 2 or 3 carbon atoms, F, Cl, Br, I, CN, OR(13), NR(14)R(15), —(CH₂)ₙ—(CF₂)ₒ—CF₃;

R(13), R(14) and R(15)

independently of one another are hydrogen or alkyl having 1, 2, 3 or 4 carbon atoms;

n is zero or 1;

o is zero, 1 or 2;

and their pharmacologically acceptable salts;

(HOE 95/F 253—NZ 299 682)

ap) sulfonimidamides of the formula I

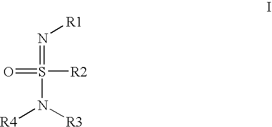

in which:

at least one of the three substituents R(1), R(2) and R(3) is a benzoylguanidine,

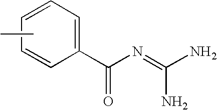

which is unsubstituted or substituted in the phenyl moiety by 1-4 radicals selected from the group consisting of alkyl having 1, 2, 3, 4, 5, 6, 7 or 8 carbon atoms, alkenyl having 2, 3, 4, 5, 6, 7 or 8 carbon atoms, —(CH₂)ₘ—R(14), F, Cl, Br, I, —C≡N, CF₃, R(22)SO₂—, R(23)R(24)N—CO—, R(25)-CO—, R(26)R(27)N—SO₂, —OR(35), —SR(35) or —NR(35)R(36);

m is zero, 1 or 2;

R(1 4)

is —(C₃-C₈)-cycloalkyl or phenyl,

which is not substituted or is substituted by 1-3 substituents selected from the group consisting of F and Cl, —CF₃, methyl, methoxy and —NR(15)R(1 6);

R(15) and R(16)

independently of one another are hydrogen or —CH₃;

R(22), R(23), R(25) and R(26) independently of one another are alkyl having 1, 2, 3, 4, 5, 6, 7 or 8 carbon atoms, alkenyl having 2, 3, 4, 5, 6, 7 or 8 carbon atoms, (CH₂) R(29) or —CF₃;

n is zero, 1, 2, 3 or 4;

R(29) is —(C₃-C₇)-cycloalkyl or phenyl,

which is not substituted or is substituted by 1-3 substituents selected from the group consisting of F, Cl, —CF₃, methyl, methoxy and —NR(30)R(31);

R(30) and R(31)

are hydrogen or alkyl having 1, 2, 3 or 4 carbon atoms;

or

R(23), R(25) and R(26)

are hydrogen;

R(24) and R(27)

independently of one another are hydrogen or alkyl having 1, 2, 3 or 4 carbon atoms;

or

R(23) and R(24), and also R(26) and R(27)

together are 5 or 6 methylene groups, of which one CH₂ group can be replaced by oxygen, —S—, —NH—, —NCH₃ or —N-benzyl;

R(35) and R(36)

independently of one another are hydrogen or alkyl having 1, 2, 3, 4, 5 or 6 carbon atoms;

or

R(35) and R(36)

together are 4-7 methylene groups, of which one CH₂ group can be replaced by oxygen, —S—, —NH—, —NCH₃ or —N-benzyl;

or

R(35)

is phenyl,

which is not substituted or is substituted by 1-3 substituents selected from the group consisting of F, Cl, —CF₃, methyl, methoxy, SO₂R(5), SO₂NR(6)R(7) and —NR(32)R(33);

R(5) is alkyl having 1, 2, 3, 4, 5 or 6 carbon atoms

R(6) and R(7)

independently of one another are hydrogen or alkyl having 1, 2, 3 or 4 carbon atoms;

R(32) and R(33)

independently of one another are hydrogen or alkyl having 1, 2, 3 or 4 carbon atoms;

or

R(35)

is C₁-C₉-heteroaryl,

which is unsubstituted or substituted by 1-3 substituents selected from the group consisting of F, Cl, CF₃, CH₃, methoxy, hydroxyl, amino, methylamino and dimethylamino;

and the other substituents R(1), R(2) and R(3) in each case

independently of one another are alkyl having 1, 2, 3, 4, 5, 6, 7 or 8 carbon atoms, (CH₂)ₚR(10)

p is zero, 1,2,3or4;

R(10) is phenyl,

which is not substituted or is substituted by 1-3 substituents selected from the group consisting of F, Cl, —CF₃, methyl, methoxy, —SO₂NR(17)R(8) and —SO₂R(9);

R(17) and R(8)

independently of one another are hydrogen or alkyl having 1, 2, 3 or 4 carbon atoms;

R(9) is alkyl having 1, 2, 3 or 4 carbon atoms;

or the other radical R(1) and R(3) in each case are hydrogen,

R(4) is hydrogen or alkyl having 1, 2, 3 or 4 carbon atoms; and their pharmaceutically tolerable salts;

(HOE 95/F 265—NZ 299 739)

ao) benzoylguanidines of the formula I

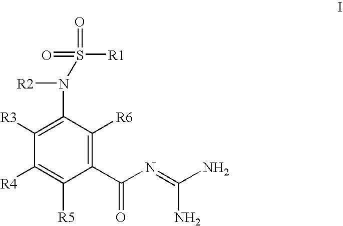

in which:

R(1) is alkyl having 1, 2, 3, 4, 5, 6, 7 or 8 carbon atoms or NR(7)R(8);

R(7) and R(8)

independently of one another are hydrogen or alkyl having 1, 2, 3,4, 5, 6, 7 or 8 carbon atoms;

R(2) is hydrogen, alkyl having 1, 2, 3, 4, 5, 6, 7 or 8 carbon atoms or —SO₂R(9);

R(9) independently is defined as R(1);

R(3) is hydrogen, —SR(25), —OR(25), —NR(25)R(26) or —CR(25)R(26)R(27);

R(25) is hydrogen, alkyl having 1, 2, 3, 4, 5, 6, 7 or 8 carbon atoms,

or phenyl,

which is unsubstituted or substituted by 1-3 substituents selected from the group consisting of F, Cl, CF₃, CH₃, methoxy, hydroxyl, amino, methylamino and dimethylamino;

or

R(25)

is —(C₁-C₉)-heteroaryl,

which is unsubstituted or substituted by 1-3 substituents selected from the group consisting of F, Cl, CF₃, CH₃, methoxy, hydroxyl, amino, methylamino and dimethylamino;

R(26) and R(27)

independently of one another are defined as R(25) or are hydrogen or alkyl having 1, 2, 3, 4, 5, 6, 7 or 8 carbon atoms;

R(4) is hydrogen, F, Cl, Br, I, OH, —C≡N, CF₃, alkyl having 1, 2, 3, 4, 5, 6, 7 or 8 carbon atoms, alkenyl having 2, 3, 4, 5, 6, 7 or 8 carbon atoms or —(CH₂)ₘR(14);

m is zero, 1 or 2;

R(14) is —(C₃-C₈)-cycloalkyl or phenyl,

which is not substituted or is substituted by 1-3 substituents selected from the group consisting of F and Cl, —CF₃, methyl, methoxy and —NR(15)R(16);

R(15) and R(16)

independently of one another are hydrogen or —CH₃;

R(5) and R(6)

independently of one another are hydrogen, alkyl having 1, 2, 3 or 4 carbon atoms, F, Cl, —OR(32), —NR(33)R(34) or CF₃;

R(32), R(33) and R(34)

independently of one another are hydrogen or alkyl having 1, 2, 3 or 4 carbon atoms;

and their pharmaceutically tolerable salts;

(HOE 95/F 269 K)

ar) benzenedicarboxylic acid diguanidides of the formula I

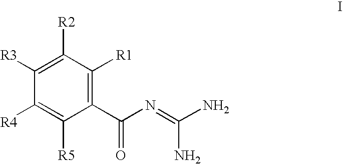

in which:

one of the radicals R(1), R(2), R(3) and R(4)

is —CO—N═C(NH₂)₂;

and the other radicals R(1), R(2), R(3) and R(4) in each case are:

R(1) is hydrogen, alkyl having 1, 2, 3 or 4 carbon atoms, F, Cl, Br, I, —OR(32), —NR(33)R(34) or CF₃;

R(32), R(33) and R(34)

independently of one another are hydrogen or alkyl having 1, 2, 3 or 4 carbon atoms;

R(2) and R(4)

independently of one another are hydrogen, F, Cl, Br, I, OH, —CN, CF₃, —CO—N═C(NH₂)₂, alkyl having 1, 2, 3, 4, 5, 6, 7 or 8 carbon atoms, alkenyl having 2, 3, 4, 5, 6, 7 or 8 carbon atoms or —(CH₂)ₘR(14);

m is zero, 1 or 2;

R(14) is —(C₃-C₈)-cycloalkyl or phenyl,

which is not substituted or is substituted by 1-3 substituents selected from the group consisting of F and C₁, —CF₃, methyl, methoxy and —NR(15)R(16);

R(15) and R(16)

are hydrogen or —CH₃;

or

R(2) and R(4)

independently of one another are pyrrol-1-yl, pyrrol-2-yl or pyrrol-3-yl,

not which is not substituted or is substituted by 1-4 substituents selected from the group consisting of F, Cl, Br, I, —CN, (C₂-C₈)-alkanoyl, (C₂-C₈)-alkoxycarbonyl, formyl, carboxyl, —CF₃, methyl, methoxy;

or

R(2) and R(4)

independently of one another are R(22)-SO₂—, R(23)R(24)N—CO—, R(28)-CO— or R(29)R(30)N—SO₂;

R(22) and R(28)

independently of one another are methyl or —CF₃;

R(23), R(24), R(29) and R(30)

independently of one another are hydrogen or methyl;

or

R(2) and R(4)

independently of one another are —OR(35) or —NR(35)R(36);

R(35) and R(36)

independently of one another are hydrogen or alkyl having 1, 2, 3, 4, 5 or 6 carbon atoms;

or

R(35) and R(36)

together are 4-7 methylene groups, of which one CH₂ group can be replaced by oxygen, —S—, —NH—, —NCH₃ or —N-benzyl;

R(3) is hydrogen, —SR(25), —OR(25), —NR(25)R(26), —CR(25)R(26)R(27);

R(25) is hydrogen, alkyl having 1, 2, 3, 4, 5, 6, 7 or 8 carbon atoms or phenyl,

which is unsubstituted or substituted by 1-3 substituents selected from the group consisting of F, Cl, CF₃, CH₃, methoxy, hydroxyl, amino, methylamino and dimethylamino;

or

R(25) is —(C₁-C₉)-heteroaryl,

which is unsubstituted or substituted by 1-3 substituents selected from the group consisting of F, Cl, CF₃, CH₃, methoxy, hydroxyl, amino, methylamino and dimethylamino;

R(26) and R(27)

independently of one another are defined as R(25) or are hydrogen or alkyl having 1, 2, 3, 4, 5, 6, 7 or 8 carbon atoms;

R(5) is alkyl having 1, 2, 3 or 4 carbon atoms, F, Cl, Br, I, X—(CH₂)_{y}—CF₃ or phenyl,

which is not substituted or is substituted by 1-3 substituents selected from the group consisting of F and Cl, —CF₃, methyl, methoxy and —NR(6)R(7);

R(6) and R(7)

independently of one another are hydrogen or —CH₃;

X is a bond or oxygen;

y is zero, 1 or 2;

and their pharmaceutically tolerable salts;

(HOE 95/F 269 BK)

as) benzenedicarboxylic acid diguanidides of the formula I

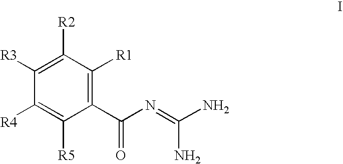

in which:

one of the radicals R(1), R(2), R(3) and R(5) is —CO—N═C(NH₂)₂;

and the other radicals R(1), R(2), R(3) and R(5) in each case are:

R(1) and R(5)

independently of one another are hydrogen, alkyl having 1, 2, 3 or 4 carbon atoms, F, Cl, —OR(32), —NR(33)R(34) or CF₃;

R(32), R(33) and R(34)

independently of one another are hydrogen or alkyl having 1, 2, 3 or 4 carbon atoms;

R(2) is hydrogen, F, Cl, Br, I, OH, —C≡N, CF₃, —CO—N═C(NH₂)₂, alkyl having 1, 2, 3, 4, 5, 6, 7 or 8 carbon atoms, alkenyl having 2, 3, 4, 5, 6, 7 or 8 carbon atoms or —(CH₂)ₘR(14);

m is zero, 1 or 2;

R(14) is —(C₃-C₈)-cycloalkyl or phenyl,

which is not substituted or is substituted by 1-3 substituents selected from the group consisting of F and Cl, —CF₃, methyl, methoxy and —NR(15)R(16);

R(15) and R(16)

independently of one another are hydrogen or —CH₃;

or

R(2) is R(22)-SO₂—, R(23)R(24)N—CO—, R(28)-CO— or R(29)R(30)N—SO₂;

R(22) and R(28)

independently of one another are methyl or —CF₃;

R(23), R(24), R(29) and R(30)

independently of one another are hydrogen or methyl;

or

R(2) is —OR(35) or —NR(35)R(36);

R(35) and R(36)

independently of one another are hydrogen or alkyl having 1, 2, 3, 4, 5 or 6 carbon atoms;

or

R(35) and R(36)

together are 4-7 methylene groups, of which one CH₂ group can be replaced by oxygen, —S—, —NH—, —NCH₃ or —N-benzyl;

R(3) is hydrogen, —SR(25), —OR(25), —NR(25)R(26), —CR(25)R(26)R(27);

R(25) is hydrogen, alkyl having 1, 2, 3, 4, 5, 6, 7 or 8 carbon atoms or phenyl,

which is unsubstituted or substituted by 1-3 substituents selected from the group consisting of F, Cl, CF₃, CH₃, methoxy, hydroxyl, amino, methylamino and dimethylamino;

or

R(25) is —(C₁-C₉)-heteroaryl,

which is unsubstituted or substituted by 1-3 substituents selected from the group consisting of F, Cl, CF₃, CH₃, methoxy, hydroxyl, amino, methylamino and dimethylamino;

R(26) and R(27)

independently of one another are defined as R(25) or are hydrogen or alkyl having 1, 2, 3, 4, 5, 6, 7 or 8 carbon atoms;

R(4) is CF₃, alkyl having 1, 2, 3, 4, 5, 6, 7 or 8 carbon atoms, alkenyl having 2, 3, 4, 5, 6, 7 or 8 carbon atoms, —(C₃-C₈)-cycloalkyl or —(CH₂)ₘR(14);

m is 1 or 2;

R(14) is —(C₃-C₈)-cycloalkyl or phenyl,

which is not substituted or is substituted by 1-3 substituents selected from the group consisting of F and Cl, —CF3, methyl, methoxy and —NR(15)R(16);

R(15) and R(16)

independently of one another are hydrogen or —CH₃;

or

R(4) is phenyl,

which is substituted by 2, 3, 4 or five substituents selected from the group consisting of F, Cl, CF₃, methyl, methoxy and —NR(15)R(16);

R(15) and R(16)

independently of one another are hydrogen or CH₃;

and their pharmaceutically tolerable salts;

(HOE 96/F 013)

at) diaryldicarboxylic acid diguanidides of the formula I

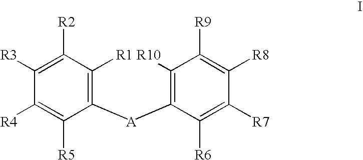

in which:

one of the radicals R(1), R(2), R(3), R(4) and R(5) is —CO—N═C(NH₂)₂;

the other radicals R(1) and R(5) in each case

independently of one another are hydrogen, alkyl having 1, 2, 3 or 4 carbon atoms, F, Cl, —OR(32), —NR(33)R(34) or CF₃;

R(32), R(33) and R(34)

independently of one another are hydrogen or alkyl having 1, 2, 3 or 4 carbon atoms;

the other radicals R(2) and R(4) in each case

independently of one another are hydrogen, F, Cl, Br, I, OH, —CN, CF₃, —CO—N═C(NH₂)₂, alkyl having 1, 2, 3, 4, 5, 6, 7 or 8 carbon atoms, alkenyl having 2, 3, 4, 5, 6, 7 or 8 carbon atoms or —(CH₂)ₘR(14);

m is zero, 1 or 2;

R(14) is —(C₃-C₈)-cycloalkyl or phenyl,

which is not substituted or is substituted by 1-3 substituents selected from the group consisting of F and Cl, —CF₃, methyl, methoxy and —NR(15)R(16);

R(15) and R(16)

are hydrogen or —CH₃;

or

the other radicals R(2) and R(4) in each case

independently of one another are pyrrol-1-yl, pyrrol-2-yl or pyrrol-3-yl,

which is not substituted or is substituted by 1-4 substituents selected from the group consisting of F, Cl, Br, I, —CN, (C₂-C₈)-alkanoyl, (C₂-C₈)-alkoxycarbonyl, formyl, carboxyl, —CF₃, methyl, methoxy;

or

the other radicals R(2) and R(4) in each case are R(22)-SO₂—, R(23)R(24)N—CO—, R(28)-CO— or R(29)R(30)N—SO₂;

R(22) and R(28)

independently of one another are methyl or —CF₃;

R(23), R(24), R(29) and R(30)

independently of one another are hydrogen or methyl;

or

the other radicals R(2) and R(4) in each case

independently of one another are —OR(35) or —NR(35)R(36);

R(35) and R(36)

independently of one another are hydrogen or alkyl having 1, 2, 3, 4, 5 or 6 carbon atoms;

or

R(35) and R(36)

together are 4-7 methylene groups, of which one CH₂ group can be replaced by oxygen, —S—, —NH—, —NCH₃ or —N-benzyl;

the other radical R(3) in each case

is hydrogen, —SR(25), —OR(25), —NR(25)R(26), —CR(25)R(26)R(27);

R(25) is hydrogen, alkyl having 1, 2, 3, 4, 5, 6, 7 or 8 carbon atoms or phenyl,

which is unsubstituted or substituted by 1-3 substituents selected from the group consisting of F, Cl, CF₃, CH₃, methoxy, hydroxyl, amino, methylamino and dimethylamino;

or

R(25) is —(C₁-C₉)-heteroaryl,

which is unsubstituted or substituted by 1-3 substituents selected from the group consisting of F, Cl, CF₃, CH₃, methoxy, hydroxyl, amino, methylamino and dimethylamino;

R(26) and R(27)

independently of one another are defined as R(25) or are hydrogen or alkyl having 1, 2, 3, 4, 5, 6, 7 or 8 carbon atoms;

one of the radicals R(6), R(7), R(8), R(9) and R(10)

is —CO—N═C(NH₂)₂;

the other radicals R(6) and R(10) in each case

independently of one another are hydrogen, alkyl having 1, 2, 3 or 4 carbon atoms, F, Cl, —OR(132), —NR(133)R(134) or CF₃;

R(132), R(133) and R(134)

independently of one another are hydrogen or alkyl having 1, 2, 3 or 4 carbon atoms;

the other radicals R(7) and R(9) in each case

independently of one another are hydrogen, F, Cl, Br, I, OH, —CN, CF₃, —CO—N═C(NH₂)₂, alkyl having 1, 2, 3, 4, 5, 6, 7 or 8 carbon atoms, alkenyl having 2, 3, 4, 5, 6, 7 or 8 carbon atoms or —(CH₂)ₘₘR(114);

mm is zero, 1 or 2;

R(114)

is —(C₃-C₈)-cycloalkyl or phenyl,

which is not substituted or is substituted by 1-3 substituents selected from the group consisting of F and Cl, —CF₃, methyl, methoxy and —NR(115)R(116);

R(115) and R(116)

are hydrogen or —CH₃;

or

the other radicals R(7) and R(9) in each case

independently of one another are pyrrol-1-yl, pyrrol-2-yl or pyrrol-3-yl,

which is not substituted or is substituted by 1-4 substituents selected from the group consisting of F, Cl, Br, I, —CN, (C₂-C₈)-alkanoyl, (C₂-C₈)-alkoxycarbonyl, formyl, carboxyl, —CF₃, methyl and methoxy;

or

the other radicals R(7) and R(9) in each case

are R(122)-SO₂—, R(123)R(124)N—CO—, R(128)-CO— or R(129)R(130)N—SO₂;

R(122) and R(128)

independently of one another are methyl or —CF₃;

R(123), R(124), R(129) and R(130)

independently of one another are hydrogen or methyl;

or

the other radicals R(7) and R(9) in each case

independently of one another are —OR(135) or —NR(135)R(136);

R(135) and R(136)

independently of one another are hydrogen or alkyl having 1, 2, 3, 4, 5 or 6 carbon atoms;

or

R(135) and R(136)

together are 4-7 methylene groups, of which one CH₂ group can be replaced by oxygen, —S—, —NH—, —NCH₃ or —N-benzyl;

the other radical R(8) in each case

is hydrogen, —SR(125), —OR(125), —NR(125)R(126) or —CR(125)R(126)R(127);

R(125)

is hydrogen, alkyl having 1, 2, 3, 4, 5, 6, 7 or 8 carbon atoms or phenyl,

which is unsubstituted or substituted by 1-3 substituents selected from the group consisting of F, Cl, CF₃, CH₃, methoxy, hydroxyl, amino, methylamino and dimethylamino;

or

R(125)

is —(C₁-C₉)-heteroaryl,

which is unsubstituted or substituted by 1-3 substituents selected from the group consisting of F, Cl, CF3, CH₃, methoxy, hydroxyl, amino, methylamino and dimethylamino;

R(126) and R(127)

independently of one another are defined as R(125) or are hydrogen or alkyl having 1, 2, 3, 4, 5, 6, 7 or 8 carbon atoms;

A is absent or is —NR(11)-CO—, —NR(12)-CO—NR(13)-, —NR(17)-CO—NR(18)-SO₂—, —NR(19)-SO₂—, —SO₂—NR(1 )-SO₂—, —SO₂—NR(19)-CO—, —O—CO—NR(19)-SO₂— or —CR(20)=CR(21)-;

R(11), R(12), R(13), R(17), R(18), R(19), R(20) and R(21)

independently of one another are hydrogen or alkyl having 1, 2, 3, 4, 5, 6, 7 or 8 carbon atoms

and their pharmaceutically tolerable salts;

(HOE 961F 026)

au) substituted thiophenylalkenylcarboxylic acid guanidides of the formula I

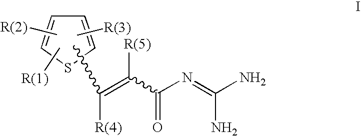

in which:

at least one of the substituents R(1), R(2) and R(3)

is —Oₚ—(CH₂)ₛ—C_{q}F_{2q+1}, R(40)CO— or R(31 )SOₖ—;

p is zero or 1;

s is zero, 1, 2, 3 or 4;

q is 1, 2, 3, 4, 5, 6, 7 or 8;

k is zero, 1 or 2;

R(40) is alkyl having 1, 2, 3, 4, 5, 6, 7 or 8 carbon atoms, perfluoroalkyl having 1, 2, 3, 4, 5, 6, 7 or 8 carbon atoms, cycloalkyl having 3, 4, 5, 6, 7 or 8 carbon atoms, or phenyl,

which is not substituted or is substituted by 1-3 substituents selected from the group consisting of F, Cl, CF₃, methyl and methoxy;

R(31) is alkyl having 1, 2, 3, 4, 5, 6, 7 or 8 carbon atoms, perfluoroalkyl having 1, 2, 3, 4, 5, 6, 7 or 8 carbon atoms, cycloalkyl having 3, 4, 5, 6, 7 or 8 carbon atoms, or phenyl,

which is not substituted or is substituted by 1-3 substituents selected from the group consisting of F, Cl, CF₃, methyl or methoxy;

or

R(31) is NR(41)R(42);

R(41) and R(42)

independently of one another are hydrogen, alkyl having 1, 2, 3 or 4 carbon atoms, perfluoroalkyl having 1, 2, 3 or 4 carbon atoms,

or

R(41) and R(42)

together are 4 or 5 methylene groups, of which one CH₂ group can be replaced by oxygen, S, NH, N—CH₃ or N-benzyl;

and the other substituents R(1), R(2) and R(3) in each case independently of one another are H, F, Cl, Br, I, CN, —Oₙₐ—CₘₐH₂ₘₐ₊₁ or —O_{ga}CᵣₐH₂ᵣₐR(10);

na is zero or 1;

ma is zero, 1,2,3,4,5,6,7or8;

ga is zero or 1;

ra is zero, 1, 2, 3 or 4;

R(10) is cycloalkyl having 3, 4, 5, 6, 7 or 8 carbon atoms or phenyl,

Where the phenyl is not substituted or is substituted by 1-3 substituents selected from the group consisting of F, Cl, CF₃, methyl and methoxy;

R(4) and R(5)

independently of one another are hydrogen, F, Cl, Br, I, CN, alkyl having 1, 2, 3, 4, 5, 6, 7 or 8 carbon atoms, perfluoroalkyl having 1, 2, 3, 4, 5, 6, 7 or 8 carbon atoms, cycloalkyl having 3, 4, 5, 6, 7 or 8 carbon atoms or phenyl,

which is not substituted or is substituted by 1-3 substituents selected from the group consisting of F, Cl, CF₃, methyl, methoxy and NR(14)R(15);

R(14) and R(15)

independently of one another are H, alkyl having 1, 2, 3 or 4 carbon atoms or perfluoroalkyl having 1, 2, 3 or 4 carbon atoms;

and their pharmaceutically tolerable salts;

(HOE 96/F 032)

av) ortho-substituted benzoylguanidines of the formula I

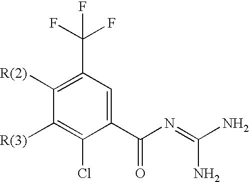

in which:

R(2) and R(3)

independently of one another are hydrogen, Cl, Br, I, (C₁-C₈)-alkyl, (C₃-C₈)-cycloalkyl or —OR(5);

R(5) is (C₁-C₈)-alkyl or C_{d}H_{2d}—(C₃-C₈)-cycloalkyl;

d is zero, 1 or 2;

where one of the two substituents R(2) and R(3) is always hydrogen but both substituents R(2) and R(3) are not simultaneously hydrogen, and their pharmaceutically tolerable salts;

(HOE 96/F 042)

ax) benzoylguanidines of the formula I

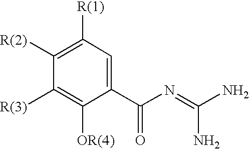

in which:

R(1) is H, F, Cl, Br, I, CN, NO₂, alkyl having 1, 2, 3, 4, 5, 6, 7 or 8 carbon atoms, alkoxy having 1, 2, 3, 4, 5, 6, 7 or 8 carbon atoms, cycloalkyl having 3, 4, 5, 6, 7 or 8 carbon atoms, cycloalkoxy having 3, 4, 5, 6, 7 or 8 carbon atoms or Xₐ—(CH₂)_{b}—(CF₂)_{c}—CF₃;

X is oxygen, S, NR(5),

a is zero or 1;

b is zero, 1 or 2;

c is zero, 1, 2 or 3;

R(5) is H, alkyl having 1, 2, 3 or 4 carbon atoms or —C_{d}H_{2d}R(6);

d is zero, 1, 2, 3 or 4;

R(6) is cycloalkyl having 3, 4, 5, 6, 7 or 8 carbon atoms, phenyl, biphenylyl or naphthyl,

where the aromatics phenyl, biphenylyl or naphthyl are not substituted or are substituted by 1-3 substituents selected from the group consisting of F, Cl, CF₃, methyl, methoxy and NR(7)R(8);

R(7) and R(8)

independently are H or alkyl having 1, 2, 3 or 4 carbon atoms;

or

R(1) is —SR(10), OR(10) or —CR(10)R(11)R(12);

R(10) is —C_{f}H_{2f}-cycloalkyl having 3, 4, 5, 6, 7 or 8 carbon atoms in the cycloalkyl ring, or phenyl,

where phenyl is unsubstituted or substituted by 1-3 substituents selected from the group consisting of F, Cl, CF₃₁ CH₃, methoxy, hydroxyl, amino, methylamino and dimethylamino;

f is zero, 1 or 2;

R(11) and R(12)

independently of one another are defined as R(10) or are hydrogen or alkyl having 1, 2, 3 or 4 carbon atoms;

or

R(1) is phenyl, naphthyl, biphenylyl or heteroaryl having 1, 2, 3, 4, 5, 6, 7, 8 or 9 carbon atoms, with the latter being linked via a carbon atom or a nitrogen atom of the ring,

which are in each case unsubstituted or substituted by 1-3 substituents selected from the group consisting of F, Cl, CF₃, CH₃, methoxy, hydroxyl, amino, methylamino and dimethylamino,

or

R(1) is —SR(13), —OR(13), —NHR(13), —NR(13)R(14), —CHR(13)R(15), —C[R(15)R(16)OH], —C≡CR(18), —C[R(19)]=CHR(18), —C[R(20)R(21)]ₖ-(CO)—[CR(22)R(23)]ₗ-R(24),

k is zero, 1, 2, 3 or 4;

l is zero, 1, 2, 3 or 4;

R(13) and R(14)

identically or differently are —(CH₂)_{g}—(CHOH)ₕ—(CH₂)ᵢ—(CHOH)ⱼ—R(17) or —(CH₂)_{g}—O—(CH₂—CH₂O)ₕ—R(24);

R(17) is hydrogen or methyl,

g, h and i

identically or differently are zero, 1, 2, 3 or 4;

j is 1, 2, 3 or 4;

R(15) and R(16)

identically or differently are hydrogen, alkyl having 1, 2, 3, 4, 5 or 6 carbon atoms or, together with the carbon atom carrying them, are cycloalkyl having 3, 4, 5, 6, 7 or 8 carbon atoms;

R(18)

is phenyl,

which is unsubstituted or substituted by 1-3 substituents selected from the group consisting of F, Cl, CF₃, methyl, methoxy and NR(25)R(26);

R(25) and R(26)

are H or alkyl having 1, 2, 3 or 4 carbon atoms;

or

R(18) is heteroaryl having 1, 2, 3, 4, 5, 6, 7, 8 or 9 carbon atoms,

which is unsubstituted or substituted as phenyl;

or

R(18) is alkyl having 1, 2, 3, 4, 5 or 6 carbon atoms,

which is unsubstituted or substituted by 1-3 OH;

or

R(18)

is cycloalkyl having 3, 4, 5, 6, 7 or 8 carbon atoms;

R(19), R(20), R(21), R(22) and R(23)

identically or differently are hydrogen or methyl;

R(24) is H, alkyl having 1, 2, 3, 4, 5 or 6 carbon atoms, cycloalkyl having 3, 4, 5, 6, 7 or 8 carbon atoms or —CₘH₂ₘ—R(18);

m is1,2,3or4;

R(2) and R(3)

are defined as R(1);

R(4) is alkyl having 1, 2, 3 or 4 carbon atoms;

and their pharmaceutically tolerable salts;

(HOE 96/F 043)

av) ortho-substituted benzoylguanidines of the formula I

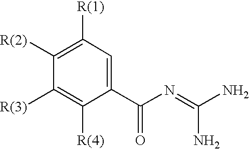

in which:

R(1) is H, F, Cl, Br, I, CN, NO₂, alkyl having 1, 2, 3, 4, 5, 6, 7 or 8 carbon atoms, alkoxy having 1, 2, 3, 4, 5, 6, 7 or 8 carbon atoms, cycloalkyl having 3, 4, 5, 6, 7 or 8 carbon atoms, cycloalkoxy having 3, 4, 5, 6, 7 or 8 carbon atoms or Xₐ—(CH₂)_{b}—(CF₂)_{c}—CF₃;

X is oxygen, S, NR(5),

a is zero or 1;

b is zero, 1 or 2;

c is zero, 1, 2 or 3;

R (5) is H, alkyl having 1, 2, 3 or 4 carbon atoms or —C_{d}H_{2d}R(⁶);

d is zero, 1, 2, 3 or 4;

R(6) is cycloalkyl having 3, 4, 5, 6, 7 or 8 carbon atoms, phenyl, biphenylyl or naphthyl,

where the aromatics phenyl, biphenylyl or naphthyl are unsubstituted or substituted by 1-3 substituents selected from the group consisting of F, Cl, CF₃, methyl, methoxy and NR(7)R(8);

R(7) and R(8)

independently are H or alkyl having 1, 2, 3 or 4 carbon atoms;

or

R(1) is —SR(10), —OR(10) or —CR(10)R(11)R(12);

R(10) is —C_{f}H_{2f}—Cycloalkyl having 3, 4, 5, 6, 7 or 8 carbon atoms In the cycloalkyl ring, or phenyl,

where phenyl is unsubstituted or substituted by 1-3 substituents selected from the group consisting of F, Cl, CF₃, CH₃, methoxy, hydroxyl, amino, methylamino and dimethylamino;

f is zero, 1 or 2;

R(11) and R(12)

independently of one another are defined as R(10), or hydrogen or alkyl having 1, 2, 3 or 4 carbon atoms;

or

R(1) is phenyl, naphthyl, biphenylyl or heteroaryl having 1, 2, 3, 4, 5, 6, 7, 8 or 9 carbon atoms, with the latter being linked via a carbon atom or a nitrogen atom of the ring,

which are in each case unsubstituted or substituted by 1-3 substituents selected from the group consisting of F, Cl, CF₃, CH₃, methoxy, hydroxyl, amino, methylamino and dimethylamino,

or

R(1) is —SR(13), —OR(13), —NHR(13), —NR(13)R(14), —CHR(13)R(15), —C[R(15)R(16)OH], —C≡CR(18), —C[R(19)]=CHR(18), —C[R(20)R(21)]ₖ-(CO)—[CR(22)R(23)]ₗ-R(24),

k is zero, 1, 2, 3 or 4;

l is zero, 1, 2, 3 or 4;

R(13) and R(14)

identically or differently are —(CH₂)_{g}—(CHOH)ₕ—(CH₂)ᵢ—(CHOH)ⱼ—R(17) or —(CH₂)_{g}—O—(CH₂—CH₂O)ₕ—R(24);

R(17) is hydrogen or methyl,

g, h and i

identically or differently are zero, 1, 2, 3 or 4;

is 1, 2, 3 or 4;

R(15) and R(16)

identically or differently are hydrogen, alkyl having 1, 2, 3, 4, 5 or 6 carbon atoms or, together with the carbon atom carrying them, are cycloalkyl having 3, 4, 5, 6, 7 or 8 carbon atoms;

R(1 8)

is phenyl,

which is unsubstituted or substituted by 1-3 substituents selected from the group consisting of F, Cl, CF₃, methyl, methoxy and NR(25)R(26);

R(25) and R(26)

are H or alkyl having 1, 2, 3 or 4 carbon atoms;

or

R(18) is heteroaryl having 1, 2, 3, 4, 5, 6, 7, 8 or 9 carbon atoms,

which is unsubstituted or substituted as phenyl;

or

R(18) is alkyl having 1, 2, 3, 4, 5 or 6 carbon atoms,

which is unsubstituted or substituted by 1-3 OH;

or

R (18)

is cycloalkyl having 3, 4, 5, 6, 7 or 8 carbon atoms;

R(19), R(20), R(21), R(22) and R(23) identically or differently are hydrogen or methyl;

R(24) is H, alkyl having 1, 2, 3, 4, 5 or 6 carbon atoms, cycloalkyl having 3, 4, 5, 6, 7 or 8 carbon atoms or —CₘH₂ₘ—R(18);

m is 1, 2, 3 or 4;

one of the two substituents R(2) and R(3)

is hydroxyl;

and

the other of the substituents R(2) and R(3) in each case is defined as R(1);

R(4) is alkyl having 1, 2, 3 or 4 carbon atoms; alkoxy having 1, 2, 3 or 4 carbon atoms, F, Cl, Br, I or —(CH₂)ₙ—(CF₂)ₒ—CF₃;

n is zero or 1;

o is zero or 1;

and their pharmaceutically tolerable salts.

(DE 195 02 895, DE 44 30 212, EP 667 341, DE 44 04 183, EP 708 088, EP 723 963, EP 0 694 537, DE 44 21 495, EP 699 660, EP 699 663, EP 699 666, DE 43 37 611, EP 0719 766, WO 94/26709, WO 96 04 241, EP 726 254, U.S. Pat. No. 4 251 545, DE 35 02 629, WO 84/00875, Kumamoto et al., Pharm. Bull. [1966], 7-13; U.S. Pat. No. 3,780,027, JP 8225513)

II. Also suitable are compounds of the formula

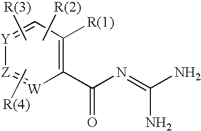

in which:

W, Y and Z

are a nitrogen atom or a carbon atom substituted by R(2) or R(3) or R(4);

R(1) is hydrogen, A, Hal, —CF₃, —CH₂F, —CHF₂, —CH₂CF₃, —C₂F₅, —CN,

—NO₂, -ethynyl, or an X—R′;

A is alkyl having 1 to 6 carbon atoms;

Hal is F, Cl, Br or I;

X is oxygen, S or NR″;

R″ is hydrogen, A or a cyclic methylene chain having 3 to 7 carbon atoms;

R′ is H, A, HO—A—, HOOC—A—, (C₃-C₇)-cycloalkyl, (C₆-C₈)-cycloalkylalkyl, CF₃, CH₂F, CHF₂, CH₂—CF₃, Ph, —CH₂-Ph or Het;

Ph is phenyl, naphthyl or biphenylyl which is unsubstituted or mono-, di- or trisubstituted by A, OA, NR′R″, Hal, CF₃;

Het is a mono- or binuclear saturated, unsaturated or aromatic heterocycle having 1 to 4 nitrogen, oxygen and/or sulfur atoms,

which is unsubstituted or mono-, di- or trisubstituted by Hal, CF₃, A, OH, OA, —X—R′, —CN, —NO₂, and/or carbonyl oxygen,

where Het is bonded via N or an alkylene chain CₘH₂ₘ

where m=zero to 6;

or

R′ and R″

together are alkylene having 4-5 carbon atoms, in which one CH₂ group can also be replaced by oxygen, S, NH, N-A, N-Ph and N—CH₂-Ph;

R(2) and R(3)

independently of one another are hydrogen, Hal, A, HO-A-, X—R′, —C(═N—OH)-A, A-O—CO—(C₁-C₄)-alkyl-, CN, NO₂, COOH, halogen-substituted A, in particular CF₃, CH₂F, CHF₂, C₂Fs, CH₂CF₃, or S(O)ₙR′″;

R′″ is A, Ph or -Het;

n is zero, 1 or 2;

or

R(2) and R(3)

independently of one another are SO₂NR′R″, Ph or —O-Ph, —O—CH₂-Ph, —CO-A, —CHO, —COOA, —CSNR′R″, CONR′R−, —CH═CH—COOH, —CH═CH—COOA, indenyl, indanyl, decahydronaphthyl, cyclopentenyl, dihydrothienyl, dihydrofuryl, heterobicyclyl, alkylthienyl, halothienyl, haloalkylthienyl, acylthienyl, halofuryl, haloalkylfuryl or pyrrolyl;

or

R(2) and R(3)

independently of one another are R(5)-O—;

R(5) is hydrogen, A, (C₁-C₆)-alkenyl or (C₃-C₇)-cycloalkyl;

R(4) is Ph, Het, —O-Het; CF₃, S(O)ₙR′″, —SO₂NR′R″, alk;

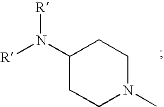

or

two of the substituents R(1) to R(4)

together are a group —O—CR(6)R(7)-CO—NR(8)-, or

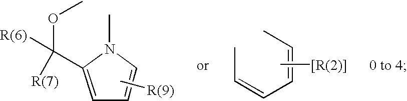

where R(2) has the meaning indicated;

R(6), R(7), R(8) and R(9)

independently of one another are H or A;

or

R(8) is (C₅-C₇)-cycloalkyl;

or

R(9) is cyano;

alk is straight-chain or branched (C₁-C₈)-alkyl or (C₃-C₈)-cycloalkyl, which is unsubstituted or mono-, di- or trisubstituted by A;

or

alk is an ethenyl or ethynyl radical which is substituted by H, A, Ph or Het:

[DE 41 27 026, DE 4337609, JP 07025768, Edward J. Cragoe, Jr., DIURETICS (Chemistry, Pharmacology and Medicine), J. Wiley & Sons (1983), 303-341]

III. Compounds of the formula

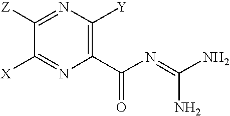

in which:

X is H, Hal, (Hal)₃C—, (C₁-C₆)-alkyl, (C₃-C₆)-cycloalkyl, substituted phenyl, (C₁-C₅)-alkyl-S— or (C₁-C₅)-alkyl-SO₂—;

Y is NH₂ or substituted amino;

or

X and Z

together are a —(CH₂)₄— or a 1,3-butadienylene chain;

or

Z is H, Hal, OH, HS, (C₁-C₅)-alkyl, (C₃-C₆)-cycloalkyl, substituted phenyl;

or

Z is an amino group —NR(1)R(2);

R(1) is H, straight- or branched-chain, optionally substituted (C₁-C₈)-alkyl,

which can be interrupted by oxygen;

or

R(1) is (C₃-C₈)-alkenyl, (C₃-C₈)-alkynyl, (C₃-C₇)-cycloalkyl or OH-substituted phenyl or OH-substituted phenyl-(C₁-C₄)-alkyl or OH-substituted (C₃-C₇)-cycloalkyl;

R(2) is 1-morpholino, hydrogen or a straight or branched (C₁-C₈)-alkyl chain,

which can be interrupted by oxygen or an amino group,

which straight or branched (C₁-C₈)-alkyl chain is unsubstituted or substituted by

a substituted or unsubstituted mono- or polynuclear heterocycle which contains nitrogen, oxygen or sulfur atoms;

or

which alkyl chain is substituted by phenyl,

optionally mono- or polysubstituted by (C₁-C₄)-alkoxy, optionally substituted by OH, alkylamino, alkyl or phenyl;

or

by an aminocarbonyl group

or

by hydroxyl or (C₁-C₄)-alkoxy groups,

or

R(2) is phenyl,

unsubstituted or substituted by alkyl, alkoxy, an amino group, which as substituents carries:

H, a mono- or polynuclear heterocycle which contains nitrogen, oxygen or sulfur atoms,

which is unsubstituted or substituted by H, Hal or (C₁-C₄)-alkyl:

a phenyl radical,

unsubstituted or substituted by a substituent selected from the group consisting of (C₁-₄)-alkyl, (C₁-C₄)-alkoxy, Hal and OH;

or

R(2) is 1-piperidino,

unsubstituted or substituted in the 4-position by an acyl radical of an aliphatic, alicyclic, aromatic or heteroaromatic carboxylic acid, (C₁-C₈)-alkyl, which for its part can be substituted by OH or (C₁-C₄)-alkoxy or a (C₁-C₄)-alkoxy-substituted phenyl radical;

or

R(2) is amidino,

which is unsubstituted or substituted by phenyl,

which is unsubstituted or substituted by Hal or alkyl;

or

R(2) is an acyl radical of an aliphatic, alicyclic, aromatic or heteroaromatic carboxylic acid,

or

R(2) is a (C₁-C₈)-alkyl chain, which can be substituted by a phenyl radical carrying OH, alkoxy or alkyl radicals,

or

R(1) and R(2)

together with the nitrogen atom to which they are bonded, are a piperazine ring,

which is unsubstituted or via a (C₁-C₆)-methylene chain carries a mono- or polynuclear heterocycle,

which contains nitrogen, oxygen or sulfur,

Hal is F, Cl, Br or I.

(EP 708 091, EP 622 356, JP 5-125085)

IV. Likewise suitable are indoloylguanidine derivatives of the formula

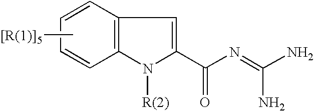

in which

R(2) is hydrogen, unsubstituted or substituted (C₁-C₈)-alkyl, (C₃-C₇)-cycloalkyl, OH, (C₁-C₆)-alkyl-O—, an aromatic radical or a group —CH₂—R(20);

R(20) is (C₂-C₆)-alkenyl or (C₂-C₆)-alkynyl;

R(1) is 1 to 5 identical or different substituents, which are:

hydrogen, unsubstituted or substituted (C₁-C₈)-alkyl, (C₂-C₆)-alkenyl, (C₂-C₆)-alkynyl, (C₃-C₇)-cycloalkyl, halogen, —NO₂, (C₂-C₈)-alkanoyl, arylalkanoyl having up to 10 carbon atoms, aroyl having up to 11 carbon atoms, —COOH, (C₂-C₆)-alkoxycarbonyl, an aromatic group or one of the following mentioned groups: —OR(3), —NR(6)R(7) or —S(O)ₙR(40);

R(3) is hydrogen, (C₁-C₈)-alkyl, substituted (C₁-C₈)-alkyl, (C₃-C₇)-cycloalkyl, an aromatic radical or a group CH₂—R(30) R(30) is alkenyl or alkynyl;

R(6) and R(7)

independently of one another are hydrogen, unsubstituted or substituted (C₁-C₈)-alkyl, (C₃-C₇)-cycloalkyl, (C₂-C₈)-alkanoyl, an arylalkanoyl group having up to 10 carbon atoms, an aroyl group having up to 11 carbon atoms, an aromatic group or —CH₂—R(60);

R(60) is (C₂-C₆)-alkenyl or (C₂-C₆)-alkynyl;

or

R(6) and R(7)

together with the nitrogen atom are a 5-7-membered cyclic amine, which can additionally contain further heteroatoms in the ring;

n is zero, 1 or 2;

R(40) is unsubstituted or substituted (C₁-C₈)-alkyl, or an aromatic group, or a group

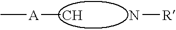

A is oxygen, —S(O)ₙ— or —N(R50)-;

R(50) is hydrogen or (C₁-C₈)-alkyl;

R′ is hydrogen, unsubstituted or substituted (C₁-C₈)-alkyl, in which the ring represents a saturated 3-8-membered heterocycle having a nitrogen atom,

said substituted alkyl carries one or more groups selected from the group consisting of halogen, —OH, (C₁-C₆)-alkoxy, —CN, —COOH, (C₂-C₆)-alkoxycarbonyl, (C₂-C₈)-alkanoyl, arylalkanoyl having up to 10 carbon atoms, aroyl having up to 11 carbon atoms, an aromatic group, —CONR(4)R(5),

R(4) and R(5)

identically or differently are hydrogen or (C₁-C₈)-alkyl;

or

R(4) and R(5)

are connected to one another and together form a 5-7-membered cyclic amine which can additionally contain further heteroatoms in the ring,

or said substituted alkyl carries a group

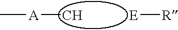

in which:

E is a nitrogen atom or a CH group;

R″ is hydrogen, (C₁-C₈)-alkyl or substituted (C₁-C₈)-alkyl which is unsubstituted or substituted by OH, (C₁-C₆)-alkoxy, —CN, —COOH, (C₂-C₆)-alkoxycarbonyl, (C₂-C₈)-alkanoyl, aralkanoyl having up to 10 carbon atoms, aroyl having up to 11 carbon atoms, an aromatic group, —NR(6)R(7), —CONR(4)R(5);

R(4) and R(5)

independently of one another are hydrogen or (C₁-C₈)-alkyl; where the cyclic system of the formula

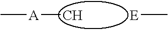

is a 3-8-membered saturated aliphatic or heterocyclic ring system having a nitrogen atom,

and where the aromatic groups mentioned are an aryl radical having up to 10 carbon atoms, a 5- or 6-membered heteroaryl radical having 1-4 nitrogen atoms, a 5- or 6-membered heteroaryl group containing 1 or 2 nitrogen atoms and a heteroatom which is oxygen or sulfur, or furyl, and where the aryl radicals mentioned can be unsubstituted or substituted by unsubstituted (C₁-C₈)-alkyl or substituted (C₁-C₈)-alkyl, halogen, —NO₂, (C₂-C₆)-alkoxycarbonyl, COOH, —OR(3), NR(6)R(7), —CONR(4)R(5), —SO₂NR(6)R(7) or S(O)ₙR(40),

where R(1) and the guanidinocarbonyl radical can be in any desired position of the 5- or 6-membered ring of the indole system, and the appropriate pharmaceutically tolerable salts.

(WO 95 04052)

V. Additionally suitable are heterocyclic guanidine derivatives of the formula

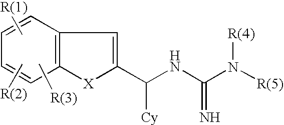

in which:

X is —O—, —S—, —NH—, —N[(C₁-C₄)-alkyl-O— or —N(phenyl)-;

R(1), R(2) and R(3)

are hydrogen, halogen, (C₁-C₄)-alkyl, (C₁-C₄)-alkyl-O—, phenyl, benzyl;

or

two of the substituents R(1), R(2) and R(3)

together with one side of the benzo system are a 4-6-membered carbocyclic ring;

R(4) and R(5)

independently of one another are hydrogen, (C₁-C₁₂)-alkyl, benzhydryl, aralkyl,

which is unsubstituted or substituted by one or more substituents from the groups halogen, (C₁-C₄)-alkyl, (C₁-C₄)-alkyl-O— or —CF₃, —(CH₂)ₘ—CH₂-T,

m is zero to 3;

T is —CO—O—T(1);

T(1) is hydrogen or (C₁-C₄)-alkyl;

Cy is a benzo-fused unsaturated or dihydro-5-membered ring heterocycle

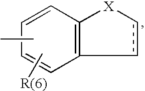

a pyrazole or imidazole ring of the formula

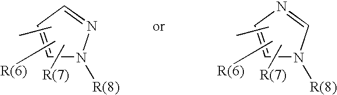

a naphthyl radical or a dihydro- or tetrahydronaphthyl radical

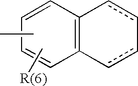

a 2-, 3- or 4-pyridyl radical

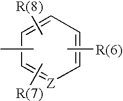

Z is N— or CH;

a thienyl radical

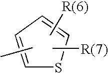

R(6) is hydrogen, halogen, hydroxyl, (C₁-C₁₀)-alkyl, (C₁-C₁₀)-alkyl-O—, phenoxy, (C₁-C₁₀)-alkyloxymethyloxy- or —(O)ₙS—R(9);

R(9) is (C₁-C₁₀)-alkyl, thienyl, pyridyl, thiazolyl, thiadiazolyl, imidazolyl, pyrazolyl or phenyl,

each of which is unsubstituted or mono- or disubstiluted by halogen, (C₁-C₄)-alkyl or (C₁-C₄)-alkyl-O—;

R(7) and R(8)

is hydrogen, halogen, hydroxyl, (C₁-C₁₀)-alkyl,

(C₁-C₁₀)-alkyl-O—, phenyl, phenoxy or

(C₁-C₁₀)-alkoxymethyloxy;

or

Cy is phenyl,

which is unsubstituted or mono- or disubstituted by halogen, (C₁-C₄)-alkyl or (C₁-C₄)-alkyl-O—;

or

Cy is -Gr-Am;

Gr is —R(13)-R(12)-(CH₂)_{q}—C[W][W(1)]-(CH₂)_{q′}—; R(13)R(14)- or —R(15)-;

R(12) is a single bond, —O—, —(O)ₙS—, —CO— or —CONH—;

R(13) is a single bond, phenyl, thienyl, pyridyl, thiazolyl, thiadiazolyl, imidazolyl or pyrazolyl;

R(14) is a single bond or SO₂—;

R(15) is (C₂-C₁₀)-alkenyl- or (C₂-C₁₀)-alkynyl;

W and W(1)

independently of one another are hydrogen, (C₁-C₄)-alkyl;

or

W and W(1)

cyclically connected to one another are a (C₃-C₈-hydrocarbon ring;

q and q′

are zero to 9;

Am is —NR(10)R(11);

R(10) is hydrogen, (C₁-C₄)-alkyl or benzyl,

R(11) is (C₁-C₄)-alkyl, phenyl or benzyl;

or

R(10) and R(11)

together are a (C₃-C₁₀)-alkylene group,

which is unsubstituted or substituted by —COOH, (C₁-C₅)-alkoxycarbonyl, (C₂-C₄)-hydroxyl-alkylene or benzyl;

or

Am is pyrrolyl, pyridyl, pyrazolyl, morpholinyl, dihydropyridyl, tetrahydropyridyl, quinuclidinyl, imidazolyl, 3-azabicyclo[3.2.1]octyl,

which is unsubstituted or substituted by (C₁-C₄)-alkyl,

or

Am is azabicyclo[3.2.2]nonyl;

or

Am is a piperazine group of the formula

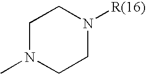

R(1 6) is hydrogen, (C₁-C₄)-alkyl, (C₃-C₆)-cycloalkyl, phenyl, tolyl, methoxyphenyl, halophenyl, diphenylmethylene, benzyl or pyridyl;

or

Am is an azido group —(O)ₜ—(CH₂)_{q}—C[W][W(1)]-(CH₂)q′—N₃;

t is zero or 1;

where W and W(1) have the previously indicated meaning; and the optical enantiomers and the pharmacologically tolerable salts.

Preferably, NHE exchange inhibitors of subtype 3 are used:

(HOE 93/F 223 K—EP 639 573, NZ 264 130)

r) benzo-fused 5-membered ring heterocycles, of the formula I

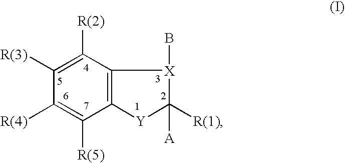

in which:

X is N or CR(6);

Y is oxygen, S or NR(7);

A, B together are a bond

or

A, B are both hydrogen, if X is CR(6) and Y is simultaneously NR(7);

one of the substituents R(1) to R(6) is a —CO—N═C(NH₂)₂ group;

the other substituents R(1) to R(6) in each case are hydrogen, F, Cl, Br, I or (C₁-C₆)-alkyl;

up to two of the other substituents R(1) to R(6) are CN, NO₂, N₃, (C₁-C₄)-alkyloxy or CF₃;

up to one of the other substituents

is R(8)-CₙH₂ₙ-Z-;

n is zero to 10;

where the alkylene chain —CₙH₂ₙ— is straight-chain or branched and where a carbon atom can be replaced by an oxygen or sulfur atom or by a nitrogen atom;

R(8) is hydrogen, (C₂-C₆)-alkenyl or (C₃-C₁₀)-cycloalkyl, which is unsubstituted or substituted by 1 to 4 methyl groups or an OH group, or can contain an ethylene group —CH═CH—, and wherein a methylene group can be replaced by an oxygen or sulfur atom or by a nitrogen atom;

or

R(8) is phenyl,

which is unsubstituted or substituted by 1 to 3 substituents selected from the group consisting of F, Cl, Br, I, CF₃, CH₃—S(O)ₛ— or R(9)-W_{y}—,

s is zero, 1 or 2;

R(9) is H, methyl, ethyl,

W is oxygen or NR(10);

R(10) is H or methyl;

y is zero or 1;

or

R(8) is CₘF₂ₘ₊₁;

m is 1 to 3;

or

R(8) is 1- or 2-naphthyl, pyridyl, quinolyl or isoquinolyl;

Z is —CO—, —CH₂— or —[CR(1)(OH)]_{q}—;

q is 1, 2or3;

R(11) is H or methyl;

or

Z is oxygen or —NR(12)-;

R(12) is H or methyl;

or

Z is —S(O)ₛ—;

s is zero, 1 or 2;

or

Z is —SO₂—NR(13)-;

R(13) is H or (C₁-C₄)-alkyl;

R(7) is hydrogen, (C₁-C₁₀)-alkyl, (C₂-C₁₀)-alkenyl or R(8)-CₙH₂ₙ—;

and their pharmaceutically tolerable salts;

(HOE 93/F 436—EP-Offenlegungsschrift 659 748), NZ 270 264)

v) acylguanidines of the formula I

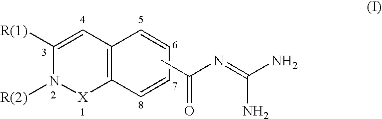

in which:

x is carbonyl, sulfonyl,

R(1) is H, (C₁-C₈)-alkyl,

unsubstituted or substituted by hydroxyl,

(C₃-C₈)-cycloalkyl, phenyl,

which is unsubstituted or substituted by 1-3 substituents from the group consisting of F, Cl, CF₃, CH₃, methoxy, hydroxyl, amino, methylamino or dimethylamino,

R(2) is H, (C₁-C₄)-alkyl,

and their pharmaceutically tolerable salts;

(HOE 94/F 014 K—EP-Offenlegungsschrift 666 252, NZ 270 370) w) phenyl-substituted alkylcarboxylic acid guanidides, carrying perfluoroalkyl groups, of the formula I

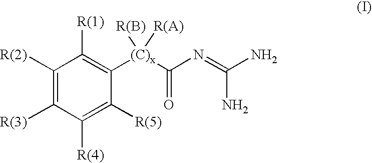

in which:

R(A) is hydrogen, F, Cl, Br, I, CN, OR(6), (C₁-C₈)-alkyl, (C₃-C₈)— cycloalkyl, Oᵣ(CH₂)₂CₐF_{2b+1} or NR(7)R(8);

r is zero or1;

a is zero, 1, 2, 3 or 4;

b is 1, 2, 3, 4, 5, 6, 7 or 8;

R(6) is hydrogen, (C₁-C₈)-alkyl, (C₃-C₈)-alkenyl, (C₃-C₈)-cycloalkyl, phenyl or benzyl,

where the aromatics are not substituted or are substituted by 1-3 substituents from the group consisting of F, Cl, CF₃, methyl, methoxy and NR(9)R(10);

R(9) and R(10)

are H, (C₁-C₄)-alkyl or (C₁-C₄)-perfluoroalkyl;

R(7) and R(8)

independently of one another are defined as R(6);

R(B) independently is defined as R(A);

X is 1, 2 or 3;

R(1) is hydrogen, (C₁-C₈)-alkyl, (C₃-C₈)-cycloalkyl, —Oₜ(CH₂)_{d}CₑF₂ₑ₊₁, F, Cl, Br, I or CN;

t is zero or 1;

d is zero, 1, 2, 3 or 4;

e is 1, 2, 3, 4, 5, 6, 7 or 8;

R(2), R(3), R(4) and R(5)

independently of one another are defined as R(1);

but with the condition that at least one of the substituents R(1), R(2), R(3), R(4), R(5), R(A) and R(B) is an —Oₜ(CH₂)_{d}CₑF₂ₑ₊₁ or an Oᵣ(CH₂)ₐC_{b}F_{2b+1} group, and their pharmaceutically tolerable salts;

(HOE 94/F 123—EP-Offenlegungsschrift 682 017, NZ 272 058)

y) bicyclic heteroaroylguanidines of the formula I

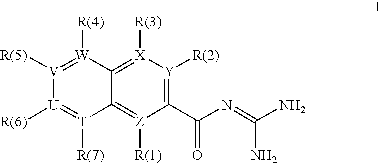

in which:

T, U, V, W, X, Y and Z

independently of one another are nitrogen or carbon;

but with the restriction that X and Z are not simultaneously nitrogen,

and that T, U, V, W, X, Y and Z carry no substituents when they are nitrogen,

and that no more than four of them are simultaneously nitrogen,

R(1) and R(2)

independently of one another are hydrogen, F, Cl, Br, I, (C₁-C₃)-alkyl, (C₁-C₃)-perfluoroalkyl, OR(8), NR(8)R(9) or C(═O)N═C(NH₂)₂;

R(8) and R(9)

independently of one another are hydrogen or (C₁-C₃)-alkyl,

or

R(8) and R(9)

together are 4 or 5 methylene groups, of which one CH₂ group can be replaced by oxygen, S, NH, N—CH₃ or N-benzyl;

R(3), R(4), R(5), R(6) and R(7)

independently of one another are hydrogen, F, Cl, Br, I, —C—N, Xₖ—(CH₂)ₚ—(C_{q}F_{2q+1}), R(10a)-SO_{bm}, R(10b)R(10c)N—CO, R(11)-CO— or R(12)R(13)N—SO₂—,

where the perfluoroalkyl group is straight-chain or branched;

X is oxygen, S or NR(14);

R(14) is H or (C₁-C₃)-alkyl;

bm is zero, 1 or 2;

p is zero, 1 or 2;

k is zero or 1;

q is 1, 2, 3, 4, 5 or 6;

R(10a), R(10b), R(11) and R(12)

independently of one another are (C₁-C₈)-alkyl, (C₃-C₆)-alkenyl, —CₙH₂ₙ—R(15) or (C₁-C₈)-perfluoroalkyl;

n is zero, 1, 2, 3 or 4;

R(1 5) is (C₃-C₇)-cycloalkyl or phenyl, which is not substituted or is substituted by 1-3 substituents selected from the group consisting of F, Cl, CF₃, methyl, methoxy and NR(16)R(17);

R(16) and R(17)

are H or C₁-C₄-alkyl;

or

R(10b), R(11) and R(12)

are hydrogen;

R(10c) and R(13)

independently are hydrogen or (C₁-C₄)-alkyl;

or

R(10b) and R(10c) and also R(12) and R(13)

together are 4 or 5 methylene groups, of which one CH₂ group can be replaced by oxygen, sulfur, NH, N—CH₃ or N-benzyl;

or

R(3), R(4), R(5), R(6) and R(7)

independently of one another are (C₁-C₈)-alkyl, —CₐₗH₂ₐₗR(18) or (C₃-C₈)-alkenyl;

al is zero, 1 or 2;

R(18) is (C₃-C₈)-cycloalkyl, phenyl, biphenylyl or naphthyl, where the aromatic are not substituted or are substituted by 1-3 substituents selected from the group consisting of F, Cl, CF₃, methyl, methoxy and NR(19a)R(19b);

R(19a) and R(19b)

are hydrogen, (C₁-C₄)-alkyl or (C₁-C₄)-perfluoroalkyl;

or

R(3), R(4), R(5), R(6) and R(7)

independently of one another are (C₁-C₉)-heteroaryl which is linked via C or N and which is unsubstituted or substituted by 1-3 substituents from the group consisting of F, Cl, CF₃, CH₃, methoxy, hydroxyl, amino, methylamino or dimethylamino;

or

R(3), R(4), R(5), R(6) and R(7)

independently of one another are

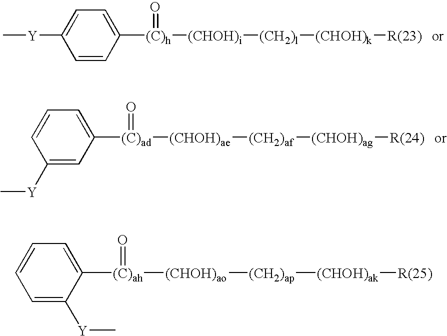

Y is oxygen, —S— or —NR(22)-;

h, ad, ah

independently of one another are zero or 1;

i, j, k, ae, af, ag, ao, ap and ak

independently of one another are zero, 1, 2, 3 or 4;

but where in each case

h, i and k are not simultaneously zero,

ad, ae and ag are not simultaneously zero, and

ah, ao and ak are not simultaneously zero,

R(23), R(24) R(25) and R(22)

independently of one another are hydrogen or (C₁-C₃)-alkyl;

or

R(3), R(4), R(5), R(6) and R(7)

independently of one another are SR(29), —OR(30), —NR(31)R(32) or —CR(33)R(34)R(35);

R(29), R(30), R(31) and R(33)

independently of one another are —CₐH₂ₐ—(C₁-C₉)-heteroaryl, which is unsubstituted or substituted by 1-3 substituents selected from the group consisting of F, Cl, CF₃, CH₃, methoxy, hydroxyl, amino, methylamino and dimethylamino;

a is zero, 1 or 2;

R(32), R(34) and R(35)

independently of one another are defined as R(29) or hydrogen, (C₁-C₄)-alkyl or (C₁-C₄)-perfluoroalkyl;

or

R(3), R(4), R(5), R(6) and R(7)

independently of one another are

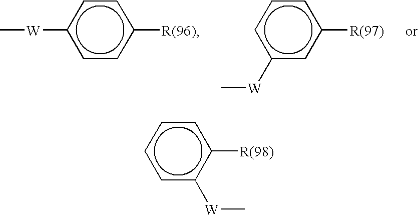

R(96), R(97) and R(98)

independently of one another are (C₁-C₉)-heteroaryl, which is linked via C or N and which is unsubstituted or substituted by 1 to 3 substituents from the group consisting of F, Cl, CF₃, CH₃, methoxy, hydroxyl, amino, methylamino, dimethylamino or benzyl;

W is oxygen, S or NR(36)-;

R(36) is H or (C₁-C₄)-alkyl;

or

R(3), R(4), R(5), R(6) and R(7)

independently of one another are R(46)X(1)-;

X(1) is oxygen, S, NR(47), (D=O)A- or NR(48)C=MN^{(*)}R(49)-;

M is oxygen or sulfur;

A is oxygen or NR(50);

D is C or SO;

R(46) is (C₁-C₈)-alkyl, (C₃-C₈)-alkenyl, (CH₂)_{b}C_{d}F_{2d+1} or —CₓH₂ₓ—R(51);

b is zero or 1;

d is 1, 2, 3, 4, 5, 6 or 7;

x is zero, 1, 2, 3 or 4;

R(51) is (C₃-C₈)-cycloalkyl, phenyl, biphenylyl or naphthyl,

where the aromatic are not substituted or are substituted by 1-3 substituents selected from the group consisting of F, Cl, CF₃, methyl, methoxy and NR(52)R(53);

R(52) and R(53)

are hydrogen, (C₁-C₄)-alkyl or (C₁-C₄)-perfluoroalkyl;

R(47), R(48) and R(50) independently are hydrogen, (C₁-C₄)-alkyl or (C₁-C₄)-perfluoroalkyl;

R(49) is defined as R(46);

or

R(46) and R(47), or R(46) and R(48)

together are 4 or 5 methylene groups, of which one CH₂ group can be replaced by oxygen, sulfur, NH, N—CH₃ or N-benzyl;

where A and N^{(*)} are bonded to the phenyl nucleus of the heteroaroylguanidine parent structure;

or

R(3), R(4), R(5), R(6) and R(7)

independently of one another are —SR(64), —OR(65), —NHR(66), —NR(67)R(68), —CHR(69)R(70) or —CR(54)R(55)OH, —C≡CR(56), —CR(58)=CR(57) or —[CR(59)R(60)]ᵤ-CO—[CR(61)R(62)]ᵥ-R(63);

R(64), R(65), R(66), R(67) and R(69)

identically or differently are —(CH₂)_{y}—(CHOH)_{z}—(CH₂)ₐₐ—(CHOH)ₜ—R(71) or —(CH₂)_{ab}—O—(CH₂—CH₂O)_{ac}—R(72);

R(71) and R(72)

independently of one another are hydrogen or methyl;

u is 1, 2, 3 or 4;

v is zero, 1, 2, 3 or 4;

y, z, aa identically or differently are zero, 1, 2, 3 or 4;

t is 1, 2, 3 or 4;

R(68), R(70), R(54) and R(55)

identically or differently are hydrogen or (C₁-C₆)-alkyl;

or

R(69) and R(70), or R(54) and R(55)

together with the carbon atom carrying them, are a (C₃-C₈)-cycloalkyl;

R(63)

is hydrogen, (C₁-C₆)-alkyl, (C₃-₈)-cycloalkyl or —CₑH₂ₑ—R(73);

e is zero, 1, 2, 3 or 4;

R(56), R(57) and R(73) independently

are phenyl, which is unsubstituted or substituted by 1-3 substituents selected from the group consisting of F, Cl, CF₃, methyl, methoxy and NR(74)R(75);

R(74) and R(75)

are hydrogen or (C₁-C₄)-alkyl;

or

R(56), R(57) and R(73) independently

are (C₁-C₉)-heteroaryl, which is unsubstituted or substituted as phenyl;

R(58), R(59), R(60), R(61) and R(62)

are hydrogen or methyl;

or

R(3), R(4), R(5), R(6) and R(7)

independently of one another are R(76)-NH—SO₂—;

R(76) is R(77)R(78)N—(C═Y′)—;

Y′ is oxygen, S or N—R(79);

R(77) and R(78)

identically or differently are hydrogen, (C₁-C₈)-alkyl, (C₃-C₆)-alkenyl or —C_{f}H_{2f}—R(80);

f is zero, 1, 2, 3 or 4;

R(80) is (C₅-C₇)-cycloalkyl or phenyl, which is unsubstituted or substituted by 1-3 substituents selected from the group consisting of F, Cl, CF₃, methoxy and (C₁-C₄)-alkyl;

or

R(77) and R(78)

together are 4 or 5 methylene groups, of which one CH₂ group can be replaced by oxygen, sulfur, NH, N—CH₃ or N-benzyl;

R(79) is defined as R(77) or is amidine;

or

R(3), R(4), R(5), R(6) and R(7)

independently of one another are NR(84a)R(85), OR(84b), SR(84c) or —CₙH₂ₙ—R(84d);

n is zero, 1, 2, 3 or 4;

R(84d) is (C₃-C₇)-cycloalkyl or phenyl, which is not substituted or is substituted by 1-3 substituents from the group consisting of F, Cl, CF₃, methyl, methoxy and NR(16)R(17);

R(16) and R(17)

are hydrogen or C₁-C₄-alkyl;

R(84a), R(84b), R(84c) and R(85)

independently of one another are hydrogen, (C₁-C₈)-alkyl, (C₁-C₈)-perfluoroalkyl or (CH₂)ₐ—R(84g);

ax is zero, 1, 2, 3 or 4;

R(84g) is (C₃-C₇)-cycloalkyl or phenyl, which is not substituted or is substituted by 1-3 substituents selected from the group consisting of F, Cl, CF₃, methyl, methoxy and NR(84u)R(84v);

R(84u) and R(84v)

are hydrogen or C₁-C₄-alkyl;

or

R(84a) and R(85)

together are 4 or 5 methylene groups, of which one CH₂ group can be replaced by oxygen, sulfur, NH, N—CH₃ or N-benzyl,

and their pharmaceutically tolerable salts;

(HOE 94/F 168—EP-Offenlegungsschrift 690 048, NZ 272 373)

ab) phenyl-substituted alkenylcarboxylic acid guanidides, carrying perfluoroalkyl groups, of the formula I

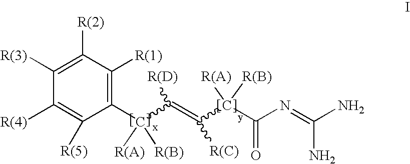

in which:

R(A) is hydrogen, F, Cl, Br, I, CN, OH, OR(6), (C₁-C₈)-alkyl, Oᵣ(CH₂)ₐC_{b}F_{2b+1}, (C₃-C₈)-cycloalkyl or NR(7)R(8);

r is zero or 1;

a is zero, 1, 2, 3 or 4;

b is 1, 2, 3, 4, 5, 6, 7 or 8;

R(6) is (C₁-C₈)-alkyl, (C₁-C₄)-perfluoroalkyl, (C₃-C₈)-alkenyl, (C₃-C₈)-cycloalkyl, phenyl or benzyl;

where the aromatic are not substituted or are substituted by 1-3 substituents from the group consisting of F, Cl, CF₃, methyl, methoxy and NR(9)R(10);

R(9) and R(10)

are H, (C₁-C₄)-alkyl or (C₁-C₄)-perfluoroalkyl;

R(7) and R(8)

independently of one another are defined as R(6);

or

R(7) and R(8)

together are 4 or 5 methylene groups, of which one CH₂ group can be replaced by oxygen, sulfur, NH, N—CH₃ or N-benzyl;

R(B) independently is defined as R(A);

X is zero, 1 or 2;

Y is zero, 1 or 2;

R(C) is hydrogen, F, Cl, Br, I, CN, OR(12), (C₁-C₈)-alkyl, Oₚ(CH₂)_{f}C_{g}F_{2g+1} or (C₃-C₈)-cycloalkyl;

p is zero or 1;

f is zero, 1, 2, 3 or 4;

g is 1, 2, 3, 4, 5, 6, 7 or 8;

R(12)

is (C₁-C₈)-alkyl, (C₁-C₄)-perfluoroalkyl, (C₃-C₈)-alkenyl, (C₃-C₈)-cycloalkyl, phenyl or benzyl;

where the aromatics phenyl or benzyl are not substituted or are substituted by 1-3 substituents from the group consisting of F, Cl, CF₃, methyl, methoxy and NR(13)R(14);

R(13) and R(14)

independently of one another are H, (C₁-C₄)-alkyl or (C₁-C₄)-perfluoroalkyl;

R(D) independently is defined as R(C),

R(1) is hydrogen, (C₁-C₈)-alkyl, —Oₜ(CH₂)_{d}CₑF₂ₑ₊₁, (C₃-C₈)-cycloalkyl, F, Cl, Br, I or CN;

t is zero or 1;

d is zero, 1, 2, 3 or 4;

e is 1, 2, 3, 4, 5, 6, 7 or 8;

R(2), R(3), R(4) and R(5)

independently of one another are defined as R(1);

but with the condition that at least one of the substituents R(A), R(B), R(C), R(D), R(1), R(2), R(4) or R(5) is a Oᵣ(CH₂)ₐC_{b}F_{2b+1}, Oₚ(CH₂)_{f}C_{g}F_{2g+1} or Oₜ(CH₂)_{d}CₑF₂ₑ₊₁ group and R(3) is not a Oₜ(CH₂)_{d}CₑF₂ₑ₊₁ group;

and their pharmaceutically tolerable salts;

(HOE 95/F 007 K—EP-Offenlegungsschrift 723 956, NZ 280 887)

ah) benzoylguanidines of the formula I

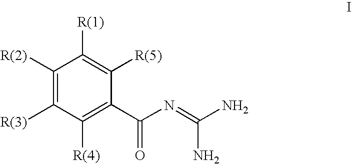

in which:

one of the three substituents R(1), R(2) and R(3)

is R(6)-A-B-D;

R(6) is a basic protonatable radical, i.e. an amino group —NR(7)R(8), an amidino group R(7)R(8)N—C[═N—R(9)]— or a guanidino group

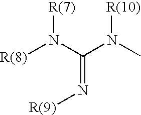

R(7), R(8), R(9) and R(10)

independently of one another are hydrogen or alkyl having 1, 2, 3 or 4 carbon atoms;

or

R(7) and R(8)

together are C₈H₂ₐ;

a is 4, 5, 6 or 7;

where if a=5, 6 or 7 a methylene group of the group CₐH₂ₐ can be replaced by a heteroatom group O, SOₘ or NR(11),

or

R(8) and R(9) or R(9) and R(10) or R(7) and R(10)

are a group CₐH₂ₐ;

a is 2, 3, 4 or 5;

where if a=3, 4 or 5 a methylene group of the group CₐH₂ₐ can be replaced by a heteroatom group O, SOₘ or NR(11);

m is zero, 1 or 2;

R(11) is hydrogen or methyl;

or

R(6) is a basic heteroaromatic ring system having 1-9 carbon atoms;

A is C_{b}H_{2b};

b is 1, 2, 3, 4, 5, 6, 7, 8, 9 or 10;

where in the group C_{b}H_{2b} one or two methylene groups can be replaced by one of the groupings selected from the group consisting of —O—, —CO—, —CH[OR(20)]—, —SOₘ—, —NR(20)-, —NR(20)-CO—, —NR(20)-CO—NH—, —NR(20)-CO—NH—SO₂—

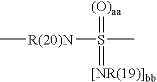

and —SOₐₐ[NR(19)]_{bb}-;

and where in the group C_{b}H_{2b} a methylene group can be replaced by —CH—R(99), where R(99) together with R(7) forms a pyrrolidine or piperidine ring;

aa is 1 or 2;

bb is 0 or 1;

aa+bb=2;

R(19) is hydrogen or alkyl having 1, 2, 3 or 4 carbon atoms,

R(20) is hydrogen or methyl;

or —NR(18)-SO₂—;

R(18) is hydrogen or methyl;

v is zero, 1 or 2;

R(17) is hydrogen, cycloalkyl having 3, 5 or 6 carbon atoms or CₖF₂ₖ₊₁—;

k is 1, 2 or 3,

or

R(17) is pyrrol-1-yl, pyrrol-2-yl or pyrrol-3-yl,

which is not substituted or is substituted by 1-4 substituents selected from the group consisting of F, Cl, Br, I, —CN, (C₂-C₈)-alkanoyl, (C₂-C₈)-alkoxycarbonyl, formyl, carboxyl, —CF₃, methyl and methoxy;

or

R(1 7) is —(C₃-C₈)-cycloalkyl or phenyl,

which is not substituted or is substituted by 1-3 substituents selected from the group consisting of F and Cl, —CF₃, methyl, hydroxyl, methoxy, —NR(37)R(38), CH₃SO₂— and H₂NO₂S—;

R(37) and R(38)

are hydrogen or —CH₃;

R(4) and R(5)

independently of one another are hydrogen, alkyl having 1, 2, 3 or 4 carbon atoms, F, Cl, —OR(32), —NR(33)R(34) or —CᵣF₂ᵣ₊₁;

R(32), R(33) and R(34)

independently of one another are hydrogen or alkyl having 1, 2 or 3 carbon atoms;

r is 1, 2, 3 or 4;

and their pharmacologically tolerable salts;

(HOE 95/F 072—EP-Offenlegungsschrift 738 712, NZ 286 380

ai) indenoylguanidines of the formula I

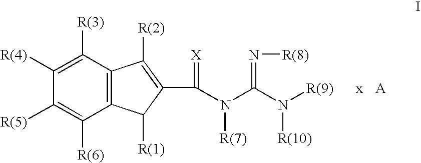

in which:

R(1) and R(2)

independently of one another are hydrogen, alkyl having 1, 2, 3, 4, 5, 6, 7, 8, 9 or 10 carbon atoms, cycloalkyl having 3, 4, 5 or 6 carbon atoms, O-alkyl having 1, 2, 3 or 4 carbon atoms, O—C(═O)— alkyl having 1, 2, 3 or 4 carbon atoms or CₘH₂ₘ—NR(12)R(13);

R(12) and R(13)

independently of one another are hydrogen or alkyl having 1, 2, 3 or 4 carbon atoms;

m is zero, 2, 3 or 4;

NH—C(═O)—NH₂, C(═O)—O-alkyl having 1, 2, 3 or 4 carbon atoms, C(═O)—NH₂, C(═O)—NH-alkyl having 1, 2, 3 or 4 carbon atoms, C(═O)—N(alkyl)₂ having 1, 2, 3 or 4 carbon atoms in each alkyl group, alkenyl having 2, 3, 4, 5, 6, 7, 8, 9 or 10 carbon atoms, alkynyl having 2, 3, 4, 5, 6, 7, 8, 9 or 10 carbon atoms, alkylaryl having 1, 2, 3 or 4 carbon atoms in the alkyl group, alkenylaryl having 2, 3, 4, 5, 6, 7, 8, 9 or 10 carbon atoms in the alkenyl group, alkynylaryl having 2, 3, 4, 5, 6, 7, 8, 9 or 10 carbon atoms in the alkynyl group, C₁-C₄-alkyl-substituted aryl, C₁-C₄-alkylheteroaryl, C₁-C₄-alkenylheteroaryl, aminoalkylaryl having 1, 2, 3 or 4 carbon atoms in the alkyl group, substituted aryl, heteroaryl and substituted heteroaryl;

R(3), R(4), R(5) and R(6)

independently of one another are hydrogen, alkyl having 1, 2, 3, 4, 5, 6, 7, 8, 9 or 10 carbon atoms, O-alkyl having 1, 2, 3, 4, 5, 6, 7, 8, 9 or 10 carbon atoms, halogen, (such as F, Cl, Br, I), OH, aryl, substituted aryl, heteroaryl, substituted heteroaryl, O-lower-alkyl, O-aryl, O-lower-alkylaryl, O-substituted aryl, O-lower-alkyl-substituted aryl, O—C(═O)—C₁-C₄-alkylaryl, O—C(═O)—NH—C₁-C₄-alkyl, O—C(═O)—N(C₁-C₄-alkyl)₂, NO₂, CN, CF₃, NH₂, NH—C(═O)—C₁-C₄-alkyl, NH—C(═O)—NH₂, COOH, C(═O)—O—C₁-C₄-alkyl, C(═O)—NH₂, C(═O)—NH—C₁-C₄-alkyl, C(═O)—N(C₁-C₄-alkyl)₂, C₁-C₄-COOH, C₁-C₄-alkyl-C(═O)—O—C₁-C₄-alkyl, SO₃H, SO₂-alkyl, SO₂-alkylaryl, SO₂—N-(alkyl)₂, SO₂—N(alkyl)(alkylaryl), C(═O)—R(11), C₁-C₁₀-alkyl-C(═O)-R(11), C₂-C₁₀-alkenyl-C(═O)—R(11), C₂-C₁₀-alkynyl-C(═O)—R(11), NH—C(═O)—C₁-C₁₀-alkyl-C(═O)—R(11), O—C₁-C₁ -alkyl-C(═O)—R(11);

R(11) is C₁-C₄-alkyl, C₁-C₄-alkynyl, aryl, substituted aryl, NH₂, NH-C₁-C₄-alkyl, N—(C₁-C₄-alkyl)₂, SO₃H, SO₂-alkyl, SO₂-alkylaryl, SO₂—N-(alkyl)₂, SO₂—N(alkyl)(alkylaryl);

x is O, S or NH;

R(7), R(8), R(9) and R(10)

independently of one another are hydrogen, alkyl, cycloalkyl, aryl, alkylaryl;

or

R(8) and R(9)

together are part of a 5, 6 or 7-membered heterocyclic ring;

A is absent or is a nontoxic organic or inorganic acid.

(HOE 95/F 109—EP 748 795, NZ 286 583)

ak) benzyloxycarbonylguanidines of the formula I

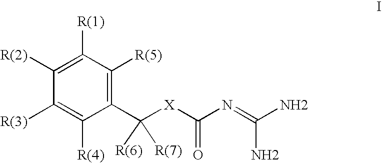

in which:

R(1), R(2) and R(3)

independently of one another are —Y-[4-R(8)-phenyl], —Y-[3-R(8)-phenyl] or —Y-[2-R(8)-phenyl],

where the phenyl in each case is unsubstituted or substituted by 1-2 substituents from the group consisting of F, Cl, —CF₃, methyl, hydroxyl, methoxy and —NR(96)R(97);

R(96) and R(97)

independently of one another are hydrogen or —CH₃;

Y is a bond, CH₂, oxygen, —S— or —NR(9);

R(9) is hydrogen or alkyl having 1, 2, 3 or 4 carbon atoms;

R(8) is SOₐ[NR(98)]_{b}NR(99)R(10);

a is 1 or 2;

b is 0 or 1;

a+b=2;

R(98), R(99) and R(10)

independently of one another are hydrogen, —(C₁-C₈)-alkyl, benzyl, —(C₂-C₈)-alkylene-NR(11 )R(12), (C₂-C₈-alkylene-NR(13)-(C₂-C₈)-alkylene-NR(37)R(38) or (C₀-C₈)-alkylene-CR(39)R(40)CR(41 )R(42)(C₀-C₈)-alkylene-NR(43)R(44);

R(11), R(12), R(13), R(37), R(38), R(43) and R(44) independently of one another are hydrogen, —(C₁-C₈-alkyl or benzyl:

R(39), R(40), R(41) and R(42)

independently of one another are hydrogen, —(C₁-C₈)-alkyl or —(C₀-C₃)-alkylenephenyl,

where the phenyl is not substituted or is substituted by 1-3 substituents selected from the group consisting of F, Cl, —CF₃, methyl and methoxy;

or

R(99) and R(10)

together are 4-6 methylene groups, of which one CH₂ group can be replaced by oxygen, —S—, —NH—, —N—CH₃ or —N-benzyl;

or

R(8) is SOₐ[NR(98)]_{b}NR(95)-C[═N—R(94)]-NR(93)R(92);

R(92), R(93), R(94) and R(95)

independently of one another are hydrogen or alkyl having 1, 2, 3 or 4 carbon atoms;

or

R(1), R(2) and R(3)

independently of one another are pyrrol-1-yl, pyrrol-2-yl or pyrrol-3-yl,

which is not substituted or is substituted by 1-4 substituents selected from the group consisting of F, Cl, Br, I, —CN, (C₂-C₈)-alkanoyl, (C₂-C₈)-alkoxycarbonyl, formyl, carboxyl, —CF₃, methyl, methoxy;

or

R(1), R(2) and R(3)

independently of one another are hydrogen, —(C₁-C₈)-alkyl, —(C₂-C₈)-alkenyl or —(CH₂)ₘR(14);

m is zero, 1 or 2;

R(14) is —(C₃-C₈)-cycloalkyl or phenyl,

which is not substituted or is substituted by 1-3 substituents selected from the group consisting of F and Cl, —CF₃, methyl, methoxy and —NR(15)R(16);

R(15) and R(16)

are hydrogen or —CH₃;

or

R(1), R(2) and R(3)

independently of one another are -Q-4-[(CH₂)ₖ—CHR(17)-(C═O)R(20)]-phenyl, -Q-3-(CH₂)ₖ—CHR(17)-(C═O)R(20)]-phenyl or -Q-2-[(CH₂)ₖ—CHR(17)-(C═O)R(20)]-phenyl,

where the phenyl in each case is unsubstituted or substituted by 1-2 substituents from the group consisting of F, Cl, —CF₃, methyl, hydroxyl, methoxy and —NR(35)R(36);

R(35) and R(36)

independently of one another are hydrogen or —CH₃;

Q is a bond, oxygen, —S— or —NR(18);

R(18) is hydrogen or —(C₁-C₄)-alkyl;

R(17) is —OR(21) or —NR(21)R(22):

R(21) and R(22)

independently of one another are hydrogen, —(C₁-C₈)-alkyl, —(C₁-C₈)-alkanoyl, —(C-C₈)-alkoxycarbonyl, benzyl, benzyloxycarbonyl;

or

R(21) is trityl;

R(20) is —OR(23) or —NR(23)R(24);

R(23), R(24) independently of one another are hydrogen, —(C₁-C₈)-alkyl or benzyl;

k is zero, 1, 2, 3 or 4;

or

R(1), R(2) and R(3)

independently of one another are (C₁-C₉)-heteroaryl,

which is linked via C or N and which is unsubstituted or substituted by 1-3 substituents from the group consisting of F, Cl, CF₃, CH₃, methoxy, hydroxyl, amino, methylamino and dimethylamino;

or

R(1), R(2) and R(3)

are —SR(25), —OR(25), —NR(25)R(26), —CR(25)R(26)R(27);

R(25) is —C_{f}H_{2f}(C₁-C₉)-heteroaryl,

which is unsubstituted or substituted by 1-3 substituents from the group consisting of F, Cl, CF₃, CH₃, methoxy, hydroxyl, amino, methylamino and dimethylamino;

f is zero, 1 or 2;

R(26) and R(27)

independently of one another are defined as R(25) or are hydrogen or (C₁-C₄)-alkyl,

or

R(1), R(2) and R(3)

independently of one another are (C₁-C₉)-heteroaryl-N-oxide,

which is linked via C or N and which is unsubstituted or substituted by 1-3 substituents selected from the group —NR(49)R(50);

R(49) and R(50)

are hydrogen or alkyl having 1, 2, 3 or 4 carbon atoms;

or

R(32), R(34) and R(45)

are hydrogen;

R(33) and R(46)

independently of one another are hydrogen or alkyl having 1, 2, 3 or 4 carbon atoms;

or

R(32) and R(33) and also R(45) and R(46)

together are 5 or 6 methylene groups, of which one CH₂ group can be replaced by oxygen, —S—, —NH—, —NCH₃ or —N-benzyl;

or

R(1), R(2) and R(3)

independently of one another are R(51)-A-G-D-;

R(51) is a basic protonatable radical, i.e. an amino group —NR(52)R(53), an amidino group R(52)R(53)N—C[═N—R(54)]- or a guanidino group R(52)R(53)N—C[═N—R(54)]-NR(55)-;

R(52), R(53), R(54) and R(55)

independently of one another are hydrogen or alkyl having 1, 2, 3 or 4 carbon atoms;

or

R(52) and R(53)

are a group C₆₀ H_{2α};

α is 4, 5, 6 or 7;

where if α=5, 6 or 7 a carbon atom of the group C₆₀ H_{2α} can be replaced by a heteroatom group O, SO_{d} or NR(56),

or

R(53) and R(54) or R(54) and R(55) or R(52) and R(55)

are a group C_{y}H_{2y};

y is 2, 3, 4 or 5;

where if y=3, 4 or 5 a carbon atom of the group C_{y}H_{2y} can be replaced by a heteroatom group O, SO_{d} or NR(56);

d is zero, 1 or 2;

R(56) is hydrogen or methyl;

or

R(51) is a basic heteroaromatic ring system having 1-9 carbon atoms;

A is a group CₑH₂ₑ;

e is zero, 1, 2, 3, 4, 5, 6, 7, 8, 9 or 10;

where in the group CₑH₂ₑ a carbon atom can be replaced by one of the groupings —O—, —CO—, —CH[OR(57)]—, —SOᵣ—, —NR(57)-, —NR(57)-CO—, —NR(57)-CO—NH—, —NR(57)-CO—NH—SO₂— or —NR(57)-SO₂—;

r is zero, 1 or 2;

G is a phenylene radical,

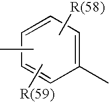

R(58) and R(59)

independently of one another are hydrogen, methyl, methoxy, F, Cl, Br, I, CF₃ or —SOₛ—R(60);

R(60) is methyl or NR(61)R(62);

R(61) and R(62)

independently of one another are hydrogen or alkyl having 1, 2, 3 or 4 carbon atoms;

D is —CᵥH₂ᵥ-E_{w}-;

v is zero, 1, 2, 3 or 4;

E is —O—, —CO—, —CH[OR(63)]—, —SOₐₐ or —NR(63)-;

w is zero or 1;

as is zero, 1 or 2

R(63) is hydrogen or methyl,

or

R(1), R(2) and R(3)

independently of one another are —CF₂R(64), —CF[R(65)][R(66)], —CF[(CF₂)_{q}—CF₃)][R(65)], —C[(CF₂)ₚ—CF₃]═CR(65)R(66);

R(64) is alkyl having 1, 2, 3 or 4 carbon atoms or cycloalkyl having 3, 4, 5, 6 or 7 carbon atoms;

R(65) and R(66) independently of one another are hydrogen or alkyl having 1, 2, 3 or 4 carbon atoms;

q is zero, 1 or 2;

p is zero, 1 or 2;

or

R(1), R(2) and R(3)

independently of one another are —OR(67) or —NR(67)R(68);

R(67) and R(68)

independently of one another are hydrogen or alkyl having 1, 2, 3, 4, 5 or 6 carbon atoms;

or

R(67) and R(68)

together are 4, 5, 6 or 7 methylene groups, of which one CH₂ group can be replaced by oxygen, —S—, SO₂, —NH—, —NCH₃ or —N-benzyl;

R(4) and R(5)

independently of one another are hydrogen, alkyl having 1, 2, 3 or 4 carbon atoms, F, Cl, —OR(69), —NR(70)R(71) or —C_{z}F_{2z+1};

R(69), R(70) and R(71)

independently of one another are hydrogen or alkyl having 1, 2 or 3 carbon atoms;

z is 1, 2, 3 or 4;

R(6) and R(7)

independently of one another are hydrogen or alkyl having 1, 2, 3 or 4 carbon atoms;

X is oxygen or NR(72);

R(72) is hydrogen or alkyl having 1, 2, 3 or 4 carbon atoms;

and their pharmaceutically tolerable salts;

(HOE 95/F 115—EP 744 397, NZ 286 622)

al) alkenylcarboxylic acid guanidides, carrying fluorophenyl groups, of the formula I

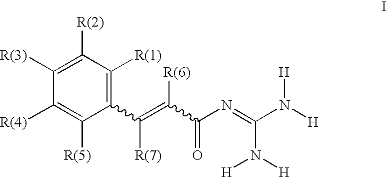

in which:

R(6) is hydrogen, (C₁-C₈)-alkyl, (C₃-C₈)-cycloalkyl or phenyl,

where the phenyl group is not substituted or is substituted by 1-3 substituents selected from the group consisting of F, Cl, CF₃, methyl, methoxy and NR(9)R(10);

R(9) and R(10)

are hydrogen, (C₁-C₄)-alkyl or (C₁-C₄)-perfluoroalkyl;

R(7) independently is defined as R(6);

R(1), R(2), R(3), R(4) and R(5)

independently of one another are hydrogen or F;

where, however, at least one of the radicals R(1), R(2), R(3), R(4) and R(5) must be fluorine;

and their pharmaceutically tolerable salts;

(HOE 95/F 173—NZ 299 052)

an) substituted cinnamic acid guanidides of the formula I

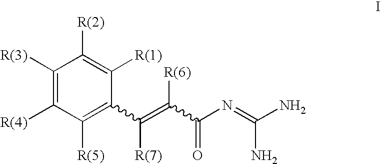

in which:

at least one of the substituents R(1), R(2), R(3), R(4) and R(5) is —Xₐ—Y_{b}-Lₙ-U;

X is CR(16)R(17), O, S or NR(18);

R(16), R(17) and R(18)

independently of one another are H, alkyl having 1, 2, 3 or 4 carbon atoms or perfluoroalkyl having 1, 2, 3 or 4 carbon atoms;

a is zero or 1;

Y is alkylene having 1, 2, 3, 4, 5, 6, 7 or 8 carbon atoms, alkylene-T having 1, 2, 3, 4, 5, 6, 7 or 8 carbon atoms in the alkylene group, T or T-alkylene having 1, 2, 3, 4, 5, 6, 7 or 8 carbon atoms in the alkylene group;

T is NR(20), O, S or phenylene,

where the phenylene is not substituted or is substituted by 1-3 substituents selected from the group consisting of F, Cl, CF₃, methyl, methoxy and NR(21 )R(22);

R(20), R(21) and R(22)

independently of one another are H, alkyl having 1, 2, 3 or 4 carbon atoms or perfluoroalkyl having 1, 2, 3 or 4 carbon atoms;

b is zero or 1;

L is O, S, NR(23) or CₖH₂ₖ;

k is 1, 2, 3, 4, 5, 6, 7, 8;

n is zero or 1;

U is NR(24)R(25) or an N-containing heterocycle having 1, 2, 3, 4, 5, 6, 7, 8 or 9 carbon atoms;

R(24) and R(25)

independently of one another are hydrogen, alkyl having 1, 2, 3, 4, 5, 6, 7 or 8 carbon atoms, perfluoroalkyl having 1, 2, 3, 4, 5, 6, 7 or 8 carbon atoms;

or

R(24) and R(25)

together are 4 or 5 methylene groups, of which one CH₂ group can be replaced by oxygen, S, NH, N—CH₃ or N-benzyl;

where the N-containing heterocycles are N- or C-bridged and are not substituted or are substituted by 1-3 substituents selected from the group consisting of F, Cl, CF₃, methyl, methoxy and NR(27)R(28);

R(23), R(27) and R(28)

independently of one another are H, alkyl having 1, 2, 3 or 4 carbon atoms or perfluoroalkyl having 1, 2, 3 or 4 carbon atoms;

and the other substituents R(1), R(2), R(3), R(4) and R(5) in each case independently of one another are H, F, Cl, Br, I, CN, —Oₙ₋CₘH₂ₘ₊₁, —Oₚ—(CH₂)ₛ—C_{q}F_{2q+1} or —CᵣH₂ᵣR(10);

n is zero or 1;

m is zero 1, 2, 3, 4, 5, 6, 7 or 8;

p is zero or 1;

q is 1, 2, 3, 4, 5, 6, 7 or 8;

s is zero, 1, 2, 3 or 4;

r is zero, 1, 2, 3 or 4;

R(10)

is cycloalkyl having 3, 4, 5, 6, 7 or 8 carbon atoms, or phenyl,

where the phenyl is not substituted or is substituted by 1-3 substituents selected from the group consisting of F, Cl, CF₃, methyl, methoxy and NR(11)R(12);

R(11) and R(12)

independently of one another are H, alkyl having 1, 2, 3 or 4 carbon atoms or perfluoroalkyl having 1, 2, 3 or 4 carbon atoms;

R(6) and R(7)

independently of one another are hydrogen, F, Cl, Br, I, CN, alkyl having 1, 2, 3, 4, 5, 6, 7 or 8 carbon atoms, perfluoroalkyl having 1, 2, 3, 4, 5, 6, 7 or 8 carbon atoms, cycloalkyl having 3, 4, 5, 6, 7 or 8 carbon atoms, or phenyl,

which is not substituted or is substituted by 1-3 substituents selected from the group consisting of F, Cl, CF₃, methyl, methoxy and NR(14)R(15); 4.

R(14) and R(15)

independently of one another are H, alkyl having 1, 2, 3 or 4 carbon atoms or perfluoroalkyl having 1, 2, 3 or 4 carbon atoms;

and their pharmaceutically tolerable salts;

(HOE 95/F 269 K)

ar) benzenedicarboxylic acid diguanidides of the formula I

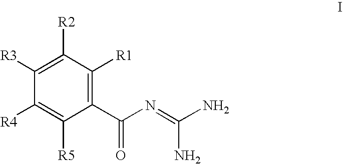

in which:

one of the radicals R(1), R(2), R(3) and R(4) is —CO—N═C(NH₂)₂;

and the other radicals R(1), R(2), R(3) and R(4) in each case are:

R(1) is hydrogen, alkyl having 1, 2, 3 or 4 carbon atoms, F, Cl, Br, I, —OR(32), —NR(33)R(34) or CF₃;

R(32), R(33) and R(34)

independently of one another are hydrogen or alkyl having 1, 2, 3 or 4 carbon atoms;

R(2) and R(4)

independently of one another are hydrogen, F, Cl, Br, I, OH, —CN, CF₃, —CO—N═C(NH₂)₂, alkyl having 1, 2, 3, 4, 5, 6, 7 or 8 carbon atoms, alkenyl having 2, 3, 4, 5, 6, 7 or 8 carbon atoms or ═(CH₂)ₘR(14);

m is zero, 1 or2;

R(14) is —(C₃-C₈)-cycloalkyl or phenyl,

which is not substituted or is substituted by 1-3 substituents selected from the group consisting of F and Cl, —CF₃, methyl, methoxy and —NR(15)R(16);

R(15) and R(16)

are hydrogen or —CH₃;

or

R(2) and R(4)

independently of one another are pyrrol-1 -yl, pyrrol-2-yl or pyrrol-3-yl,

each of which is not substituted or is substituted by 1-4 substituents selected from the group consisting of F, Cl, Br, I, —CN, (C₂-C₈)-alkanoyl, (C₂-C₈)-alkoxycarbonyl, formyl, carboxyl, —CF₃, methyl, methoxy;

or

R(2) and R(4)

independently of one another are R(22)-SO₂—, R(23)R(24)N—CO—, R(28)-CO— or R(29)R(30)N—SO₂;

R(22) and R(28)

independently of one another are methyl or —CF₃;

R(23), R(24), R(29) and R(30)

independently of one another are hydrogen or methyl;

or

R(2) and R(4)

independently of one another are —OR(35) or —NR(35)R(36);

R(35) and R(36)

independently of one another are hydrogen or alkyl having 1, 2, 3, 4, 5 or 6 carbon atoms;

or

R(35) and R(36)

together are 4-7 methylene groups, of which one CH₂ group can be replaced by oxygen, —S—, —NH—, —NCH₃ or —N-benzyl;

R(3) is hydrogen, —SR(25), —OR(25), —NR(25)R(26), —CR(25)R(26)R(27);

R(25) is hydrogen, alkyl having 1, 2, 3, 4, 5, 6, 7 or 8 carbon atoms or phenyl,

which is unsubstituted or substituted by 1-3 substituents selected from the group consisting of F, Cl, CF₃, CH₃, methoxy, hydroxyl, amino, methylamino and dimethylamino;

or

R(25) is —(C₁-C₉)-heteroaryl, which is unsubstituted or substituted by 1-3 substituents selected from the group consisting of F, Cl, CF₃, CH₃, methoxy, hydroxyl, amino, methylamino and dimethylamino;

R(26) and R(27)

independently of one another are defined as R(25) or are hydrogen or alkyl having 1, 2, 3, 4, 5, 6, 7 or 8 carbon atoms;

R(5) is alkyl having 1, 2, 3 or 4 carbon atoms, F, Cl, Br, I, X—(CH₂)_{y}—CF₃ or phenyl,

which is not substituted or is substituted by 1-3 substituents selected from the group consisting of F and Cl, —CF₃, methyl, methoxy and —NR(6)R(7);

R(6) and R(7)

independently of one another are hydrogen or —CH₃;

X is a bond or oxygen;

y is zero, 1 or 2;

and their pharmaceutically tolerable salts;

(HOE 95/F 269 BK)

as) benzenedicarboxylic acid diguanidides of the formula I

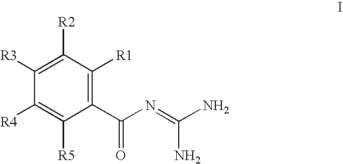

in which:

one of the radicals R(1), R(2), R(3) and R(5) is —CO—N═C(NH₂)₂;

and the other radicals R(1), R(2), R(3) and R(5) in each case are:

R(1) and R(5)

independently of one another are hydrogen, alkyl having 1, 2, 3 or 4 carbon atoms, F, Cl, —OR(32), —NR(33)R(34) or CF₃;

R(32), R(33) and R(34)

independently of one another are hydrogen or alkyl having 1, 2, 3 or 4 carbon atoms;

R(2) is hydrogen, F, Cl, Br, I, OH, —C═N, CF₃, —CO—N═C(NH₂)₂, alkyl having 1, 2, 3, 4, 5, 6, 7 or 8 carbon atoms, alkenyl having 2, 3, 4, 5, 6, 7 or 8 carbon atoms or —(CH₂)ₘR(14);

m is zero, 1 or 2;

R(14) is —(C₃-C₈)-cycloalkyl or phenyl,

which is not substituted or is substituted by 1-3 substituents selected from the group consisting of F and Cl, —CF₃, methyl, methoxy and —NR(15)R(16);

R(15) and R(16)

independently of one another are hydrogen or —CH₃;

or

R(2) is R(22)-SO₂—, R(23)R(24)N—CO—, R(28)-CO— or R(29)R(30)N—SO₂;

R(22) and R(28)

independently of one another are methyl or —CF₃;

R(23), R(24), R(29) and R(30)

independently of one another are hydrogen or methyl;

or

R(2) is —OR(35) or —NR(35)R(36);

R(35) and R(36)

independently of one another are hydrogen or alkyl having 1, 2, 3, 4, 5 or 6 carbon atoms;

or

R(35) and R(36)

together are 4-7 methylene groups, of which one CH₂ group can be replaced by oxygen, —S—, —NH—, —NCH₃ or —N-benzyl;

R(3) is hydrogen, —SR(25), —OR(25), —NR(25)R(26), —CR(25)R(26)R(27);

R(25) is hydrogen, alkyl having 1, 2, 3, 4, 5, 6, 7 or 8 carbon atoms or phenyl,

which is unsubstituted or substituted by 1-3 substituents selected from the group consisting of F, Cl, CF₃, CH₃, methoxy, hydroxyl, amino, methylamino and dimethylamino;

or

R(25) is —(C₁-C₉)-heteroaryl,

which is unsubstituted or substituted by 1-3 substituents selected from the group consisting of F, Cl, CF₃, CH3, methoxy, hydroxyl, amino, methylamino and dimethylamino;

R(26) and R(27)

independently of one another are defined as R(25) or are hydrogen or alkyl having 1, 2, 3, 4, 5, 6, 7 or 8 carbon atoms;

R(4) is CF₃, alkyl having 1, 2, 3, 4, 5, 6, 7 or 8 carbon atoms, alkenyl having 2, 3, 4, 5, 6, 7 or 8 carbon atoms, —(C₃-C₈)-cycloalkyl or —(CH₂)ₘR(1 4);

m is 1 or 2;

R(14) is —(C₃-C₈)-cycloalkyl or phenyl,

which is not substituted or is substituted by 1-3 substituents selected from the group consisting of F and Cl, —CF₃, methyl, methoxy and —NR(15)R(16);

R(15) and R(16)

independently of one another are hydrogen or —CH₃;

or

R(4) is phenyl,

which is substituted by 2, 3, 4 or five substituents selected from the group consisting of F, Cl, CF₃, methyl, methoxy and —NR(15)R(16);

R(15) and R(16)

independently of one another are hydrogen or CH₃;

and their pharmaceutically tolerable salts;

(HOE 96/F 013)

at) diaryldicarboxylic acid diguanidides of the formula I

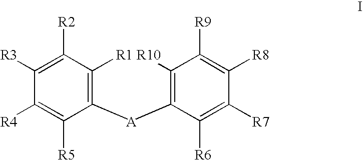

in which:

one of the radicals R(1), R(2), R(3), R(4) and R(5) is —CO—N═C(NH₂)₂;

the other radicals R(1) and R(5) in each case

independently of one another are hydrogen, alkyl having 1, 2, 3 or 4 carbon atoms, F, Cl, —OR(32), —NR(33)R(34) or CF₃;

R(32), R(33) and R(34)

independently of one another are hydrogen or alkyl having 1, 2, 3 or 4 carbon atoms;

the other radicals R(2) and R(4) in each case

independently of one another are hydrogen, F, Cl, Br, I, OH, —CN, CF₃, —CO—N═C(NH₂)₂, alkyl having 1, 2, 3, 4, 5, 6, 7 or 8 carbon atoms, alkenyl having 2, 3, 4, 5, 6, 7 or 8 carbon atoms or —(CH₂)ₘR(14);

m is zero, 1 or 2;

R(14) is —(C₃-C₈)-cycloalkyl or phenyl,

which is not substituted or is substituted by 1-3 substituents selected from the group consisting of F and Cl, —CF₃, methyl, methoxy and —NR(15)R(16);

R(15) and R(16)

are hydrogen or —CH₃;

or

the other radicals R(2) and R(4) in each case

independently of one another are pyrrol-1-yl, pyrrol-2-yl or pyrrol-3-yl,

which is not substituted or is substituted by 1-4 substituents selected from the group consisting of F, Cl, Br, I, —CN, (C₂-C₈)-alkanoyl, (C₂-C₈)-alkoxycarbonyl, formyl, carboxyl, —CF₃, methyl, methoxy;

or

the other radicals R(2) and R(4) in each case

are R(22)-SO₂—, R(23)R(24)N—CO—, R(28)-CO or R(29)R(30)N—SO₂;

R(22) and R(28)

independently of one another are methyl or —CF₃;

R(23), R(24), R(29) and R(30)

independently of one another are hydrogen or methyl;

or

the other radicals R(2) and R(4) in each case

independently of one another are —OR(35) or —NR(35)R(36);

R(35) and R(36)

independently of one another are hydrogen or alkyl having 1, 2, 3, 4, 5 or 6 carbon atoms;

or

R(35) and R(36)

together are 4-7 methylene groups, of which one CH₂ group can be replaced by oxygen, —S—, —NH—, —NCH₃ or —N-benzyl;

the other radical R(3) in each case

is hydrogen, —SR(25), —OR(25), —NR(25)R(26), —CR(25)R(26)R(27);

R(25) is hydrogen, alkyl having 1, 2, 3, 4, 5, 6, 7 or 8 carbon atoms or phenyl,

which is unsubstituted or substituted by 1-3 substituents selected from the group consisting of F, Cl, CF₃, CH₃, methoxy, hydroxyl, amino, methylamino and dimethylamino;

or

R(25) is —(C₁-C₉)-heteroaryl,

which is unsubstituted or substituted by 1-3 substituents selected from the group consisting of F, Cl, CF₃, CH₃, methoxy, hydroxyl, amino, methylamino and dimethylamino;

R(26) and R(27)

independently of one another are defined as R(25) or are hydrogen or alkyl having 1, 2, 3, 4, 5, 6, 7 or 8 carbon atoms;

one of the radicals R(6), R(7), R(8), R(9) and R(10) is —CO—N═C(NH₂)₂;

the other radicals R(6) and R(10) in each case

independently of one another are hydrogen, alkyl having 1, 2, 3 or 4 carbon atoms, F, Cl, —OR(132), —NR(133)R(134) or CF₃;

R(132), R(133) and R(134)

independently of one another are hydrogen or alkyl having 1, 2, 3 or 4 carbon atoms;

the other radicals R(7) and R(9) in each case

independently of one another are hydrogen, F, Cl, Br, I, OH, —CN, CF₃, —CO—N═C(NH₂)₂, alkyl having 1, 2, 3, 4, 5, 6, 7 or 8 carbon atoms, alkenyl having 2, 3, 4, 5, 6, 7 or 8 carbon atoms or —(CH₂)ₘₘR(114);

mm is zero, 1 or 2;

R( 114)

is —(C₃-C₈)-cycloalkyl or phenyl,

which is not substituted or is substituted by 1-3 substituents selected from the group consisting of F and Cl, —CF₃, methyl, methoxy and —NR(1 15)R(1 16);

R(115) and R(116)

are hydrogen or —CH₃;

or

the other radicals R(7) and R(9) in each case independently of one another are pyrrol-1-yl, pyrrol-2-yl or pyrrol-3-yl,

which is not substituted or is substituted by 1-4 substituents selected from the group consisting of F, Cl, Br, I, —CN, (C₂-C₈)-alkanoyl, (C₂-C₈)-alkoxycarbonyl, formyl, carboxyl, —CF₃, methyl and methoxy;

or

the other radicals R(7) and R(9) in each case

are R(122)-SO₂—, R(123)R(124)N—CO—, R(128)CO— or R(129)R(130)N—SO₂;

R(122) and R(128)

independently of one another are methyl or —CF₃;

R(123), R(124), R(129) and R(130)

independently of one another are hydrogen or methyl;

or

the other radicals R(7) and R(9) in each case

independently of one another are —OR(135) or —NR(135)R(136);

R(135) and R(136)

independently of one another are hydrogen or alkyl having 1, 2, 3, 4, 5 or 6 carbon atoms;

or

R(135) and R(136)

together are 4-7 methylene groups, of which one CH₂ group can be replaced by oxygen, —S—, —NH—, —NCH₃ or —N-benzyl;

the other radical R(8) in each case

is hydrogen, —SR(125), —OR(125), —NR(125)R(126) or —CR(125)R(126)R(127);

R(125)

is hydrogen, alkyl having 1, 2, 3, 4, 5, 6, 7 or 8 carbon atoms or phenyl,

which is unsubstituted or substituted by 1-3 substituents selected from the group consisting of F, Cl, CF₃, CH₃, methoxy, hydroxyl, amino, methylamino and dimethylamino;

or

R(125)

is —(C₁-C₉)-heteroaryl,

which is unsubstituted or substituted by 1-3 substituents selected from the group consisting of F, Cl, CF₃, CH₃, methoxy, hydroxyl, amino, methylamino and dimethylamino;

R(126) and R(127)

independently of one another are defined as R(125) or are hydrogen or alkyl having 1, 2, 3, 4, 5, 6, 7 or 8 carbon atoms;

A is absent or is —NR(11)-CO—, —NR(12)-CO—NR(13)-, —NR(17)-CO—NR(18)-SO₂—, —NR(19)-SO₂—, —SO₂—NR(19)-SO₂—, —SO₂—NR(19)-CO—, —O—CO—NR(19)-SO₂— or —CR(20)=CR(21)-;

R(11), R(12), R(13), R(17), R(18), R(19), R(20) and R(21)

independently of one another are hydrogen or alkyl having 1, 2, 3, 4, 5, 6, 7 or 8 carbon atoms

and their pharmaceutically tolerable salts;

(HOE 96/F 026)

au) substituted thiophenylalkenylcarboxylic acid guanidides of the formula I

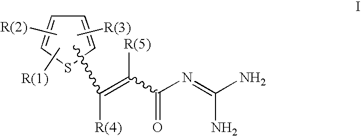

in which:

at least one of the substituents R(1), R(2) and R(3) is —Oₚ—(CH₂)ₛ—C_{q}F₂ₐ₊₁, R(40)CO— or R(31 )SOₖ—;

p is zero or 1;

s is zero, 1, 2, 3 or 4;

q is 1, 2, 3, 4, 5, 6, 7 or 8;

k is zero, 1 or 2;

R(40) is alkyl having 1, 2, 3, 4, 5, 6, 7 or 8 carbon atoms, perfluoroalkyl having 1, 2, 3, 4, 5, 6, 7 or 8 carbon atoms, cycloalkyl having 3, 4, 5, 6, 7 or 8 carbon atoms, or phenyl,

which is not substituted or is substituted by 1-3 substituents selected from the group consisting of F, Cl, CF₃, methyl and methoxy;

R(31) is alkyl having 1, 2, 3, 4, 5, 6, 7 or 8 carbon atoms, perfluoroalkyl having 1, 2, 3, 4, 5, 6, 7 or 8 carbon atoms, cycloalkyl having 3, 4, 5, 6, 7 or 8 carbon atoms, or phenyl,

which is not substituted or is substituted by 1-3 substituents selected from the group consisting of F, Cl, CF₃, methyl or methoxy;

or

R(31) is NR(41)R(42);

R(41)and R(42)

independently of one another are hydrogen, alkyl having 1, 2, 3 or 4 carbon atoms, perfluoroalkyl having 1, 2, 3 or 4 carbon atoms,

or

R(41) and R(42)

together are 4 or 5 methylene groups, of which one CH₂ group can be replaced by oxygen, S, NH, N—CH₃ or N-benzyl;

and the other substituents R(1), R(2) and R(3) in each case independently of one another are H, F, Cl, Br, I, CN, —OₙₐCₘₐH₂ₘₐ₊₁ or —O_{ga}CᵣₐH₂ᵣₐR(10);

na is zero or 1;

ma is zero, 1, 2, 3, 4, 5, 6, 7 or 8;

ga is zero or 1;

ra is zero, 1, 2, 3 or 4;

R(10) is cycloalkyl having 3, 4, 5, 6, 7 or 8 carbon atoms or phenyl,

where the phenyl is not substituted or is substituted by 1-3 substituents selected from the group consisting of F, Cl, CF₃, methyl and methoxy;

R(4) and R(5)

independently of one another are hydrogen, F, Cl, Br, I, CN, alkyl having 1, 2, 3, 4, 5, 6, 7 or 8 carbon atoms, perfluoroalkyl having 1, 2, 3, 4, 5, 6, 7 or 8 carbon atoms, cycloalkyl having 3, 4, 5, 6, 7 or 8 carbon atoms or phenyl,

which is not substituted or is substituted by 1-3 substituents selected from the group consisting of F, Cl, CF₃, methyl, methoxy and NR(14)R(15);

R(14) and R(15)

independently of one another are H, alkyl having 1, 2, 3 or 4 carbon atoms or perfluoroalkyl having 1, 2, 3 or 4 carbon atoms;

and their pharmaceutically tolerable salts.

For inhibitors of Na⁺/H⁺ exchange of this type, numerous medicinal uses have already been described, such as, for example, forms of illness which occur due to chronic or acute undersupply of blood to an organ (ischemia), in particular of the heart. They are therefore suitable, for example, for the treatment of is chemically induced arrhythmias, different forms of angina pectoris, in heart transplants, in cardiac surgery and in angioplastic surgical interventions. Other indications described for NHE inhibitors are stroke and cerebral edema, shock and proliferation-related diseases, such as atherosclerosis, diabetic late damage, fibrotic disorders and organ hypertrophy.

It has now surprisingly been found that NHE inhibitors have a favorable effect on serum lipoproteins. It is generally recognized that for the formation of arterioscierotic vascular changes, in particular of coronary heart disease, excessively high blood lipid values, so-called hyperlipoproteinemias, are a significant risk factor. For the prophylaxis and regression of atherosclerotic changes, the lowering of raised serum lipoproteins therefore has extreme importance. Beside the reduction of serum total cholesterol, the lowering of the proportion of specific atherogenic lipid fractions of this total cholesterol, in particular of the low density lipoproteins (LDL) and of the very low density lipoproteins (VLDL) has particular importance, since these lipid fractions are an atherogenic risk factor. However, the high density lipoproteins are ascribed a protective function against coronary heart disease. Accordingly, hypolipidemics should be able to lower not only total cholesterol, but in particular the VLDL and LDL serum cholesterol fractions. It has now surprisingly been found that NHE inhibitors have valuable therapeutically utilizable properties with respect to affecting the serum lipid level. Thus they significantly reduce the raised serum concentration of LDL and VLDL, such as is to be observed, for example, as a result of increased dietetic uptake of a cholesterol- and lipid-rich diet or in the case of pathological metabolic changes, for example genetically related hyperlipidemias. They can therefore be used for the prophylaxis and for the regression of atherosclerotic changes by eliminating a causal risk factor. These include not only the primary hyperlipidemias, but also certain secondary hyperlipidemias, such as occur, for example, in diabetes. Moreover, the NHE inhibitors lead to a marked reduction in the infarcts induced by metabolic anomalies and in particular to a significant reduction in the induced infarct size and its degree of severity. Furthermore, NHE inhibitors result in effective protection against endothelial damage induced by metabolic anomalies. With this protection of the vessels against the endothelial dysfunction syndrome, NHE inhibitors are valuable pharmaceuticals for the prevention and treatment of coronary vascular spasms, of atherogenesis and of atherosclerosis, of left ventricular hypertrophy and of dilated cardiomyopathy, and thrombotic disorders.

This antihyperlipidemic action is shown for an NHE inhibitor, 4-isopropyl-3-methylsulfonylbenzoylguanidine methanesulfonate (Hoe 642), in the following table, male New Zealand white rabbits (n=28) having a weight of 3-4 kg being used and divided into four groups of 7 animals in each case randomized as described below: 1) standard rabbit feed (®Altromin 2834), 2) atherogenic diet having a content of 0.25% cholesterol and 3% coconut oil, 3) standard rabbit feed+Hoe 642 (0.1%), 4) atherogenic diet+Hoe 642 (0.1%).

Blood samples were taken once after 30 days feeding by puncture of the auricular artery. Serum was obtained from the blood. The total cholesterol was measured from the serum by means of the CHOD (cholesterol oxidase)-PAP method (Merckotest, Merck Diagnostics, E. Merck, 64271 Darmstadt, Germany). The separation of the lipoproteins was carried out, likewise from the serum, by FPLC (fast protein liquid chromatography) method (März et al. 1993, Clin. Chem. 39/11:2276-2281).

Results

Total cholesterol values, and the subtractions VLDL,
LDL and HDL from the rabbit sera:

| (mmol/l) | Cholesterol | VLDL | LDL | HDL |
|---|---|---|---|---|
| Normal diet | 0.65 ± 0.08 | 0.05 ± 0.01 | 0.12 ± 0.02 | 0.48 ± |
| | | | | 0.06 |
| Normal diet + | 0.69 ± 0.1 | 0.05 ± 0.02 | 0.23 ± 0.04 | 0.41 ± |
| HOE 642 | | | | 0.17 |
| Cholesterol diet | 17.85 ± 3.71 | 5.68 ± 1.38 | 9.31 ± 1.88 | 2.86 ± |
| | | | | 0.73 |
| Cholesterol diet + | 7.95 ±0.89* | 4.6 ± 0.6 | 2.1 ± 0.2* | 1.5 ± |
| HOE 642 | | | | 0.2 |

The values are indicated as mean values ± SEM (standard error of the mean).
Significances: *p < 0.05 cholesterol diet + HOE 642 vs cholesterol diet. There are no significant differences between the two normal diet groups. Both normal diet groups are significantly different from the two cholesterol groups.

The compounds used according to the invention are therefore advantageously used for the production of a medicament for the treatment of hypercholesterolemia; for the production of a medicament for the prevention of atherogenesis; for the production of a medicament for the prevention and treatment of atherosclerosis, for the production of a medicament for the prevention and treatment of illnesses which are caused by increased cholesterol levels, for the production of a medicament for the prevention and treatment of illnesses which are caused by endothelial dysfunction, for the production of a medicament for the prevention and treatment of atherosclerosis-induced hypertension, for the production of a medicament for the prevention and treatment of atherosclerosis-induced thromboses, for the production of a medicament for the prevention and treatment of hypercholesterolemia- and endothelial dysfunction-induced ischemic damage and postischemic reperfusion damage, for the production of a medicament for the prevention and treatment of hypercholesterolemia- and endothelial dysfunction-induced cardiac hypertrophies and cardiomyopathies, for the production of a medicament for the prevention and treatment of hypercholesterolemia- and endothelial dysfunction-induced coronary vascular spasms and myocardial infarcts, for the production of a medicament for the treatment of the conditions mentioned in combination with hypotensive substances, preferably with angiotensin converting enzyme (ACE) inhibitors and angiotensin receptor antagonists, a combination of an NHE inhibitor with a blood lipid level-lowering active compound, preferably with an HMG-CoA reductase inhibitor, e.g. lovastatin or pravastatin, the latter causing a hypolipidemic action and thereby increasing the hypolipidemic properties of the NHE inhibitor, proving to be a favorable combination with increased action and reduced use of active compound.

The administration of sodium/proton exchange inhibitors as novel pharmaceuticals for lowering increased blood lipid levels is claimed, as well as the combination of sodium/proton exchange inhibitors with pharmaceuticals having a hypolensive and/or hypolipidemic action.

1. Use of an Na⁺/H⁺ exchange inhibitor for the production of a medicament for the treatment of raised blood lipid levels.

2. The use I as claimed in claim 1, which comprises employing as Na⁺/H⁺ exchange inhibitor:
I. a) benzoylguanidines of the formula I 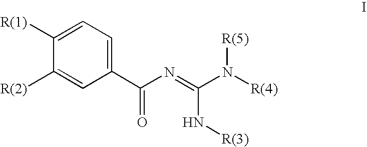
in which:
R(1) or R(2)
is R(6)-S(O)ₙ— or R(7)R(8)N—O₂S—;
and the other substituent R(1) or R(2) in each case
is H, F, Cl, Br, (C₁-C₄)-alkyl, (C₁-C₄)-alkoxy or phenoxy,
which is unsubstituted or substituted by 1-3 substituents selected from the group consisting of fluorine, chorine, methyl and methoxy;
or the other substituent R(1) or R(2) in each case
is R(6)-S(O)ₙ or R(7)R(8)N—;
n is zero, 1 or 2;
R(6) is (C₁-C₆)-alkyl, (C₅-C₇)-cycloalkyl, cyclopentylmethyl, cyclohexylmethyl or phenyl,
which is unsubstituted or substituted by 1-3 substituents selected from the group consisting of fluorine, chlorine, methyl and methoxy;
R(7) and R(8)
identically or differently are H or (C₁-C₆)-alkyl;
or
R(7) is phenyl-(CH₂)ₘ;
m is 1-4;
or
R(7) is phenyl,
which is unsubstituted or substituted by 1-2 substituents selected from the group consisting of fluorine, chlorine, methyl and methoxy;
or
R(7) and R(8)
together are a straight-chain or branched (C₄-C₇)-chain, where the chain can additionally be interrupted by O, S or NR(9);
R(9) is H or methyl;
or
R(7) and R(8)
together with the nitrogen atom to which they are bonded, are a dihydroindole, tetrahydroquinoline or tetrahydroisoquinoline system;
R(3), R(4) and R(5)
independently of one another are H or (C₁-C₂)-alkyl,
or
R(3) and R(4)
together are a (C₂-C₄)-alkylene chain;
or
R(4) and R(5)
together are a (C₄-C₇)-alkylene chain;
and their pharmaceutically tolerable salts;
b) benzoylguanidines of the formula I 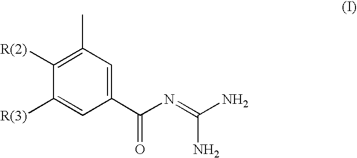
in which:
R(1) is R(4)-SOₘ or R(5)R(6)N—SO₂—;
m is zero, 1 or 2;
R(4) and R(5)
are C₁-C₈-alkyl, C₃-C₆-alkenyl or —CₙH₂ₙ—R(7);
n is zero, 1, 2, 3or 4;
R(7) is C₅-C₇-cycloalkyl or phenyl,
which is unsubstituted or substituted by 1-3 substituents selected from the group consisting of F, Cl, CF₃, methyl, methoxy and NR(8)R(9);
R(8) and R(9)
are H or C₁-C₄-alkyl;
or
R(5) is H;
R(6) is H or C₁-C₄-alkyl,
or
R(5) and R(6)
together are 4 or 5 methylene groups, of which one CH₂ group can be replaced by an O, S, NH, N—CH₃ or N-benzyl;
R(2) is hydrogen, F, Cl, Br, (C₁-C₄)-alkyl-, O—(CH₂)ₘCₚF₂ₚ₊₁ or —X—R(10);
m is zero or 1;
p is 1, 2 or3;
X is O, S or NR(11);
R(10) is H, C₁-C₆-alkyl, C₅-C₇-cycloalkyl, cyclohexylmethyl, cyclopentylmethyl or —CₙH₂ₙ—R(12);
n is zero, 1, 2, 3 or 4;
R(12) is phenyl,
which is unsubstituted or substituted by 1-3 substituents selected from the group consisting of F, Cl, CF₃, methyl, methoxy and NR(8)R(9);
R(8) and R(9)
are H or C₁-C₄-alkyl;
R(11) is hydrogen or C₁-C₃-alkyl;
or
R(10) and R(11)
together are 4 or 5 methylene groups, of which one CH₂ group can be replaced by O, S, NH, N—CH₃ or N-benzyl;
R(3) is defined as R(1), or is C₁-C₆-alkyl, nitro, cyano, trifluoromethyl, F, Cl, Br, I or —X—R(10);
X is O, S or NR(11);
R(10) is H, C₁-C₆-alkyl, C₅-C₇-cycloalkyl, cyclohexylmethyl, cyclopentylmethyl or —CₙH₂ₙ—R(12);
n is zero to 4;
R(12) is phenyl,
which is unsubstituted or substituted by 1-3 substituents selected from the group consisting of F, Cl, CF₃, methyl, methoxy and NR(8)R(9);
R(8) and R(9)
are H or C₁-C₄-alkyl;
R(11) is C₁-C₃-alkyl,
or
R(10) and R(11)
together are 4 or 5 methylene groups, of which one CH₂ group can be replaced by O, S, NH, N—CH₃ or N-benzyl;
and their pharmaceutically tolerable salts;
c) ortho-substituted benzoylguanidines of the formula I 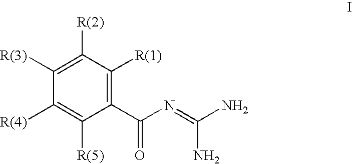
in which:
R(1) is F, Cl, Br, I, C₁-C₆-alkyl or —X—R(6);
X is O, S, NR(7) or Y-ZO;
Y is O or NR(7);
Z is C or SO;
R(6) is H, C₁-C₆-alkyl, C₅-C₇-cycloalkyl, cyclohexylmethyl, cyclopentylmethyl, —(CH₂)ₘCₚF₂ₚ₊₁ or —CₙH₂ₙ—R(B);
m is zero or 1;
p is 1-3;
n is zero to 4;
R(8) is phenyl,
which is unsubstituted or substituted by 1-3 substituents selected from the group consisting of the groups F, Cl, CF₃, methyl, methoxy and NR(9)R(10);
R(9) and R(10)
are H or C₁-C₄-alkyl;
R(7) is H or C₁-C₃-alkyl;
or
R(6) and R(7)
together are 4 or 5 methylene groups, of which one CH₂ group can be replaced by O, S, NH, N—CH₃ or N-benzyl;
R(3) is H or —X—R(6);
X is O, S, NR(7) or Y-ZO;
R(7) is H or C₁-C₃-alkyl;
Y is O or NR(7);
where Y is bonded to the phenyl radical of the formula I,
Z is C or SO;
R(6) is H, C₁-C₆-alkyl, C₅-C₇-cycloalkyl, cyclohexylmethyl, cyclopentylmethyl, —(CH₂)ₘCₚF₂ₚ₊₁ or —CₙH₂ₙ—R(8);
m is zero or 1;
p is 1-3;
n is zero to4;
R(8) is phenyl,
which is unsubstituted or substituted by 1-3 substituents selected from the group consisting of the groups F, Cl, CF₃, methyl, methoxy and NR(9)R(10);
R(9) and R(10)
are H or C₁-C₄-alkyl;
or
R(6) and R(7)
together are 4 or 5 methylene groups, of which one CH₂ group can be replaced by O, S, NH, N—CH₃ or N-benzyl;
R(2) and R(4)
identically or differently are R(11)-SO_{q}— or R(12)R(13)N—SO₂—;
q is zero 2;
R(11) is C₁-C₄-alkyl,
which is unsubstituted or carries phenyl as a substituent, where phenyl is unsubstituted or substituted by 1-3 substituents selected from the group consisting of F, Cl, CF₃, methyl, methoxy and NR(9)R(10);
R(9) and R(10)
are H or C₁-C₄-alkyl;
R(12) and R(13)
are defined as R(6) and R(7);
or one of the two radicals R(2) or R(4)
is hydrogen or is defined as R(1);
R(5) is H, methyl, F, Cl or methoxy,
and their pharmaceutically tolerable salts;
d) benzoylguanidines of the formula I 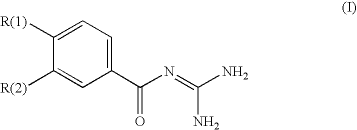
in which:
R(1) or R(2)
is an amino group —NR(3)R(4);
R(3) and R(4)
identically or differently are H, C₁-C₆-alkyl or C₃-C₇-cycloalkyl;
or
R(3) is phenyl-(CH₂)ₚ—;
p is 0,1,2,3 or 4;
or
R(3) is phenyl,
where the phenyl in each case is unsubstituted or carries one to two substituents selected from the group consisting of fluorine, chlorine, methyl and methoxy;
or
R(3) and R(4)
together can be a straight-chain or branched C₄-C₇-methylene chain, where one —CH₂— member of the methylene chain can be replaced by oxygen, S or NR(5);
R(5) is H or lower alkyl;
the other substituent R(1) or R(2) in each case
is H, F, Cl, C₁-C₄-alkyl, C₁-C₄-alkoxy, CF₃, CₘF₂ₘ₊₁—CH₂—, benzyl or phenoxy,
where the respective phenyl radical is unsubstituted or carries one to two substituents selected from the group consisting of methyl, methoxy, fluorine and chlorine;
m is 1,2or 3;
and their pharmaceutically tolerable salts;
e) benzoylguanidines of the formula I 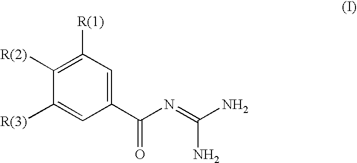
in which:
R(1) is R(4)-SOₘ or R(5)R(6)N—SO₂—;
m is zero, 1 or 2;
R(4) and R(5)
are C₁-C₈-alkyl, C₃-C₆-alkenyl or —CₙH₂ₙ—R(7);
n is zero, 1, 2, 3 or 4;
R(7) is C₅-C₇-cycloalkyl or phenyl,
which is unsubstituted or substituted by 1-3 substituents selected from the group consisting of F, Cl, CF₃, methyl, methoxy and NR(8)R(9);
R(8) and R(9)
are H or C₁-C₄-alkyl;
or
R(5) is H;
R(6) is H or C₁-C₄-alkyl;
or
R(5) and R(6)
together are 4 or 5 methylene groups, of which one CH₂ group can be replaced by an O, S, NH, N—CH₃ or N-benzyl;
R(2) is hydrogen, straight-chain or branched (C₅-C₈)-alkyl, —CR(13)═CHR(12) or —C═CR(12);
R(12) is phenyl,
which is unsubstituted or substituted by 1-3 substituents selected from the group consisting of F, Cl, CF₃, methyl, methoxy and NR(14)R(15);
R(14) and R(15)
are H or (C₁-C₄)-alkyl;
or
R(12) is (C₁-C₉)-heteroaryl,
which is unsubstituted or substituted as phenyl,
or
R(12) is (C₁-C₆)-alkyl,
which is unsubstituted or substituted by 1-3 OH,
or
R(12) is (C₃-C₈)-cycloalkyl;
R(13) is hydrogen or methyl,
or
R(12) is (C₃-C₈)-cycloalkyl, (C₃-C₈)-cycloalkyl-(C₁-C₄)-alkyl, phenyl, C₆H₅-(C₁-C₄)-alkyl, naphthyl, biphenylyl, 1,1-diphenyl-(C₁-C₄)-alkyl, cyclopentadienyl, pyridyl, pyrrolyl, furanyl, thienyl, thiazolyl, oxazolyl, indenyl, quinolyl, indolyl, benzofuranyl, benzothienyl, benzothiazolyl, benzoxazolyl, imidazolyl, pyrazolyl, triazolyl, tetrazolyl, isoxazolyl, isothiazolyl, pyrazinyl, pyrimidinyl, pyridazinyl, indazolyl, isoquinolyl, phthalazinyl, quinoxalinyl, quinazolinyl or cinnolinyl;
R(3) is defined as R(2);
and where the aromatic substituents R(2) and R(3) are unsubstituted or substituted by 1-3 substituents from the groups F, Cl, CF₃, (C₁-C₄)-alkyl or -alkoxy, or NR(10)R(11) with R(10) and R(11) being H or (C₁-C₄)-alkyl;
and their pharmaceutically tolerable salts;
f) benzoylguanidines of the formula I 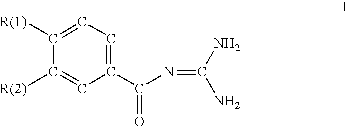
in which:
R(1) or R(2)
is R(3)-S(O)ₙ— or 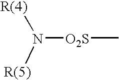
the other substituent R(1) or R(2) in each case
is H, OH, F, Cl, Br, I, C₁-C₄-alkyl, C₁-C₄-alkoxy, benzyloxy or phenoxy,
which is unsubstituted or carries one to three substituents selected from the group consisting of fluorine, chlorine, methyl, methoxy, hydroxyl or benzyloxy,
R(3)-S(O)ₙ, —NR4)R(5) or 3,4-dehydropiperidine
R(3) is C₁-C₆-alkyl, C₅-C₇-cycloalkyl, cyclopentylmethyl, cyclohexylmethyl or phenyl,
which is unsubstituted or substituted by one to three substituents selected from the group consisting of fluorine, chlorine, methyl and methoxy;
R(4) and R(5)
identically or differently, are H or C₁-C₆-alkyl;
or
R(4) is phenyl-(CH₂)m—;
m is 1, 2, 3 or 4;
or
R(4) is phenyl,
which is unsubstituted or carries one to two substituents selected from the group consisting of fluorine, chlorine, methyl and methoxy;
or
R(4) and R(5)
together are a straight-chain or branched C₄-C₇-chain, where the chain can additionally be interrupted by O, S or NR(6),
R(6) is H or methyl;
or
R(4) and R(5)
together with the nitrogen atom to which they are bonded, are a dihydroindole, tetrahydroquinoline or tetrahydroisoquinoline system;
n is zero, 1 or 2;
and their pharmaceutically tolerable salts;
g) isoquinolines of the formula I 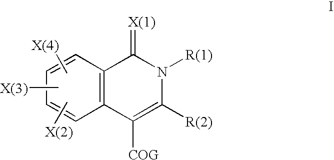
in which:
R(1) is hydrogen, alkyl, cycloalkyl, arylalkyl, alkenyl, substituted aminoalkyl or an aryl or heteroaryl ring;
where the rings are unsubstituted or substituted by 1-3 groups selected from the group consisting of halogen, nitro, amino, mono(lower alkyl)amino, di(lower alkyl)amino, lower alkyl, lower alkoxy, benzyloxy, phenoxy, hydroxyl, trifluoromethyl,
R(2) is hydrogen, halogen, alkyl or aryl;
which is unsubstituted or substituted by 1-3 groups selected from the group consisting of halogen, nitro, amino, mono(lower alkyl)amino, di(lower alkyl)amino, lower alkyl, lower alkoxy, benzyloxy, phenoxy, hydroxyl,
G is 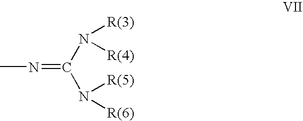
X(2), X(3) and X(4)
independently of one another are hydrogen, halogen, nitro, amino, alkyl, sulfonamide, mono(lower alkyl)amino, di(lower alkyl)amino, lower alkyl, benzyloxy, hydroxyl;
X(1) is hydrogen, oxygen, sulfur or NR(7);
R(7) is hydrogen, alkyl, cycloalkyl, arylalkyl, alkenyl, substituted aminoalkyl or an aryl or a heteroaryl ring;
which rings are unsubstituted or substituted by 1-3 groups selected from the group consisting of halogen, nitro, amino, mono(lower alkyl)amino, di(lower alkyl)amino, lower alkyl, lower alkoxy, benzyloxy, phenoxy, hydroxyl and trifluoromethyl;
in which substituents each alkyl chain or alkenyl chain can be interrupted by oxygen, sulfur or NR(8);
R(8) is hydrogen, alkyl, cycloalkyl, arylalkyl, alkenyl, substituted aminoalkyl or an aryl or heteroaryl ring,
which rings are unsubstituted or substituted by 1-3 groups selected from the group consisting of halogen, nitro, amino, mono(lower alkyl)amino, di(lower alkyl)amino, lower alkyl, lower alkoxy, benzyloxy, phenoxy, hydroxyl and trifluoromethyl;
and their pharmaceutically acceptable salts;
h) compounds of the formula I 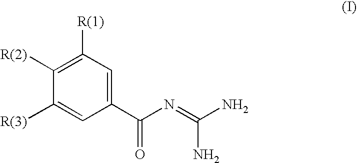
in which:
R(1) is hydrogen, F, Cl, Br, I, —NO₂, —C≡N, —CF₃, R(4)-SOₘ or
R(5)R(6)N—SO₂—;
m is zero, 1 or 2;
R(4) and R(5)
are (C₁-C₈)-alkyl, (C₃-C₆)-alkenyl, —CₙH₂ₙ—R(7) or CF₃;
n is zero, 1, 2, 3 or 4;
R(7) is (C₃-C₇)-cycloalkyl or phenyl,
which is not substituted or is substituted by 1-3 substituents selected from the group consisting of F, Cl, CF₃, methyl, methoxy and NR(8)R(9);
R(8) and R(9)
are H or C₁-C₄-alkyl;
or
R(5) is H;
R(6) is H or (C₁-C₄)-alkyl;
or
R(5) and R(6)
together are 4 or 5 methylene groups, of which one CH₂ group can be replaced by oxygen, S, NH, N—CH₃ or N-benzyl;
R(2) is —SR(10), —OR(10), —NHR(10), —NR(10)R(11), CHR(10)R(12), —[CR(12)R(13)OR(13′)], —(C—[CH₂—OR(13′)]R(12)R(13)} or —[CR(18)R(17)]ₚ—(CO)—[CR(19)R(20)]_{q}—R(14);
R(10), R(11)
identically or differently
are —[CHR(16)]₅—(CH₂)ₚ—(CHOH)_{q}—(CH₂)ᵣ—(CHOH)ₜ—R(21) or —(CH₂)ₚ—O—(CH₂—CH₂O)_{q}—R(21),
R(21) is hydrogen, methyl,
p, q, r identically or differently
are zero, 1, 2, 3 or 4;
s is zero or 1;
t is 1, 2, 3 or 4;
R(12) and R(13)
identically or differently are hydrogen, (C₁-C₆)-alkyl or, together with the carbon atom carrying them, are a (C₃-C₈)-cycloalkyl,
R(13′) is hydrogen or (C₁-C₄)-alkyl;
R(14) is H, (C₁-C₆)-alkyl, (C₃-C₈)-cycloalkyl or —CₐH₂ₐ—R(15);
a is zero, 1, 2, 3 or 4;
R(15) is phenyl,
which is unsubstituted or substituted by 1-3 substituents selected from the group consisting of F, Cl, CF₃, methyl, methoxy and NR(8)R(9);
R(8) and R(9)
are H or (C₁-C₄)-alkyl;
or
R(15) is (C₁-C₉)-heteroaryl,
which is unsubstituted or substituted as phenyl,
or
R(15) is (C₁-C₆)-alkyl,
which is unsubstituted or substituted by 1-3 OH;
R(16), R(17), R(18), R(19) and R(20)
are hydrogen or (C₁-C₃)-alkyl;
R(3) is defined as R(1),
or
R(3) is (C₁-C₆)-alkyl or —X—R(22);
X is oxygen, S or NR(16);
R(16) is H or (C₁-C₃)-alkyl;
or
R(22) and R(16)
together are 4 or 5 methylene groups, of which one CH₂ group can be replaced by oxygen, S, NH, N—CH₃ or N-benzyl;
R(22) is defined as R(14);
and their pharmaceutically tolerable salts;
i) benzoylguanidines of the formula I 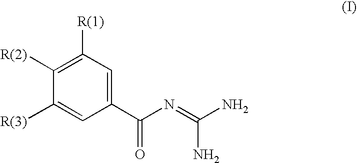
in which:
R(1) is hydrogen, F, Cl, Br, I, —NO₂, —C≡N, R(16)-CₚH₂ₚ—O_{q}, R(4)-SOₘ or R(5)R(6)N—SO₂—;
m is zero, 1 or 2;
p is zero or 1;
q is zero, 1, 2 or 3;
R(16) is CᵣF₂ᵣ₊₁;
r is 1, 2 or 3;
R(4) and R(5)
are (C₁-C₈)-alkyl, (C₃-C₆)-alkenyl, —CₙH₂ₙ—R(7) or CF₃;
n is zero, 1, 2, 3 or 4;
R(7) is (C₃-C₇)-cycloalkyl or phenyl,
which is not substituted or is substituted by 1-3 substituents selected from the group consisting of F, Cl, CF₃, methyl, methoxy and NR(8)R(9);
R(8) and R(9)
are H or C₁-C₄-alkyl;
or
R(5) is H;
R(6) is H or (C₁-C₄)-alkyl;
or
R(5) and R(6)
together are 4 or 5 methylene groups, of which one CH₂ group can be replaced by oxygen, S, NH, N—CH₃ or N-benzyl,
R(2) is (C₁-C₉)-heteroaryl,
which is linked via C or N and which is unsubstituted or substituted by 1-3 substituents selected from the group consisting of F, Cl, CF₃, CH₃, methoxy, hydroxyl, amino, methylamino and dimethylamino;
or
R(2) is —SR(10), —OR(10), —NR(10)R(11), —CR(10)R(11)R(12);
R(10) is —CₐH₂ₐ—(C₁-C₉)-heteroaryl,
which is unsubstituted or substituted by 1-3 substituents selected from the group consisting of F, Cl, CF₃, CH₃, methoxy, hydroxyl, amino, methylamino and dimethylamino;
a is zero, 1 or 2;
R(11) and R(12)
independently of one another are defined as R(10) or are hydrogen or (C₁-C₄)-alkyl;
R(3) is defined as R(1), or is (C₁-C₆)-alkyl or —X—R(13);
X is oxygen, S, or NR(14);
R(14) is H or (C₁-C₃)-alkyl;
R(13) is H, (C₁-C₆)-alkyl, (C₃-C₈)-cycloalkyl or —C_{b}H_{2b}—R(15);
b is zero, 1, 2, 3 or 4;
or
R(13) and R(14)
together are 4 or 5 methylene groups, of which one CH₂ group can be replaced by oxygen, S, NH, N—CH₃ or N-benzyl;
R(15) is phenyl,
which is unsubstituted or substituted by 1-3 substituents selected from the group consisting of F, Cl, CF₃, methyl, methoxy and NR(8)R(9);
R(8) and R(9)
are H or (C₁-C₄)-alkyl;
and their pharmaceutically tolerable salts;
k) benzoylguanidines of the formula I 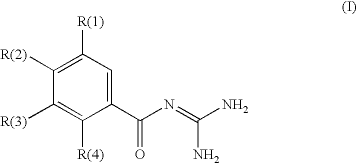
in which:
one of the substituents R(1), R(2), R(3) or R(4): is an amino group 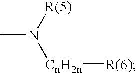
R(5) is hydrogen or C₍₁₋₆₎-alkyl;
n is zero, 1, 2, 3 or 4;
R(6) is H or C₍₁₋₄₎-alkyl;
in which one CH₂ group can be replaced by 1 sulfur atom or a group NR(7);
R(7) is hydrogen, methyl or ethyl;
or
R(6) is C₍₃₋₈₎-cycloalkyl or phenyl,
which is unsubstituted or carries 1, 2 or 3 substituents selected from the group consisting of F, Cl, Br, methyl, methoxy, —NR(8)R(9);
R(8) and R(9)
are H, methyl or ethyl;
or
R(5) and R(6)
together with the nitrogen atom are a 5-, 6- or 7-membered ring, in which 1 carbon atom can be replaced by oxygen, S or NR(10);
R(10) is H, C₍₁₋₃₎-alkyl or benzyl;
and the other substituents R(1), R(2), R(3), R(4) in each case are:
hydrogen, F, Cl, Br, I, CN, CF₃, NO₂, CF₃—O—, CₘF₂ₘ₊₁—CH₂—O— or R(11)-C_{q}H_{2q}—Xₚ—;
m is 1, 2 or 3;
q is zero, 1, 2, 3 or 4;
p zero or 1;
X is oxygen or NR(12):
R(12) is H or C₍₁₋₃₎-alkyl;
R(11) is hydrogen, C₍₁₋₆₎-alkyl, C₍₃₋₈₎-cycloalkyl or phenyl,
which is unsubstituted or substituted by 1, 2 or 3 substituents selected from the group consisting of F, Cl, CH₃, CH₃—O— and NR(13)R(14);
R(13), R(14)
are H, methyl or ethyl;
and their pharmaceutically tolerable salts;
I) benzoylguanidines of the formula I 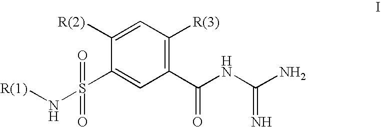
in which
R(1) is R(4)R(5)N—C(X)—;
X is oxygen, S or N—R(6);
R(4) and R(5)
identically or differently, are H, (C₁-C₈)-alkyl, (C₃-C₆)-alkenyl or —CₙH₂ₙ—R(7);
n is zero, 1, 2, 3 or 4;
R(7) is (C₅-C₇)-cycloalkyl or phenyl,
which is unsubstituted or substituted by 1-3 substituents selected from the group consisting of F, Cl, CF₃, methoxy and (C₁-C₄)-alkyl;
or
R(4) and R(5)
together are 4 or 5 methylene groups, of which one CH₂ group can be replaced by oxygen, S, NH, N—CH₃ or N-benzyl;
R(6) is defined as R(4) or is amidine;
R(2) is H, F, Cl, Br, I, (C₁-C₈)-alkyl, 1-alkenyl or 1-alkynyl, (C₃-C₈)-cycloalkyl, (C₃-C₈)-cycloalkyl-(C₁-C₄)-alkyl, phenyl, C₆H₅—(C₁-C₄)-alkyl, naphthyl, biphenylyl, 1,1-diphenyl-(C₁-C₄)-alkyl, cyclopentadienyl, pyridyl, thiopyridyl, pyrrolyl, furanyl, thienyl, thiazolyl, oxazolyl, indenyl, quinolyl, indolyl, benzofuranyl, benzothienyl, benzothiazolyl, benzoxazolyl or —W—R(8);
W is oxygen, S or NR(9);
R(8) is H, (C₁-C₆)-alkyl, (C₅-C₇)-cycloalkyl,
cyclohexylmethyl, cyclopentylmethyl, —(CH₂)ₘCₚF₂ₚ₊ or —C_{q}H_{2q}—R(10);
m is zero or 1;
p is 1, 2 or 3;
q is zero, 1, 2, 3 or 4;
R(10) is phenyl,
which is unsubstituted or substituted by 1-3 substituents selected from the group consisting of F, Cl, CF₃, methyl, methoxy and NR(11)R(12);
R(11) and R(12)
are H or (C₁-C₄)-alkyl;
R(9) is H or (C₁-C₃)-alkyl;
or
R(8) and R(9)
together are 4 or 5 methylene groups, of which one CH₂ group can be replaced by oxygen, S, NH, N—CH₃ or N-benzyl;
R(3) is H, F, Cl, Br, I, (C₁-C₆)-alkyl or —W—R(8) as defined for R(2), and their pharmaceutically acceptable salts;
m) benzoylguanidines of the formula I 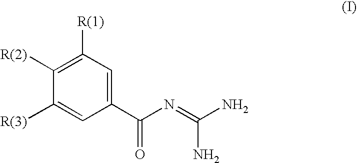
in which:
R(1), R(2), R(3)
are hydrogen, F, Cl, Br, I or (C₁-C₁₂)-alkyl;
one of the substituents R(1), R(2) or R(3)
is N₃, CN, OH or (C₁-C₁₀)-alkyloxy, if at least one of the remaining substituents R(1), R(2) or R(3) is a sufficiently lipophilic alkyl radical having 3 to 12 carbon atoms;
or
one of the substituents R(1), R(2) and R(3)
is R(⁴)—CₙH₂ₙ—Oₘ—;
m is zero or 1;
n is zero, 1, 2 or 3;
R(4) is CₚF₂ₚ₊₁;
p is 1,2 or 3, if n is zero or 1;
or
R(4) is (C₃-C₁₂)-cycloalkyl, phenyl, pyridyl, quinolyl or isoquinolyl,
where the aromatic and heteroaromatic ring systems are unsubstituted or substituted by a substituent selected from the group consisting of F, Cl, CF₃, methyl, methoxy and NR(5)R(6);
R(5) and R(6)
are hydrogen or (C₁-C₄)-alkyl;
or one of the substituents R(1), R(2) and R(3)
is —C≡CR(5) or —C[R(6)]=CR(5);
R(5) is phenyl,
which is unsubstituted or substituted by 1-3 substituents selected from the group consisting of F, Cl, CF₃, methyl, methoxy, hydroxyl, amino, methylamino and dimethylamino, (C₁-C₉)-heteroaryl,
which is unsubstituted or substituted as phenyl,
or
R(5) is (C₁-C₆)-alkyl,
which is unsubstituted or substituted by 1-3 OH;
or
R(5) is (C₃-C₈)-cycloalkyl,
R(6) is hydrogen or methyl;
and their pharmacologically acceptable salts;
o) benzoylguanidines of the formula I 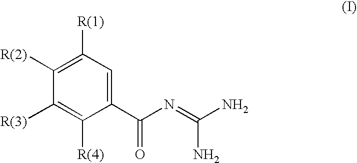
in which:
R(1) is hydrogen, F, Cl, Br, I, —NO₂, —C≡N, Xₒ—(CH₂)ₚ—(CF₂)_{q}—CF₃,
R(5)—SOₘ, R(6)-CO— or R(6)R(7)N—SO₂—, where
X is oxygen, S or NR(14);
m is zero, 1 or 2;
o is zero or 1;
p is zero, 1 or 2;
q is zero, 1, 2, 3, 4, 5 or 6;
R(5) and R(6)
are (C₁-C₈)-alkyl, (C₃-C₆)-alkenyl, —CₙH₂ₙ—R(8) or CF₃;
n is zero, 1, 2, 3 or 4;
R(8) is (C₃-C₇)-cycloalkyl or phenyl,
which is not substituted or is substituted by 1-3 substituents selected from the group consisting of F, Cl, CF₃, methyl, methoxy and NR(9)R(10);
R(9) and R(10)
are H or C₁-C₄-alkyl;
or
R(6) is H;
R(7) is H or (C₁-C₄)-alkyl;
or
R(6) and R(7)
together are 4 or 5 methylene groups, of which one CH₂ group can be replaced by oxygen, S, NH, N—CH₃ or N-benzyl;
R(2) is 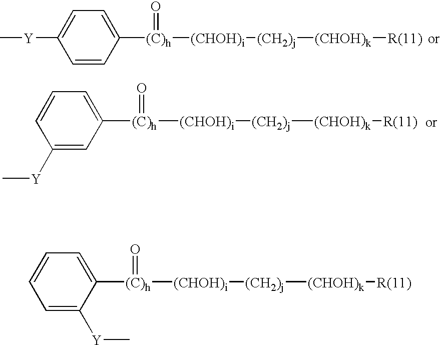
Y is oxygen, —S— or —NR(12)-;
R(11) and R(12)
are hydrogen or (C₁-C₃)-alkyl;
h is zero or 1;
i, j and k
independently are zero, 1, 2, 3 or 4;
but where h, i and k are not simultaneously zero,
R(3) is defined as R(1), or is (C₁-C₆)-alkyl or —X—R(13);
X is oxygen, S or NR(14);
R(14) is H or (C₁-C₃)-alkyl;
R(13) is H, (C₁-C₆)-alkyl, (C₃-C₈)cycloalkyl or —C_{b}H_{2b}—R(15);
b is zero, 1, 2, 3 or 4;
or
R(13) and R(14)
together are 4 or 5 methylene groups, where one CH₂ group can be replaced by oxygen, S, NH, N—CH₃ or N-benzyl;
R(15) is phenyl,
which is unsubstituted or substituted by 1-3 substituents selected from the group consisting of F, Cl, CF₃, methyl, methoxy and NR(9)R(10);
R(9) and R(10)
are H or (C₁-C₄)-alkyl;
R(4) is hydrogen, —OR(16) or —NR(16)R(17);
R(16) and R(17)
independently are hydrogen or (C₁-C₃)-alkyl;
and their pharmaceutically tolerable salts;
p) benzoylguanidines of the formula I 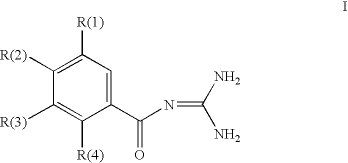
in which;
R(1) is R(6)-CO or R(7)R(8)N—CO;
R(6) is (C₁-C₈)-alkyl, (C₁-C₈)-perfluoroalkyl, (C₃-C₈)-alkenyl or —CₙH₂ₙ—R(9);
n is zero, 1, 2, 3 or 4;
R(9) is (C₃-C₈)-cycloalkyl, phenyl, biphenylyl or naphthyl,
where the aromatics are not substituted or are substituted by 1-3 substituents selected from the group consisting of F, Cl, CF₃, methyl, methoxy and NR(10)R(11);
R(10) and R(11)
are H, (C₁-C₄)-alkyl or (C₁-C₄)-perfluoroalkyl;
R(7) is H, (C₁-C₈)-alkyl, (C₁-C₈)-perfluoroalkyl, (C₃-C₈)-alkenyl or —CₙH₂ₙ—R(12);
n is zero, 1, 2, 3 or 4;
R(1 2) is (C₃-C₈)-cycloalkyl, phenyl, biphenylyl or naphthyl,
where the aromatics are not substituted or are substituted by 1-3 substituents selected from the group consisting of F, Cl, CF₃ methyl, methoxy and NR(13)R(14);
R(13) and R(14)
are H, (C₁-C₄)-alkyl or (C₁-C₄)-perfluoroalkyl;
R(8) is H, (C₁-C₄)-alkyl or (C₁-C₄)-perfluoroalkyl;
or
R(7) and R(8)
together are 4 or 5 methylene groups, of which one CH₂ group can be replaced by oxygen, S, NH, N—CH₃ or N-benzyl;
R(2) is defined [lacuna] R(1), or is H, F, Cl, Br, I, CN, NO₂, (C₁-C₈)-alkyl,
(C₁-C₈)-perfluoroalkyl, (C₃-C₈)-alkenyl or —CₙH₂ₙR(15);
n is zero 1, 2, 3, 4;
R(15) is (C₃-C₈)-cycloalkyl, phenyl, biphenylyl or naphthyl,
where the aromatics are not substituted or are substituted by 1-3 substituents selected from the group consisting of F, Cl, CF₃, methyl, methoxy and
NR(16)R(17);
R(16) and R(17)
are H, (C₁-C₄)-alkyl or (C₁-C₄)-perfluoroalkyl;
or
R(2) is (C₁-C₉)-heteroaryl,
which is linked via C or N and which is unsubstituted or substituted by 1-3 substituents selected from the group consisting of F, Cl, CF₃, CH₃, methoxy, hydroxyl, amino, methylamino and dimethylamino;
or
R(2) is SR(18), —OR(18), —NR(18)R(19), —CR(18)R(19)R(20);
R(18) is —CₐH₂ₐ—(C₁-C₉)-heteroaryl,
which is unsubstituted or substituted by 1-3 substituents selected from the group consisting of F, Cl, CF₃, CH₃, methoxy, hydroxyl, amino, methylamino and dimethylamino;
a is zero, 1 or 2;
R(19) and R(20)
independently of one another are defined as R(18) or are hydrogen, (C₁-C₄)-alkyl or (C₁-C₄)-perfluoroalkyl;
or
R(2) is R(21)-SOₘ or R(22)R(23)N—SO₂—;
m is 1 or 2;
R(21) is (C₁-C₈)-alkyl, (C₁-C₈)-perfluoroalkyl, (C₃-C₈)-alkenyl, —CₙH₂ₙ—R(24),
n is zero, 1, 2, 3 or 4;
R(24) is (C₃-C₈)-cycloalkyl, phenyl, biphenylyl or naphthyl,
where the aromatics are not substituted or are substituted by 1-3 substituents selected from the group consisting of F, Cl, CF₃, methyl, methoxy and NR(27)R(28);
R(27) and R(28)
are H, (C₁-C₄)-alkyl or (C₁-C₄)-perfluoroalkyl;
R(22) is H, (C₁-C₈)-alkyl, (C₁-C₈)-perfluoroalkyl, (C₃-C₈)-alkenyl, —CₙH₂ₙ—R(29);
n is zero, 1, 2, 3 or 4;
R(29) is (C₃-C₈)-cycloalkyl, phenyl, biphenylyl or naphthyl,
where the aromatics are not substituted or are substituted by 1-3 substituents selected from the group consisting of F, Cl, CF₃, methyl, methoxy and NR(30)R(31);
R(30) and R(31)
are H, (C₁-C₄)-alkyl or (C₁-C₄)-perfluoroalkyl;
R(23) is H, (C₁-C₄)-alkyl or (C₁-C₄)-perfluoroalkyl;
or
R(22) and R(23)
together are 4 or 5 methylene groups, of which one CH₂ group can be replaced by oxygen, S, NH, N—CH₃ or N-benzyl;
or
R(2) is R(33)X-;
x is oxygen, S, NR(34), (D═O)A—, NR(34)C═MN^{(*)}R(35)-;
M is oxygen or S;
A is oxygen or NR(34);
D is C or SO;
R(33) is (C₁-C₈)-alkyl, (C₃-C₈)-alkenyl, (CH₂)_{b}C_{d}F_{2d+1}, —CₙH₂ₙ—R(36),
b is zero or 1;
d is 1, 2, 3, 4, 5, 6 or 7;
n is zero, 1, 2, 3 or 4;
R(36) is (C₃-C₈)-cycloalkyl, phenyl, biphenylyl or naphthyl,
where the aromatics are not substituted or are substituted by 1-3 substituents selected from the group consisting of F, Cl, CF₃₁ methyl, methoxy and NR(37)R(38);
R(37) and R(38)
are H, (C₁-C₄)-alkyl or (C₁-C₄)-perfluoroalkyl;
R(34) is H, (C₁-C₄)-alkyl or (C₁-C₄)-perfluoroalkyl;
R(35) is defined as R(33);
or
R(33) and R(34)
together are 4 or 5 methylene groups, of which one CH₂ group can be replaced by oxygen, S, NH, N—CH₃ or N-benzyl;
where A and N^{(*)} are bonded to the phenyl nucleus of the benzoylguanidine parent structure;
or
R(2) is —SR(40), —OR(40), —NHR(40), —NR(40)R(41), —CHR(40)R(42), —C[R(42)R(43)OH], —C≡CR(45), —CR(46)═CHR(45), —[CR(47)R(48)]ᵤ—(CO)-[CR49)R(50)]ᵥ—R(44);
R(40), R(41)
identically or differently are —(CH₂)ₚ—(CHOH)_{q}—(CH₂)ᵣ—(CHOH)ₜ—R(51) or —(CH₂)ₚ—O—(CH₂—CH₂O)_{q}—R(51);
R(51) is hydrogen or methyl;
u is 1, 2, 3 or 4;
v is zero, 1, 2, 3 or 4;
p, q, r
identically or differently are zero, 1, 2, 3 or 4;
t is 1, 2, 3 or 4;
R(42) and R(43)
identically or differently are hydrogen or (C₁-C₆)-alkyl;
or
R(42) and R(43)
together with the carbon atom carrying them form a (C₃-C₈)-cycloalkyl;
R(44) is H, (C₁-C₆)-alkyl, (C₃-C₈)-cycloalkyl or —CₑH₂ₑ—R(⁴⁵);
e is zero, 1, 2, 3 or 4;
R(45) is phenyl,
which is unsubstituted or substituted by 1-3 substituents from the group consisting of F, Cl, CF₃, methyl, methoxy and NR(52)R(53) where
R(52) and R(53) are H or (C₁-C₄)-alkyl, or
R(45) is (C₁-C₉)-heteroaryl,
which is unsubstituted or substituted as phenyl;
or
R(45) is (C₁-C₆)-alkyl,
which is unsubstituted or substituted by 1-3 OH;
R(46), R(47), R(48), R(49) and R(50)
are hydrogen or methyl;
or
R(2) is R(55)-NH—SO₂—;
R(55) is R(56)R(57)N—(C′Y)—;
Y is oxygen, S or N—R(58);
R(56) and R(57)
identically or differently are H, (C₁-C₈)-alkyl, (C₃-C₆)-alkenyl or —C_{f}H_{2f}—R(59);
f is zero, 1, 2, 3 or 4;
R(59) is (C₅-C₇)-cycloalkyl or phenyl,
which is unsubstituted or substituted by 1-3 substituents selected from the group consisting of F, Cl, CF₃, methoxy and (C₁-C₄)-alkyl;
or
R(56) and R(57)
together are 4 or 5 methylene groups, of which one CH₂ group can be replaced by oxygen, S, NH, N—CH₃ or N-benzyl;
R(58) is defined as R(56) or is amidine;
R(3), R(4) and R(5)
independently of one another are defined as R(1) or R(2);
and their pharmaceutically tolerable salts;
q) benzoylguanidines of the formula I 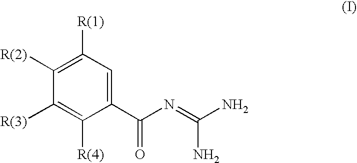
in which:
R(1) is hydrogen, F, Cl, Br, I, —NO₂, —C≡N, —Xₒ—(CH₂)ₚ—(CF₂)_{q}—CF₃, R(5)-SOₘ—, R(6)-CO—, R(6)R(7)N—CO— or R(6)R(7)N—SO₂—;
X is oxygen, —S— or NR(14);
m is zero, 1 or 2;
o is zero or 1;
p is zero, 1 or 2;
q is zero, 1, 2, 3, 4, 5 or 6;
R(5) and R(6)
are (C₁-C₈)-alkyl, (C₃-C₆)-alkenyl, —CₙH₂ₙ—R(8) or CF₃;
n is zero, 1, 2, 3 or 4;
R(8) is (C₃-C₇)-cycloalkyl, phenyl,
which is not substituted or is substituted by 1 to 3 substituents selected from the group consisting of F, Cl, CF₃, methyl, methoxy and NR(9)R(10);
R(9) and R(10)
are H or (C₁-C₄)-alkyl;
or
R(6) is hydrogen;
R(7) is hydrogen or (C₁-C₄)-alkyl;
or
R(6) and R(7)
together are 4 or 5 methylene groups, of which one CH₂ group can be replaced by oxygen, S, NH, N—CH₃ or N-benzyl;
R(2) is 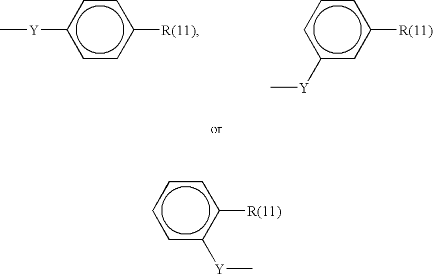
R(11) is (C₁-C₉)-heteroaryl,
which is linked via C or N and which is unsubstituted or substituted by 1 to 3 substituents selected from the group consisting of F, Cl, CF₃, CH₃, methoxy, hydroxyl, amino, methylamino, dimethylamino and benzyl;
Y is oxygen, —S— or NR(12);
R(12) is H or (C₁-C₄)-alkyl;
R(3) is defined as R(1);
or
R(3) is (C₁-C₆)-alkyl or —X—R(13);
X is oxygen, —S— or NR(14);
R(14) is H or (C₁-C₃)-alkyl;
R(13) is H, (C₁-C₆)-alkyl, (C₃-C₈)-cycloalkyl or —C_{b}H_{2b}—R(15);
b is zero, 1, 2, 3 or 4;
or
R(13) and R(14)
together are 4 or 5 methylene groups, of which one CH₂ group can be replaced by oxygen, S, NH, N—CH₃ or N-benzyl;
R(1 5) is phenyl,
which is unsubstituted or substituted by 1-3 substituents selected from the group consisting of F, Cl, CF₃, methyl, methoxy and NR(9)R(10);
R(9) and R(10)
are H or (C₁-C₄)-alkyl;
R(4) is hydrogen, —OR(16), —NR(16)R(17) or CᵣF₂ᵣ₊₁;
R(16) and R(17)
independently are hydrogen or (C₁-C₃)-alkyl;
r is 1, 2, 3 or 4;
and their pharmaceutically tolerable salts;
r) benzo-fused 5-membered ring heterocycles of the formula I 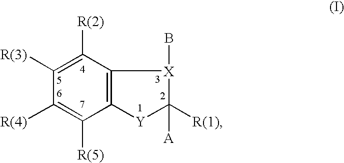
in which:
X is N or CR(6);
Y is oxygen, S or NR(7);
A, B together are a bond
or
A, B are both hydrogen, if X is simultaneously CR(6) and Y is NR(7);
one of the substituents R(1) to R(6) is a —CO—N═C(NH₂)₂ group;
the other substituents R(1) to R(6) in each case
are hydrogen, F, Cl, Br, I or (C₁-C₆)-alkyl;
up to two of the other substituents R(1) to R(6)
are CN, NO₂, N₃, (C₁-C₄)-alkyloxy or CF₃;
up to one of the other substituents
is R(8)-CₙH₂ₙ-Z-;
n is zero to 10;
where the alkylene chain —CₙH₂ₙ— is straight-chain or branched and where one carbon atom can be replaced by an oxygen or sulfur atom or by a nitrogen atom;
R(8) is hydrogen, (C₂-C₆)-alkenyl or (C₃-C₁₀)-cycloalkyl,
which is unsubstituted or substituted by 1 to 4 methyl groups or an OH group, or can contain an ethylene group —CH═CH—, and in which one methylene group can be replaced by an oxygen or sulfur atom or by a nitrogen atom;
or
R(8) is phenyl,
which is unsubstituted or substituted by 1 to 3 substituents selected from the group consisting of F, Cl, Br, I, CF₃, CH₃—S(O)ₛ— or R(9)—W_{y}—;
s is zero, 1 or 2;
R(9) is H, methyl, ethyl,
W is oxygen or NR(10);
R(10) is H or methyl;
y is zero or 1;
or
R(8) is CₘF₂ₘ₊₁;
m is 1 to 3;
or
R(8) is 1- or 2-naphthyl, pyridyl, quinolyl or isoquinolyl;
Z is —CO—, —CH₂— or —[CR(11)(OH)]_{q}—;
q is 1, 2 or 3;
R(11) is H or methyl;
or
Z is oxygen or —NR(12)-;
R(12) is H or methyl;
or
Z is —S(O)ₛ—;
s is zero, 1 or 2;
or
Z is —SO₂—NR(13)-;
R(13) is H or (C₁-C₄)-alkyl;
R(7) is hydrogen, (C₁-C₁₀)-alkyl, (C₂-C₁₀)-alkenyl or R(8)-CₙH₂ₙ—;
and their pharmaceutically tolerable salts;
s) benzoylguanidines of the formula I 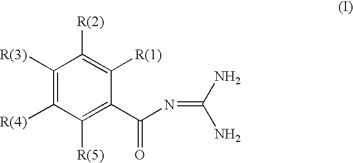
in which:
R(1), R(3) or R(4)
is —NR(6) C=X NR(7)R(8);
X is oxygen or S;
R(6) is hydrogen, (C₁-C₈)-alkyl, (C₁-C₈)-perfluoroalkyl, (C₃-C₈)-alkenyl or —CₙH₂ₙ—R(9);
n is zero, 1, 2, 3 or 4;
R(9) is (C₃-C₈)-cycloalkyl, phenyl, biphenylyl or naphthyl,
where the aromatics are not substituted or are substituted by 1-3 substituents selected from the group consisting of F, Cl, CF₃, methyl, methoxy and NR(10)R(11);
R(10) and R(11)
are H, (C₁-C₄)-alkyl or (C₁-C₄)-perfluoroalkyl;
R(7) is hydrogen, (C₁-C₈)-alkyl, (C₁-C₈)-perfluoroalkyl,
(C₃-C₈)-alkenyl or —CₒH₂ₒ—R(12);
o is zero, 1, 2, 3 or 4;
R(12) is (C₃-C₈)-cycloalkyl, phenyl, biphenylyl or naphthyl,
where the aromatics are not substituted or are substituted by 1-3 substituents selected from the group consisting of F, Cl, CF₃, methyl, methoxy and NR(13)R(14);
R(13) and R(14)
are H, (C₁-C₄)-alkyl or (C₁-C₄)-perfluoroalkyl;
R(8) is defined as R(7);
or
R(7) and R(8)
together are 4 or 5 methylene groups, of which one CH₂ group can be replaced by oxygen, S, NH, N—CH₃ or N-benzyl;
the remaining substituents R(2), R(3), R(4), R(5) or R(1), R(2), R(4), R(5) or R(1), R(2), R(3), R(5) in each case
independently of one another are hydrogen, F, Cl, Br, I, —Oₜₐ(C₁-C₈)-alkyl, —O_{tb}(C₃-C₈)-alkenyl,
—O_{tc}(CH₂)_{b}C_{d}F_{2d+1}, —O_{td}CₚH₂ₚR(18),
or up to 2 groups CN, NO₂, NR(16)R(17),
b is zero or 1;
d is 1, 2, 3, 4, 5, 6 or 7;
ta is zero or 1;
tb is zero or 1;
tc is zero or 1;
td is zero or 1;
p is zero, 1, 2, 3 or 4;
R(1 8) is (C₃-C₈)-cycloalkyl, phenyl, biphenylyl or naphthyl,
where the aromatics are not substituted or are substituted by 1-3 substituents selected from the group consisting of F, Cl, CF₃, methyl, methoxy and
NR(19)R(20);
R(19) and R(20)
are hydrogen or (C₁-C₄)-alkyl or (C₁-C₄)-perfluoroalkyl;
R(16) is hydrogen, (C₁-C₈)-alkyl, (C₁-C₈)-perfluoroalkyl, (C₃-C₈)-alkenyl, —C_{q}H_{2q}—R(²¹),
q is zero, 1, 2, 3 or 4;
R(21) is (C₃-C₈)-cycloalkyl, phenyl, biphenylyl or naphthyl,
where the aromatics are not substituted or are substituted by 1-3 substituents from the group F, Cl, CF₃, methyl, methoxy or NR(22)R(23), R(22) and R(23) are hydrogen, (C₁-C₄)-alkyl or (C₁-C₄)-perfluoroalkyl;
R(17) is hydrogen, (C₁-C₈)-alkyl, (C₁-C₈)-perfluoroalkyl, (C₃-C₈)-alkenyl, —CᵣH₂ᵣ—R(24);
r is zero, 1, 2, 3 or 4;
R(24) is (C₃-C₈)-cycloalkyl, phenyl, biphenylyl or naphthyl,
where the aromatics are not substituted or are substituted by 1-3 substituents selected from the group consisting of F, Cl, CF₃, methyl, methoxy and NR(25)R(26);
R(25) and R(26)
are hydrogen, (C₁-C₄)-alkyl or (C₁-C₄)-perfluoroalkyl;
or
R(16) and R(17)
together are 4 or 5 methylene groups, of which one CH₂ group can be replaced by oxygen, S, NH, N—CH₃ or N-benzyl;
and their pharmaceutically tolerable salts;
t) diacyl-substituted guanidines of the formula I 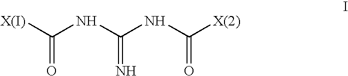
in which:
X(1) and X(2) are 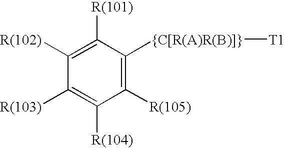
T1 is zero, 1, 2, 3 or 4;
R(A) and R(B)
independently of one another are hydrogen, F, Cl, Br, I, CN, OR(106), (C₁-C₈)-alkyl, (C₃-C₈)-cycloalkyl, O_{zk}(CH₂)_{zl}C_{zm}F_{2zm+1},
NR(107)R(108), phenyl or benzyl,
where the aromatics are not substituted or are substituted by 1-3 substituents selected from the group consisting of F, Cl, CF₃, methyl, methoxy and NR(109)R(110);
R(109) and R(110)
are hydrogen, (C₁-C₄)-alkyl or (C₁-C₄)-perfluoroalkyl;
zl is zero, 1, 2, 3 or 4;
zk is zero or 1;
zm is 1, 2, 3, 4, 5, 6, 7 or 8;
R(106)
is hydrogen, (C₁-C₈)-alkyl, (C₁-C₈)-perfluoroalkyl, (C₃-C₈)-alkenyl, (C₃-C₈)-cycloalkyl, phenyl or benzyl,
where the aromatics are not substituted or are substituted by 1-3 substituents selected from the group consisting of F, Cl, CF₃, methyl, methoxy and NR(111)R(112);
R(111) and R(112)
are hydrogen, (C₁-C₄)-alkyl or (C₁-C₄)-perfluoroalkyl;
R(107) and R(108)
independently of one another are defined as R(106),
or
R(107) and R(108)
together are 4 or 5 methylene groups, of which one CH₂ group can be replaced by oxygen, S, NH, N—CH₃ or N-benzyl;
or
X(1) and X(2) are 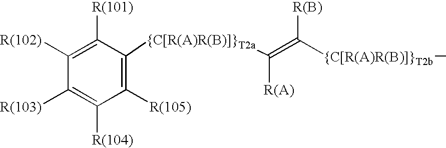
T2a and T2b
independently of one another are zero, 1 or 2;
where the double bond can have the (E)- or (Z)-configuration;
or
X(1) and X(2) are 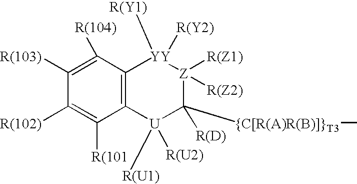
T3 is zero, 1 or 2;
U, YY and Z
independently of one another are C or N, where U, YY, Z can carry the following number of substituents:

| U, YY | Bonded in the ring to | Number of permitted |
|---|---|---|
| or Z | a double bond | substituents |
| C | yes | 1 |
| C | no | 2 |
| N | yes | 0 |
| N | no | 1 |

R(D) is hydrogen, (C₁-C₈)-alkyl or (C₁-C₈)-perfluoroalkyl,
R(U1), R(U2), R(Y1), R(Y2), R(Z1), R(Z2)
independently of one another are hydrogen, F, Cl, Br, I, CN, OR(114), (C₁-C₈)-alkyl, (C₃-C₈)-cycloalkyl, O_{zka}(CH₂)_{zla}C_{zma}F_{2zma+1}, NR(115)R(116), phenyl or benzyl,
where the aromatics are not substituted or are substituted by 1-3 substituents selected from the group consisting of F, Cl, CF₃, methyl, methoxy, NR(117)R(118), R(117) and R(118)
are hydrogen, (C₁-C₄)-alkyl or (C₁-C₄)-perfluoroalkyl,
zka is zero or 1;
zla is zero, 1, 2, 3 or 4;
zma is 1, 2, 3, 4, 5, 6, 7 or 8;
R(114)
is hydrogen, (C₁-C₈)-alkyl, (C₁-C₈)-perfluoroalkyl, (C₃-C₈)-alkenyl, (C₃-C₈)-cycloalkyl, phenyl or benzyl,
where the aromatics are not substituted or are substituted by 1-3 substituents selected from the group consisting of F, Cl, CF₃, methyl, methoxy and NR(119)R(120);
R(119) and R(120)
are hydrogen, (C₁-C₄)-alkyl or (C₁-C₄)-perfluoroalkyl;
R(115) and R(116) independently of one another are defined as R(114);
or
R(115) and R(116)
together are 4 or 5 methylene groups, of which one CH₂ group can be replaced by oxygen, S, NH, N—CH₃ or N-benzyl;
but where the constitution of U is nitrogen (N), YY is nitrogen (N) and Z is carbon (C) is excluded,
R(101), R(102), R(103), R(104) and R(105)
independently of one another are hydrogen, F, Cl, Br, I, —C≡N, X_{zoa}—(CH₂)_{zpa}—(C_{zqa}F_{2zqa+1)}, R(110a)—SO_{zbm}, R(110b)R( 110c)N—CO, R(111a)—CO— or R(112a)R(113a)N—SO₂—,
where the perfluoroalkyl group is straight-chain or branched,
X is oxygen, S or NR(114a);
R(114a)
is H or (C₁-C₃)-alkyl;
zoa is zero or1;
zbm is zero, 1 or2;
zpa is zero, 1, 2, 3 or 4;
zqa is 1, 2, 3, 4, 5, 6, 7 or 8;
R(110a), R(111b), R(111a) and R(112a)
independently of one another are (C₁-C₈)-alkyl, (C₃-C₈)-alkenyl, —C_{zn}H_{2zn}—R(115a) or (C₁-C₈)-perfluoroalkyl;
zn is zero, 1, 2, 3 or 4;
R(115a)
is (C₃-C₈)-cycloalkyl, phenyl, biphenylyl or naphthyl, where the aromatics are not substituted or are substituted by 1-3 substituents selected from the group consisting of F, Cl, CF₃, methyl, methoxy and NR(116a)R(117a);
R(116a) and R(117a)
are hydrogen, (C₁-C₄)-perfluoroalkyl or (C₁-C₄)-alkyl;
or
R(110b), R(111a) and R(112a)
are hydrogen;
R(110c) and R(113a)
independently are hydrogen, (C₁-C₄)-perfluoroalkyl or (C₁-C₄)-alkyl;
or
R(110b) and R(110c) and R(112a) and R(113a)
together are 4 or 5 methylene groups, of which one CH₂ group can be replaced by oxygen, sulfur, NH, N—CH₃ or N-benzyl;
or
R(101), R(102), R(103), R(104), R(105)
independently of one another are (C₁-C₈)-alkyl, —C_{zal}H_{2zal}R(118a) or (C₃-C₈)-alkenyl,
zal is zero, 1, 2, 3 or 4;
R(118a)
is (C₃-C₈)-cycloalkyl, phenyl, biphenylyl or naphthyl, where the aromatics are not substituted or are substituted by 1-3 substituents from the group consisting of F, Cl, CF₃, methyl, methoxy or NR(119a)R(119b);
R(119a) and R(119b)
are hydrogen, (C₁-C₄)-alkyl or (C₁-C₄)-perfluoroalkyl;
or
R(101), R(102), R(103), R(104), R(105)
independently of one another are (C₁-C₉)-heteroaryl,
which is linked via C or N and which is unsubstituted or substituted by 1-3 substituents from the group consisting of F, Cl, CF₃, CH₃, methoxy, hydroxyl, amino, methylamino and dimethylamino;
or
R(101), R(102), R(103), R(104), R(105)
independently of one another are —C≡C—R(193);
R(1 93)
is phenyl which is not substituted or is substituted by 1-3 substituents from the group consisting of F, Cl, CF₃, methyl, methoxy or NR(194)R(195);
R(194) and R(195)
are hydrogen or CH₃;
or
R(101), R(102), R(103), R(104), R(105)
independently of one another are
—Y-para-C₆H₄—(CO)_{zh}—(CHOH)_{zi}—(CH₂)_{zj}—(CHOH)_{zk}—R(123),
—Y-meta-C₆H₄—(CO)_{zad}—(CHOH)_{zae}—(CH₂)_{zaf}—(CHOH)_{zag}—R(₁₂₄)
or
—Y-ortho-C₆H₄—(CO)_{zah}—(CHOH)_{zao}—(CH₂)_{zap}—(CHOH)_{zak}—R(125);
Y is oxygen, —S— or —NR(122d)-;
zh, zad, zah
independently are zero or 1;
zi, zj, zk, zae, zaf, zag, zao, zap and zak
independently are zero, 1, 2, 3 or 4;
but where in each case
zh, zi and zk are not simultaneously zero,
zad, zae and zag are not simultaneously zero, and
zah, zao and zak are not simultaneously zero,
R(123), R(124) R(125) and R(122d)
independently are hydrogen or (C₁-C₃)-alkyl;
or
R(101), R(102), R(103), R(104) and R(105)
independently of one another are SR(129), —OR(130), —NR(131)R(132) or —CR(133)R(134)R(135);
R(129), R(130), R(131) and R(133)
independently are —C_{zab}H_{2zab}—(C₁-C₉)-heteroaryl,
which is unsubstituted or substituted by 1-3 substituents from the group consisting of F, Cl, CF₃, CH₃, methoxy, hydroxyl, amino, methylamino and dimethylamino;
zab is zero, 1 or 2;
R(132), R(134) and R(135)
independently of one another are defined as R(129) or are
hydrogen, (C₁-C₄)-alkyl or (C₁-C₄)-perfluoroalkyl;
or
R(101), R(102), R(103), R(104) and R(105)
independently of one another are —W-para-(C₆-H₄)—R(196), —W-meta-(C₆H₄)—R(197) or —W-ortho-(C₆H₄)—R(198);
R(196), R(197) and R(198)
independently are (C₁-C₉)-heteroaryl,
which is linked via C or N and which is unsubstituted or substituted by 1 to 3 substituents from the group consisting of F, Cl, CF₃, CH₃, methoxy, hydroxyl, amino, methylamino, dimethylamino and benzyl;
W is oxygen, S or NR(136)-;
R(136)
is hydrogen or (C₁-C₄)-alkyl;
or
R(101), R(102), R(103), R(104) and R(105)
independently of one another are R(146)X(1a)-;
X(1a)
is oxygen, S, NR(147), (D═O)A-, NR(148)C═MN^{(*)}R(149)-;
M is oxygen or sulfur;
A is oxygen or NR(150);
D is C or SO;
R(146)
is (C₁-C₈)-alkyl, (C₃-C₈)-alkenyl, (CH₂)_{zbz}C_{zdz}F_{2zdz+1} or —C_{zxa}H_{2zxa}—R(151);
zbz is zero or 1;
zdz is 1, 2, 3, 4, 5, 6 or 7;
zxa is zero, 1, 2, 3 or 4;
R(151)
is (C₃-C₈)-cycloalkyl, phenyl, biphenylyl or naphthyl,
where the aromatics are not substituted or are substituted by 1-3 substituents from the group consisting of F, Cl, CF₃, methyl, methoxy and NR(152)R(153);
R(152) and R(153)
are hydrogen, (C₁-C₄)-alkyl or (C₁-C₄)-perfluoroalkyl;
R(147), R(148) and R(150)
independently are hydrogen, (C₁-C₄)-alkyl, (C₁-C₄)-perfluoroalkyl;
R(149) is defined as R(146),
or
R(146) and R(147), or R(146) and R(148)
together are 4 or 5 methylene groups, of which one CH₂ group can be replaced by oxygen, sulfur, NH, N—CH₃ or N-benzyl;
where A and N^{(*)} are bonded to the phenyl nucleus of the alkanoyl parent structure;
or
R(101), R(102), R(103), R(104) and R(105)
independently of one another are —SR(164), —OR(165), —NHR(166), —NR(1 67)R(168), —CHR(169)R(170), —CR(154)R(155)OH, —C≡CR(156), —CR(158)═CR(157) or —[CR(159)R(160)]_{zu}—(C═O)—[CR(161 )R(162)]_{zv}—R(163); R(164), R(165), R(166), R(167), R(169)
identically or differently are —(CH₂)_{zy}—(CHOH)_{zz}—(CH₂)_{zaa}—(CHOH)_{zt}—R(171) or —(CH₂)_{zab}—O—(CH₂—CH₂O)_{zac}—R(172);
R(171) and R(172)
are hydrogen or methyl;
zu is 1, 2, 3 or 4;
zv is zero, 1, 2, 3 or 4;
zy, zz, zaa, zab, zac
identically or differently are zero, 1, 2, 3 or 4;
zt is 1, 2, 3 or 4;
R(168), R(170), R(154), R(155)
identically or differently are hydrogen or (C₁-C₆)-alkyl,
or
R(169) and R(170), or R(154) and R(155)
together with the carbon atom carrying them are a (C₃-C₈)-cycloalkyl;
R(1 63)
is hydrogen, (C₁-C₆)-alkyl, (C₃-C₈)-cycloalkyl or C_{zeb}H_{2zeb}—R(173);
zeb is zero, 1, 2, 3 or 4;
R(156), R(157) and R(173)
independently are phenyl which is unsubstituted or is substituted by 1-3 substituents from the group consisting of F, Cl, CF₃, methyl, methoxy and NR(174)R(175);
R(174) and R(175)
are hydrogen or (C₁-C₄)-alkyl;
or
R(156), R(157) and R(173)
independently are (C₁-C₉)-heteroaryl,
which is unsubstituted or substituted as phenyl;
R(158), R(159), R(160), R(161) and R(162)
are hydrogen or methyl,
or
R(101), R(102), R(103), R(104), R(105)
independently of one another are R(176)—NH—SO₂—;
R(176)
is R(177)R(178)N—(C═Y′)—;
Y′ is oxygen, S or N—R(179);
R(177) and R(178)
identically or differently are hydrogen, (C₁-C₈)-alkyl, (C₃-C₆)-alkenyl or —C_{zfa}H_{2zfa}—R(180);
zfa is zero, 1, 2, 3 or 4;
R(180)
is (C₅-C₇)-cycloalkyl or phenyl,
which [lacuna] unsubstituted [lacuna] by 1-3 substituents selected from the group consisting of F, Cl, CF₃, methoxy or (C₁-C₄)-alkyl;
or
R(177) and R(178)
together are 4 or 5 methylene groups, of which one CH₂ group can be replaced by oxygen, sulfur, NH, N—CH₃ or N-benzyl;
R(179)
is defined as R(177) or is amidine,
or
R(101), R(102), R(103), R(104), R(105)
independently of one another are NR(184a)R(185), OR(184b), SR(184c) or —C_{znx}H_{2znx}—R(184d);
znx is zero, 1, 2, 3 or 4;
R(184d)
is (C₃-C₇)-cycloalkyl or phenyl,
which is not substituted or substituted by 1-3 substituents from the group consisting of F, Cl, CF₃, methyl, methoxy and NR(116k)R(117k);
R(116k) and R(117k)
are hydrogen or C₁-C₄-alkyl;
R(184a), R(184b), R(184c), R(185)
independently of one another are hydrogen, (C₁-C₈)-alkyl, (C₁-C₈)-perfluoroalkyl or (CH₂)_{zao}—R(184g);
zao is zero, 1, 2, 3 or 4;
184g
is (C₃-C₇)-cycloalkyl or phenyl,
which is not substituted or substituted by 1-3 substituents from the group consisting of F, Cl, CF₃, methyl, methoxy and NR(184u)R(184v);
R(184u) and R(184v)
are hydrogen or C₁-C₄-alkyl;
or
R(184a) and R(185)
together are 4 or 5 methylene groups, of which one CH₂ group can be replaced by oxygen, sulfur, NH, N—CH₃ or N-benzyl;
and their pharmaceutically tolerable salts;
u) benzoylguanidines of the formula I 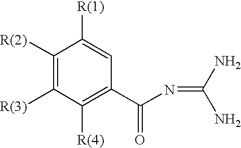
in which:
R(1) is H, F, Cl, Br, I, CN, NO₂, (C₁-C₈)-alkyl, (C₃-C₈)-cycloalkyl or Xₐ—(CH₂)_{b}—(CF₂)_{c}—CF₃;
X is oxygen, S or NR(5);
a is zero or 1;
b is zero, 1 or 2;
c is zero, 1, 2 or 3;
R(5) is H, (C₁-C₄)-alkyl or —C_{d}H_{2d}R(6);
d is zero, 1, 2, 3 or 4;
R(6) is (C₃-C₈)-cycloalkyl, phenyl, biphenylyl or naphthyl,
where the aromatics are not substituted or are substituted by 1 to 3 substituents selected from the group consisting of F, Cl, CF₃, methyl, methoxy and NR(7)R(8);
R(7) and R(8)
independently are H or (C₁-C₄)-alkyl;
or
R(1) is —SR(10), —OR(10) or —CR(10)R(11)R(12);
R(10) is —C_{f}H_{2f}—(C₃-C₈)-cycloalkyl, —(C₁-C₉)-heteroaryl or phenyl,
where the aromatic systems are unsubstituted or substituted by one to 3 substituents selected from the group consisting of F, Cl, CF₃, CH₃, methoxy, hydroxyl, amino, methylamino and dimethylamino;
f is zero, 1 or 2;
R(11) and R(12)
independently of one another are defined as R(10) or are hydrogen or (C₁-C₄)-alkyl;
or
R(1) is phenyl, naphthyl, biphenylyl or (C₁-C₉)-heteroaryl,
the latter linked via C or N,
and which are unsubstituted or substituted by 1 to 3 substituents selected from the group consisting of F, Cl, CF₃, CH₃, methoxy, hydroxyl, amino, methylamino and dimethylamino;
or
R(1) is —SR(13), —OR(13), —NHR(13), —NR(13)R(14), —CHR(13)R(15), —C[R(15)R(16)]OH, —C≡CR(18), —C[R(19)]═CR(18), —[CR(20)R(21)]ₖ—(CO)—[CR(22)R(23)R(24)];
R(13) and R(14)
identically or differently are —(CH₂)_{g}—(CHOH)ₕ—(CH₂)ᵢ—(CHOH)ⱼ—R(17),
R(17) is hydrogen or methyl;
—(CH₂)_{g}—O—(CH₂—CH₂O)ₕ—R(24),
g, h, i
identically or differently are zero, 1, 2, 3 or 4;
is 1, 2, 3 or 4;
R(15) and R(16)
identically or differently are hydrogen, (C₁-C₆)-alkyl or together with the carbon atom carrying them are a (C₃-C₈)-cycloalkyl;
R(18) is phenyl,
which is unsubstituted or substituted by 1 to 3 substituents selected from the group consisting of F, Cl, CF₃, methyl, methoxy and NR(25)R(26);
R(25) and R(26)
are H or (C₁-C₄)-alkyl;
or
R(18) is (C₁-C₉)-heteroaryl,
which is unsubstituted or substituted as phenyl;
or
R(18) is (C₁-C₆)-alkyl,
which is unsubstituted or substituted by 1 to 3 OH;
or
R(18) is (C₃-C₈)-cycloalkyl;
R(19), R(20), R(21), R(22) and R(23)
are hydrogen or methyl;
k is zero, 1, 2, 3 or 4;
l is zero, 1, 2, 3, or 4;
R(24) is H, (C₁-C₆)-alkyl, (C₃-C₈)-cycloalkyl or —CₘH₂ₘ—R(18);
m is1, 2, 3, or 4;
R(2) and R(3)
independently of one another are defined as R(1);
R(4) is (C₁-C₃)-alkyl, F, Cl, Br, I, CN or —(CH₂)ₙ—(CF₂)ₒ—CF₃;
n is zero or 1;
o is zero, 1 or 2;
and their pharmaceutically tolerable salts;
v) acylguanidines of the formula I 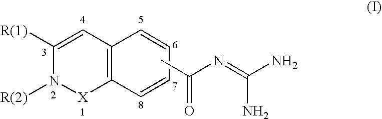
in which:
X is carbonyl, sulfonyl, R(1) is H, (C₁-C₈)-alkyl, unsubstituted or substituted by hydroxyl,
(C₃-C₈)-Cycloalkyl, phenyl,
which is unsubstituted or substituted by 1-3 substituents from the group F, Cl, CF₃, CH₃, methoxy, hydroxyl, amino, methylamino or dimethylamino,
R(2) is H, (C₁-C₄)-alkyl,
and their pharmaceutically tolerable salts;
w) phenyl-substituted alkycarboxylic acid guanidines, carrying perfluoroalkyl groups, of the formula I 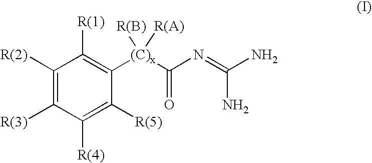
in which:
R(A) is hydrogen, F, Cl, Br, I, CN, OR(6), (C₁-C₈)-alkyl, (C₃-C₈)-cycloalkyl, Oᵣ(CH₂)ₐC_{b}F_{2b+1} or NR(7)R(8);
r is zero or 1;
a is zero, 1, 2, 3 or 4;
b is 1, 2, 3, 4, 5, 6, 7 or 8;
R(6) is hydrogen, (C₁-C₈)-alkyl, (C₃-C₈)-alkenyl, (C₃-C₈)-cycloalkyl, phenyl or benzyl,
where the aromatics are not substituted or are substituted by 1-3 substituents selected from the group consisting of F, Cl, CF₃, methyl, methoxy and NR(9)R(10);
R(9) and R(10)
are H, (C₁-C₄)-alkyl or (C₁-C₄)-perfluoroalkyl;
R(7) and R(8)
independently of one another are defined as R(6);
R(B) independently is defined as R(A);
X is 1, 2 or 3;
R(1) is hydrogen, (C₁-C₈)-alkyl, (C₃-C₈)Cycloalkyl, —Oₜ(CH₂)_{d}CₑF₂ₑ₊₁, F, Cl, Br, I or CN;
t is zero or 1;
d is zero, 1, 2, 3, or 4;
e is 1, 2, 3, 4, 5, 6, 7 or 8;
R(2), R(3), R(4) and R(5)
independently of one another are defined as R(1);
but with the condition
that at least one of the substituents R(1), R(2), R(3), R(4), R(5), R(A) and R(B) is an —Oₜ(CH₂)_{d}CₑF₂ₑ₊₁ or an Oᵣ(CH₂)ₐC_{b}F_{2b+1} group,
and their pharmaceutically tolerable salts;
x) heteroaroylguanidines of the formua I 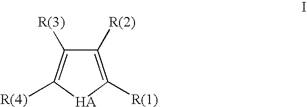
in which:
HA is SOₘ, O or NR(5);
m is zero, 1 or 2;
R(5) is hydrogen, (C₁-C₈)-alkyl or —CₐₘH₂ₐₘR(81);
am is zero, 1 or 2;
R(81) is (C₃-C₈)-cycloalkyl or phenyl,
which is not substituted or is substituted by 1-3 substituents selected from the group consisting of F, Cl, CF₃, methyl, methoxy and NR(82)R(83);
R(82) and R(83)
is H or CH₃;
or
R(81) is (C₁-C₉)-heteroaryl,
which is linked via C or N and which is unsubstituted or substituted by 1-3 substituents selected from the group consisting of F, Cl, CF₃, CH₃, methoxy, hydroxyl, amino, methylamino and dimethylamino:
one of the two substituents R(1) and R(2)
is —CO—N═C(NH₂)₂;
and the other in each case
is hydrogen, F, Cl, Br, I, (C₁-C₃)-alkyl, —OR(6), CᵣF₂ᵣ₊₁, —CO—N═C(NH₂)₂ or —NR(6)R(7);
R(6) and R(7)
independently are hydrogen or (C₁-C₃)-alkyl;
r is1, 2, 3, or 4;
R(3) and R(4)
independently of one another are hydrogen, F, Cl, Br, I, —C≡N, X—(CH₂)ₚ—(C_{q}—F_{2q+1}), R(8)—SO_{bm}, R(9)R(10)N—CO, R(11)—CO— or R(12)R(13)N—SO₂—,
where the perfluoroalkyl group is straight-chain or branched,
X is oxygen, S or NR(14);
R(14) is H or (C₁-C₃)-alkyl;
bm is zero, 1 or 2;
p is zero, 1 or 2;
q is zero, 1, 2, 3, 4, 5 or 6;
R(8), R(9), R(11) and R(12)
independently are (C₁-C₈)-alkyl, (C₃-C₆)-alkenyl, —CₙH₂ₙ—R(15), CF₃;
n is zero, 1, 2, 3 or 4;
R(15) is (C₃-C₇)-cycloalkyl or phenyl;
which is not substituted or is substituted by 1-3 substituents selected from the group consisting of F, Cl, CF₃, methyl, methoxy or NR(16)R(17);
R(16) and R(17)
are H or C₁-C₄-alkyl;
or
R(9), R(11) and R(12)
are H;
R(10) and R(13)
independently are H or (C₁-C₄)-alkyl;
or
R(9) and R(10), and R(12) and R(13)
together are 4 or 5 methylene groups, of which one CH₂ group can be replaced by oxygen, S, NH, N—CH₃ or N-benzyl,
or
R(3) and R(4)
independently of one another are (C₁-C₈)-alkyl or —CₐₗH₂ₐₗR(¹⁸);
al is zero, 1 or 2;
R(18) is (C₃-C₈)-cycloalkyl or phenyl;
which is not substituted or is substituted by 1-3 substituents selected from the group consisting of F, Cl, CF₃, methyl, methoxy and NR(19)R(20);
R(1 9) and R(20)
are H or CH₃;
or
R(3) and R(4)
independently of one another are (C₁-C₉)-heteroaryl,
which is linked via C or N and which is unsubstituted or substituted by 1-3 substituents selected from the group consisting of F, Cl, CF₃, CH₃, methoxy, hydroxyl, amino, methylamino and dimethylamino;
or
R(3) and R(4)
independently of one another are 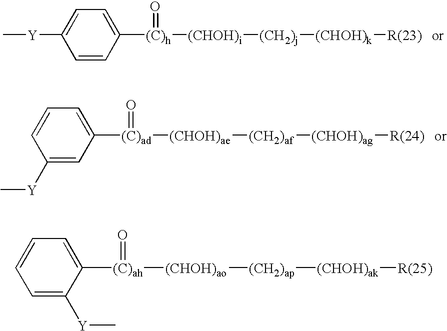
Y is oxygen, —S— or —NR(22)-;
h, ad, ah independently are zero or 1;
i, j, k, ae, af, ag, ao, ap and ak independently are zero, 1, 2, 3, 4, but where in each case
h, i and k are not simultaneously zero,
ad, ae and ag are not simultaneously zero,
ah, ao and ak are not simultaneously zero,
R(23), R(24) R(25) and R(22)
independently are hydrogen or (C₁-C₃)-alkyl;
or
R(3) and R(4)
independently of one another are hydrogen, F, Cl, Br, I, CN, (C₁-C₈)-alkyl, (C₁-C₈)-perfluoroalkyl, (C₃-C₈)-alkenyl or —C_{g}H_{2g}R(26);
g is zero, 1, 2, 3 or 4;
R(26) is (C₃-C₈)-cycloalkyl, phenyl, biphenylyl or naphthyl,
where the aromatics are not substituted or are substituted by 1-3 substituents selected from the group consisting of F, Cl, CF₃, methyl, methoxy and NR(27)R(28);
R(27) and R(28)
are H, (C₁-C₄)-alkyl or (C₁-C₄)-perfluoroalkyl;
or
R(3) and R(4)
independently of one another are SR(29), —OR(30), —NR(31)R(32) or —CR(33)R(34)R(35);
R(29), R(30), R(31) and R(33)
independently are —CₐH₂ₐ—(C₁-C₉)-heteroaryl,
which is unsubstituted or substituted by 1-3 substituents selected from the group consisting of F, Cl, CF₃, CH₃, methoxy, hydroxyl, amino, methylamino and dimethylamino;
a is zero, 1 or 2;
R(32), R(34) and R(35)
independently of one another are defined as R(29) or are hydrogen, (C₁-C₄)-alkyl or (C₁-C₄)-perfluoroalkyl;
or
R(3) and R(4)
independently of one another are 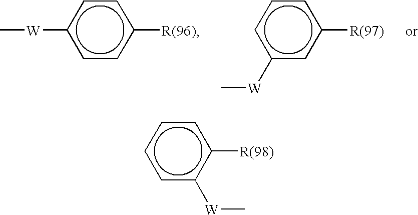
R(96), R(97) and R(98)
independently are (C₁-C₉)-heteroaryl,
which is linked via C or N and which is unsubstituted or substituted by 1 to 3 substituents selected from the group consisting of F, Cl, CF₃, CH₃, methoxy, hydroxyl, amino, methylamino, dimethylamino or benzyl;
W is oxygen, S or NR(36)-;
R(36) is H or (C₁-C₄)-alkyl;
or
R(3) and R(4)
independently of one another are R(37)—SO_{cm} or R(38)R(39)N—SO₂—;
cm is 1 or 2;
R(37) is (C₁-C₈)-alkyl, (C₁-C₈)-perfluoroalkyl, (C₃-C₈)-alkenyl or —CₛH₂ₛR(40);
s is zero, 1, 2, 3 or 4;
R(40) is (C₃-C₈)-cycloalkyl, phenyl, biphenylyl or naphthyl,
where the aromatics are not substituted or are substituted by 1-3 substituents selected from the group consisting of F, Cl, CF₃, methyl, methoxy and NR(41)R(42);
R(41) and R(42)
are H, (C₁-C₄)-alkyl or (C₁-C₄)-perfluoroalkyl;
R(38) is H, (C₁-C₈)-alkyl, (C₁-C₈)-perfluoroalkyl, (C₃-C₈)-alkenyl or —C_{w}H_{2w}—R(43);
w is zero, 1, 2, 3 or 4;
R(43) is (C₃-C₈)-cycloalkyl, phenyl, biphenylyl or naphthyl,
where the aromatics are not substituted or are substituted by 1-3 substituents selected from the group consisting of F, Cl, CF₃, methyl, methoxy and NR(44)R(45);
R(44) and R(45)
are H, (C₁-C₄)-alkyl or (C₁-C₄)-perfluoroalkyl;
R(39) is H, (C₁-C₄)-alkyl or (C₁-C₄)-perfluoroalkyl;
or
R(38) and R(39)
together are 4 or 5 methylene groups, of which one CH₂ group can be replaced by oxygen, S, NH, N—CH₃ or N-benzyl;
or
R(3) and R(4)
independently of one another are R(46)X(1)-;
X(1) is oxygen, S, NR(47), (D═O)A—, NR(48)C═MN^{(*)}R(49)-,
M is oxygen or S;
A is oxygen or NR(50);
D is C or SO;
R(46) is (C₁-C₈)-alkyl, (C₃-C₈)-alkenyl, (CH₂)_{b}C_{d}F_{2d+1} or —CₓH₂ₓ—
R(51);
b is zero or 1;
d is 1, 2, 3, 4, 5, 6 or 7;
x is zero, 1, 2, 3, or 4;
R(51) is (C₃-C₈)-cycloalkyl, phenyl, biphenylyl or naphthyl,
where the aromatics are not substituted or are substituted by 1-3 substituents selected from the group consisting of F, Cl, CF₃, methyl, methoxy and NR(52)R(53);
R(52) and R(53)
are H, (C₁-C₄)-alkyl or (C₁-C₄)-perfluoroalkyl;
R(47), R(48) and R(50)
independently are hydrogen, (C₁-C₄)-alkyl or (C₁-C₄)-perfluoroalkyl;
R(49) is defined as R(46);
or
R(46) and R(47), or R(46) and R(48)
together are 4 or 5 methylene groups, of which one CH₂ group can be replaced by oxygen, S, NH, N—CH₃ or N-benzyl,
where A and N^{(*)} are bonded to the phenyl nucleus of the benzoylguanidine parent structure;
or
R(3) and R(4)
independently of one another are —SR(64), —OR(65), —NHR(66), —NR(67)R(68), —CHR(69)R(70), —C(OH)R(54)R(55), —C≡CR(56), —CR(58)═CHR(57), —[CR(59)R(60)]ᵤ—(CO)-[CR(61 )R(62)]ᵥ—R(63);
R(64), R(65), R(66), R(67) and R(69)
identically or differently are —(CH₂)_{y}—(CHOH)_{z}—(CH₂)ₐₐ—(CH₂OH)ₜ—R(71) or —(CH₂)_{ab}—O—(CH₂—CH₂O)_{ac}—R(72), R(71) and R(72)
are hydrogen or methyl;
u is 1, 2, 3 or 4;
v is zero, 1, 2, 3 or 4;
y, z, aa
identically or differently are zero, 1, 2, 3 or 4;
t is 1, 2, 3, or 4;
R(68), R(70), R(54) and R(55)
identically or differently are hydrogen, (C₁-C₆)-alkyl;
or
R(69) and R(70), or R(54) and R(55)
together with the carbon atom carrying them are a (C₃-C₈)-cycloalkyl;
R(63) is H, (C₁-C₆)-alkyl, (C₃-C₈)-cycloalkyl or —CₑH₂ₑ—R(73);
e is zero, 1, 2, 3 or 4;
R(56), R(57) and R(73)
independently are phenyl,
which are unsubstituted or substituted by 1-3 substituents selected from the group consisting of F, Cl, CF₃, methyl, methoxy and NR(74)R(75);
R(74) and R(75)
are H or (C₁-C₄)-alkyl;
or
R(56), R(57) and R(73)
independently are (C₁-C₉)-heteroaryl,
which is unsubstituted or substituted as phenyl;
R(58), R(59), R(60), R(61) and R(62)
are hydrogen or methyl,
or
R(3) and R(4)
independently of one another are R(76)-NH—SO₂—;
R(76) is R(77)R(78)N—C═Y′);
Y′ is oxygen, S or N—R(79);
R(77) and R(78)
identically or differently are H, (C₁-C₈)-alkyl, (C₃-C₆)alkenyl, —C_{f}H_{2f}R(80);
f is zero, 1, 2, 3, or 4;
R(80) is (C₅-C₇)-cycloalkyl or phenyl,
which is unsubstituted or substituted by 1-3 substituents selected from the group consisting of F, Cl, CF₃, methoxy and (C₁-C₄)alkyl;
or
R(77) and R(78)
together are 4 or 5 methylene groups, of which one CH₂ group can be replaced by oxygen, S, NH, N—CH₃ or N-benzyl, R(79)
is defined as R(77) or is amidine;
or
R(3) and R(4)
independently of one another are NR(84)R(85);
R(84) and R(85)
independently of one another are H, (C₁-C₄)-alkyl, or together are 4 or 5 methylene groups,
of which one CH₂ group can be replaced by oxygen, S, NH, N—CH₃ or N-benzyl; or of which one or two CH₂ groups can be replaced by CH—C_{dm}H_{2dm+1},
and their pharmaceutically tolerable salts;
y) bicyclic heteroaroylguanidines of the formula I 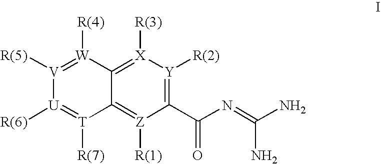
in which:
T, U, V, W, X, Y and Z
independently of one another are nitrogen or carbon;
but with the restriction
that X and Z are not simultaneously nitrogen,
and that T, U, V, W, X, Y and Z carry no substituents if they are nitrogen,
and that no more than four of them are simultaneously nitrogen,
R(1) and R(2)
independently of one another are hydrogen, F, Cl, Br, I, (C₁-C₃)-alkyl, (C₁-C₃)-perfluoroalkyl, OR(8), NR(8)R(9) or C(═O)N═C(NH₂)₂;
R(8) and R(9)
independently of one another are hydrogen or (C₁-C₃)-alkyl,
or
R(8) and R(9)
together are 4 or 5 methylene groups, of which one CH₂ group can be replaced by oxygen, S, NH, N—CH₃ or N-benzyl;
R(3), R(4), R(5), R(6) and R(7)
independently of one another are hydrogen, F, Cl, Br, I, —C≡N, Xₖ—(CH₂)ₚ—(C_{q}F_{2q+1}), R(10a)—SO_{bm}, R(10b)R(10c)N—CO, R(11)-CO— or R(12)R(13)N—SO₂—,
where the perfluoroalkyl group is straight-chain or branched;
X is oxygen, S or NR(14);
R(14) is H or (C₁-C₃)-alkyl;
bm is zero, 1 or 2;
p is zero, 1 or 2;
k is zero or 1;
q 1, 2, 3, 4, 5 or 6;
R(10a), R(10b), R(11) and R(12)
independently of one another are (C₁-C₈)-alkyl, (C₃-C₆)-alkenyl, —CₙH₂ₙ—R(15) or (C₁-C₈)-perfluoroalkyl;
n is zero, 1, 2, 3 or 4;
R(15) is (C₃-C₇)-cycloalkyl or phenyl, which is not substituted
or is substituted by 1-3 substituents selected from the group consisting of F, Cl, CF₃, methyl, methoxy and NR(16)R(17);
R(16) and R(17)
are H or C₁-C₄-alkyl;
or
R(10b), R(11) and R(12)
are hydrogen;
R(10c) and R(13)
independently are hydrogen or (C₁-C₄)-alkyl;
or
R(10b) and R(10c) and R(12) and R(13)
together are 4 or 5 methylene groups, of which one CH₂ group can be replaced by oxygen, sulfur, NH, N—CH₃ or N-benzyl;
or
R(3), R(4), R(5), R(6) and R(7)
independently of one another are (C₁-C₈)-alkyl, —CₐₗH₂ₐₗR(18) or (C₃-C₈)-alkenyl;
al is zero, 1 or 2;
R(18) is (C₃-C₈)-cycloalkyl, phenyl, biphenylyl or naphthyl, where
the aromatics are not substituted or are substituted by 1-3 substituents selected from the group consisting of F, Cl, CF₃, methyl, methoxy and NR(19a)R(19b);
R(19a) and R(19b)
are hydrogen, (C₁-C₄)-alkyl or (C₁-C₄)-perfluoroalkyl;
or
R(3), R(4), R(5), R(6) and R(7)
independently of one another are (C₁-C₉)-heteroaryl, which is linked via C or N and which is unsubstituted or substituted by 1-3 substituents from the group consisting of F, Cl, CF₃, CH₃, methoxy, hydroxyl, amino, methylamino or dimethylamino;
or
R(3), R(4), R(5), R(6) and R(7)
independently of one another are 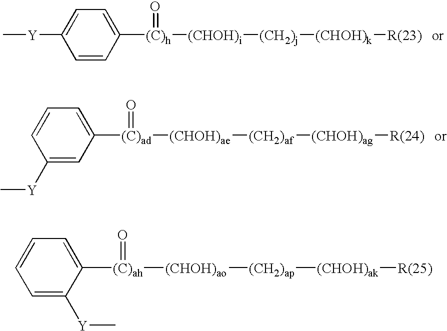
Y is oxygen, —S— or —NR(22)-;
h, ad, ah
independently of one another are zero or 1;
i, j, k, ae, af, ag, ao, ap and ak
independently of one another are zero, 1, 2, 3 or 4;
but where in each case
h, i and k are not simultaneously zero,
ad, ae and ag are not simultaneously zero, and
ah, ao and ak are not simultaneously zero,
R(23), R(24) R(25) and R(22)
independently of one another are hydrogen or (C₁-C₃)-alkyl;
or
R(3), R(4), R(5), R(6) and R(7)
independently of one another are SR(29), —OR(30), —NR(31)R(32) or —CR(33)R(34)R(35);
R(29), R(30), R(31) and R(33)
independently of one another are —CₐH₂ₐ—(C₁-C₉)-heteroaryl, which is unsubstituted or substituted by 1-3 substituents selected from the group consisting of F, Cl, CF₃, CH₃, methoxy, hydroxyl, amino, methylamino and dimethylamino;
a is zero, 1 or 2;
R(32), R(34) and R(35)
independently of one another are defined as R(29) or are hydrogen, (C₁-C₄)-alkyl or (C₁-C₄)-perfluoroalkyl;
or
R(3), R(4), R(5), R(6) and R(7)
independently of one another are 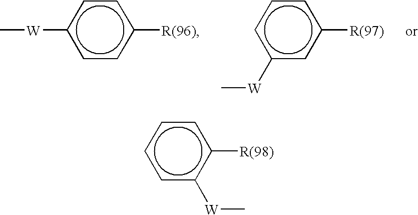
R(96), R(97) and R(98)
independently of one another are (C₁-C₉)-heteroaryl, which is linked via C or N and which is unsubstitued or substituted by 1 to 3 substituents from the group consisting of F, Cl, CF₃, CH₃, methoxy, hydroxyl, amino, methylamino, dimethylamino or benzyl;
W is oxygen, S or NR(36)-;
R(36) is H or (C₁-C₄)-alkyl;
or
R(3), R(4), R(5), R(6) and R(7)
independently of one another are R(46)X(1)-;
X(1) is oxygen, S, NR(47), (D=O)A- or NR(48)C=MN^{(*)}R(49)-;
M is oxygen or sulfur;
A is oxygen or NR(50);
D is C or SO;
R(46) is (C₁-C₈)-alkyl, (C₃-C₈)alkenyl, (CH₂)_{b}C_{d}F_{2d+1} or —CₓH₂ₓ—
R(51);
b is zero or 1;
d is 1, 2, 3, 4, 5, 6 or 7;
x is zero, 1, 2, 3 or 4;
R(51) is (C₃-C₈)-cycloalkyl, phenyl, biphenylyl or naphthyl,
where the aromatics are not substituted or are substituted by 1-3 substituents selected from the group consisting of F, Cl, CF₃, methyl, methoxy and NR(52)R(53);
R(52) and R(53)
are hydrogen, (C₁-C₄)-alkyl or (C₁-C₄)-perfluoroalkyl;
R(47), R(48) and R(50) independently
are hydrogen, (C₁-C₄)-alkyl or (C₁-C₄)-perfluoroalkyl;
R(49) is defined as R(46);
or
R(46) and R(47), or R(46) and R(48)
together are 4 or 5 methylene groups, of which one CH₂ groups can be replaced by oxygen, sulfur, NH, N—CH₃ or N— benzyl;
where A and N^{(*)} are bonded to the phenyl nucleus of the heteroaroylguanidine parent structure:
or
R(3), R(4), R(5), R(6) and R(7)
independently of one another are —SR(64), —OR(65), —NHR(66), —NR(67)R(68), —CHR(69)R(70) or —CR(54)R(55)OH, —C≡CR(56), —CR(58)=CR(57) or —[CR(59)R(60)]ᵤ—CO—[CR(61)R(62)]ᵥ—R(63);
R(64), R(65), R(66), R(67) and R(69)
identically or differently are
—(CH₂)_{y}—(CHOH)_{z}—(CH₂)ₐₐ—(CHOH)ₜ—R(71) or —(CH₂)_{ab}—O—(CH₂—CH₂O)_{ac}—R(72);
R(71) and R(72)
independently of one another are hydrogen or methyl;
u is 1, 2, 3, or 4;
v is zero, 1, 2, 3, or 4;
y, z, aa identically or differently
are zero, 1, 2, 3 or 4;
t is 1, 2, 3 or 4;
R(68), R(70), R(54) and R(55)
identically or differently are hydrogen or (C₁-C₆)-alkyl;
or
R(69) and R(70), or R(54) and R(55)
together with the carbon atom carrying them are (C₃-C₈)-cycloalkyl;
R(63)
is hydrogen, (C₁-C₆)-alkyl, (C₃-C₈)-cycloalkyl or —CₑH₂ₑ—R(⁷³);
e is zero, 1, 2, 3 or 4;
R(56), R(57) and R(73) independently
are phenyl, which is unsubstituted or substituted by 1-3 substituents selected from the group consisting of F, Cl, CF₃, methyl, methoxy and NR(74)R(75);
R(74) and R(75)
are hydrogen or (C₁-C₄)-alkyl;
or
R(56), R(57) and R(73) independently
are (C₁-C₉)-heteroaryl, which is unsubstituted or substituted as phenyl;
R(58), R(59), R(60), R(61) and R(62)
are hydrogen or methyl;
or
R(3), R(4), R(5), R(6) and R(7)
independently of one another are R(76)-NH—SO₂—;
R(76) is R(77)R(78)N—(C═Y′)—;
Y′ is oxygen, S or N—R(79);
R(77) and R(78)
identically or differently are hydrogen, (C₁-C₈)-alkyl, (C₃-C₆)-alkenyl or —C_{f}H_{2f}—R(80);
f is zero, 1, 2, 3, or 4;
R(80) is (C₅-C₇)-cycloalkyl or phenyl, which [lacuna] unsubstituted [lacuna] by 1-3 substituents selected from the group consisting of F, Cl, CF₃, methoxy and (C₁-C₄)-alkyl;
or
R(77) and R(78)
together are 4 or 5 methylene groups, of which one CH₂ group can be replaced by oxygen, sulfur, NH, N—CH₃ or N-benzyl;
R(79) is defined as R(77) or is amidine;
or
R(3), R(4), R(5), R(6) and R(7)
independently of one another are NR(84a)R(85), OR(84b), SR(84c)
or —CₙH₂ₙ—R(84d);
n is zero, 1, 2, 3, or 4;
R(84d) is (C₃-C₇)-cycloalkyl or phenyl, which is not substituted or is substituted by 1-3 substituents from the group consisting of F, Cl, CF₃, methyl, methoxy and NR(16)R(17);
R(16) and R(17)
are hydrogen or C₁-C₄-alkyl;
R(84a), R(84b), R(84c) and R(85)
independently of one another are hydrogen, (C₁-C₈)-alkyl, (C₁-C₈)-perfluoroalkyl or (CH₂)ₐₓ—R(84g);
ax is zero, 1, 2, 3 or 4;
R(84g) is (C₃-C₇)-cycloalkyl or phenyl, which is not substituted or is substituted by 1-3 substituents selected from the group consisting of F, Cl, CF₃, methyl, methoxy and NR(84u)R(84v);
R(84u) and R(84v)
are hydrogen or C₁-C₄-alkyl;
or
R(84a) and R(85)
together are 4 or 5 methylene groups, of which one CH₂ group can be replaced by oxygen, sulfur, NH, N—CH₃ or N-benzyl,
and their pharmaceutically tolerable salts;
z) benzoylguanidines of the formula I 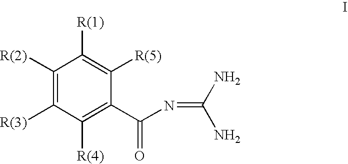
in which:
R(1) is R(⁶)—SOₘ;
m is zero, 1 or 2;
R(6) is perfluoroalkyl having 1, 2, 3, 4, 5 or 6 carbon atoms, which is straight-chain or branched;
R(2) and R(3)
independently of one another are hydrogen, F, Cl, Br, I, alkyl having 1, 2, 3 or 4 carbon atoms, alkoxy having 1, 2, 3 or 4 carbon atoms or phenoxy,
which is unsubstituted or substituted by 1-3 substituents selected from the group consisting of F, Cl, methyl and methoxy;
or
R(2) and R(3)
independently of one another are pyrrol-1-yl, pyrrol-2-yl or pyrrol-3-yl,
which is not substituted or is substituted by 1 to 4 substituents selected from the group consisting of F, Cl, Br, I, CN, alkanoyl having 2, 3, 4, 5, 6, 7 or 8 carbon atoms, alkoxycarbonyl having 2, 3, 4, 5, 6, 7 or 8 carbon atoms, formyl, carboxyl, CF₃, methyl and methoxy;
R(4) and R(5)
independently of one another are hydrogen, alkyl having 1, 2 or 3 carbon atoms, F, Cl, Br, I, CN, OR(7), NR(8)R(9) or —(CH₂)ₙ—CF₂)ₒ—CF₃;
R(7), R(8) and R(9)
independently of one another are hydrogen or alkyl having 1, 2, 3 or 4 carbon atoms;
n is zero or 1;
o is zero, 1 or 2;
and their pharmacologically acceptable salts;
ab) phenyl-substituted alkenylcarboxylic acid guanidines, carrying perfluoroalkyl groups, of the formula I 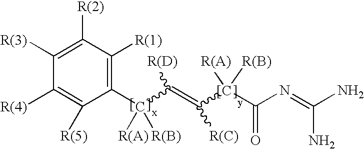
in which:
R(A) is hydrogen, F, Cl, Br, I, CN, OH, OR(6), (C₁-C₈)-alkyl, Oᵣ(CH₂)ₐC_{b}F_{2b+1},(C₃-C₈)-cycloalkyl or NR(7)R(8);
r is zero or 1;
a is zero, 1, 2, 3 or 4;
b is 1, 2, 3, 4, 5, 6, 7 or 8;
R(6) is (C₁-C₈)-alkyl, (C₁-C₄)-perfluoroalkyl, (C₃-C₈)-alkenyl, (C₃-C₈)-cycloalkyl, phenyl or benzyl;
where the aromatics are not substituted or are substituted by 1-3 substituents from the group consisting of F, Cl, CF₃, methyl, methoxy and NR(9)R(10);
R(9) and R(10)
are H, (C₁-C₄)-alkyl or (C₁-C₄)-perfluoroalkyl;
R(7) and R(8)
independently of one another are defined as R(6);
or
R(7) and R(8)
together are 4 or 5 methylene groups, of which one CH₂ group can be replaced by oxygen, sulfur, NH, NCH₃ or N-benzyl;
R(B) independently is defined as R(A);
x is zero, ₁or 2;
y is zero, 1 or 2;
R(C) is hydrogen, F, Cl, Br, I, CN, OR(12), (C₁-C₈)-alkyl, Oₚ(CH₂)_{f}C_{g}F_{2g+1} or (C₃-C₈)-cycloalkyl;
p is zero or 1;
f is zero, 1, 2, 3, or 4;
g is 1, 2, 3, 4, 5, 6, 7 or 8;
R(12)
is (C₁-C₈)-alkyl, (C₁-C₄)-perfluoroalkyl, (C₃-C₈)-alkenyl, (C₃-C₈)-cycloalkyl, phenyl or benzyl;
where the aromatics phenyl or benzyl are not substituted or are substituted by 1-3 substituents from the group consisting of F, Cl, CF₃, methyl, methoxy and NR(13)R(14);
R(13) and R(14)
independently of one another are H, (C₁-C₄)-alkyl or (C₁-C₄)-perfluoroalkyl;
R(D) independently is defined as R(C),
R(1) is hydrogen, (C₁-C₈)-alkyl, —Oₜ(CH₂)_{d}CₑF₂ₑ₊₁, (C₃-C₈)-cycloalkyl, F, Cl, Br, I or CN;
t is zero or 1;
d is zero, 1, 2, 3, or 4;
e is 1, 2, 3, 4, 5, 6, 7 or 8;
R(2), R(3), R(4) and R(5)
independently of one another are defined as R(1);
but with the condition
that at least one of the substituents R(A), R(B), R(C), R(D), R(1), R(2), R(4) or R(5) is a Oᵣ(CH₂)ₐC_{b}F_{2b+1}, Oₚ(CH₂)_{f}C_{g}F_{2g+1} or Oₜ(CH₂)_{d}CₑF₂ₑ₊₁ group and R(3) is not a Oₜ(CH₂)_{d}CₑF₂ₑ₊₁ group;
and their pharmaceutically tolerable salts;
ac) ortho-amino-substituted benzoylguanidines of the formula I 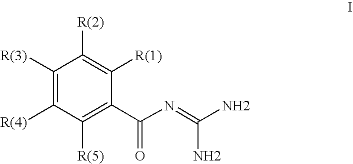
in which:
R(1) is NR(50)R(6),
R(50) and R(6)
independently of one another are hydrogen, (C₁-C₈)-alkyl or (C₁-C₈)-perfluoroalkyl;
R(2), R(3), R(4) and R(5)
independently of one another are R(10)—SOₐ—, R(11)R(12)N—CO—, R(13)—CO— or R(14)R(15)N—SO₂—;
a is zero, 1 or 2,
R(10), R(11), R(12), R(13), R(14) and R(15)
independently of one another are (C₁-C₈)-alkyl, (C₁-C₈)-perfluoroalkyl, (C₃-C₆)-alkenyl or —C_{ab}H_{2ab}—R(16);
ab is zero, 1, 2, 3 or 4;
R(16) is (C₃-C₇)-cycloalkyl, phenyl,
which is not substituted or substituted by 1-3 substituents selected from the group consisting of F, Cl, CF₃, methyl, methoxy or NR(17)R(18);
R(17) and R(18)
independently of one another are H, CF₃ or (C₁-C₄)-alkyl;
or
R(11), R(12), and also R(14) and R(15)
together are 4 or 5 methylene groups, of which one CH₂ group can be replaced by oxygen, S, NH, N—CH₃ or N-benzyl;
or
R(11), R(12), R(14) and R(15)
independently of one another are hydrogen;
or
R(2), R(3), R(4) and R(5)
independently of one another are SR(21), —OR(22), —NR(23)R(24) or —CR(25)R(26)R(27);
R(21), R(22), R(23) and R(25)
independently of one another are —C_{b}H_{2b}—(C₁-C₉)-heteroaryl, which is unsubstituted or substituted by 1-3 substituents selected from the group consisting of F, Cl, CF₃, CH₃, methoxy, hydroxyl, amino, methylamino and dimethylamino;
b is zero, 1 or 2;
R(24), R(26) and R(27)
independently of one another are hydrogen, (C₁-C₄)-alkyl or (C₁-C₄)-perfluoroalkyl;
or
R(2), R(3), R(4) and R(5)
independently of one another are hydrogen, F, Cl, Br, I, CN, —(Xa)_{dg}—C_{da}H_{2da+1}, —(Xb)_{dh}—(CH₂)_{db}—C_{de}F_{2de+1}, (C₃-C₈)-alkenyl or —C_{df}H_{2df}R(30);
(Xa) is O, S or NR(33);
R(33)
is H, (C₁-C₄)-alkyl or (C₁-C₄)-perfluoroalkyl;
dg is zero or 1;
(Xb) is O, S or NR(34);
R(34)
is H, (C₁-C₄)-alkyl or (C₁-C₄)-perfluoroalkyl;
dh is zero or 1;
da is zero, 1, 2, 3, 4, 5, 6, 7, 8;
db is zero, 1, 2, 3, 4;
de is zero, 1, 2, 3, 4, 5, 6, 7;
df is zero, 1, 2, 3, 4;
R(30)
is (C₃-C₈)-cycloalkyl, phenyl, biphenylyl or naphthyl, where the aromatics phenyl, biphenylyl or naphthyl are not substituted or are substituted by 1-3 substituents selected from the group consisting of F, Cl, CF₃, methyl, methoxy and NR(31 )R(32);
R(31) and R(32)
are H, (C₁-C₄)-alkyl or (C₁-C₄)-perfluoroalkyl;
or
R(2), R(3), R(4) and R(5)
independently of one another are NR(40)R(41) or —(Xe)—(CH₂)_{eb}R(45);
R(40) and R(41)
independently of one another are hydrogen, (C₁-C₈)-alkyl, (C₁-C₈)-perfluoroalkyl or (CH₂)ₑ—R(42);
e is zero, 1, 2, 3 or 4;
R(42)
is (C₃-C₇)-cycloalkyl, phenyl, which is not substituted or is substituted by 1-3 substituents selected from th group consisting of F, Cl, CF₃, methyl, methoxy and NR(43)R(44);
R(43) and R(44)
independently of one another are H, CF₃ or (C₁-C₄)-alkyl;
or
R(40) and R(41)
together are 4 or 5 methylene groups, of which one CH₂ group can be replaced by oxygen, sulfur, NH, N—CH₃ or N-benzyl;
(Xe) is O, S or NR(47);
R(47)
is H, (C₁-C₄)-alkyl or (C₁-C₄)-perfluoroalkyl;
eb is zero, 1, 2, 3 or 4;
R(45) is (C₃-C₇)-cycloalkyl, phenyl, which is not substituted or is substituted by 1-3 substituents selected from the group consisting of F, Cl, CF₃,methyl, methoxy, NR(50)R(51) and —(Xfa)-(CH₂)_{ed}—(Xfb)R(46);
Xfa is CH₂, O, S or NR(48);
Xfb is O, S or NR(49);
ed is 1, 2, 3 or 4;
R(46)
is H, (C₁-C₄)-alkyl or (C₁-C₄)-perfluoroalkyl;
R(48), R(49), R(50) and R(51)
independently of one another are H or (C₁-C₄)-alkyl or (C₁-C₄)-perfluoroalkyl;
where R(3) and R(4), however, cannot be hydrogen,
and their pharmaceutically tolerable salts;
ad) benzoylguanidines of the formula I 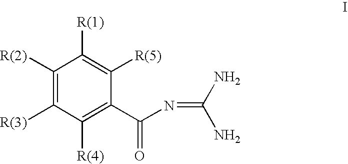
in which:
one of the three substituents R(1), R(2) and R(3)
is (C₁-C₉)-heteroaryl-N-oxide,
which is linked via C or N and which is unsubstituted or substituted by 1-3 substituents selected from the group consisting of F, Cl, CF₃, CH₃, methoxy, hydroxyl, amino, methylamino and dimethylamino;
or
one of the three substituents R(1), R(2) and R(3)
is —SR(10), —OR(10), —NR(10)R(11) or —CR(10)R(11)R(12);
R(10)
is —CₐH₂ₐ—(C₁-C₉)-heteroaryl-N-oxide,
which is unsubstituted or substituted by 1-3 substituents selected from the group consisting [lacuna] F, Cl, CF₃, CH₃, methoxy, hydroxyl, amino, methylamino and dimethylamino;
a is zero, 1 or 2;
R(11) and R(12)
independently of one another are defined as R(10), are hydrogen or (C₁-C₄)-alkyl;
and the other substituents R(1), R(2) and R(3) in each case
independently of one another are (C₁-C₈)-alkyl, (C₂-C₈)-alkenyl or —CₘH₂ₘR(14);
m is zero, 1 or 2;
R(14) is (C₃-C₈)-cycloalkyl or phenyl,
which is not substituted or is substituted by I -3 substituents selected from the group consisting of F, Cl, CF₃, methyl, methoxy and NR(15)R(16),
R(15) and R(16)
are hydrogen or CH₃;
or
the other substituents R(1), R(2) and R(3) in each case
independently of one another are hydrogen, F, Cl, Br, I, —C≡N, X—(CH₂)ₚ—(C_{q}F_{2q+1}), R(22)-SOᵤ, R(23)R(24)N—CO, R(25)-CO— or R(26)R(27)N—SO₂—,
where the perfluoroalkyl group is straight-chain or branched;
X is a bond, oxygen, S or NR(28);
u is zero, 1 or 2;
p is zero, 1 or 2;
q is zero, 1, 2, 3, 4, 5 or 6;
R(22), R(23), R(25) and R(26)
independently are (C₁-C₈)-alkyl, (C₂-C₆)-alkenyl, —CₙH₂ₙ—R(29) or CF₃;
n is zero, 1, 2, 3, or 4;
R(28) is hydrogen or (C₁-C₃)-alkyl;
R(29) is (C₃-C₇)-cycloalkyl or phenyl;
which is not substituted or is substituted by 1-3 substituents selected from the group consisting of F, Cl, CF₃, methyl, methoxy and NR(30)R(31);
R(30) and R(31)
are hydrogen or C₁-C₄-alkyl,
or
R(23), R(25) and R(26)
are also hydrogen;
R(24) and R(27)
independently of one another are hydrogen or (C₁-C₄)-alkyl;
or
R(23) and R(24), and also R(26) and R(27) together are 4 or 5 methylene groups, of which one CH₂ group can be replaced by oxygen, S, NH, N—CH₃ or N-benzyl;
or
the other substituents R(1), R(2) and R(3) in each case independently of one another are OR(35) or NR(35)R(36);
R(35) and R(36)
independently of one another are hydrogen or (C₁-C₆)-alkyl;
or
R(35) and R(36)
together are 4-7 methylene groups, of which one CH₂ group can be replaced by oxygen, S, NH, N—CH₃ or N-benzyl,
R(4) and R(5)
independently of one another are hydrogen, (C₁-C₄)-alkyl, F, Cl, —OR(32), —NR(33)R(34) or CᵣF₂ᵣ₊₁;
R(32), R(33) and R(34)
independently of one another are hydrogen or (C₁-C₃)-alkyl;
r is 1, 2, 3 or 4;
and their pharmaceutically tolerable salts;
ad) benzoylguanidines of the formula I 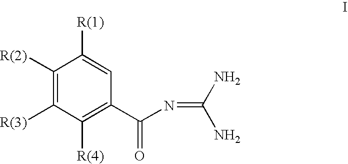
in which:
R(1) is hydrogen, F, Cl, Br, I, CN, NO₂, OH, (C₁-C₈)-alkyl, (C₃-C₈)-cycloalkyl, Oₐ—(CH₂)_{b}—(CF₂)_{c}—CF₃;
a is zero or 1;
b is zero, 1 or 2;
c is zero, 1, 2 or 3;
or
R(1) is R(5)-SOₘ or R(6)R(7)N—SO₂—;
m is zero, 1 or 2;
R(5) and R(6) independently of one another
are (C₁-C₈)-alkyl, (C₃-C₆)-alkenyl, CF₃ or —CₙH₂ₙ—R(8);
n is zero, 1,2, 3 or 4;
R(7) is hydrogen or (C₁-C₄)-alkyl;
R(8) is (C₃-C₇)-cycloalkyl or phenyl,
which is not substituted or is substituted by 1-3 substituents selected from the group consisting of F, Cl, CF₃, methyl, methoxy and NR(9)R(10);
R(9) and R(10) independently of one another
are hydrogen or (C₁-C₄)-alkyl;
or
R(6) is H;
or R(6) and R(7)
together are 4 or 5 methylene groups, of which one CH₂ group can be replaced by oxygen, S, NH, N—CH₃ or N-benzyl,
or
R(1) is —SR(11), —OR(11) or —CR(11)R(12)R(13);
R(11) is —CₚH₂ₚ—(C₃-C₈)-cycloalkyl, —(C₁-C₉)-heteroaryl or phenyl,
where the aromatic systems are unsubstituted or substituted by 1-3 substituents selected from the group consisting of F, Cl, CF₃, CH₃, methoxy, hydroxyl, amino, methylamino and dimethylamino;
R(12), R(13) independently of one another
are defined as R(11) or are hydrogen or (C₁-C₄)-alkyl;
p is zero, 1 or 2;
or
R(1) is phenyl, naphthyl, biphenylyl or (C₁-C₉)-heteroaryl, the latter linked via C or N,
which are unsubstituted or substituted by 1-3 substituents selected from the group consisting of F, Cl, CF₃, CH₃, methoxy, hydroxyl, amino, methylamino and dimethylamino;
R(2) is —CF₂R(14), —CF[R(15)][R(16)], —CF[(CF₂)_{q}—CF₃)][R(15)], —C[(CF₂)ᵣ—CF₃]═CR(15)R(16);
R(14) is (C₁-C₄)-alkyl or (C₃-C₆)-cycloalkyl;
R(15) and R(16) independently of one another
are hydrogen or (C₁-C₄)-alkyl;
q is zero, 1 or 2;
r is zero, 1 or 2;
R(3) is defined as R(1);
R(4) is hydrogen, (C₁-C₃)alkyl, F, Cl, Br, I, CN, —(CH₂)ₛ—(CF₂)ₜ—CF₃;
s is zero or 1;
t is zero, 1 or 2;
and their pharmaceutically tolerable salts;
af) benzoylguanidines of the formula I 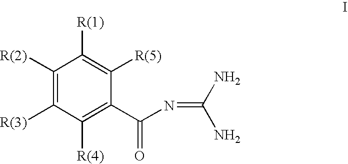
in which:
one of the three substituents R(1), R(2) and R(3)
is —Y-4-[(CH₂)ₖ—CHR(7)-(C═O)R(8)]-phenyl, —Y-3-(CH₂)ₖ—CHR(7)-(C═O)R(8)]-phenyl or
Y-2-[(CH₂)ₖ—CHR(7)-(C═O)R(8)]-phen
where the phenyl in each case is unsubstituted or substituted by 1-2 substituents from the group F, Cl, —CF₃, methyl, hydroxyl, methoxy, or —NR(37)R(38);
R(37) and R(38)
independently of one another are hydrogen or —CH₃;
Y is a bond, oxygen, —S— or —NR(9);
R(9) is hydrogen or —(C₁-C₄)-alkyl;
R(7) is —OR(10) or —NR(10)R(11);
R(10) and R(11)
independently of one another are hydrogen, —(C₁-C₈)-alkyl, —(C₁-C₈)-alkanoyl, —(C₁-C₈)-alkoxycarbonyl, benzyl, benzyloxycarbonyl;
or
R(10) is trityl;
R(8) is —OR(12) or —NR(12)R(13);
R(12) and R(13)
independently of one another are hydrogen, —(C₁-C₈)-alkyl or benzyl;
k is zero, 1, 2, 3 or 4;
and the other radicals R(1), R(2) and R(3) in each case
independently of one another are —(C₁-C₈)-alkyl, —(C₂-C₈)-alkenyl or —(CH₂)ₘR(14);
m is zero, 1 or 2;
R(14) is —(C₃-C₈)-cycloalkyl or phenyl,
which is not substituted or is substituted by 1-3 substituents selected from the group consisting of F, Cl, —CF₃, methyl, methoxy and —NR(15)R(16);
R(15) and R(16)
are hydrogen or —CH₃;
or
the other radicals R(1), R(2) and R(3) in each case
independently of one another are R(18)R(19)N—(C═Y′)—NH—SO₂—;
Y′ is oxygen, —S— or —N—R(20);
R(18) and R(19)
independently of one another are hydrogen, —(C₁-C₈)-alkyl, —(C₃-C₆)-alkenyl or —(CH₂)ₜ—R(21);
t is zero, 1, 2, 3, or 4;
R(21) is —(C₅-C₇)-cycloalkyl or phenyl,
which is unsubstituted or substituted by 1-3 substituents selected from the groups consisting of F, Cl, —CF₃, methoxy and —(C₁-C₄)-alkyl;
or
R(18) and R(19)
together are 4 or 5 methylene groups, of which one CH₂ group can be replaced by oxygen, —S—, —NH—, —N—CH₃ or —N-benzyl;
R(20)
is defined as R(18) or is amidine;
or
the other radicals R(1), R(2) and R(3) in each case
independently of one another are hydrogen, F, Cl, Br, I, —C≡N, X—(CH₂)ₚ—(C_{q}F_{2q+1}), R(22)-SOᵤ—, R(23)R(24)N—CO—, R(25)—CO— or R(26)R(27)N—SO₂—, where the perfluoroalkyl group is straight-chain or branched;
X is a bond, oxygen, —S— or —NR(28);
u is zero, 1 or 2;
p is zero, 1 or 2;
q is 1, 2, 3, 4, 5 or 6;
R(22), R(23), R(25) and R(26)
independently of one another are —(C₁-C₈)-alkyl, —(C₃-C₆)-alkenyl, —(CH₂)ₙ—R(29) or —CF₃;
n is zero, 1, 2, 3 or 4;
R(28) is hydrogen or —(C₁-C₃)-alkyl;
R(29) is —(C₃-C₇)-cycloalkyl or phenyl,
which is not substituted or is substituted by 1-3 substituents selected from the group consisting of F, Cl, —CF₃, methyl, methoxy and —NR(30)R(31);
R(30) and R(31)
are hydrogen or —(C₁-C₄)-alkyl;
or
R(23), R(25) and R(26)
are hydrogen;
R(24) and R(27)
independently of one another are hydrogen or —(C₁-C₄)-alkyl;
or
R(23) and R(24), and also R(26) and R(27)
together are 4 or 5 methylene groups, of which one CH₂ group can be replaced by oxygen, —S—, —NH—, —N—CH₃ or —N-benzyl;
or
the other radicals R(1), R(2) and R(3) in each case
independently of one another are —OR(35) or —NR(35)R(36);
R(35) and R(36)
independently of one another are hydrogen or —(C₁-C₆)-alkyl;
or
R(35) and R(36)
together are 4-7 methylene groups, of which one CH₂ group can be replaced by oxygen, —S—, —NH—, —N—CH₃ or —N-benzyl;
R(4) and R(5)
independently of one another are hydrogen, —(C₁-C₄)-alkyl, F, Cl, —OR(32), —NR(33)R(34) or —CᵣF₂ᵣ₊₁;
R(32), R(33) and R(34)
independently of one another are hydrogen or —(C₁-C₃)-alkyl;
r is 1, 2, 3, or 4;
and their pharmaceutically tolerable salts;
ag) benzoylguanidines of the formula I 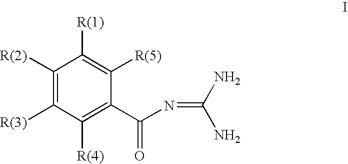
in which:
R(1) is R(6)—CO or R(7)R(8)N—CO;
R(6) is (C₁-C₈)-alkyl, (C₁-C₈)-perfluoroalkyl, (C₃-C₈)-alkenyl or —CₙH₂ₙ—R(9),
n is zero, 1, 2, 3 or 4;
R(9) is (C₃-C₈)-cycloalkyl, phenyl, biphenylyl or naphthyl,
where the aromatics are not substituted or are substituted by 1-3 substituents selected from the group consisting of F, Cl, CF₃, methyl, methoxy and NR(10)R(11),
R(10) and R(11)
are H, (C₁-C₄)-alkyl or (C₁-C₄)-perfluoroalkyl;
R(7) is H, (C₁-C₈)-alkyl, (C₁-C₈)-perfluoroalkyl, (C₃-C₈)-alkenyl or —CₙH₂ₙ—R(12);
n is zero, 1, 2, 3 or 4;
R(12) is (C₃-C₈)-cycloalkyl, phenyl, biphenylyl or naphthyl,
where the aromatics are not substituted or are substituted by 1-3 substituents selected from the group consisting of F, Cl, CF₃, methyl, methoxy and NR(13)R(14);
R(13) and R(14)
are H, (C₁-C₄)-alkyl or (C₁-C₄)-perfluoroalkyl;
R(8) is H, (C₁-C₄)-alkyl or (C₁-C₄)-perfluoroalkyl;
or
R(7) and R(8) together
are 4 or 5 methylene groups, of which one CH₂ group can be replaced by oxygen, S, NH, N—CH₃ or N-benzyl;
R(2) is defined as R(1), or is H, OH, F, Cl, Br, I, CN, NO₂, (C₁-C₈)-alkyl, (C₁-C₈)-perfluoroalkyl, (C₃-C₈)-alkenyl or —CₙH₂ₙR(15);
n is zero, 1, 2, 3 or 4;
R(15) is (C₃-C₈)-cycloalkyl, phenyl, biphenylyl or naphthyl,
where the aromatics are not substituted or are substituted by 1-3 substituents selected from the group consisting of F, Cl, CF₃, methyl, methoxy and NR(16)R(17);
R(16) and R(17)
are H, (C₁-C₄)-alkyl or (C₁-C₄)-perfluoroalkyl;
or
R(2) is (C₁-C₉)-heteroaryl,
which is linked via C or N and which is unsubstituted or substituted by 1-3 substituents selected from the group consisting of F, Cl, CF₃, CH₃, methoxy, hydroxyl, amino, methylamino and dimethylamino;
or
R(2) is SR(18), —OR(18), —NR(18)R(19) or-CR(18)R(19)R(20);
R(18) is CₐH₂ₐ—(C₁-C₉)-heteroaryl,
which is unsubstituted or substituted by 1-3 substituents selected from the group consisting of F, Cl, CF₃, CH₃, methoxy, hydroxyl, amino, methylamino, dimethylamino;
a is zero, 1 or 2;
R(19) and R(20)
independently of one another are defined as R(18) or are hydrogen, (C₁-C₄)-alkyl or (C₁-C₄)-perfluoroalkyl;
or
R(2) is R(21 )-SOₘ or R(22)R(23)N—SO₂—;
m is 1 or 2;
R(21) is (C₁-C₈)-alkyl, (C₁-C₈)-perfluoroalkyl, (C₃-C₈)-alkenyl or —CₙH₂ₙ—R(24);
n is zero, 1, 2, 3, or 4;
R(24) is (C₃-C₈)-cycloalkyl, phenyl, biphenylyl or naphthyl,
where the aromatics are not substituted or are substituted by 1-3 substituents selected from the group consisting of F, Cl, CF₃, methyl, methoxy and NR(27)R(28);
R(27) and R(28)
are H, (C₁-C₄)-alkyl or (C₁-C₄)-perfluoroalkyl;
R(22) is H, (C₁-C₈)-alkyl, (C₁-C₈)-perfluoroalkyl, (C₃-C₈)-alkenyl or —CₙH₂ₙ—R(29);
n is zero, 1, 2, 3 or 4;
R(29) is (C₃-C₈)-cycloalkyl, phenyl, biphenylyl or naphthyl,
where the aromatics are not substituted or are substituted by 1-3 substituents selected from the group consisting of F, Cl, CF₃, methyl, methoxy and NR(30)R(31);
R(30) and R(31)
are H, (C₁-C₄)-alkyl or (C₁-C₄)-perfluoroalkyl;
R(23) is hydrogen, (C₁-C₄)-alkyl or (C₁-C₄)-perfluoroalkyl;
or
R(22) and R(23)
together are 4 or 5 methylene groups, of which one CH₂
group can be replaced by oxygen, S, NH, N—CH₃ or N-benzyl;
or
R(2) is R(33)X—;
X is oxygen, S, NR(34), (D=O)A- or NR(34)C=MN^{(*)}R(35)-;
M is oxygen or S;
A is oxygen or NR(34);
D is C or SO;
R(33) is (C₁-C₈)-alkyl, (C₃-C₈)-alkenyl, (CH₂)_{b}C_{d}F_{2d+1} or —CₙH₂ₙ—R(36);
b is zero or 1;
d is 1, 2, 3, 4, 5, 6 or 7;
n is zero, 1, 2, 3, or 4;
R(36) is (C₃-C₈)-cycloalkyl, phenyl, biphenylyl or naphthyl,
where the aromatics are not substituted or are substituted by 1-3 substituents selected from the group consisting of F, Cl, CF₃, methyl, methoxy and NR(37)R(38);
R(37) and R(38)
are H, (C₁-C₄)-alkyl or (C₁-C₄)-perfluoroalkyl;
R(34) is hydrogen, (C₁-C₄)-alkyl or (C₁-C₄)-perfluoroalkyl;
R(35) is defined as R(33);
or
R(33) and R(34)
together are 4 or 5 methylene groups, of which one CH₂ group can be replaced by oxygen, S, NH, N—CH₃ or N-benzyl;
where A and N^{(*)} are bonded to the phenyl nucleus of the benzoylguanidine parent structure;
or
R(2) is —SR(40), —OR(40), —NHR(40), —NR(40)R(41), —CHR(40)R(42), —CR(42)R(43)OH, —C≡CR(45), —CR(46)=CR(45) or —[CR(47)R(48)]ᵤ—CO—[C(R49)R(50)]ᵥ—R(44);
R(40) and R(41)
independently of one another are —(CH₂)ₚ—(CHOH)_{q}—(CH₂)ᵣ—(CHOH)ₜ—R(51) or —(CH₂)ₚ—O—(CH₂—CH₂O)_{q}—R(51);
R(51) is hydrogen or methyl;
u is 1, 2, 3, or 4;
v is zero, 1,2, 3 or 4;
p, q and r
independently of one another are zero, 1, 2, 3 or 4;
t is 1, 2, 3, or 4;
R(42) and R(43)
independently of one another are hydrogen or (C₁-C₆)-alkyl;
or
R(42) and R(43)
together with the carbon atom carrying them are a (C₃-C₈)-cycloalkyl;
R(44) is hydrogen, (C₁-C₆)-alkyl, (C₃-C₈)-cycloalkyl, —CₑH₂ₑ—R(⁴⁵);
e is zero, 1, 2, 3 or 4;
R(45) is phenyl,
which is unsubstituted or substituted by 1-3 substituents selected from the group consisting of F, Cl, CF₃, methyl, methoxy and NR(52)R(53);
R(52) and R(53)
are H or (C₁-C₄)-alkyl;
or
R(45) is (C₁-C₉)-heteroaryl,
which is unsubstituted or substituted as phenyl;
or
R(45) is (C₁-C₆)-alkyl,
which is unsubstituted or substituted by 1-3 OH;
R(46), R(47), R(48), R(49) and R(50)
independently of one another are hydrogen or methyl;
or
R(2) is R(55)-NH—SO₂—;
R(55) is R(56)R(57)N-(C═Y)—;
Y is oxygen, S or N—R(58);
R(56) and R(57)
independently of one another are hydrogen, (C₁-C₈)-alkyl, (C₃-C₆)-alkenyl or —C_{f}H₂—R(59);
f is zero, 1, 2, 3 or 4;
R(59) is (C₅-C₇)-cycloalkyl, phenyl,
which is unsubstituted or substituted by 1-3 substituents selected from the group consisting of F, Cl, CF₃, methoxy and (C₁-C₄)-alkyl;
or
R(56) and R(57)
together are 4 or 5 methylene groups, of which one CH₂ group can be replaced by oxygen, S, NH, N—CH₃ or N-benzyl;
R(58)
is defined as R(56) or is amidine;
R(3), R(4) and R(5) are independently of one another defined as R(1) or R(2), but where at least one of the substituents R(2), R(3), R(4) and R(5) must be OH;
and their pharmaceutically tolerable salts;
ah) benzoylguanidines of the formula I 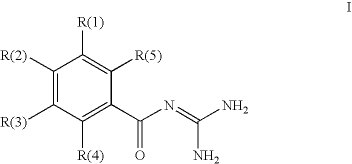
in which:
one of the three substituents R(1), R(2) and R(3)
is R(6)-A-B-D-;
R(6) is a basic protonatable radical, i.e. an amino group
—NR(7)R(8), an amidino group R(7)R(8)N—C[═N—R(9)]- or a guanidino group 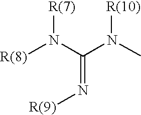
R(7), R(8), R(9) and R(10)
independently of one another are hydrogen or alkyl having 1, 2, 3 or 4 carbon atoms;
or
R(7) and R(8)
together are CₐH₂ₐ;
a is 4, 5, 6 or 7;
where if a=5, 6 or 7 a methylene group of the group CₐH₂ₐ can be replaced by a heteroatom group O, SOₘ or NR(11),
or
R(8) and R(9) or R(9) and R(10) or R(7) and R(10)
are a group C H₂ ;
a is 2, 3, 4 or 5;
where if a=3, 4 or 5 a methylene group of the group CₐH₂ₐ can be replaced by a heteroatom group O, SOₘ or NR(11);
m is zero, 1 or 2;
R(11) is hydrogen or methyl;
or
R(6) is a basic heteroaromatic ring system having 1-9 carbon atoms;
A is C_{b}H_{2b};
b is 1, 2, 3, 4, 5, 6, 7, 8, 9 or 10;
where in the group C_{b}H_{2b} one or two methylene groups can be replaced by one of the groupings selected from the group consisting of —O—, —CO—, —CH[OR(20)]-, —SOₘ—, —NR(20)-, —NR(20)—CO—, —NR(20)-CO—NH—, —NR(20)-CO—NH—SO₂— 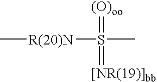
and —SOₐₐ[NR(¹⁹)]_{bb}-;
and where in the group C_{b}H_{2b} a methylene group can be replaced by —CH—R(99), where R(99) together with R(7) forms a pyrrolidine or piperidine ring;
aa is 1 or 2;
bb is 0 or 1;
aa+bb=2;
R(19) is hydrogen or alkyl having 1, 2, 3 or 4 carbon atoms;
R(20) is hydrogen or methyl;
B is a phenylene or naphthylene radical 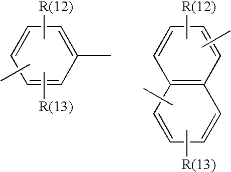
R(12) and R(13)
independently of one another are hydrogen, methyl, F, Cl, Br, I, CF₃ or —SO_{w}—R(14);
R(14) is methyl or NR(15)R(16);
R(15) and R(16)
independently of one another are hydrogen or alkyl having 1, 2, 3 or 4 carbon atoms;
w is zero, 1 or 2;
D is —C_{d}H_{2d}—X_{f}—;
d is zero, 1, 2, 3 or 4;
X is —O—, —CO—, —CH[OR(21)]-, —SOₘ— or —NR(21)-;
f is zero or 1;
R(21) is hydrogen or methyl;
m is zero, 1 or 2;
and the other substituents R(1) and R(2) and R(3) in each case
independently of one another are hydrogen, F, Cl, Br, I, —CN, —(C₁-C₈)-alkyl, —(C₂-C₈)-alkenyl, —NR(35)R(36) or R(17)-C_{g}H_{2g}-Zₕ-;
g is zero, 1, 2, 3 or 4;
h is zero or 1;
R(35) and R(36)
independently of one another are hydrogen or alkyl having 1, 2, 3, 4, 5 or 6 carbon atoms;
or
R(35) and R(36)
together are 4-7 methylene groups, of which one CH₂ group can be replaced by oxygen, —S—, —NH—, —NCH₃ or —N-benzyl;
Z is —O—, —CO—, —SOᵥ—, —NR(18)-, —NR(18)—CO—, —NR(18)—CO—NH—
or —NR(18)-SO₂—;
R(18) is hydrogen or methyl;
v is zero, 1 or 2;
R(17) is hydrogen, cycloalkyl having 3, 5 or 6 carbon atoms or CₖF₂ₖ₊₁—;
k is 1, 2 or 3,
or
R(17) is pyrrol-1-yl, pyrrol-2-yl or pyrrol-3-yl,
which is not substituted or is substituted by 1-4 substituents selected from the group consisting of F, Cl, Br, I, —CN, (C₂-C₈)-alkanoyl, (C₂-C₈)-alkoxycarbonyl, formyl, carboxyl, —CF₃, methyl and methoxy;
or
R(17) is (C₃-C₈)-cycloalkyl or phenyl,
which is not substituted or is substituted by 1-3 substituents selected from the group consisting of F and Cl, —CF₃, methyl, hydroxyl, methoxy, —NR(37)R(38), CH₃SO₂— and H₂NO₂S—;
R(37) and R(38)
are hydrogen or —CH₃;
R(4) and R(5)
independently of one another are hydrogen, alkyl having 1, 2, 3 or 4 carbon atoms, F, Cl, —OR(32), —NR(33)R(34) or —CᵣF₂ᵣ₊₁;
R(32), R(33) and R(34)
independently of one another are hydrogen or alkyl having 1, 2 or 3 carbon atoms;
r is 1, 2, 3, or 4;
and their pharmacologically tolerable salts;
ai) indenoylguanidines of the formula I 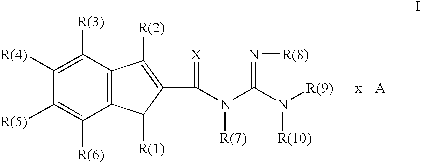
in which:
R(1) and R(2)
independently of one another are hydrogen, alkyl having 1, 2, 3, 4, 5, 6, 7, 8, 9 or 10 carbon atoms, cycloalkyl having 3, 4, 5 or 6 carbon atoms, O-alkyl having 1, 2, 3 or 4 carbon atoms, O—C(═O)-alkyl having 1, 2, 3 or 4 carbon atoms or CₘH₂ₘ—NR(12)R(13);
R(12) and R(13)
independently of one another are hydrogen or alkyl having 1, 2, 3 or 4 carbon atoms;
m is zero, 2, 3 or 4;
NH—C(═O)—NH₂, C(═O)—O-alkyl having 1, 2, 3 or 4 carbon atoms, C(═O)—NH₂, C(═O)—NH-alkyl having 1, 2, 3 or 4 carbon atoms, C(═O)-N(alkyl)₂ having 1, 2, 3 or 4 carbon atoms in each alkyl group, alkenyl having 2, 3, 4, 5, 6, 7, 8, 9 or 10 carbon atoms, alkynyl having 2, 3, 4, 5, 6, 7, 8, 9 or 10 carbon atoms, alkylaryl having 1, 2, 3 or 4 carbon atoms in the alkyl group, alkenylaryl having 2, 3, 4, 5, 6, 7, 8, 9 or 10 carbon atoms in the alkenyl group, alkynylaryl having 2, 3, 4, 5, 6, 7, 8, 9 or 10 carbon atoms in the alkynyl group, C₁-C₄-alkyl-substituted aryl, C₁-C₄-alkylheteroaryl, C₁-C₄-alkenylheteroaryl, aminoalkylaryl having 1, 2, 3 or 4 carbon atoms in the alkyl group, substituted aryl, heteroaryl and substituted heteroaryl;
R(3), R(4), R(5) and R(6)
independently of one another are hydrogen, alkyl having 1, 2, 3, 4, 5, 6, 7, 8, 9 or 10 carbon atoms, O-alkyl having 1, 2, 3, 4, 5, 6, 7, 8, 9 or 10 carbon atoms, halogen, (such as F, Cl, Br, I), OH, aryl, substituted aryl, heteroaryl, substituted heteroaryl, O-lower alkyl, O-aryl, O-lower alkylaryl, O-substituted aryl, O-lower alkyl-substituted aryl, O—C(═O)—C₁-C₄-alkylaryl, O—C(═O)—NH—C₁-C₄-alkyl, O—C(═O)—N(C₁-C₄-alkyl)₂, NO₂, CN, CF₃, NH₂, NH—C(═O)—C₁-C₄-alkyl, NH—C(═O)—NH₂, COOH, C(═O)—O—C₁-C₄-alkyl, C(═O)—NH₂, C(═O)—NH—C₁-C₄-alkyl, C(═O)—N(C₁-C₄-alkyl)₂, C₁-C₄—COOH, C₁-C₄-alkyl-C(═O)—O—C₁-C₄-alkyl, SO₃H, SO₂-alkyl, SO₂-alkylaryl, SO₂—N—(alkyl)₂, SO₂—N(alkyl)(alkylaryl), C(═O)—R(11), C₁-C₁₀-alkyl-C(═O)—R(11), C₂-C₁₀-alkenyl-C(═O)—R(11), C₂-C₁₀-alkynyl-C(═O)—R(11), NH—C(═O)—C₁-C₁₀-alkyl-C(═O)-R(11), O—C₁-C₁₁-alkyl-C(═O)—R(11);
R(11) is C₁-C₄-alkyl, C₁-C₄-alkynyl, aryl, substituted aryl, NH₂, NH—
C₁-C₄-alkyl, N—(C₁-C₄-alkyl)₂, SO₃H, SO₂-alkyl, SO₂-alkylaryl, SO₂—N-(alkyl)₂, SO₂—N(alkyl)(alkylaryl);
X is O, S or NH;
R(7), R(8), R(9) and R(10)
independently of one another are hydrogen, alkyl, cycloalkyl, aryl, alkylaryl;
or
R(8) and R(9)
together are part of a 5, 6 or 7-membered heterocyclic ring;
A is absent or is a nontoxic organic or inorganic acid.
ak) benzyloxycarbonylguanidines of the formula I 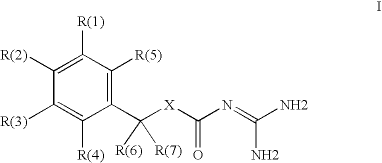
in which:
R(1), R(2) and R(3)
independently of one another are —Y-[4-R(8)-phenyl], —Y-[3-R(8)-phenyl] or —Y-[2-R(8)-phenyl],
where the phenyl is in each case unsubstituted or substituted by 1-2 substituents from the group consisting of F, Cl, —CF₃, methyl, hydroxyl, methoxy and —NR(96)R(97);
R(96) and R(97)
independently of one another are hydrogen or —CH₃;
Y is a bond, CH₂, oxygen, —S— or —NR(9);
R(9) is hydrogen or alkyl having 1, 2, 3 or 4 carbon atoms;
R(8) is SOₐ[NR(98)]_{b}NR(99)R(10);
a is 1 or 2;
b is 0 or 1;
a+b=2;
R(98), R(99) and R(10)
independently of one another are hydrogen, —(C₁-C₈)-alkyl, benzyl, —(C₂-C₈)-alkylene-NR(11)R(12), (C₂-C₈)-alkylene-NR(13)-(C₂-C₈)-alkylene-NR(37)R(38) or (C₀-C₈)-alkylene-CR(39)R(40)CR(41 )R(42)(C₀-C₈)-alkylene-NR(43)R(44);
R(11), R(12), R(13), R(37), R(38), R(43) and R(44)
independently of one another are hydrogen, —(C₁-C₈)-alkyl or benzyl:
R(39), R(40), R(41) and R(42)
independently of one another are hydrogen, —(C₁-C₈)-alkyl or —(C₀-C₃)-alkylenephenyl,
where the phenyl is not substituted or is substituted by 1-3 substituents selected from the group consisting of F, Cl, —CF₃, methyl and methoxy;
or
R(99) and R(10)
together are 4-6 methylene groups, of which one CH₂ group can be replaced by oxygen, —S—, —NH—, —N—CH₃ or —N-benzyl;
or
R(8) is SOₐ[NR(98)]_{b}NR(95)-C[=N—R(94)]-NR(93)R(92);
R(92), R(93), R(94) and R(95)
independently of one another are hydrogen or alkyl having 1, 2, 3 or 4 carbon atoms;
or
R(1), R(2) and R(3)
independently of one another are pyrrol-1-yl, pyrrol-2-yl or pyrrol-3-yl,
which is not substituted or is substituted by 1-4 substituents selected from the group consisting of F, Cl, Br, I, —CN, (C₂-C₈)-alkanoyl, (C₂-C₈)-alkoxycarbonyl, formyl, carboxyl, —CF₃, methyl, methoxy;
or
R(1), R(2) and R(3)
independently of one another are hydrogen, —(C₁-C₈)-alkyl, —(C₂-C₈)-alkenyl or —(CH₂)ₘR(14);
m is zero, 1 or 2;
R(14) is —(C₃-C₈)-cycloalkyl or phenyl,
which is not substituted or is substituted by 1-3 substituents selected from the group consisting of F and —Cl, —CF₃, methyl, methoxy and —NR(15)R(16);
R(15) and R(16)
are hydrogen or —CH₃;
or
R(1), R(2) and R(3)
independently of one another are -Q4-[(CH₂)ₖ—CHR(17)-(C═O)R(20)]-phenyl, -Q-3-(CH₂)ₖ—CHR(17)-(C═O)R(20)]-phenyl or -Q-2-[(CH₂)ₖ—CHR(17)-(C═O)R(20)]-phenyl,
where the phenyl in each case is unsubstituted or substituted by 1-2 substituents from the group F, Cl, —CF₃, methyl,
hydroxyl, methoxy and —NR(35)R(36);
R(35) and R(36)
independently of one another are hydrogen or —CH₃;
Q is a bond, oxygen, —S— or —NR(18);
R(18) is hydrogen or —(C₁-C₄)-alkyl;
R(17) is —OR(21) or —NR(21)R(22);
R(21) and R(22)
independently of one another are hydrogen, —(C₁-C₈)-alkyl, —(C₁-C₈)-alkanoyl, —(C₁-C₈)-alkoxycarbonyl, benzyl, benzyloxycarbonyl;
or
R(21) is trityl;
R(20) is —OR(23) or —NR(23)R(24);
R(23), R(24) independently of one another are hydrogen, —(C₁-C₈)-alkyl or benzyl;
k is zero, 1, 2, 3 or 4;
or
R(1), R(2) and R(3)
independently of one another are (C₁-C₉)-heteroaryl,
which is linked via C or N and which is unsubstituted or substituted by 1-3 substituents from the group F, Cl, CF₃, CH₃, methoxy, hydroxyl, amino, methylamino and dimethylamino;
or
R(1), R(2) and R(3)
are —SR(25), —OR(25), —NR(25)R(26), —CR(25)R(26)R(27);
R(25) is —C_{f}H_{2f}—(C₁-C₉)-heteroaryl,
which is unsubstituted or substituted by 1-3 substituents from the group F, Cl, CF₃, CH₃, methoxy, hydroxyl, amino, methylamino and dimethylamino;
f is zero, 1or 2;
R(26) and R(27)
independently of one another are defined as R(25) or are hydrogen or (C₁-C₄)-alkyl,
or
R(1), R(2) and R(3)
independently of one another are (C₁-C₉)-heteroaryl-N-oxide,
which is linked via C or N and which is unsubstituted or substituted by 1-3 substituents selected from the group consisting of F, Cl, CF₃, CH₃, methoxy, hydroxyl, amino, methylamino and dimethylamino;
or
R(1), R(2) and R(3)
independently of one another are —SR(28), —OR(28), —NR(28)R(29) or —CR(28)R(29)R(30);
R(28) is —C_{g}H_{2g}—(C₁-C₉)-heteroaryl-N-oxide,
which is unsubstituted of substituted by 1-3 substituents selected from the group consisting of F, Cl, CF₃, CH₃, methoxy, hydroxyl, amino, methylamino and dimethylamino;
g is zero, 1 or 2;
R(29), R(30)
independently of one another are defined as R(28), hydrogen or (C₁-C₄)-alkyl:
or
R(1), R(2) and R(3)
independently of one another are hydrogen, F, Cl, Br, I, —C≡N, T-(CH₂)ₕ—(CᵢF₂ᵢ₊₁), R(31)SO_{I}^{-, R(}32)R(33)N—CO—, R(34)—CO— or R(45)R(46)N—SO₂, where the perfluoroalkyl group is straight-chain or branched;
T is a bond, oxygen, —S— or —NR(47);
I is zero, 1 or 2;
h is zero, 1 or 2;
i is 1, 2, 3, 4, 5 or 6;
R(31), R(32), R(34) and R(45)
independently of one another are —(C₁-C₈)-alkyl, —(C₃-C₆)-alkenyl, (CH₂)ₙR(48) or —CF₃;
n is zero, 1, 2, 3, or 4;
R(47) is hydrogen or alkyl with 1, 2 or 3 carbon atoms;
R(48) is —(C₃-C₇)-cycloalkyl or phenyl, which is not substituted or is substituted by 1-3 substituents selected from the group consisting of F, Cl, —CF₃, methyl, methoxy and —NR(49)R(50);
R(49) and R(50)
are hydrogen or alkyl having 1, 2, 3 or 4 carbon atoms;
or
R(32), R(34) and R(45)
are hydrogen;
R(33) and R(46)
independently of one another are hydrogen or alkyl having 1, 2, 3 or 4 carbon atoms;
or
R(32) and R(33), and R(45) and R(46)
together are 5 or 6 methylene groups, of which one CH₂ group can be replaced by oxygen, —S—, —NH—, —NCH₃ or —N-benzyl;
or
R(1), R(2) and R(3)
independently of one another are R(51)-A-G-D-;
R(51) is a basic protonatable radical, i.e. an amino group
—NR(52)R(53), an amidino group R(52)R(53)N—C[═N—R(54)]- or a guanidino group R(52)R(53)N—C[═N—R(54)]—NR(55)-;
R(52), R(53), R(54) and R(55)
independently of one another are hydrogen or alkyl having 1, 2, 3 or 4 carbon atoms;
or
R(52) and R(53) are
a group C_{α}H_{2α};
α is 4, 5, 6 or 7;
where if α=5, 6 or 7 a carbon atom of the group C_{α}H_{2α} can be replaced by a heteroatom group O, SO_{d} or NR(56),
or
R(53) and R(54) or R(54) and R(55) or R(52) and R(55) are
a group C_{y}H_{2y};
y is 2, 3, 4 or 5;
where if y=3, 4 or 5 a carbon atom of the group C_{y}H_{2y} can be replaced by a heteroatom group O, SO_{d} or NR(56);
d is zero, 1 or 2;
R(56) is hydrogen or methyl;
or
R(51) is a basic heteroaromatic ring system having 1-9 carbon atoms;
A is a group CₑH₂ₑ;
e is zero, 1, 2, 3, 4, 5, 6, 7, 8, 9 or 10;
where in the group CₑH₂ₑ a carbon atom can be replaced by one of the groupings —O—, —CO—, —CH[OR(57)]-, —SOᵣ—, —NR(57)-, —NR(57)-CO—, —NR(57)-CO—NH—, —NR(57)-CO—NH—SO₂— or —NR(57)-SO₂—;
r is zero, 1 or 2;
G is a phenylene radical 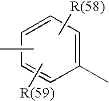
R(58) and R(59)
independently of one another are hydrogen, methyl, methoxy, F, Cl, Br, I, CF₃ or —SOₛ—R(60);
R(60) is methyl or NR(61)R(62);
R(61) and R(62)
independently of one another are hydrogen or alkyl having 1, 2, 3 or 4 carbon atoms;
D is —CᵥH₂ᵥ-E_{w}-;
v is zero, 1, 2, 3 or 4;
E is —O—, —CO—, —CH[OR(63)]-, —SOₐₐ— or —NR(63)-;
w is zero or 1;
aa is zero, 1 or 2
R(63) is hydrogen or methyl,
or
R(1), R(2) and R(3)
independently of one another are —CF₂R(64), —CF[R(65)][R(66)], —CF[(CF₂)_{q}—CF₃][R(65)], —C[(CF₂)ₚ—CF₃]═CR(65)R(66);
R(64) is alkyl having 1, 2, 3 or 4 carbon atoms or cycloalkyl having 3, 4, 5, 6 or 7 carbon atoms;
R(65) and R(66) independently of one another are hydrogen or alkyl having 1, 2, 3 or 4 carbon atoms;
q is zero, 1 or 2;
p is zero, 1 or 2;
or
R(1), R(2) and R(3)
independently of one another are —OR(67) or —NR(67)R(68);
R(67) and R(68)
independently of one another are hydrogen or alkyl having 1, 2, 3, 4, 5 or 6 carbon atoms;
or
R(67) and R(68)
together are 4, 5, 6 or 7 methylene groups, of which one CH₂ group can be replaced by oxygen, —S— SO₂, —NH—, —NCH₃ or —N-benzyl;
R(4) and R(5)
independently of one another are hydrogen, alkyl having 1, 2, 3 or 4 carbon atoms, F, Cl, —OR(69), —NR(70)R(71) or —C_{z}F_{2z+1};
R(69), R(70) and R(71)
independently of one another are hydrogen or alkyl having 1, 2 or 3 carbon atoms;
z is 1, 2, 3, or 4;
R(6) and R(7)
independently of one another are hydrogen or alkyl having 1, 2, 3 or 4 carbon atoms;
X is oxygen or NR(72);
R(72) is hydrogen or alkyl having 1, 2, 3 or 4 carbon atoms;
and their pharmaceutically tolerable salts;
al) alkenylcarboxylic acid guanidines, carrying fluorophenyl groups, of the formula I 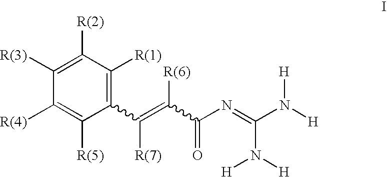
in which:
R(6) is hydrogen, (C₁-C₈)-alkyl, (C₃-C₈)-cycloalkyl or phenyl,
where the phenyl group is not substituted or is substituted by 1-3 substituents selected from the group consisting of F, Cl, CF₃, methyl, methoxy and NR(9)R(10);
R(9) and R(10)
are hydrogen, (C₁-C₄)-alkyl or (C₁-C₄)-perfluoroalkyl;
R(7) independently is defined as R(6);
R(1), R(2), R(3), R(4) and R(5)
independently of one another are hydrogen or F;
where, however, at least one of the radicals R(1), R(2), R(3), R(4) and R(5) must be fluorine;
and their pharmaceutically tolerable salts;
am) benzoylguanidines of the formula I 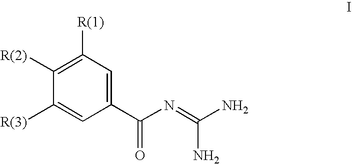
in which:
R(1) is R(⁴)—SOₘ
or R(5)R(6)N—SO₂—;
m is 1 or 2;
R(4) and R(5)
independently of one another are alkyl having 1, 2, 3, 4, 5, 6, 7 or 8 carbon atoms, alkenyl having 3, 4, 5 or 6 carbon atoms, CF₃ or —CₙH₂ₙ—R(7);
n is zero, 1, 2, 3 or 4;
R(6) is H or alkyl having 1, 2, 3 or 4 carbon atoms;
R(7) is cycloalkyl having 3, 4, 5, 6, 7 or 8 carbon atoms or phenyl,
which is not substituted or is substituted by 1-3 substituents selected from the group consisting of F, Cl, CF₃, methyl, methoxy and NR(8)R(9);
R(8) and R(9)
are H or alkyl having 1, 2, 3 or 4 carbon atoms;
or
R(5) is also hydrogen;
or
R(5) and R(6)
together are 4 or 5 methylene groups, of which a CH₂ group can be replaced by oxygen, S, NH, N—CH₃ or N-benzyl;
or
R(1) is —Oₚ—(CH₂)_{q}—(CF₂)ᵣ—CF₃;
p is zero or 1;
q is zero, 1 or 2;
r is zero, 1, 2 or 3;
or
R(1) is —SR(10), —OR(10) or —CR(10)R(11)R(12);
R(10), R(11) and R(12)
independently of one another are hydrogen, alkyl having 1, 2, 3, 4, 5, 6, 7 or 8 carbon atoms, —CₛH₂ₛ—(C₃-C₈)-cycloalkyl or an aromatic system selected from the group consisting of pyridyl, pyrrolyl, quinolyl, isoquinolyl, imidazolyl or phenyl;
s is zero, 1 or 2;
where the aromatic systems pyridyl, pyrrolyl, quinolyl, isoquinolyl, imidazolyl and phenyl are unsubstituted or substituted by 1-3 substituents selected from the group consisting of F, Cl, CF₃, CH₃, methoxy, hydroxyl, amino, methylamino and dimethylamino;
R(2) is —(CH₂)ᵤ—(CF₂)ₜ—CF₃;
t is zero, 1, 2 or 3;
u is zero or 1;
R(3) is hydrogen or independently is defined as R(1);
and their pharmaceutically tolerable salts;
an) substituted cinnamic acid guanidines of the formula I 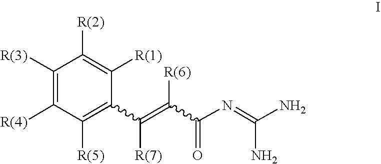
in which:
at least one of the substituents R(1), R(2), R(3), R(4) and R(5) is —Xₐ—Y_{b}-Lₙ-U;
X is CR(16)R(17), O, S or NR(18);
R(16), R(17) and R(18)
independently of one another are H, alkyl having 1, 2, 3 or 4 carbon atoms or perfluoroalkyl having 1, 2, 3 or 4 carbon atoms;
a is zero or 1;
Y is alkylene having 1, 2, 3, 4, 5, 6, 7 or 8 carbon atoms, alkylene-T having 1, 2, 3, 4, 5, 6, 7 or 8 carbon atoms in the alkylene group, T, T-alkylene having 1, 2, 3, 4, 5, 6, 7 or 8 carbon atoms in the alkylene group;
T is NR(20), O, S or phenylene,
where the phenylene is not substituted or is substituted by 1-3 substituents selected from the group consisting of F, Cl, CF₃, methyl, methoxy and NR(21)R(22);
R(20), R(21) and R(22)
independently of one another are H, alkyl having 1, 2, 3 or 4 carbon atoms or perfluoroalkyl having 1, 2, 3 or 4 carbon atoms;
b is zero or 1;
L is O, S, NR(23) or CₖH₂ₖ;
k is 1, 2, 3, 4, 5, 6, 7, 8;
n is zero or 1;
U is NR(24)R(25) or an N-containing heterocycle having 1, 2, 3, 4, 5, 6, 7, 8 or 9 carbon atoms;
R(24) and R(25)
independently of one another are hydrogen, alkyl having 1, 2, 3, 4, 5, 6, 7 or 8 carbon atoms or perfluoroalkyl having 1, 2, 3, 4, 5, 6, 7 or 8 carbon atoms;
or
R(24) and R(25)
together are 4 or 5 methylene groups, of which one CH₂ group can be replaced by oxygen, S, NH, N—CH₃ or N-benzyl;
where the N-containing heterocycles are N- or C-bridged and are not substituted or are substituted by 1-3 substituents selected from the group consisting of F, Cl, CF₃, methyl, methoxy and NR(27)R(28);
R(23), R(27) and R(28)
independently of one another are H, alkyl having 1, 2, 3 or 4 carbon atoms or perfluoroalkyl having 1, 2, 3 or 4 carbon atoms:
and the other substituents R(1), R(2), R(3), R(4) and R(5) in each case independently of one another are H, F, Cl, Br, I, CN, —Oₙ—CₘH₂ₘ₊₁, —Oₚ—(CH₂)ₛ—C_{q}F_{2q+1} or —CᵣH₂ᵣR(10);
n is zero or 1;
m is zero 1, 2, 3, 4, 5, 6, 7 or 8;
p is zero or 1;
q is 1, 2, 3, 4, 5, 6, 7 or 8;
s is zero, 1, 2, 3 or 4;
r is zero, 1, 2, 3 or 4;
R(10)
is cycloalkyl having 3, 4, 5, 6, 7 or 8 carbon atoms, or phenyl,
where the phenyl is not substituted or is substituted by 1-3 substituents selected from the group consisting of F, Cl, CF₃, methyl, methoxy and NR(11)R(12);
R(11) and R(12)
independently of one another are H, alkyl having 1, 2, 3 or 4 carbon atoms or perfluoroalkyl having 1, 2, 3 or 4 carbon atoms;
R(6) and R(7)
independently of one another are hydrogen, F, Cl, Br, I, CN, alkyl having 1, 2, 3, 4, 5, 6, 7 or 8 carbon atoms, perfluoroalkyl having 1, 2, 3, 4, 5, 6, 7 or 8 carbon atoms, cycloalkyl having 3, 4, 5, 6, 7 or 8 carbon atoms, or phenyl,
which is not substituted or is substituted by 1-3 substituents selected from the group consisting of F, Cl, CF₃, methyl, methoxy and NR(14)R(15);
R(14) and R(15)
independently of one another are H, alkyl having 1, 2, 3 or 4 carbon atoms or perfluoroalkyl having 1, 2, 3 or 4 carbon atoms;
and their pharmaceutically tolerable salts:
ao) benzoylguanidines of the formula I 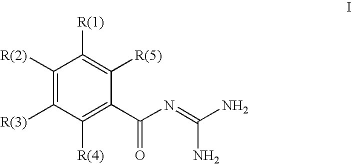
in which:
at least one of the substituents R(1), R(2) and R(3)
is R(6)-C(OH)₂—;
R(6) is perfluoroalkyl having 1, 2 or 3 carbon atoms, which is straight-chain or branched;
and the other substituents R(1), R(2) and R(3)
independently of one another are hydrogen, OH, F, Cl, Br, I, alkyl having 1, 2, 3, 4, 5 or 6 carbon atoms, cycloalkyl with 3, 4, 5 or 6 carbon atoms, alkoxy having 1, 2, 3 or 4 carbon atoms or phenoxy,
which is unsubstituted or is substituted by 1-3 substituents selected from the group consisting of F, Cl, methyl and methoxy;
or
the other substituents R(1), R(2) and R(3)
independently of one another are alkyl-SOₓ, —CR(7)=CR(8)R(9) or —C≡CR(9);
x is zero, 1 or 2;
R(7) is hydrogen or methyl;
R(8) and R(9)
independently of one another are hydrogen, alkyl having 1, 2, 3 or 4 carbon atoms, cycloalkyl having 3, 4, 5, 6, 7 or 8 carbon atoms or phenyl,
which is unsubstituted or is substituted by 1-3 substituents selected from the group consisting of F, Cl, CF₃, methyl and methoxy;
or
the other substituents R(1), R(2) and R(3)
independently of one another are phenyl, C₆H₅—(C₁-C₄)-alkyl, naphthyl, biphenylyl, quinolinyl, isoquinolinyl or imidazolyl,
where quinolinyl, isoquinolinyl or imidazolyl are bonded via C or N and where phenyl, C₆H₅—(C₁-C₄)-alkyl, naphthyl, biphenylyl, quinolinyl, isoquinolinyl and imidazolyl are unsubstituted or are substituted by 1-3 substituents selected from the group consisting of F, Cl, CF₃, CH₃, methoxy, hydroxyl, amino, methylamino and dimethylamino;
or
the other substituents R(1), R(2) and R(3)
independently of one another are SR(10), —OR(10), —CR(10)R(11)R(12);
R(10)
is —C_{f}H_{2f}—(C₃-C₈)-cycloalkyl, quinolinyl, isoquinolinyl, pyridinyl, imidazolyl or phenyl,
where the aromatic systems quinolinyl, isoquinolinyl, pyridinyl, imidazolyl and phenyl are unsubstituted or are substituted by 1-3 substituents selected from the group consisting of F, Cl, CF₃, CH₃, methoxy, hydroxyl, amino, methylamino and dimethylamino;
f is zero, 1 or 2;
R(11) and R(12)
independently of one another are defined as R(10), hydrogen or alkyl having 1, 2, 3 or 4 carbon atoms;
R(4) and R(5)
independently of one another are hydrogen, alkyl having 1, 2 or 3 carbon atoms, F, Cl, Br, I, CN, OR(13), NR(14)R(15), —(CH₂)ₙ—(CF₂)ₒ—CF₃;
R(13), R(14) and R(15)
independently of one another are hydrogen or alkyl having 1, 2, 3 or 4 carbon atoms;
n is zero or 1;
o is zero, 1 or 2;
and their pharmacologically acceptable salts;
ap) sulfonimidamides of the formula I 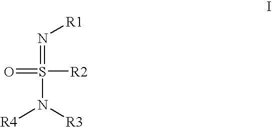
in which;
at least one of the three substituents R(1), R(2) and R(3)
is a benzoylguanidine, 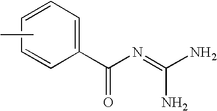
which is unsubstituted or substituted in the phenyl moiety by 1-4 radicals selected from the group consisting of alkyl having 1, 2, 3, 4, 5, 6, 7 or 8 carbon atoms, alkenyl having 2, 3, 4, 5, 6, 7 or 8 carbon atoms, —(CH₂)ₘ—R(14), F, Cl, Br, I, —C≡N, CF₃, R(22)SO₂—, R(23)R(24)N—CO—, R(25)—CO—, R(26)R(27)N—SO₂, —OR(35), —SR(35) or —NR(35)R(36);
m is zero, 1 or 2;
R(14)
is —(C₃-C₈)-cycloalkyl or phenyl,
which is not substituted or is substituted by 1-3 substituents selected from the group consisting of F and Cl, —CF₃, methyl, methoxy and —NR(15)R(16);
R(15) and R(16)
independently of one another are hydrogen or —CH₃;
R(22), R(23), R(25) and R(26)
independently of one another are alkyl having 1, 2, 3, 4, 5, 6, 7 or 8 carbon atoms, alkenyl having 2, 3, 4, 5, 6, 7 or 8 carbon atoms, (CH₂)ₙR(29) or —CF₃;
n is zero, 1, 2, 3 or 4;
R(29) is —(C₃-C₇)-cycloalkyl or phenyl,
which is not substituted or is substituted by 1-3 substituents selected from the group consisting of F, Cl, —CF₃, methyl, methoxy and —NR(30)R(31);
R(30) and R(31)
are hydrogen or alkyl having 1, 2, 3 or 4 carbon atoms;
or
R(23), R(25) and R(26)
are hydrogen;
R(24) and R(27)
independently of one another are hydrogen or alkyl having 1, 2, 3 or 4 carbon atoms;
or
1R(23) and R(24), and also R(26) and R(27)
together are 5 or 6 methylene groups, of which one CH₂ group can be replaced by oxygen, —S—, —NH—, —NCH₃ or —N-benzyl;
R(35) and R(36)
independently of one another are hydrogen or alkyl having 1, 2, 3, 4, 5 or 6 carbon atoms;
or
R(35) and R(36)
together are 4-7 methylene groups, of which one CH₂ group can be replaced by oxygen, —S—, —NH—, —NCH₃ or —N-benzyl;
or
R(35)
is phenyl,
which is not substituted or is substituted by 1-3 substituents selected from the group consisting of F, Cl, —CF₃, methyl, methoxy, SO₂R(5), SO₂NR(6)R(7) and —NR(32)R(33);
R(5) is alkyl having 1, 2, 3, 4, 5 or 6 carbon atoms
R(6) and R(7)
independently of one another are hydrogen or alkyl having 1, 2, 3 or 4 carbon atoms;
R(32) and R(33)
independently of one another are hydrogen or alkyl having 1, 2, 3 or 4 carbon atoms;
or
R(35)
is C₁-C₉-heteroaryl,
which is unsubstituted or substituted by 1-3 substituents selected from the group consisting of F, Cl, CF₃, CH₃, methoxy, hydroxyl, amino, methylamino and dimethylamino;
and the other substituents R(1), R(2) and R(3) in each case
independently of one another are alkyl having 1, 2, 3. 4, 5, 6, 7 or 8 carbon atoms, (CH₂)ₚR(10)
p is zero, 1, 2, 3 or 4;
R(10) is phenyl,
which is not substituted or is substituted by 1-3 substituents selected from the group consisting of F, Cl, —CF₃, methyl, methoxy, —SO₂NR(17)R(8) and —SO₂R(9);
R(17) and R(8)
independently of one another are hydrogen or alkyl having 1, 2, 3 or 4 carbon atoms;
R(9) is alkyl having 1, 2, 3 or 4 carbon atoms;
or the other radicals R(1) and R(3) in each case
are hydrogen,
R(4) is hydrogen or alkyl having 1, 2, 3 or 4 carbon atoms;
and their pharmaceutically tolerable salts;
aq) benzoylguanidines of the formula I 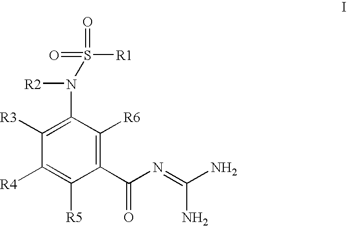
in which:
R(1) is alkyl having 1, 2, 3, 4, 5, 6, 7 or 8 carbon atoms or NR(7)R(8);
R(7) and R(8)
independently of one another are hydrogen or alkyl having 1, 2, 3, 4, 5, 6, 7 or 8 carbon atoms;
R(2) is hydrogen alkyl having 1, 2, 3, 4, 5, 6, 7 or 8 carbon atoms or —SO₂R(9);
R(9) independently is defined as R(1);
R(3) is hydrogen, —SR(25), —OR(25), —NR(25)R(26) or —CR(25)R(26)R(27);
R(25) is hydrogen, alkyl having 1, 2, 3, 4, 5, 6, 7 or 8 carbon atoms, or phenyl,
which is unsubstituted or substituted by 1-3 substituents selected from the group consisting of F, Cl, CF₃, CH₃, methoxy, hydroxyl, amino, methylamino and dimethylamino;
or
R(25)
is —(C₁-C₉)-heteroaryl,
which is unsubstituted or substituted by 1-3 substituents selected from the group consisting of F, Cl, CF₃, CH₃, methoxy, hydroxyl, amino, methylamino and dimethylamino;
R(26) and R(27)
independently of one another are defined as R(25) or are hydrogen or alkyl having 1, 2, 3, 4, 5, 6, 7 or 8 carbon atoms;
R(4) is hydrogen, F, Cl, Br, I, OH, —C≡N, CF₃, alkyl having 1, 2, 3, 4, 5, 6, 7 or 8 carbon atoms, alkenyl having 2, 3, 4, 5, 6, 7 or 8 carbon atoms or —(CH₂)ₘR(14);
m is zero, 1 or 2;
R(14) is —(C₃-C₈)-cycloalkyl or phenyl,
which is not substituted or is substituted by 1-3 substituents selected from the group consisting of F and Cl, —CF₃, methyl, methoxy and —NR(15)R(16);
R(15) and R(16)
independently of one another are hydrogen or —CH₃;
R(5) and R(6)
independently of one another are hydrogen, alkyl having 1, 2, 3 or 4 carbon atoms, F, Cl, —OR(32), —NR(33)R(34) or CF₃;
R(32), R(33) and R(34)
independently of one another are hydrogen or alkyl having 1, 2, 3 or 4 carbon atoms;
and their pharmaceutically tolerable salts;
ar) benzenedicarboxylic acid diguanidides of the formula I 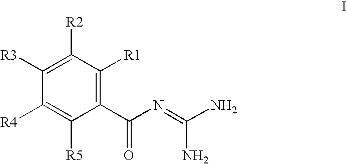
in which:
one of the radicals R(1), R(2), R(3) and R(4)
is —CO—N═C(NH₂)₂;
and the other radicals R(1), R(2), R(3) and R(4) in each case are:
R(1) is hydrogen, alkyl having 1, 2, 3 or 4 carbon atoms, F, Cl, Br, I,
—OR(32), —NR(33)R(34) or CF₃;
R(32), R(33) and R(34)
independently of one another are hydrogen or alkyl having 1, 2, 3 or 4 carbon atoms;
R(2) and R(4)
independently of one another are hydrogen, F, Cl, Br, I, OH, —CN, CF₃, —CO—N═C(NH₂)₂, alkyl having 1, 2, 3, 4, 5, 6, 7 or 8 carbon atoms, alkenyl having 2, 3, 4, 5, 6, 7 or 8 carbon atoms or —(CH₂)ₘR(1 4);
m is zero, 1 or 2;
R(14) is —(C₃-C₈)-cycloalkyl or phenyl,
which is not substituted or is substituted by 1-3 substituents selected from the group consisting of F and Cl, —CF₃, methyl, methoxy and —NR(15)R(16);
R(15) and R(16)
are hydrogen or —CH₃;
or
R(2) and R(4)
independently of one another are pyrrol-1-yl, pyrrol-2-yl or pyrrol-3-yl,
each of which is not substituted or is substituted by 1-4 substituents selected from the group consisting of F, Cl, Br, I, —CN, (C₂-C₈)-alkanoyl, (C₂-C₈)-alkoxycarbonyl, formyl, carboxyl, —CF₃, methyl, methoxy;
or
R(2) and R(4)
independently of one another are R(22)-SO₂—, R(23)R(24)N—CO—, R(28)-CO— or R(29)R(30)N—SO₂;
R(22) and R(28)
independently of one another are methyl or —CF₃;
R(23), R(24), R(29) and R(30)
independently of one another are hydrogen or methyl;
or
R(2) and R(4)
independently of one another are —OR(35) or —NR(35)R(36);
R(35) and R(36)
independently of one another are hydrogen or alkyl having 1, 2, 3, 4, 5 or 6 carbon atoms;
or
R(35) and R(36)
together are 4-7 methylene groups, of which one CH₂ group can be replaced by oxygen, —S—, —NH—, —NCH₃ or —N-benzyl;
R(3) is hydrogen, —SR(25), —OR(25), —NR(25)R(26), —CR(25)R(26)R(27);
R(25) is hydrogen, alkyl having 1, 2, 3, 4, 5, 6, 7 or 8 carbon atoms or phenyl,
which is unsubstituted or substituted by 1-3 substituents selected from the group consisting of F, Cl, CF₃, CH₃, methoxy, hydroxyl, amino, methylamino and dimethylamino;
or
R(25) is —(C₁-C₉)-heteroaryl,
which is unsubstituted or substituted by 1-3 substituents selected from the group consisting of F, Cl, CF₃, CH₃, methoxy, hydroxyl, amino, methylamino and dimethylamino;
R(26) and R(27)
independently of one another are defined as R(25) or are hydrogen or alkyl having 1, 2, 3, 4, 5, 6, 7 or 8 carbon atoms;
R(5) is alkyl having 1, 2, 3 or 4 carbon atoms, F, Cl, Br, I, X—(CH₂)_{y}—CF₃ or phenyl,
which is not substituted or is substituted by 1-3 substituents selected from the group consisting of F and Cl, —CF₃, methyl, methoxy and —NR(6)R(7);
R(6) and R(7)
independently of one another are hydrogen or —CH₃;
X is a bond or oxygen;
y is zero, 1 or 2;
and their pharmaceutically tolerable salts;
as) benzenedicarboxylic acid diguanidides of the formula I 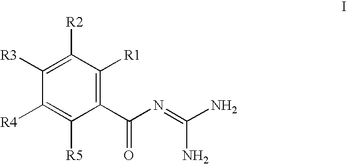
in which:
one of the radicals R(1), R(2), R(3) and R(5)
is —CO—N═C(NH₂)₂;
and the other radicals R(1), R(2), R(3) and R(5) in each case are:
R(1) and R(5)
independently of one another are hydrogen, alkyl having 1, 2, 3 or 4 carbon atoms, F, Cl, —OR(32), —NR(33)R(34) or CF₃;
R(32), R(33) and R(34)
independently of one another are hydrogen or alkyl having 1, 2, 3 or 4 carbon atoms;
R(2) is hydrogen, F, Cl, Br, I, OH, —C^{o}N, CF₃, —CO—N═C(NH₂)₂, alkyl having 1, 2, 3, 4, 5, 6, 7 or 8 carbon atoms, alkenyl having 2, 3, 4, 5, 6, 7 or 8 carbon atoms or —(CH₂)ₘR(14);
m is zero, 1 or 2;
R(14) is —(C₃-C₈)-cycloalkyl or phenyl,
which is not substituted or is substituted by 1-3 substituents selected from the group consisting of F and Cl, —CF₃, methyl, methoxy and —NR(15)R(16);
R(15) and R(16)
independently of one another are hydrogen or —CH₃;
or
R(2) is R(22)-SO₂—, R(23)R(24)N—CO—, R(28)-CO— or R(29)R(30)N—SO₂;
R(22) and R(28)
independently of one another are methyl or —CF₃;
R(23), R(24), R(29) and R(30)
independently of one another are hydrogen or methyl;
or
R(2) is —OR(35) or —NR(35)R(36);
R(35) and R(36)
independently of one another are hydrogen or alkyl having 1, 2, 3, 4, 5 or 6 carbon atoms;
or
R(35) and R(36)
together are 4-7 methylene groups, of which one CH₂ group can be replaced by oxygen, —S—, —NH—, —NCH₃ or —N-benzyl:
R(3) is hydrogen, —SR(25), —OR(25), —NR(25)R(26), —CR(25)R(26)R(27);
R(25) is hydrogen, alkyl having 1, 2, 3, 4, 5. 6, 7 or 8 carbon atoms or phenyl,
which is unsubstituted or substituted by 1-3 substituents selected from the group consisting of F, Cl, CF₃, CH₃, methoxy, hydroxyl, amino, methylamino and dimethylamino;
or
R(25) is —(C₁-C₉)-heteroaryl,
which is unsubstituted or substituted by 1-3 substituents selected from the group consisting of F, Cl, CF₃, CH₃, methoxy, hydroxyl, amino, methylamino and dimethylamino;
R(26) and R(27)
independently of one another are defined as R(25) or are hydrogen or alkyl having 1, 2, 3, 4, 5, 6, 7 or 8 carbon atoms;
R(4) is CF₃, alkyl having 1, 2, 3, 4, 5, 6, 7 or 8 carbon atoms, alkenyl [symbol]
R(32), R(33) and R(34)
independently of one another are hydrogen or alkyl having 1, 2, 3 or 4 carbon atoms;
the other radicals R(2) and R(4) in each case
independently of one another are hydrogen, F, Cl, Br, I, OH, —CN, CF₃, —CO—N═C(NH₂)₂, alkyl having 1, 2, 3, 4, 5, 6, 7 or 8 carbon atoms, alkenyl having 2, 3, 4, 5, 6, 7 or 8 carbon atoms or —(CH₂)ₘR(14);
m is zero, 1 or 2;
R(14) is —(C₃-C₈)-cycloalkyl or phenyl,
which is not substituted or is substituted by 1-3 substituents selected from the group consisting of F and Cl, —CF₃, methyl, methoxy and —NR(15)R(16);
R(15) and R(16)
are hydrogen or —CH₃;
or
the other radicals R(2) and R(4) in each case
independently of one another are pyrrol-1-yl, pyrrol-2-yl or pyrrol-3-yl,
which is not substituted or is substituted by 1-4 substituents selected from the group consisting of F, Cl, Br, I, —CN, (C₂-C₈)-alkanoyl, (C₂-C₈)-alkoxycarbonyl, formyl, carboxyl, —CF₃, methyl, methoxy;
or
the other radicals R(2) and R(4) in each case
are R(22)-SO₂—, R(23)R(24)N—CO—, R(28)—CO— or R(29)R(30)N—SO₂;
R(22) and R(28)
independently of one another are methyl or —CF₃;
R(23), R(24), R(29) and R(30)
independently of one another are hydrogen or methyl;
or
the other radicals R(2) and R(4) in each case
independently of one another are —OR(35) or —NR(35)R(36);
R(35) and R(36)
independently of one another are hydrogen or alkyl having 1, 2, 3, 4, 5 or 6 carbon atoms;
or
R(35) and R(36)
together are 4-7 methylene groups, of which one CH₂ group can be replaced by oxygen,—S—, —NH—, —NCH₃ or —N-benzyl;
the other radical R(3) in each case
is hydrogen, —SR(25), —OR(25), —NR(25)R(26), —CR(25)R(26)R(27);
R(25) is hydrogen, alkyl having 1, 2, 3, 4, 5, 6, 7 or 8 carbon atoms or phenyl,
which is unsubstituted or substituted by 1-3 substituents selected from the group consisting of F, Cl, CF₃, CH₃, methoxy, hydroxyl, amino, methylamino and dimethylamino;
or
R(25) is —(C₁-C₉)-heteroaryl,
which is unsubstituted or substituted by 1-3 substituents selected from the group consisting of F, Cl, CF₃, CH₃, methoxy, hydroxyl, amino, methylamino and dimethylamino;
R(26) and R(27)
independently of one another are defined as R(25) or are hydrogen or alkyl having 1, 2, 3, 4, 5, 6, 7 or 8 carbon atoms;
one of the radicals R(6), R(7), R(8), R(9) and R(10)
is —CO—N═C(NH₂)₂;
the other radicals R(6) and R(10) in each case
independently of one another are hydrogen, alkyl having 1, 2, 3 or 4 carbon atoms, F, Cl, —OR(132), —NR(133)R(134) or CF₃;
R(132), R(133) and R(134)
independently of one another are hydrogen or alkyl having 1, 2, 3 or 4 carbon atoms;
the other radicals R(7) and R(9) in each case
independently of one another are hydrogen, F, Cl, Br, I, OH, —CN, CF₃, —CO—N═C(NH₂)₂, alkyl having 1, 2, 3, 4, 5, 6, 7 or 8 carbon atoms, alkenyl having 2, 3, 4, 5, 6, 7 or 8 carbon atoms or —(CH₂)ₘₘR(114);
mm is zero, 1 or 2;
R(114)
is —(C₃-C₈)-cycloalkyl or phenyl,
which is not substituted or is substituted by 1-3 substituents selected from the group consisting of F and Cl, —CF₃₁ methyl, methoxy and —NR(115)R(116);
R(115) and R(116)
are hydrogen or —CH₃;
or
the other radicals R(7) and R(9) in each case
independently of one another are pyrrol-1-yl, pyrrol-2-yl or pyrrol-3-yl,
which is not substituted or is substituted by 1-4 substituents selected from the group consisting of F, Cl, Br, I, —CN, (C₂-C₈)-alkanoyl, (C₂-C₈)-alkoxycarbonyl, formyl, carboxyl, —CF₃, methyl and methoxy;
or
the other radicals R(7) and R(9) in each case
are R(122)-SO₂—, R(123)R(124)N—CO—, R(128)-CO— or R(129)R(130)N—SO₂;
R(122) and R(128)
independently of one another are methyl or —CF₃;
R(123), R(124), R(129) and R(130)
independently of one another are hydrogen or methyl;
or
the other radicals R(7) and R(9) in each case
independently of one another are —OR(135) or —NR(135)R(136);
R(135) and R(136)
independently of one another are hydrogen or alkyl having 1, 2, 3, 4, 5 or 6 carbon atoms;
or
R(135) and R(136)
together are 4-7 methylene groups, of which one CH₂ group can be replaced by oxygen,—S—, —NH—, —NCH₃ or —N-benzyl;
the other radical R(8) in each case
is hydrogen, —SR(125), —OR(125), —NR(125)R(126) or —CR(125)R(126)R(127);
R(125)
is hydrogen, alkyl having 1, 2, 3, 4, 5, 6, 7 or 8 carbon atoms or phenyl,
which is unsubstituted or substituted by 1-3 substituents selected from the group consisting of F, Cl, CF₃, CH₃, methoxy, hydroxyl, amino, methylamino and dimethylamino;
or
R(125)
is —(C₁-C₉)-heteroaryl,
which is unsubstituted or substituted by 1-3 substituents selected from the group consisting of F, Cl, CF₃, CH₃, methoxy, hydroxyl, amino, methylamino and dimethylamino;
R(126) and R(127)
independently of one another are defined as R(125) or are hydrogen or alkyl having 1, 2, 3, 4, 5, 6, 7 or 8 carbon atoms;
A is absent or is —NR(11)-CO—, —NR(12)-CO—NR(13)-, —NR( 17)-CO—NR(18)-SO₂—, —N R(19)-SO2—, —SO₂—NR(19)-SO₂—, —SO₂—NR(19)-CO—, —O—CO—NR(19)-SO₂— or —CR(20)=CR(21)-;
R(11), R(12), R(13), R(17), R(18), R(19), R(20) and R(21)
independently of one another are hydrogen or alkyl having 1, 2, 3, 4, 5, 6, 7 or 8 carbon atoms
and their pharmaceutically tolerable salts;
au) substituted thiophenylalkenylcarboxylic acid guanidides of the formula I 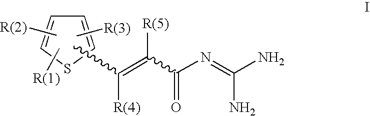
in which:
at least one of the substituents R(1), R(2) and R(3)
is —Oₚ—(CH₂)ₛ—C_{q}F_{2q+1}, R(40)CO— or R(31)SOₖ—;
p is zero or 1;
s is zero, 1, 2, 3 or 4,
q is 1, 2, 3, 4, 5, 6, 7 or 8;
k is zero, 1 or 2;
R(40) is alkyl having 1, 2, 3, 4, 5, 6, 7 or 8 carbon atoms,
perfluoroalkyl having 1, 2, 3, 4, 5, 6, 7 or 8 carbon atoms, cycloalkyl having 3, 4, 5, 6, 7 or 8 carbon atoms, or phenyl,
which is not substituted or is substituted by 1-3 substituents selected from the group consisting of F, Cl, CF₃, methyl and methoxy;
R(31) is alkyl having 1, 2, 3, 4, 5, 6, 7 or 8 carbon atoms,
perfluoroalkyl having 1, 2, 3, 4, 5, 6, 7 or 8 carbon atoms, cycloalkyl having 3, 4, 5, 6, 7 or 8 carbon atoms, or phenyl,
which is not substituted or is substituted by 1-3 substituents selected from the group consisting of F, Cl, CF₃, methyl or methoxy;
or
R(31) is NR(41)R(42);
R(41) and R(42)
independently of one another are hydrogen, alkyl having 1, 2, 3 or 4 carbon atoms, perfluoroalkyl having 1, 2, 3 or 4 carbon atoms,
or
R(41) and R(42)
together are 4 or 5 methylene groups, of which one CH₂ group can be replaced by oxygen, S, NH, N—CH₃ or N-benzyl;
and the other substituents R(1), R(2) and R(3) in each case
independently of one another are H, F, Cl, Br, I, CN, —Oₙₐ—CₘₐH₂ₘₐ₊₁ or —O_{ga}CᵣₐH₂ᵣₐR(10);
na is zero or 1;
ma is zero, 1, 2, 3, 4, 5, 6, 7 or 8;
ga is zero or 1;
ra is zero, 1, 2, 3 or 4;
R(10) is cycloalkyl having 3, 4, 5, 6, 7 or 8 carbon atoms or phenyl,
where the phenyl is not substituted or is substituted by 1-3 substituents selected from the group consisting of F, Cl, CF₃, methyl and methoxy;
R(4) and R(5)
independently of one another are hydrogen, F, Cl, Br, 1, CN, alkyl having 1, 2, 3, 4, 5, 6, 7 or 8 carbon atoms, perfluoroalkyl having 1, 2, 3, 4, 5, 6, 7 or 8 carbon atoms, cycloalkyl having 3, 4, 5, 6, 7 or 8 carbon atoms or phenyl,
which is not substituted or is substituted by 1-3 substituents selected from the group consisting of F, Cl, CF₃, methyl, methoxy and NR(14)R(15);
R(14) and R(15)
independently of one another are H, alkyl having 1, 2, 3 or 4 carbon atoms or perfluoroalkyl having 1, 2, 3 or 4 carbon atoms;
and their pharmaceutically tolerable salts;
av) ortho-substituted benzoylguanidines of the formula I 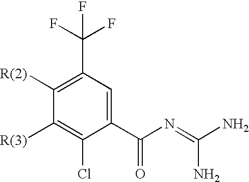
in which:
R(2) and R(3)
independently of one another are hydrogen, Cl, Br, I, (C₁-C₈)-alkyl, (C3-C8)-cycloalkyl or —OR(5);
R(5) is (C₁-C₈)-alkyl or —C_{d}H_{2d}—(C3-C8)-cycloalkyl;
d is zero, 1 or 2;
where one of the two substituents R(2) and R(3) is always hydrogen but both substituents R(2) and R(3) are not simultaneously hydrogen, and their pharmaceutically tolerable salts;
ax) benzoylguanidines of the formula I 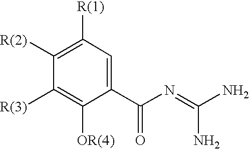
in which:
R(1) is H, F, Cl, Br, I, CN, NO₂, alkyl having 1, 2, 3, 4, 5, 6, 7 or 8 carbon atoms, alkoxy having 1, 2, 3, 4, 5, 6, 7 or 8 carbon atoms, cycloalkyl having 3, 4, 5, 6, 7 or 8 carbon atoms, cycloalkoxy having 3, 4, 5, 6, 7 or 8 carbon atoms or Xₐ—(CH₂)_{b}—(CF₂)_{c}—CF₃;
X is oxygen, S, NR(5),
a is zero or 1;
b is zero, 1 or 2;
c is zero, 1, 2or 3;
R(5) is H, alkyl having 1, 2, 3 or 4 carbon atoms or —C_{d}H_{2d}R(⁶);
d is zero, 1, 2, 3 or 4;
R(6) is cycloalkyl having 3, 4, 5, 6, 7 or 8 carbon atoms, phenyl, biphenylyl or naphthyl,
where the aromatics phenyl, biphenylyl or naphthyl are not substituted or are substituted by 1-3 substituents selected from the group consisting of F, Cl, CF₃, methyl, methoxy and NR(7)R(8);
R(7) and R(8)
independently are H or alkyl having 1, 2, 3 or 4 carbon atoms;
or
R(1) is —SR(10), —OR(10) or —CR(10)R(11)R(12);
R(10) is —C_{f}H_{2f}-cycloalkyl having 3, 4, 5, 6, 7 or 8 carbon atoms in the cycloalkyl ring, or phenyl,
where phenyl is unsubstituted or substituted by 1-3 substituents selected from the group consisting of F, Cl, CF₃, CH₃, methoxy, hydroxyl, amino, methylamino and dimethylamino;
f is zero, 1 or 2;
R(11) and R(12)
independently of one another are defined as R(10) or are hydrogen or alkyl having 1, 2, 3 or 4 carbon atoms;
or
R(1) is phenyl, naphthyl, biphenylyl or heteroaryl having 1, 2, 3, 4, 5, 6, 7, 8 or 9 carbon atoms, with the latter being linked via a carbon atom or a nitrogen atom of the ring,
which are in each case unsubstituted or substituted by 1-3 substituents selected from the group consisting of F, Cl, CF₃, CH₃, methoxy, hydroxyl, amino, methylamino and dimethylamino,
or
R(1) is —SR(13), —OR(13), —NHR(13), —NR(13)R(14), —CHR(13)R(15), —C[R(15)R(16)OH₁], —C≡CR(18), —C[R(19)]=CHR(18), —C[R(20)R(21 )]ₖ-(CO)—[CR(22)R(23)]ₗ-R(24),
k is zero, 1, 2, 3 or 4;
l is zero, 1, 2, 3 or 4;
R(13) and R(14)
identically or differently are —(CH2)_{g}—(CHOH)ₕ—(CH₂)ᵢ—(CHOH)ⱼ—R(17) or —(CH₂)_{g}—O—(CH₂—CH₂O)ₕ—R(24);
R(17) is hydrogen or methyl,
g, h and i
identically or differently are zero, 1, 2, 3 or 4;
j is 1, 2, 3 or 4;
R(15) and R(16)
identically or differently are hydrogen, alkyl having 1, 2, 3, 4, 5 or 6 carbon atoms or, together with the carbon atom carrying them, are cycloalkyl having 3, 4, 5, 6, 7 or 8 carbon atoms;
R(18)
is phenyl,
which is unsubstituted or substituted by 1-3 substituents selected from the group consisting of F, Cl, CF₃, methyl, methoxy and NR(25)R(26);
R(25) and R(26)
are H or alkyl having 1, 2, 3 or 4 carbon atoms;
or
R(18) is heteroaryl having 1, 2, 3, 4, 5, 6, 7, 8 or 9 carbon atoms,
which is unsubstituted or substituted as phenyl;
or
R(18) is alkyl having 1, 2, 3, 4, 5 or 6 carbon atoms,
which is unsubstituted or substituted by 1-3 OH;
or
R(18)
is cycloalkyl having 3, 4, 5, 6, 7 or 8 carbon atoms;
R(19), R(20), R(21), R(22) and R(23)
identically or differently are hydrogen or methyl;
R(24) is H, alkyl having 1, 2, 3, 4, 5 or 6 carbon atoms, cycloalkyl having 3, 4, 5, 6, 7 or 8 carbon atoms or —CₘH₂ₘ—R(¹⁸);
?? very broad with all R(18)s
m is 1, 2, 3 or 4;
R(2) and R(3)
are defined as R(1);
R(4) is alkyl having 1, 2, 3 or 4 carbon atoms; and their pharmaceutically tolerable salts;
ay) ortho-substituted benzoylguanidines of the formula I 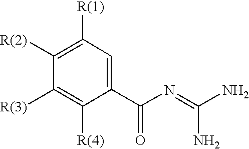
in which:
R(1) is H, F, Cl, Br, I, CN, NO₂, alkyl having 1, 2, 3, 4, 5, 6, 7 or 8 carbon atoms, alkoxy having 1, 2, 3, 4, 5, 6, 7 or 8 carbon atoms, cycloalkyl having 3, 4, 5, 6, 7 or 8 carbon atoms, cycloalkoxy having 3, 4, 5, 6, 7 or 8 carbon atoms or Xₐ—(CH₂)_{b}—(CF₂)_{c}—CF₃;
X is oxygen, S, NR(5),
a is zero or 1;
b is zero, 1 or 2;
c is zero, 1, 2 or 3;
R (5) is H, alkyl having 1, 2, 3 or 4 carbon atoms or —C_{d}H_{2d}R(6);
d is zero, 1, 2, 3 or 4;
R(6) is cycloalkyl having 3, 4, 5, 6, 7 or 8 carbon atoms, phenyl, biphenylyl or naphthyl,
where the aromatics phenyl, biphenylyl or naphthyl are unsubstituted or substituted by 1-3 substituents selected from the group consisting of F, Cl, CF₃, methyl, methoxy and NR(7)R(8);
R(7) and R(8)
independently are H or alkyl having 1, 2, 3 or 4 carbon atoms;
or
R(1) is —SR(10), —OR(10) or —CR(10)R(11)R(12);
R(10) is —C_{f}H_{2f}-cycloalkyl having 3, 4, 5, 6, 7 or 8 carbon atoms in the cycloalkyl ring, or phenyl,
where phenyl is unsubstituted or substituted by 1-3 substituents selected from the group consisting of F, Cl, CF₃, CH₃, methoxy, hydroxyl, amino, methylamino and dimethylamino;
f is zero, 1 or 2;
R(11) and R(12)
independently of one another are defined as R(10), or are hydrogen or alkyl having 1, 2, 3 or 4 carbon atoms;
or
R(1) is phenyl, naphthyl, biphenylyl or heteroaryl having 1, 2, 3, 4, 5, 6, 7, 8 or 9 carbon atoms, with the latter being linked via a carbon atom or a nitrogen atom of the ring,
which are in each case unsubstituted or substituted by 1-3 substituents selected from the group consisting of F, Cl, CF₃, CH₃, methoxy, hydroxyl, amino, methylamino and dimethylamino,
or
R(1) is —SR(13), —OR(13), —NHR(13), —NR(13)R(14), —CHR(13)R(15), —C[R(15)R(16)OH], —C≡CR(18), —C[R(19)]=CHR(18), —C[R(20)R(21)]ₖ-(CO)—[CR(22)R(23)]ₗ-R(24),
k is zero, 1, 2, 3 or 4;
l is zero, 1, 2, 3 or 4;
R(13) and R(14)
identically or differently are —(CH₂)_{g}—(CHOH)ₕ—(CH₂)ᵢ—(CHOH)ⱼ—R(17) or —(CH₂)_{g}—O—(CH₂—CH₂O)ₕ—R(24);
R(17) is hydrogen or methyl,
g, h and i
identically or differently are zero, 1, 2, 3 or 4;
is 1, 2, 3 or 4;
R(15) and R(16)
identically or differently are hydrogen, alkyl having 1, 2, 3, 4, 5 or 6 carbon atoms or, together with the carbon atom carrying them, are cycloalkyl having 3, 4, 5, 6, 7 or 8 carbon atoms;
R(18)
is phenyl,
which is unsubstituted or substituted by 1-3 substituents selected from the group consisting of F, Cl, CF₃, methyl, methoxy and NR(25)R(26);
R(25) and R(26)
are H or alkyl having 1, 2, 3 or 4 carbon atoms;
or
R(18) is heteroaryl having 1, 2, 3, 4, 5, 6, 7, 8 or 9 carbon atoms,
which is unsubstituted or substituted as phenyl;
or
R(18) is alkyl having 1, 2, 3, 4, 5 or 6 carbon atoms,
which is unsubstituted or substituted by 1-3 OH;
or
R(18)
is cycloalkyl having 3, 4, 5, 6, 7 or 8 carbon atoms;
R(19), R(20), R(21), R(22) and R(23)
identically or differently are hydrogen or methyl;
R(24) is H, alkyl having 1, 2, 3, 4, 5 or 6 carbon atoms, cycloalkyl having 3, 4, 5, 6, 7 or 8 carbon atoms or —CₘH₂ₘ—R(18);
m is 1, 2, 3 or 4;
one of the two substituents R(2) and R(3)
is hydroxyl;
and
the other of the substituents R(2) and R(3) in each case
is defined as R(1);
R(4) is alkyl having 1, 2, 3 or 4 carbon atoms; alkoxy having 1, 2, 3 or 4 carbon atoms, F, Cl, Br, I or —(CH₂)ₙ—(CF₂)ₒ—CF₃;
n is zero or 1;
o is zero or 1;
and their pharmaceutically tolerable salts.
II. Compounds of the formula 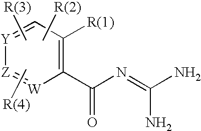
in which:
W, Y and Z
are a nitrogen atom or a carbon atom substituted by R(2) or R(3) or R(4);
R(1) is hydrogen, A, Hal, —CF₃, —CH₂F, —CHF₂, —CH₂CF₃, —C₂F₅, —CN, —NO₂, -ethynyl, or an X—R′;
A is alkyl having 1 to 6 carbon atoms;
Hal is F, Cl, Br or I;
x is oxygen, S or NR″;
R″ is hydrogen, A or a cyclic methylene chain having 3 to 7 carbon atoms;
R′ is H, A, HO-A-, HOOC-A-, (C₃-C₇)cycloalkyl, (C₆-C₈)-cycloalkylalkyl, CF₃, CH₂F, CHF₂, CH₂—CF₃, Ph, —CH₂—Ph or Het;
Ph is phenyl, naphthyl or biphenylyl, which is unsubstituted or mono-, di- or trisubstituted by A, OA, NR′R″, Hal, CF₃;
Het is a mono- or binuclear saturated, unsaturated or aromatic heterocycle having 1 to 4 nitrogen, oxygen and/or sulfur atoms,
which is unsubstituted or mono-, di- or trisubstituted by Hal, CF₃, A, OH, OA, —X—R′, —CN, —NO₂, and/or carbonyl oxygen,
where Het is bonded via N or an alkylene chain CₘH₂ₘ where m =zero to 6;
or
R′ and R″
together are alkylene having 4-5 carbon atoms, in which one CH₂ group can also be replaced by oxygen, S, NH, N-A, N—Ph and N—CH₂—Ph;
R(2) and R(3)
independently of one another are hydrogen, Hal, A, HO-A-, X—R′, —C(═N—OH)-A, A-O—CO—(C₁-C₄)-alkyl-, CN, NO₂, COOH, halogen-substituted A, in particular CF₃, CH₂F, CHF₂, C₂F₅, CH₂CF₃, or S(O)ₙR′″;
R′″ is A, Ph or -Het;
n is zero, 1 or 2;
or
R(2) and R(3)
independently of one another are SO₂NR′R″, Ph or —O—Ph, —O—CH₂—Ph, —CO-A, —CHO, —COOA, —CSNR′R″, CONR′R″, —CH═CH—COOH, —CH═CH—COOA, indenyl, indanyl, decahydronaphthyl, cyclopentenyl, dihydrothienyl, dihydrofuryl, heterobicyclyl, alkylthienyl, halothienyl, haloalkylthienyl, acytthienyl, halofuryl, haloalkylfuryl or pyrrolyl;
or
R(2) and R(3)
independently of one another are R(5)-O—;
R(5) is hydrogen, A, (C₁-C₆)-alkenyl or (C₃-C₇)-cycloalkyl;
R(4) is Ph, Het, —O-Het; CF₃, S(O)ₙR′″, —SO₂NR′R″, alk; 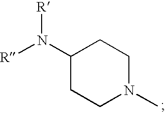
or
two of the substituents R(1) to R(4)
together are a group —O—CR(6)R(7)-CO—NR(8)-,
or 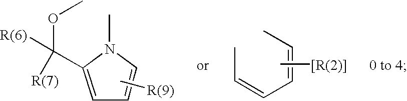
where R(2) has the meaning indicated;
R(6), R(7), R(8) and R(9)
independently of one another are H or A;
or
R(8) is (C₅-C₇)-cycloalkyl;
or
R(9) is cyano;
alk is straight-chain or branched (C₁-C₈)-alkyl or (C₃-C₈)-cycloalkyl, which is unsubstituted or mono-, di- or trisubstituted by A;
or
alk is an ethenyl or ethynyl radical which is substituted by H, A, Ph or Het.
III. Indoloylguanidine derivatives of the formula 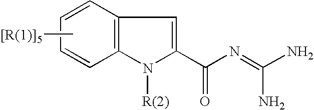
in which
R(2) is hydrogen, unsubstituted or substituted (C₁-C₈)-alkyl, (C₃-C₇)-cycloalkyl, OH, (C₁-C₆)-alkyl-O—, an aromatic radical or a group —CH₂—R(20);
R(20) is (C₂-C₆)-alkenyl or (C₂-C₆)-alkynyl;
R(1) is 1 to 5 identical or different substituents, which are:
hydrogen, unsubstituted or substituted (C₁-C₈)-alkyl, (C₂-C₆)-alkenyl, (C₂-C₆)-alkynyl, (C₃-C₇)-cycloalkyl, halogen, —NO₂, (C₂-C₈)-alkanoyl, arylalkanoyl having up to 10 carbon atoms, aroyl having up to 11 carbon atoms, —COOH, (C₂-C₆)-alkoxycarbonyl, an aromatic group or one of the following mentioned groups: —OR(3), —NR(6)R(7) or —S(O)ₙR(40);
R(3) is hydrogen, (C₁-C₈)-alkyl, substituted (C₁-C₈)-alkyl, (C₃-C₇)-cycloalkyl, an aromatic radical or a group —CH₂—R(30) R(30) is alkenyl or alkynyl;
R(6) and R(7)
independently of one another are hydrogen, unsubstituted or substituted (C₁-C₈)-alkyl, (C₃-C₇)-cycloalkyl, (C₂-C₈)-alkanoyl, an arylalkanoyl group having up to 10 carbon atoms, an aroyl group having up to 11 carbon atoms, an aromatic group or —CH₂—R(60);
R(60) is (C₂-C₆)-alkenyl or (C₂-C₆)-alkynyl;
or
R(6) and R(7)
together with the nitrogen atom are a 5-7-membered cyclic amine, which can additionally contain further heteroatoms in the ring;
n is zero, 1 or 2;
R(40) is unsubstituted or substituted (C₁-C₈)-alkyl, or an aromatic group, or a group 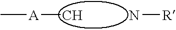
A is oxygen, —S(O)ₙ— or —N(R50)—;
R(50) is hydrogen or (C₁-C₈)-alkyl;
R′ is hydrogen, unsubstituted or substituted (C₁-C₈)-alkyl, in which the ring represents a saturated 3-8-membered heterocycle having a nitrogen atom,
said substituted alkyl carries one or more groups selected from the group consisting of halogen, —OH, (C₁-C₆)-alkoxy, —CN, —COOH, (C₂-C₆)alkoxycarbonyl, (C₂-C₈)-alkanoyl, arylalkanoyl having up to 10 carbon atoms, aroyl having up to 11 carbon atoms, an aromatic group, —CONR(4)R(5),
R(4) and R(5)
identically or differently are hydrogen or (C₁-C₈-alkyl;
or
R(4) and R(5)
are connected to one another and together form a 5-7-membered cyclic amine which can additionally contain further heteroatoms in the ring,
or said substituted alkyl carries a group 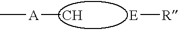
in which:
E is a nitrogen atom or a CH group;
R″ is hydrogen, (C₁-C₈)-alkyl which is unsubstituted or substituted by OH, (C₁-C₆)-alkoxy, —CN, —COOH, (C₂-C₆)-alkoxycarbonyl, (C₂-C₈)-alkanoyl, aralkanoyl having up to 10 carbon atoms, aroyl having up to 11 carbon atoms, an aromatic group, —NR(6)R(7), —CONR(4)R(5);
R(4) and R(5)
independently of one another are hydrogen or (C₁-C₈)-alkyl;
where the cyclic system of the formula 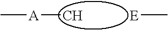
is a 3-8-membered saturated aliphatic or heterocyclic ring system having a nitrogen atom,
and where the aromatic groups mentioned are an aryl radical having up to 10 carbon atoms, a 5- or 64[membered heteroaryl radical having 1-4 nitrogen atoms, a 5- or 6-membered heteroaryl group containing 1 or 2 nitrogen atoms and a heteroatom which is oxygen or sulfur, or furyl, and where the aryl radicals mentioned can be unsubstituted or substituted by unsubstituted (C₁-C₈)-alkyl or substituted (C₁-C₈)-alkyl, halogen, —NO₂, (C₂-C₆)-alkoxycarbonyl, COOH, —OR(3), NR(6)R(7), —CONR(4)R(5), —SO₂NR(6)R(7) or S(O)ₙR(40),
where R(1) and the guanidinocarbonyl radical can be in any desired position of the 5- or 6-membered ring of the indole system, and the appropriate pharmaceutically tolerable salts.
IV. Heterocyclic guanidine derivatives of the formula 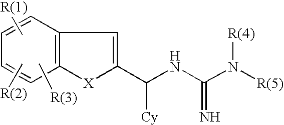
in which:
X is —O—, —S—, —NH—, —N[(C₁-C₄)-alkyl]- or —N(phenyl)-;
R(1), R(2) and R(3)
are hydrogen, halogen, (C₁-C₄)-alkyl, (C₁-C₄)-alkyl-O—, phenyl, benzyl;
or
two of the substituents R(1), R(2) and R(3)
together with one side of the benzo system are a 4-6-membered carbocyclic ring;
R(4) and R(5)
independently of one another are hydrogen, (C₁-C₁₂)-alkyl, benzhydryl, aralkyl,
which is unsubstituted or substituted by one or more substituents from the groups halogen, (C₁-C₄)-alkyl, (C₁-C₄)-alkyl-O— or —CF₃, —(CH₂)ₘ—CH₂-T,
m is zero to 3;
T is —CO—O-T(1);
T(1) is hydrogen or (C₁-C₄)-alkyl;
Cy is a benzofused unsaturated or dihydro-5-membered ring heterocycle 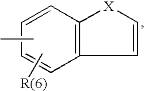
a pyrazole or imidazole ring of the formula 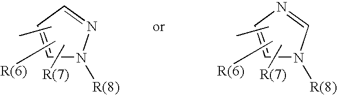
a naphthyl radical or a dihydro- or tetrahydronaphthyl radical 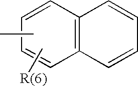
a 2-, 3- or 4-pyridyl radical 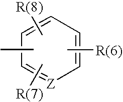
Z is N— or CH;
a thienyl radical 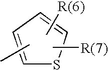
R(6) is hydrogen, halogen, hydroxyl, (C₁-C₁₀)-alkyl, (C₁-C₁₀)-alkyl-O—, phenoxy, (C₁-C₁₀)-alkyloxymethyloxy- or —(O)ₙS—R(9);
R(9) is (C₁-C₁₀)-alkyl, thienyl, pyridyl, thiazolyl, thiadiazolyl, imidazolyl, pyrazolyl or phenyl,
each of which is unsubstituted or mono- or disubstituted by halogen, (C₁-C₄)-alkyl or (C₁-C₄)-alkyl-O—;
R(7) and R(8)
is hydrogen, halogen, hydroxyl, (C₁-C₁₀)-alkyl, (C₁-C₁₀)-alkyl-O—, phenyl, phenoxy or (C₁-C₁₀)-alkoxymethyloxy;
or
Cy is phenyl,
which is unsubstituted or mono- or disubstituted by halogen, (C₁-C₄)-alkyl or (C₁-C₄)-alkyl-O—;
or
Cy is -Gr-Am;
Gr is —R(13)-R(12)-(CH₂)_{q}—C[W]W(1)]-(CH₂)_{q}—; R(13)R(14)- or —R(15)-;
R(12) is a single bond, —O—, —(O)ₙS—, —CO— or —CONH—;
R(13) is a single bond, phenyl, thienyl, pyridyl, thiazolyl, thiadiazolyl, imidazolyl or pyrazolyl;
R(14) is a single bond or SO₂—;
R(15) is (C₂-C₁₀)-alkenyl- or (C₂-C₁₀)-alkynyl;
W and W(1)
independently of one another are hydrogen, (C₁-C₄)-alkyl;
or
W and W(1)
cyclically connected to one another are a (C₃-C₈)-hydrocarbon ring;
q and q′
are zero to 9;
Am is —NR(10)R(11);
R(10) is hydrogen, (C₁-C₄)-alkyl or benzyl,
R(11) is (C₁-C₄)-alkyl, phenyl or benzyl,
or
R(10) and R(11)
together are a (C₃-C₁₀)-alkylene group,
which is unsubstituted or substituted by —COOH, (C₁-C₅)-alkoxycarbonyl, (C₂-C₄)-hydroxyl-alkylene or benzyl;
or
Am is pyrrolyl, pyridyl, pyrazolyl, morpholinyl, dihydropyridyl, tetrahydropyridyl, quinuclidinyl, imidazolyl, 3-azabicyclo[3.2.1 ]octyl,
which is unsubstituted or substituted by (C₁-C₄)-alkyl,
or
Am is azabicyclo[3.2.2]nonyl;
or
Am is a piperazine group of the formula 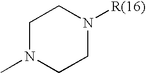
R(16) is hydrogen, (C₁-C₄)alkyl, (C₃-C₆)-cycloalkyl, phenyl, tolyl, methoxyphenyl, halophenyl, diphenylmethylene, benzyl or pyridyl;
or
Am is an azido group —(O)ₜ—(CH₂)q-C[W][W(1)]-(CH₂)q′-N₃;
t is zero or 1;
where W and W(1) have the previously indicated meaning; and the optical enantiomers and the pharmacologically tolerable salts.

3. The use as claimed in claim 1, which comprises using:
r) benzo-fused heterocycles, having a 5-membered ring, of the formula I 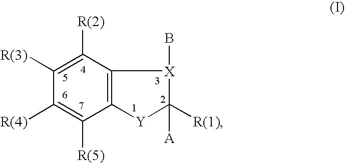
in which:
x is N or CR(6);
Y is oxygen, S or NR(7);
A, B together are a bond
or
A, B are both hydrogen, if X is CR(6) and Y is simultaneously NR(7);
one of the substituents R(1) to R(6) is a -CO—N═C(NH₂)₂ group;
the other substituents R(1) to R(6) in each case
are hydrogen, F, Cl, Br, I or (C₁-C₆)-alkyl;
up to two of the other substituents R(1) to R(6)
are CN, NO₂, N₃, (C₁-C₄)-alkyloxy or CF₃;
up to one of the other substituents
is R(8)-CₙH₂n-Z-;
n is zero to 10;
where the alkylene chain —CₙH₂ₙ— is straight-chain or branched and where a carbon atom can be replaced by an oxygen or sulfur atom or by a nitrogen atom;
R(8) is hydrogen, (C₂-C₆)-alkenyl or (C₃-C₁₀)-cycloalkyl,
which is unsubstituted or substituted by 1 to 4 methyl groups or an OH group, or can contain an ethylene group —CH═CH—, and where a methylene group can be replaced by an oxygen or sulfur atom or by a nitrogen atom;
or
R(8) is phenyl,
which is unsubstituted or substituted by 1 to 3 substituents selected from the group consisting of F, Cl, Br, I, CF₃, CH₃—S(O)ₛ— or R(9)-W_{y}—;
s is zero, 1 or 2;
R(9) is H, methyl, ethyl,
W is oxygen or NR(10);
R(10) is H or methyl;
y is zero or 1;
or
R(8) is CₘF₂ₘ₊₁;
m is 1to 3;
or
R(8) is 1- or 2-naphthyl, pyridyl, quinolyl or isoquinolyl;
Z is —CO—, —CH₂— or —[CR(11)(OH)]_{q}—;
q is 1, 2 or 3;
R(11) is H or methyl;
or
Z is oxygen or —NR(12)-;
R(12) is H or methyl;
or
Z is —S(O)ₛ—;
s is zero, 1 or 2;
or
Z is —SO₂—NR(13)-;
R(13) is H or (C₁-C₄)alkyl;
R(7) is hydrogen, (C₁-C₁₀)-alkyl, (C₂-C₁₀)-alkenyl or R(8)-CₙH₂ₙ—;
and their pharmaceutically tolerable salts;
v) acylguanidines of the formula I 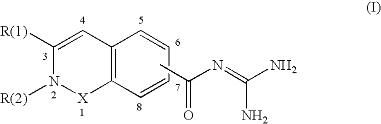
in which;
X is carbonyl, sulfonyl,
R(1) is H, (C₁-C₈)-alkyl,
unsubstituted or substituted by hydroxyl,
(C₃-C₈)-cycloalkyl, phenyl,
which is unsubstituted or substituted by 1-3 substituents from the group consisting of F, Cl, CF₃, CH₃, methoxy, hydroxyl, amino, methylamino or dimethylamino,
R(2) is H, (C₁-C₄)-alkyl,
and their pharmaceutically tolerable salts;
w) phenyl-substituted alkylcarboxylic acid guanidides, carrying perfluoroalkyl groups, of the formula I 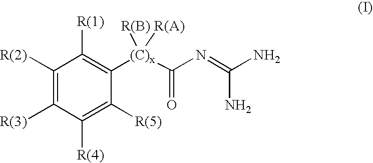
in which:
R(A) is hydrogen, F, Cl, Br, I, CN, OR(6), (C₁-C₈)-alkyl, (C₃-C₈)-cycloalkyl, Oᵣ(CH₂)ₐC_{b}F_{2b+1} or NR(7)R(8);
r is zero or 1;
a is zero, 1, 2, 3 or 4;
b is 1, 2, 3, 4, 5, 6, 7 or 8;
R(6) is hydrogen, (C₁-C₈)-alkyl, (C₃-C₈)-alkenyl, (C₃-C₈)-cycloalkyl, phenyl or benzyl,
where the aromatics are not substituted or are substituted by 1-3 substituents from the group consisting of F, Cl, CF₃, methyl, methoxy and NR(9)R(10);
R(9) and R(10)
are H, (C₁-C₄)-alkyl or (C₁-C₄)-perfuoroalkyl;
R(7) and R(8)
independently of one another are defined as R(6);
R(B) independently is defined as R(A);
x is 1, 2 or 3;
R(1) is hydrogen, (C₁-C₈)-alkyl, (C₃-C₈)-cycloalkyl, —Oₜ(CH₂)_{d}CₑF₂ₑ₊₁, F, Cl, Br, I or CN;
t is zero or 1;
d is zero, 1, 2, 3 or 4;
e is 1, 2, 3, 4, 5, 6, 7 or 8;
R(2), R(3), R(4) and R(5)
independently of one another are defined as R(1);
but with the condition
that at least one of the substituents R(1), R(2), R(3), R(4), R(5), R(A) and R(B) is an —Oₜ(CH₂)_{d}CₑF₂ₑ₊₁ or an Oᵣ(CH₂)ₐC_{b}F_{2b+1}-group,
and their pharmaceutically tolerable salts;
y) bicyclic heteroaroylguanidines of the formula I 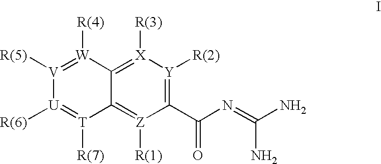
in which:
T, U, V, W, X, Y and Z
independently of one another are nitrogen or carbon;
but with the restriction
that X and Z are not simultaneously nitrogen,
and that T, U, V, W, X, Y and Z carry no substituents when they are nitrogen,
and that no more than four of them are simultaneously nitrogen,
R(1) and R(2)
independently of one another are hydrogen, F, Cl, Br, I, (C₁-C₃)-alkyl, (C₁-C₃)-perfluoroalkyl, OR(8), NR(8)R(9) or C(═O)N═C(NH₂)₂;
R(8) and R(9)
independently of one another are hydrogen or (C₁-C₃)-alkyl,
or
R(8) and R(9)
together are 4 or 5 methylene groups, of which one CH₂ group can be replaced by oxygen, S, NH, N—CH₃ or N-benzyl;
R(3), R(4), R(5), R(6) and R(7)
independently of one another are hydrogen, F, Cl, Br, I, —C≡N, Xₖ—(CH₂)ₚ—(C_{q}F_{2q+1}), R(10a)-SO_{bm}, R(10b)R(10c)N—CO, R(11)-CO— or R(12)R(13)N—SO₂—,
where the perfluoroalkyl group is straight-chain or branched;
X is oxygen, S or NR(14);
R(14) is H or (C₁-C₃)-alkyl;
bm is zero, 1 or 2;
p is zero, 1 or 2;
k is zero or 1;
q is 1, 2, 3, 4, 5 or 6;
R(10a), R(10b), R(11) and R(12)
independently of one another are (C₁-C₈)-alkyl, (C₃-C₆)-alkenyl, —CₙH₂ₙ—R(15) or (C₁-C₈)-perfluoroalkyl;
n is zero, 1, 2, 3 or 4;
R(15) is (C₃-C₇)-cycloalkyl or phenyl, which is not substituted or is substituted by 1-3 substituents selected from the group consisting of F, Cl, CF₃, methyl, methoxy and NR(16)R(17);
R(16) and R(17)
are H or C₁-C₄-alkyl;
or
R(10b), R(11) and R(12)
are hydrogen;
R(10c) and R(13)
independently are hydrogen or (C₁-C₄)-alkyl;
or
R(10b) and R(10c) and also R(12) and R(13)
together are 4 or 5 methylene groups, of which one CH₂ group can be replaced by oxygen, sulfur, NH, N—CH₃ or N-benzyl;
or
R(3), R(4), R(5), R(6) and R(7)
independently of one another are (C₁-C₈)-alkyl, —CₐₗH₂ₐₗR(18) or (C₃-C₈)-alkenyl;
al is zero, 1 or 2;
R(18) is (C₃-C₈)-cycloalkyl, phenyl, biphenylyl or naphthyl, where the aromatics are not substituted or are substituted by 1-3 substituents selected from the group consisting of F, Cl, CF₃, methyl, methoxy and NR(19a)R(19b);
R(19a) and R(19b)
are hydrogen, (C₁-C₄)-alkyl or (C₁-C₄)-perfluoroalkyl;
or
R(3), R(4), R(5), R(6) and R(7)
independently of one another are (C₁-C₉)-heteroaryl which is linked via C or N and which is unsubstituted or substituted by 1-3 substituents from the group consisting of F, Cl, CF₃, CH₃, methoxy, hydroxyl, amino, methylamino or dimethylamino;
or
R(3), R(4), R(5), R(6) and R(7)
independently of one another are 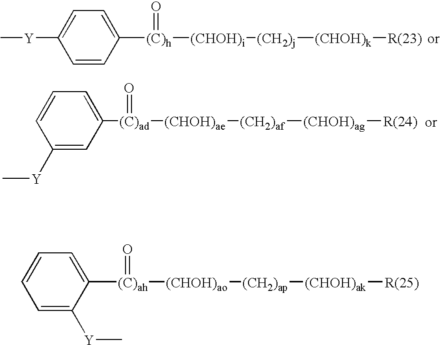
Y is oxygen, —S— or —NR(22)-;
h, ad, ah
independently of one another are zero or 1;
i, j, k, ae, af, ag, ao, ap and ak
independently of one another are zero, 1, 2, 3 or 4;
but where in each case
h, i and k are not simultaneously zero,
ad, ae and ag are not simultaneously zero, and
ah, ao and ak are not simultaneously zero,
R(23), R(24) R(25) and R(22)
independently of one another are hydrogen or (C₁-C₃)-alkyl;
or
R(3), R(4), R(5), R(6) and R(7)
independently of one another are SR(29), —OR(30), —NR(31)R(32) or —CR(33)R(34)R(35);
R(29), R(30), R(31) and R(33)
independently of one another are —CₐH₂ₐ—(C₁-C₉)-heteroaryl, which is unsubstituted or substituted by 1-3 substituents selected from the group consisting of F, Cl, CF₃, CH₃, methoxy, hydroxyl, amino, methylamino and dimethylamino;
a is zero, 1 or 2;
R(32), R(34) and R(35)
independently of one another are defined as R(29) or hydrogen, (C₁-C₄)-alkyl or (C₁-C₄)-perfluoroalkyl;
or
R(3), R(4), R(5), R(6) and R(7)
independently of one another are 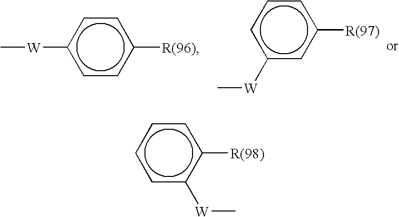
R(96), R(97) and R(98)
independently of one another are (C₁-C₉)-heteroaryl, which is linked via C or N and which is unsubstituted or substituted by 1 to 3 substituents from the group consisting of F, Cl, CF₃, CH₃, methoxy, hydroxyl, amino, methylamino, dimethylamino or benzyl;
W is oxygen, S or NR(36)-;
R(36) is H or (C₁-C₄)-alkyl;
or
R(3), R(4), R(5), R(6) and R(7)
independently of one another are R(46)X(1)-;
X(1) is oxygen, S, NR(47), (D=O)A- or NR(48)C=MN^{(*)}R(49)-;
M is oxygen or sulfur;
A is oxygen or NR(50);
D is C or SO;
R(46) is (C₁-C₈)-alkyl, (C₃-C₈)-alkenyl, (CH₂)_{b}C_{d}F_{2d+1} or —CₓH₂ₓ—R(51);
b is zero or 1;
d is1, 2, 3, 4, 5, 6 or 7;
x is zero, 1, 2, 3 or 4;
R(51) is (C₃-C₈)-cycloalkyl, phenyl, biphenylyl or naphthyl,
where the aromatics are not substituted or are substituted by 1-3 substituents selected from the group consisting of F, Cl, CF₃, methyl, methoxy and NR(52)R(53);
R(52) and R(53)
are hydrogen, (C₁-C₄)-alkyl or (C₁-C₄)-perfluoroalkyl;
R(47), R(48) and R(50) independently
are hydrogen, (C₁-C₄)-alkyl or (C₁-C₄)-perfluoroalkyl;
R(49) is defined as R(46);
or
R(46) and R(47), or R(46) and R(48)
together are 4 or 5 methylene groups, of which one CH₂ group can be replaced by oxygen, sulfur, NH, N—CH₃ or N-benzyl;
where A and N^{(*)} are bonded to the phenyl nucleus of the heteroaroylguanidine parent structure;
or
R(3), R(4), R(5), R(6) and R(7)
independently of one another are —SR(64), —OR(65), —NHR(66), —NR(67)R(68), —CHR(69)R(70) or —CR(54)R(55)OH, —C≡CR(56), —CR(58)=CR(57) or —[CR(59)R(60)]ᵤ-CO—[CR(61)R(62)]ᵥ-R(63);
R(64), R(65), R(66), R(67) and R(69)
identically or differently
are —(CH₂)_{y}—(CHOH)_{z}—(CH₂)ₐₐ—(CHOH)ₜ—R(71) or —(CH₂)_{ab}—O—(CH₂—CH₂O)_{ac}—R(72);
R(71) and R(72)
independently of one another are hydrogen or methyl;
u is 1, 2, 3 or 4;
v is zero, 1, 2, 3 or 4;
y, z, aa identically or differently
are zero, 1, 2, 3 or 4;
t is 1, 2, 3 or 4;
R(68), R(70), R(54) and R(55)
identically or differently are hydrogen or (C₁-C₆)-alkyl;
or
R(69) and R(70), or R(54) and R(55),
together with the carbon atom carrying them, are a (C₃-C₈)-cycloalkyl;
R(63)
is hydrogen, (C₁-C₆)-alkyl, (C₃-C₈)-cycloalkyl or —CₑH₂ₑ—R(73);
e is zero, 1, 2, 3 or 4;
R(56), R(57) and R(73) independently
are phenyl, which is unsubstituted or substituted by 1-3 substituents selected from the group consisting of F, Cl, CF₃, methyl, methoxy and NR(74)R(75);
R(74) and R(75)
are hydrogen or (C₁-C₄)-alkyl;
or
R(56), R(57) and R(73) independently
are (C₁-C₉)-heteroaryl, which is unsubstituted or substituted as phenyl;
R(58), R(59), R(60), R(61) and R(62)
are hydrogen or methyl;
or
R(3), R(4), R(5), R(6) and R(7)
independently of one another are R(76)-NH—SO₂—;
R(76) is R(77)R(78)N—(C═Y′)—;
Y′ is oxygen, S or N—R(79);
R(77) and R(78)
identically or differently are hydrogen, (C₁-C₈)-alkyl, (C₃-C₆)-alkenyl or —C_{f}H_{2f}—R(80);
f is zero, 1, 2, 3 or 4;
R(80) is (C₅-C₇)-cycloalkyl or phenyl, which is unsubstituted or substituted by 1-3 substituents selected from the group consisting of F, Cl, CF₃, methoxy and (C₁-C₄)-alkyl;
or
R(77) and R(78)
together are 4 or 5 methylene groups, of which one CH₂ group can be replaced by oxygen, sulfur, NH, N—CH₃ or N-benzyl;
R(79) is defined as R(77) or is amidine;
or
R(3), R(4), R(5), R(6) and R(7)
independently of one another are NR(84a)R(85), OR(84b), SR(84c) or —CₙH₂ₙ—R(84d);
n is zero, 1, 2, 3 or 4;
R(84d) is (C₃-C₇)-cycloalkyl or phenyl, which is not substituted or is substituted by 1-3 substituents from the group consisting of F, Cl, CF₃, methyl, methoxy and NR(16)R(17);
R(16) and R(17)
are hydrogen or C₁-C₄-alkyl;
R(84a), R(84b), R(84c) and R(85)
independently of one another are hydrogen, (C₁-C₈)-alkyl, (C₁-C₈)-perfluoroalkyl or (CH₂)ₐₓ—R(84g);
ax is zero, 1, 2, 3 or 4;
R(84g) is (C₃-C₇)-cycloalkyl or phenyl, which is not substituted or is substituted by 1-3 substituents selected from the group consisting of F, Cl, CF₃, methyl, methoxy and NR(84u)R(84v);
R(84u) and R(84v)
are hydrogen or C₁-C₄-alkyl;
or
R(84a) and R(85)
together are 4 or 5 methylene groups, of which one CH₂ group can be replaced by oxygen, sulfur, NH, N—CH₃ or N-benzyl,
and their pharmaceutically tolerable salts;
ab) phenyl-substituted alkenylcarboxylic acid guanidides, carrying perfluoroalkyl groups, of the formula I 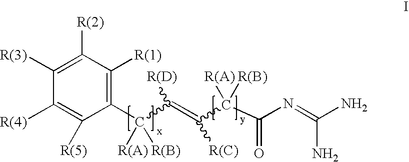
in which:
R(A) is hydrogen, F, Cl, Br, I, CN, OH, OR(6), (C₁-C₈)-alkyl,
Oᵣ(CH₂)ₐC_{b}F_{2b+1}, (C₃-C₈)-cycloalkyl or NR(7)R(8);
r is zero or 1;
a is zero, 1, 2, 3 or 4;
b is 1, 2, 3, 4, 5, 6, 7 or 8;
R(6) is (C₁-C₈)-alkyl, (C₁-C₄)-perfluoroalkyl, (C₃-C₈)-alkenyl, (C₃-C₈)-cycloalkyl, phenyl or benzyl;
where the aromatics are not substituted or are substituted by 1-3 substituents from the group consisting of F, Cl, CF₃, methyl, methoxy and NR(9)R(10);
R(9) and R(10)
are H, (C₁-C₄)-alkyl or (C₁-C₄)-perfluoroalkyl;
R(7) and R(8)
independently of one another are defined as R(6);
or
R(7) and R(8)
together are 4 or 5 methylene groups, of which one CH₂ group can be replaced by oxygen, sulfur, NH, N—CH₃ or N-benzyl;
R(B) independently is defined as R(A);
X is zero, 1 or 2;
Y is zero,1or 2;
R(C) is hydrogen, F, Cl, Br, I, CN, OR(12), (C₁-C₈)-alkyl, Oₚ(CH₂)_{f}C_{g}F_{2g+1} or (C₃-C₈)-cycloalkyl;
p is zero or 1;
f is zero, 1, 2, 3 or 4;
g is 1, 2, 3, 4, 5, 6, 7 or 8;
R(12)
is (C₁-C₈)-alkyl, (C₁-C₄)-perfluoroalkyl, (C₃-C₈)-alkenyl, (C₃-C₈)-cycloalkyl, phenyl or benzyl;
where the aromatics phenyl or benzyl are not substituted or are substituted by 1-3 substituents from the group consisting of F, Cl, CF₃, methyl, methoxy and NR(13)R(14);
R(13) and R(14)
independently of one another are H, (C₁-C₄)-alkyl or (C₁-C₄)-perfluoroalkyl;
R(D) independently is defined as R(C),
R(1) is hydrogen, (C₁-C₈)-alkyl, —Oₜ(CH₂)_{d}CₑF₂ₑ₊₁, (C₃-C₈)-cycloalkyl, F, Cl, Br, I or CN;
t is zero or 1;
d is zero, 1, 2, 3 or 4;
e is 1, 2, 3, 4, 5, 6, 7 or 8;
R(2), R(3), R(4) and R(5)
independently of one another are defined as R(1);
but with the condition
that at least one of the substituents R(A), R(B), R(C), R(D), R(1), R(2), R(4) or R(5) is a Or(CH₂)ₐC_{b}F_{2b+1}, Oₚ(CH₂)_{f}C_{g}F_{2g+1} or Oₜ(CH₂)_{d}CₑF₂ₑ₊₁ group and R(3) is not a Oₜ(CH₂)_{d}CₑF₂ₑ₊₁ group;
and their pharmaceutically tolerable salts;
ah) benzoylguanidines of the formula I 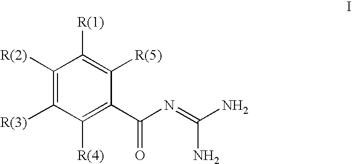
in which:
one of the three substituents R(1), R(2) and R(3)
is R(6)-A-B-D-;
R(6) is a basic protonatable radical, i.e. an amino group —NR(7)R(8), an amidino group R(7)R(8)N—C[═N—R(9)]- or a guanidino group 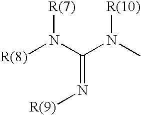
R(7), R(8), R(9) and R(10)
independently of one another are hydrogen or alkyl having 1, 2, 3 or 4 carbon atoms;
or
R(7) and R(8)
together are CₐH₂ₐ;
a is 4, 5, 6 or 7;
where if a=5, 6 or 7 a methylene group of the group CₐH₂ₐ can be replaced by a heteroatom group O, SOₘ or NR(11),
or
R(8) and R(9) or R(9) and R(10) or R(7) and R(10)
are a group CₐH₂ₐ;
a is 2, 3, 4 or 5;
where if a=3, 4 or 5 a methylene group of the group CₐH₂ₐ can be replaced by a heteroatom group O, SOₘ or NR(11);
m is zero, 1 or 2;
R(11) is hydrogen or methyl;
or
R(6) is a basic heteroaromatic ring system having 1-9 carbon atoms;
A is C_{b}H2b;
b is 1, 2, 3, 4, 5, 6,7, 8, 9 or 10;
where in the group C_{b}H_{2b} one or two methylene groups can be replaced by one of the groupings selected from the group consisting of —O—, —CO—, —CH[OR(20)]-, —SOₘ—, —NR(20)-, —NR(20)-CO—, —NR(20)-CO—NH—, —NR(20)-CO—NH—SO₂— 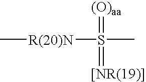
and —SOₐₐ[NR(1 )]_{bb}-;
and where in the group C_{b}H_{2b} a methylene group can be replaced by —CH—R(99), where R(99) together with R(7) forms a pyrrolidine or piperidine ring;
aa is 1 or 2;
bb is 0 or 1;
aa+bb=2;
R(19) is hydrogen or alkyl having 1, 2, 3 or 4 carbon atoms;
R(20) is hydrogen or methyl;
B is a phenylene or naphthylene radical, 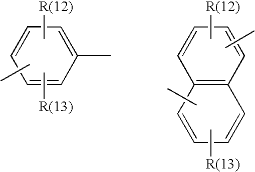
R(12) and R(13)
independently of one another are hydrogen, methyl, F, Cl, Br, I, CF₃ or —SO_{w}—R(14);
R(14) is methyl or NR(15)R(16);
R(15) and R(16)
independently of one another are hydrogen or alkyl having 1, 2, 3 or 4 carbon atoms;
w is zero, 1 or 2;
D is —C_{d}H_{2d}—X_{f}—;
d is zero, 1, 2, 3 or 4;
X is —O—, —CO—, —CH[OR(21)]-, —SOₘ— or —NR(21)-;
f is zero or 1;
R(21) is hydrogen or methyl;
m is zero, 1 or 2;
and the other substituents R(1) and R(2) and R(3) in each case
independently of one another are hydrogen, F, Cl, Br, I, —CN, —(C₁-C₈)-alkyl, —(C₂-C₈)-alkenyl, —NR(35)R(36) or R(17)-C_{g}H_{2g}-Zₕ-;
g is zero, 1, 2, 3 or 4;
h is zero or 1;
R(35) and R(36)
independently of one another are hydrogen or alkyl having 1, 2, 3, 4, 5 or 6 carbon atoms;
or
R(35) and R(36)
together are 4-7 methylene groups, of which one CH₂ group can be replaced by oxygen,—S—, —NH—, —NCH₃ or —N-benzyl;
Z is —O—, —CO—, —SOᵥ—, —NR(18)-, —NR(18)-CO—, —NR(18)-CO—NH— or —NR(18)-SO₂—;
R(18) is hydrogen or methyl;
v is zero, 1 or 2;
R(17) is hydrogen, cycloalkyl having 3, 5 or 6 carbon atoms or CₖF₂ₖ₊₁—;
k is 1, 2 or 3,
or
R(17) is pyrrol-1-yl, pyrrol-2-yl or pyrrol-3-yl,
which is not substituted or is substituted by 1-4 substituents selected from the group consisting of F, Cl, Br, I, —CN, (C₂-C₈)-alkanoyl, (C₂-C₈)-alkoxycarbonyl, formyl, carboxyl, —CF₃, methyl and methoxy;
or
R(17) is —(C₃-C₈)-cycloalkyl or phenyl,
which is not substituted or is substituted by 1-3 substituents selected from the group consisting of F and Cl, —CF₃, methyl, hydroxyl, methoxy, —NR(37)R(38), CH₃SO₂— and H₂NO₂S—;
R(37) and R(38)
are hydrogen or —CH₃;
R(4) and R(5)
independently of one another are hydrogen, alkyl having 1, 2, 3 or 4 carbon atoms, F, Cl, —OR(32), —NR(33)R(34) or —CᵣF₂ᵣ₊₁;
R(32), R(33) and R(34)
independently of one another are hydrogen or alkyl having 1, 2 or 3 carbon atoms;
r is 1, 2, 3 or 4;
and their pharmacologically tolerable salts;
ai) indenoylguanidines of the formula I 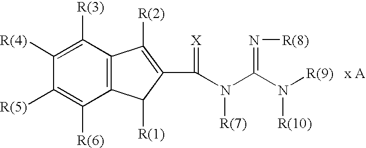
in which:
R(1) and R(2)
independently of one another are hydrogen, alkyl having 1, 2, 3, 4, 5, 6, 7, 8, 9 or 10 carbon atoms, cycloalkyl having 3, 4, 5 or 6 carbon atoms, 0-alkyl having 1, 2, 3 or 4 carbon atoms, O—C(═O)-alkyl having 1, 2, 3 or 4 carbon atoms or CₘH₂ₘ—NR(12)R(13);
R(12) and R(13)
independently of one another are hydrogen or alkyl having 1, 2, 3 or 4 carbon atoms;
m is zero, 2, 3 or 4;
NH—C(═O)—NH₂, C(═O)—O-alkyl having 1, 2, 3 or 4 carbon atoms, C(═O)—NH₂, C(═O)—NH-alkyl having 1, 2, 3 or 4 carbon atoms, C(═O)—N(alkyl)₂ having 1, 2, 3 or 4 carbon atoms in each alkyl group, alkenyl having 2, 3, 4, 5, 6, 7, 8, 9 or 10 carbon atoms, alkynyl having 2, 3, 4, 5, 6, 7, 8, 9 or 10 carbon atoms, alkylaryl having 1, 2, 3 or 4 carbon atoms in the alkyl group, alkenylaryl having 2, 3, 4, 5, 6, 7, 8, 9 or 10 carbon atoms in the alkenyl group, alkynylaryl having 2, 3, 4, 5, 6, 7, 8, 9 or 10 carbon atoms in the alkynyl group, C₁-C₄-alkyl-substituted aryl, C₁-C₄-alkylheteroaryl, C₁-C₄-alkenylheteroaryl, aminoalkylaryl having 1, 2, 3 or 4 carbon atoms in the alkyl group, substituted aryl, heteroaryl and substituted heteroaryl;
R(3), R(4), R(5) and R(6)
independently of one another are hydrogen, alkyl having 1, 2, 3, 4, 5, 6, 7, 8, 9 or 10 carbon atoms, O-alkyl having 1, 2, 3, 4, 5, 6, 7, 8, 9 or 10 carbon atoms, halogen, (such as F, Cl, Br, I), OH, aryl, substituted aryl, heteroaryl, substituted heteroaryl, O-lower-alkyl, O-aryl, O-lower-alkylaryl, O-substituted aryl, O-lower-alkyl-substituted aryl, O—C(═O)—C₁-C₄-alkylaryl, O—C(═O)—NH—C₁-C₄-alkyl, O—C(═O)—N(C₁-C₄-alkyl)₂, NO₂, CN, CF₃, NH₂, NH—C(═O)—C₁-C₄-alkyl, NH—C(═O)—NH₂, COOH, C(═O)—O—C₁-C₄-alkyl, C(═O)—NH₂, C(═O)—NH—C₁-C₄-alkyl, C(═O)—N(C₁-C₄-alkyl)₂, C₁-C₄-COOH, C₁-C₄-alkyl-C(═O)—O—C₁-C₄-alkyl, SO₃H, SO₂-alkyl, SO₂-alkylaryl, SO₂—N-(alkyl)₂, SO₂—N(alkyl)(alkylaryl), C(═O)—R(11), C₁-C₁₀-alkyl-C(═O)—R(11), C₂-C₁₀-alkenyl-C(═O)—R(11), C₂-C₁₀-alkynyl-C(═O)—R(11), NH—C(═O)—C₁-C₁₀-alkyl-C(═O)—R(11), O—C₁-C₁₁-alkyl-C(═O)—R(11);
R(11) is C₁-C₄-alkyl, C₁-C₄-alkynyl, aryl, substituted aryl, NH₂, NH—C₁-C₄-alkyl, N—(C₁-C₄-alkyl)₂, SO₃H, SO₂-alkyl, SO₂-alkylaryl, SO₂—N-(alkyl)₂, SO₂—N(alkyl)(alkylaryl);
X is O, S or NH;
R(7), R(8), R(9) and R(10)
independently of one another are hydrogen, alkyl, cycloalkyl, aryl, alkylaryl;
or
R(8) and R(9)
together are part of a 5, 6 or 7-membered heterocyclic ring;
A is absent or is a nontoxic organic or inorganic acid.
ak) benzyloxycarbonylguanidines of the formula I 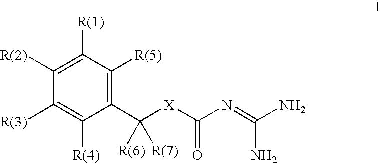
in which:
R(1), R(2) and R(3)
independently of one another are —Y-[4-R(8)-phenyl], —Y-[3-R(8)-phenyl] or —Y-[2-R(8)-phenyl],
where the phenyl in each case is unsubstituted or substituted by 1-2 substituents from the group consisting of F, Cl, —CF₃, methyl, hydroxyl, methoxy and —NR(96)R(97);
R(96) and R(97)
independently of one another are hydrogen or —CH₃;
Y is a bond, CH₂, oxygen, —S— or —NR(9);
R(9) is hydrogen or alkyl having 1, 2, 3 or 4 carbon atoms;
R(8) is SOₐ[NR(98)]_{b}NR(99)R(10);
a is 1 or 2;
b is 0 or 1;
a+b=2;
R(98), R(99) and R(10)
independently of one another are hydrogen, —(C₁-C₈)-alkyl, benzyl, —(C₂-C₈)-alkylene-NR(1)R(12), (C₂-C₈)-alkylene-NR(13)-(C₂-C₈)-alkylene-NR(37)R(38) or (C₀-C₈)-alkylene-CR(39)R(40)CR(41)R(42)(C₀-C₈)-alkylene-NR(43)R(44);
R(11), R(12), R(13), R(37), R(38), R(43) and R(44)
independently of one another are hydrogen, —(C₁-C₈)-alkyl or benzyl:
R(39), R(40), R(41) and R(42)
independently of one another are hydrogen, —(C₁-C₈)-alkyl or —(C-C₃)-alkylenephenyl,
where the phenyl is not substituted or is substituted by 1-3 substituents selected from the group consisting of F, Cl, —CF₃, methyl and methoxy;
or
R(99) and R(10)
together are 4-6 methylene groups, of which one CH₂ group can be replaced by oxygen, —S—, —NH—, —N—CH₃ or —N-benzyl;
or
R(8) is SOₐ[NR(98)]_{b}NR(95)-C[═N—R(94)]-NR(93)R(92);
R(92), R(93), R(94) and R(95)
independently of one another are hydrogen or alkyl having 1, 2, 3 or 4 carbon atoms;
or
R(1), R(2) and R(3)
independently of one another are pyrrol-1-yl, pyrrol-2-yl or pyrrol-3-yl,
which is not substituted or is substituted by 1-4 substituents selected from the group consisting of F, Cl, Br, I, —CN, (C₂-C₈)-alkanoyl, (C₂-C₈)-alkoxycarbonyl, formyl, carboxyl, —CF₃, methyl, methoxy;
or
R(1), R(2) and R(3)
independently of one another are hydrogen, —(C₁-C₈)-alkyl, —(C₂-C₈)-alkenyl or —(CH₂)ₘR(14);
m is zero, 1 or 2;
R(14) is —(C₃-C₈)-cycloalkyl or phenyl,
which is not substituted or is substituted by 1-3 substituents selected from the group consisting of F and Cl, —CF₃, methyl, methoxy and —NR(15)R(16);
R(15) and R(16)
are hydrogen or —CH₃;
or
R(1), R(2) and R(3)
independently of one another are -Q-4-[(CH₂)ₖ—CHR(17)-(C═O)R(20)]-phenyl, -Q-3-(CH₂)ₖ—CHR(17)-(C═O)R(20)]-phenyl or -Q-2-[(CH₂)ₖ—CHR(17)-(C═O)R(20)]-phenyl,
where the phenyl in each case is unsubstituted or substituted by 1-2 substituents from the group consisting of F, Cl, —CF₃, methyl, hydroxyl, methoxy and —NR(35)R(36);
R(35) and R(36)
independently of one another are hydrogen or —CH₃;
Q is a bond, oxygen, —S or —NR(18);
R(18) is hydrogen or —(C₁-C₄)-alkyl;
R(17) is —OR(21) or —NR(21)R(22);
R(21) and R(22)
independently of one another are hydrogen, —(C₁-C₈)-alkyl, —(C₁-C₈)-alkanoyl, —(C₁-C₈)-alkoxycarbonyl, benzyl, benzyloxycarbonyl;
or
R(21) is trityl;
R(20) is —OR(23) or —NR(23)R(24);
R(23), R(24) independently of one another are hydrogen, —(C₁-C₈)-alkyl or benzyl;
k is zero, 1, 2, 3 or 4;
or
R(1), R(2) and R(3)
independently of one another are (C₁-C₉)-heteroaryl,
which is linked via C or N and which is unsubstituted or substituted by 1-3 substituents from the group consisting of F, Cl, CF₃, CH₃, methoxy, hydroxyl, amino, methylamino and dimethylamino;
or
R(1), R(2) and R(3)
are —SR(25), —OR(25), —NR(25)R(26), —CR(25)R(26)R(27);
R(25) is —C_{f}H_{2f}—(C₁-C₉)-heteroaryl,
which is unsubstituted or substituted by 1-3 substituents from the group consisting of F, Cl, CF₃, CH₃, methoxy, hydroxyl, amino, methylamino and dimethylamino;
f is zero, 1or 2;
R(26) and R(27)
independently of one another are defined as R(25) or are hydrogen or (C₁-C₄)-alkyl,
or
R(1), R(2) and R(3)
independently of one another are (C₁-C₉)-heteroaryl-N-oxide,
which is linked via C or N and which is unsubstituted or substituted by 1-3 substituents selected from the group consisting of F, Cl, CF₃, CH₃, methoxy, hydroxyl, amino, methylamino and dimethylamino;
or
R(1), R(2) and R(3)
independently of one another are —SR(28), —OR(28), —NR(28)R(29) or —CR(28)R(29)R(30);
R(28) is —C_{g}H_{2g}—(C₁-C₉)-heteroaryl-N-oxide,
which is unsubstituted or substituted by 1-3 substituents selected from the group consisting of F, Cl, CF₃, CH₃, methoxy, hydroxyl, amino, methylamino and dimethylamino;
g is zero, 1 or 2;
R(29), R(30)
independently of one another are defined as R(28), or [symbol]
group can be replaced by oxygen,—S—, —NH—, —NCH₃ or —N-benzyl;
or
R(1), R(2) and R(3)
independently of one another are R(51)-A-G-D-;
R(51) is a basic protonatable radical, i.e. an amino group —NR(52)R(53), an amidino group R(52)R(53)N—C[═N—R(54)]- or a guanidino group R(52)R(53)N—C[═N—R(54)]-NR(55)-;
R(52), R(53), R(54) and R(55)
independently of one another are hydrogen or alkyl having 1, 2, 3 or 4 carbon atoms;
or
R(52) and R(53)
are a group C_{α}H_{2α};
α is 4, 5, 6 or 7;
where if α=5, 6 or 7 a carbon atom of the group C_{α}H2α can be replaced by a heteroatom group O, SO^{d} or NR(56),
or
R(53) and R(54) or R(54) and R(55) or R(52) and R(55)
are a group C_{y}H_{2y};
y is 2, 3, 4 or 5;
where if y=3, 4 or 5 a carbon atom of the group C_{y}H_{2y} can be replaced by a heteroatom group O, SO^{d} or NR(56);
d is zero, 1 or 2;
R(56) is hydrogen or methyl;
or
R(51) is a basic heteroaromatic ring system having 1-9 carbon atoms;
A is a group CₑH₂ₑ;
e is zero, 1, 2, 3, 4, 5, 6, 7, 8, 9 or 10;
where in the group CₑH₂ₑ a carbon atom can be replaced by one of the groupings —O—, —CO—, —CH[OR(57)]-, —SOᵣ—, —NR(57)-, —NR(57)-CO—, —NR(57)-CO—NH—, —NR(57)-CO—NH—SO₂— or —NR(57)-SO₂—;
r is zero, 1 or 2;
G is a phenylene radical, 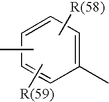
R(58) and R(59)
independently of one another are hydrogen, methyl, methoxy, F, Cl, Br, I, CF₃ or —SOₛ—R(60);
R(60) is methyl or NR(61)R(62);
R(61) and R(62)
independently of one another are hydrogen or alkyl having 1, 2, 3 or 4 carbon atoms;
D is —CᵥH₂ᵥ-E_{w}-;
v is zero, 1, 2, 3 or 4;
E is —O—, —CO—, —CH[OR(63)]-, —SOₐₐ— or —NR(63)-;
w is zero or 1;
aa is zero, 1 or 2
R(63) is hydrogen or methyl,
or
R(1), R(2) and R(3)
independently of one another are —CF₂R(64), —CF[R(65)][R(66)], —CF[(CF₂)_{q}—CF₃)][R(65)], —C[(CF₂)ₚ—CF₃]═CR(65)R(66);
R(64) is alkyl having 1, 2, 3 or 4 carbon atoms or cycloalkyl having 3, 4, 5, 6 or 7 carbon atoms;
R(65) and R(66) independently of one another are hydrogen or alkyl having 1, 2, 3 or 4 carbon atoms;
q is zero, 1 or 2;
p is zero, 1 or 2;
or
R(1), R(2) and R(3)
independently of one another are —OR(67) or —NR(67)R(68);
R(67) and R(68)
independently of one another are hydrogen or alkyl having 1, 2, 3, 4, 5 or 6 carbon atoms;
or
R(67) and R(68)
together are 4, 5, 6 or 7 methylene groups, of which one CH₂ group can be replaced by oxygen, —S—, SO₂, —NH—, —NCH₃ or —N-benzyl;
R(4) and R(5)
independently of one another are hydrogen, alkyl having 1, 2, 3 or 4 carbon atoms, F, Cl, —OR(69), —NR(70)R(71) or —C_{z}F_{2z+1};
R(69), R(70) and R(71)
independently of one another are hydrogen or alkyl having 1, 2 or 3 carbon atoms;
z is 1, 2, 3 or 4;
R(6) and R(7)
independently of one another are hydrogen or alkyl having 1, 2, 3 or 4 carbon atoms;
X is oxygen or NR(72);
R(72) is hydrogen or alkyl having 1, 2, 3 or 4 carbon atoms;
and their pharmaceutically tolerable salts;
al) alkenylcarboxylic acid guanidides, carrying fluorophenyl groups, of the formula I 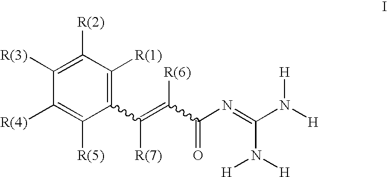
in which:
R(6) is hydrogen, (C₁-C₈)-alkyl, (C₃-C₈)-cycloalkyl or phenyl,
where the phenyl group is not substituted or is substituted by 1-3 substituents selected from the group consisting of F, Cl, CF₃, methyl, methoxy and NR(9)R(10);
R(9) and R(10)
are hydrogen, (C₁-C₄)-alkyl or (C₁-C₄)-perfluoroalkyl;
R(7) independently is defined as R(6);
R(1), R(2), R(3), R(4) and R(5)
independently of one another are hydrogen or F;
where, however, at least one of the radicals R(1), R(2), R(3), R(4) and R(5) must be fluorine;
and their pharmaceutically tolerable salts;
an) substituted cinnamic acid guanidides of the formula I 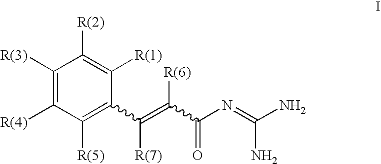
in which:
at least one of the substituents R(1), R(2), R(3), R(4) and R(5)
is —Xₐ—Y_{b}-Lₙ-U;
X is CR(16)R(17), O, S or NR(18);
R(16), R(17) and R(18)
independently of one another are H, alkyl having 1, 2, 3 or 4 carbon atoms or perfluoroalkyl having 1, 2, 3 or 4 carbon atoms;
a is zero or 1;
Y is alkylene having 1, 2, 3, 4, 5, 6, 7 or 8 carbon atoms, alkylene-T having 1, 2, 3, 4, 5, 6, 7 or 8 carbon atoms in the alkylene group, T, T-alkylene having 1, 2, 3, 4, 5, 6, 7 or 8 carbon atoms in the alkylene group;
T is NR(20), O, S or phenylene,
where the phenylene is not substituted or is substituted by 1-3 substituents selected from the group consisting of F, Cl, CF₃, methyl, methoxy and NR(21)R(22);
R(20), R(21) and R(22)
independently of one another are H, alkyl having 1, 2, 3 or 4 carbon atoms or perfluoroalkyl having 1, 2, 3 or 4 carbon atoms;
b is zero or 1;
L is O, S, NR(23) or CₖH₂ₖ;
k is 1, 2, 3, 4, 5, 6, 7, 8;
n is zero or 1;
U is NR(24)R(25) or an N-containing heterocycle having 1, 2, 3, 4, 5, 6, 7, 8 or 9 carbon atoms;
R(24) and R(25)
independently of one another are hydrogen, alkyl having 1, 2, 3, 4, 5, 6, 7 or 8 carbon atoms, perfluoroalkyl having 1, 2, 3, 4, 5, 6, 7 or 8 carbon atoms;
or
R(24) and R(25)
together are 4 or 5 methylene groups, of which one CH₂ group can be replaced by oxygen, S, NH, N—CH₃ or N-benzyl;
where the N-containing heterocycles are N- or C-bridged and are not substituted or are substituted by 1-3 substituents selected from the group consisting of F, Cl, CF₃, methyl, methoxy and NR(27)R(28);
R(23), R(27) and R(28)
independently of one another are H, alkyl having 1, 2, 3 or 4 carbon atoms or perfluoroalkyl having 1, 2, 3 or 4 carbon atoms;
and the other substituents R(1), R(2), R(3), R(4) and R(5) in each case
independently of one another are H, F, Cl, Br, I, CN, —Oₙ₋CₘH₂ₘ₊₁, —Oₚ—(CH₂)ₛ—C_{q}F_{2q+1} or —CᵣH₂ᵣR(10);
n is zero or 1;
m is zero 1, 2, 3, 4, 5, 6, 7 or 8;
p is zero or 1;
q is 1, 2, 3, 4, 5, 6, 7 or 8;
s is zero, 1, 2, 3 or 4;
r is zero, 1, 2, 3 or 4;
R(10)
is cycloalkyl having 3, 4, 5, 6, 7 or 8 carbon atoms, or phenyl,
where the phenyl is not substituted or is substituted by 1-3 substituents selected from the group consisting of F, Cl, CF₃, methyl, methoxy and NR(11)R(12);
R(11) and R(12)
independently of one another are H, alkyl having 1, 2, 3 or 4 carbon atoms or perfluoroalkyl having 1, 2, 3 or 4 carbon atoms;
R(6) and R(7)
independently of one another are hydrogen, F, Cl, Br, I, CN, alkyl having 1, 2, 3, 4, 5, 6, 7 or 8 carbon atoms, perfluoroalkyl having 1, 2, 3, 4, 5, 6, 7 or 8 carbon atoms, cycloalkyl having 3, 4, 5, 6, 7 or 8 carbon atoms, or phenyl,
which is not substituted or is substituted by 1-3 substituents selected from the group consisting of F, Cl, CF₃, methyl, methoxy and NR(14)R(15);
R(14) and R(15)
independently of one another are H, alkyl having 1, 2, 3 or 4 carbon atoms or perfluoroalkyl having 1, 2, 3 or 4 carbon atoms;
and their pharmaceutically tolerable salts;
ar) benzenedicarboxylic acid diguanidides of the formula I 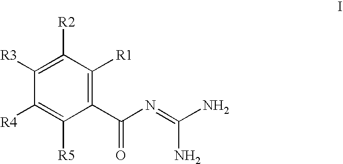
in which:
one of the radicals R(1), R(2), R(3) and R(4)
is —CO—N═C(NH₂)₂;
and the other radicals R(1), R(2), R(3) and R(4) in each case are:
R(1) is hydrogen, alkyl having 1, 2, 3 or 4 carbon atoms, F, Cl, Br, I, —OR(32), —NR(33)R(34) or CF₃;
R(32), R(33) and R(34)
independently of one another are hydrogen or alkyl having 1, 2, 3 or 4 carbon atoms;
R(2) and R(4)
independently of one another are hydrogen, F, Cl, Br, I, OH, —CN, CF₃, —CO—N═C(NH₂)₂, alkyl having 1, 2, 3, 4, 5, 6, 7 or 8 carbon atoms, alkenyl having 2, 3, 4, 5, 6, 7 or 8 carbon atoms or —(CH₂)ₘR(14);
m is zero, 1 or 2;
R(14) is —(C₃-C₈)-cycloalkyl or phenyl,
which is not substituted or is substituted by 1-3 substituents selected from the group consisting of F and Cl, —CF₃, methyl, methoxy and —NR(15)R(16);
R(15) and R(16)
are hydrogen or —CH₃;
or
R(2) and R(4)
independently of one another are pyrrol-1-yl, pyrrol-2-yl or pyrrol-3-yl,
each of which is not substituted or is substituted by 1-4 substituents selected from the group consisting of F, Cl, Br, I, —CN, (C₂-C₈)-alkanoyl, (C₂-C₈)-alkoxycarbonyl, formyl, carboxyl, —CF₃, methyl, methoxy;
or
R(2) and R(4)
independently of one another are R(22)-SO₂—, R(23)R(24)N—CO—,
R(28)-CO— or R(29)R(30)N—SO₂;
R(22) and R(28)
independently of one another are methyl or —CF₃;
R(23), R(24), R(29) and R(30)
independently of one another are hydrogen or methyl;
or
R(2) and R(4)
independently of one another are —OR(35) or —NR(35)R(36);
R(35) and R(36)
independently of one another are hydrogen or alkyl having 1, 2, 3, 4, 5 or 6 carbon atoms;
or
R(35) and R(36)
together are 4-7 methylene groups, of which one CH₂ group can be replaced by oxygen,—S—, —NH—, —NCH₃ or —N-benzyl;
R(3) is hydrogen, —SR(25), —OR(25), —NR(25)R(26), —CR(25)R(26)R(27);
R(25) is hydrogen, alkyl having 1, 2, 3, 4, 5, 6, 7 or 8 carbon atoms or phenyl,
which is unsubstituted or substituted by 1-3 substituents selected from the group consisting of F, Cl, CF₃, CH₃, methoxy, hydroxyl, amino, methylamino and dimethylamino;
or
R(25) is —(C₁-C₉)-heteroaryl,
which is unsubstituted or substituted by 1-3 substituents selected from the group consisting of F, Cl, CF₃, CH₃₁ methoxy, hydroxyl, amino, methylamino and dimethylamino;
R(26) and R(27)
independently of one another are defined as R(25) or are hydrogen or alkyl having 1, 2, 3, 4, 5, 6, 7 or 8 carbon atoms;
R(5) is alkyl having 1, 2, 3 or 4 carbon atoms, F, Cl, Br, I, X—(CH₂)_{y}—CF₃ or phenyl,
which is not substituted or is substituted by 1-3 substituents selected from the group consisting of F and Cl, —CF₃, methyl, methoxy and —NR(6)R(7);
R(6) and R(7)
independently of one another are hydrogen or —CH₃;
X is a bond or oxygen;
y is zero, 1 or 2;
and their pharmaceutically tolerable salts;
as) benzenedicarboxylic acid diguanidides of the formula I 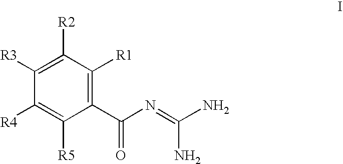
in which:
one of the radicals R(1), R(2), R(3) and R(5)
is —CO—N═C(NH₂)₂;
and the other radicals R(1), R(2), R(3) and R(5) in each case are:
R(1) and R(5)
independently of one another are hydrogen, alkyl having 1, 2, 3 or 4 carbon atoms, F, Cl, —OR(32), —NR(33)R(34) or CF₃;
R(32), R(33) and R(34)
independently of one another are hydrogen or alkyl having 1, 2, 3 or 4 carbon atoms;
R(2) is hydrogen, F, Cl, Br, I, OH, —C^{o}N, CF₃, —CO—N═C(NH₂)₂, alkyl having 1, 2, 3, 4, 5, 6, 7 or 8 carbon atoms, alkenyl having 2, 3, 4, 5, 6, 7 or 8 carbon atoms or —(CH₂)ₘR(14);
m is zero,1or 2;
R(14) is —(C₃-C₈)-cycloalkyl or phenyl,
which is not substituted or is substituted by 1-3 substituents selected from the group consisting of F and Cl, —CF₃, methyl, methoxy and —NR(15)R(16);
R(15) and R(16)
independently of one another are hydrogen or —CH₃;
or
R(2) is R(22)-SO₂—, R(23)R(24)N—CO—, R(28)-CO— or R(29)R(30)N—SO₂;
R(22) and R(28)
independently of one another are methyl or —CF₃;
R(23), R(24), R(29) and R(30)
independently of one another are hydrogen or methyl;
or
R(2) is —OR(35) or —NR(35)R(36);
R(35) and R(36)
independently of one another are hydrogen or alkyl having 1, 2, 3, 4, 5 or 6 carbon atoms;
or
R(35) and R(36)
together are 4-7 methylene groups, of which one CH₂ group can be replaced by oxygen,—S—, —NH—, —NCH₃ or —N-benzyl;
R(3) is hydrogen, —SR(25), —OR(25), —NR(25)R(26), —CR(25)R(26)R(27);
R(25) is hydrogen, alkyl having 1, 2, 3, 4, 5, 6, 7 or 8 carbon atoms or phenyl,
which is unsubstituted or substituted by 1-3 substituents selected from the group consisting of F, Cl, CF₃, CH₃, methoxy, hydroxyl, amino, methylamino and dimethylamino;
or
R(25) is —(C₁-C₉)-heteroaryl,
which is unsubstituted or substituted by 1-3 substituents selected from the group consisting of F, Cl, CF₃, CH₃, methoxy, hydroxyl, amino, methylamino and dimethylamino;
R(26) and R(27)
independently of one another are defined as R(25) or are hydrogen or alkyl having 1, 2, 3, 4, 5, 6, 7 or 8 carbon atoms;
R(4) is CF₃, alkyl having 1, 2, 3, 4, 5, 6, 7 or 8 carbon atoms, alkenyl having 2, 3, 4, 5, 6, 7 or 8 carbon atoms, —(C₃-C₈)-cycloalkyl or —(CH₂)ₘR(14);
m is 1 or 2;
R(14) is —(C₃-C₈)-cycloalkyl or phenyl,
which is not substituted or is substituted by 1-3 substituents selected from the group consisting of F and Cl, —CF₃, methyl, methoxy and —NR(15)R(16);
R(15) and R(16)
independently of one another are hydrogen or —CH₃;
or
R(4) is phenyl,
which is substituted by 2, 3, 4 or five substituents selected from the group consisting of F, Cl, CF₃, methyl, methoxy and —NR(15)R(16);
R(15) and R(16)
independently of one another are hydrogen or CH₃;
and their pharmaceutically tolerable salts;
at) diaryldicarboxylic acid diguanidides of the formula I 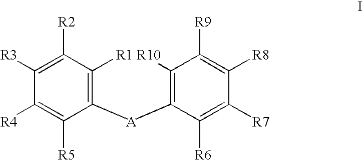
in which:
one of the radicals R(1), R(2), R(3), R(4) and R(5)
is —CO—N═C(NH₂)₂;
the other radicals R(1) and R(5) in each case
independently of one another are hydrogen, alkyl having 1, 2, 3 or 4 carbon atoms, F, Cl, —OR(32), —NR(33)R(34) or CF₃;
R(32), R(33) and R(34)
independently of one another are hydrogen or alkyl having 1, 2, 3 or 4 carbon atoms;
the other radicals R(2) and R(4) in each case
independently of one another are hydrogen, F, Cl, Br, I, OH, —CN, CF₃, —CO—N═C(NH₂)₂₁ alkyl having 1, 2, 3, 4, 5, 6, 7 or 8 carbon atoms, alkenyl having 2, 3, 4, 5, 6, 7 or 8 carbon atoms or —(CH₂)ₘR(14);
m is zero, 1 or 2;
R(14) is —(C₃-C₈)-cycloalkyl or phenyl,
which is not substituted or is substituted by 1-3 substituents selected from the group consisting of F and Cl, —CF₃₁ methyl, methoxy and —NR(15)R(16);
R(15) and R(16)
are hydrogen or —CH₃;
or
the other radicals R(2) and R(4) in each case
independently of one another are pyrrol-1-yl, pyrrol-2-yl or pyrrol-3-yl,
which is not substituted or is substituted by 1-4 substituents selected from the group consisting of F, Cl, Br, I, —CN, (C₂-C₈)-alkanoyl, (C₂-C₈)-alkoxycarbonyl, formyl, carboxyl, —CF₃, methyl, methoxy;
or
the other radicals R(2) and R(4) in each case
are R(22)-SO₂—, R(23)R(24)N—CO—, R(28)-CO— or R(29)R(30)N—SO₂;
R(22) and R(28)
independently of one another are methyl or —CF₃;
R(23), R(24), R(29) and R(30)
independently of one another are hydrogen or methyl;
or
the other radicals R(2) and R(4) in each case
independently of one another are —OR(35) or —NR(35)R(36);
R(35) and R(36)
independently of one another are hydrogen or alkyl having 1, 2, 3, 4, 5 or 6 carbon atoms;
or
R(35) and R(36)
together are 4-7 methylene groups, of which one CH₂ group can be replaced by oxygen,—S—, —NH—, —NCH₃ or —N-benzyl;
the other radical R(3) in each case
is hydrogen, —SR(25), —OR(25), —NR(25)R(26), —CR(25)R(26)R(27);
R(25) is hydrogen, alkyl having 1, 2, 3, 4, 5, 6, 7 or 8 carbon atoms or phenyl,
which is unsubstituted or substituted by 1-3 substituents selected from the group consisting of F, Cl, CF₃, CH₃, methoxy, hydroxyl, amino, methylamino and dimethylamino;
or
R(25) is —(C₁-C₉)-heteroaryl,
which is unsubstituted or substituted by 1-3 substituents selected from the group consisting of F, Cl, CF₃, CH₃, methoxy, hydroxyl, amino, methylamino and dimethylamino;
R(26) and R(27)
independently of one another are defined as R(25) or are hydrogen or alkyl having 1, 2, 3, 4, 5, 6, 7 or 8 carbon atoms;
one of the radicals R(6), R(7), R(8), R(9) and R(10)
is —CO—N═C(NH₂)₂;
the other radicals R(6) and R(10) in each case
independently of one another are hydrogen, alkyl having 1, 2, 3 or 4 carbon atoms, F, Cl, —OR(132), —NR(133)R(134) or CF₃;
R(132), R(133) and R(134)
independently of one another are hydrogen or alkyl having 1, 2, 3 or 4 carbon atoms;
the other radicals R(7) and R(9) in each case
independently of one another are hydrogen, F, Cl, Br, I, OH, —CN, CF₃, —CO—N═C(NH₂)₂, alkyl having 1, 2, 3, 4, 5, 6, 7 or 8 carbon atoms, alkenyl having 2, 3, 4, 5, 6, 7 or 8 carbon atoms or —(CH₂)ₘₘR(114);
mm is zero, 1 or 2;
R(114)
is —(C₃-C₈)-cycloalkyl or phenyl,
which is not substituted or is substituted by 1-3 substituents selected from the group consisting of F and Cl, —CF₃, methyl, methoxy and —NR(115)R(116);
R(115) and R(116)
are hydrogen or —CH₃;
or
the other radicals R(7) and R(9) in each case
independently of one another are pyrrol-1-yl, pyrrol-2-yl or pyrrol-3-yl,
which is not substituted or is substituted by 1-4 substituents selected from the group consisting of F, Cl, Br, I, —CN, (C₂-C₈)-alkanoyl, (C₂-C₈)-alkoxycarbonyl, formyl, carboxyl, —CF₃, methyl and methoxy;
or
the other radicals R(7) and R(9) in each case
are R(122)-SO₂—, R(123)R(124)N—CO—, R(128)-CO— or R(129)R(130)N—SO₂;
R(122) and R(128)
independently of one another are methyl or —CF₃;
R(123), R(124), R(129) and R(130)
independently of one another are hydrogen or methyl;
or
the other radicals R(7) and R(9) in each case
independently of one another are —OR(135) or —NR(135)R(136);
R(135) and R(136)
independently of one another are hydrogen or alkyl having 1, 2, 3, 4, 5 or 6 carbon atoms;
or
R(135) and R(136)
together are 4-7 methylene groups, of which one CH₂ group can be replaced by oxygen,—S—, —NH—, —NCH₃ or —N-benzyl;
the other radical R(8) in each case
is hydrogen, —SR(125), —OR(125), —NR(125)R(126) or —CR(125)R(126)R(127);
R(125)
is hydrogen, alkyl having 1, 2, 3, 4, 5, 6, 7 or 8 carbon atoms or phenyl,
which is unsubstituted or substituted by 1-3 substituents selected from the group consisting of F, Cl, CF₃, CH₃, methoxy, hydroxyl, amino, methylamino and dimethylamino;
or
R(125)
is —(C₁-C₉)-heteroaryl,
which is unsubstituted or substituted by 1-3 substituents selected from the group consisting of F, Cl, CF₃, CH₃, methoxy, hydroxyl, amino, methylamino and dimethylamino;
R(126) and R(127)
independently of one another are defined as R(125) or are hydrogen or alkyl having 1, 2, 3, 4, 5, 6, 7 or 8 carbon atoms;
A is absent or is —NR(11)-CO—, —NR(12)-CO—NR(13)-, —NR(17)-CO—NR(18)-SO₂—, —NR(19)-SO₂—, —SO₂—NR(19)-SO₂—, —SO₂—NR(19)-CO—, —O—CO—NR(19)-SO₂— or —CR(20)=CR(21)-;
R(11), R(12), R(13), R(17), R(18), R(19), R(20) and R(21)
independently of one another are hydrogen or alkyl having 1, 2, 3, 4, 5, 6, 7 or 8 carbon atoms
and their pharmaceutically tolerable salts;
au) substituted thiophenylalkenylcarboxylic acid guanidides of the formula I 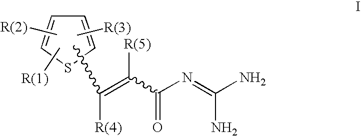
in which:
at least one of the substituents R(1), R(2) and R(3)
is —Oₚ—(CH₂)ₛ—C_{q}F_{2q+1}, R(40)CO— or R(31 )SOₖ—;
p is zero or 1;
s is zero, 1, 2, 3 or 4;
q is 1, 2, 3, 4, 5, 6, 7 or 8;
k is zero, 1 or 2;
R(40) is alkyl having 1, 2, 3, 4, 5, 6, 7 or 8 carbon atoms,
perfluoroalkyl having 1, 2, 3, 4, 5, 6, 7 or 8 carbon atoms,
cycloalkyl having 3, 4, 5, 6, 7 or 8 carbon atoms, or phenyl,
which is not substituted or is substituted by 1-3 substituents selected from the group consisting of F, Cl, CF₃₁ methyl and methoxy;
R(31) is alkyl having 1, 2, 3, 4 5, 6, 7 or B carbon atoms,
perfluoroalkyl having 1, 2, 3, 4, 5, 6, 7 or 8 carbon atoms,
cycloalkyl having 3, 4, 5, 6, 7 or 8 carbon atoms, or phenyl,
which is not substituted or is substituted by 1-3 substituents selected from the group consisting of F, Cl, CF₃, methyl or methoxy;
or
R(31) is NR(41)R(42);
R(41) and R(42)
independently of one another are hydrogen, alkyl having 1, 2, 3 or 4 carbon atoms, perfluoroalkyl having 1, 2, 3 or 4 carbon atoms,
or
R(41) and R(42)
together are 4 or 5 methylene groups, of which one CH₂ group can be replaced by oxygen, S, NH, N—CH₃ or N-benzyl;
and the other substituents R(1), R(2) and R(3) in each case
independently of one another are H, F, Cl, Br, I, CN, —Oₙₐ—CₘₐH₂ₘₐ₊₁ or —O_{ga}CᵣₐH_{2rg}R(10);
na is zero or 1;
ma is zero, 1, 2, 3, 4, 5, 6, 7 or 8;
ga is zero or 1;
ra is zero, 1, 2, 3 or 4;
R(10) is cycloalkyl having 3, 4, 5, 6, 7 or 8 carbon atoms or phenyl,
where the phenyl is not substituted or is substituted by 1-3 substituents selected from the group consisting of F, Cl, CF₃, methyl and methoxy;
R(4) and R(5)
independently of one another are hydrogen, F, Cl, Br, I, CN, alkyl having 1, 2, 3, 4, 5, 6, 7 or 8 carbon atoms, perfluoroalkyl having 1, 2, 3, 4, 5, 6, 7 or 8 carbon atoms, cycloalkyl having 3, 4, 5, 6, 7 or 8 carbon atoms or phenyl,
which is not substituted or is substituted by 1-3 substituents selected from the group consisting of F, Cl, CF₃, methyl, methoxy and NR(14)R(15);
R(14) and R(15)
independently of one another are H, alkyl having 1, 2, 3 or 4 carbon atoms or perfluoroalkyl having 1, 2, 3 or 4 carbon atoms;
and their pharmaceutically tolerable salts;

4. The use of an Na⁺/H⁺ exchange inhibitor as claimed in claims 1 to 3 for the production of a medicament for the treatment of hypercholesterolemia-related disorders of the cardiovascular system.

5. The use of an Na⁺/H⁺ exchange inhibitor as claimed in claims 1 to 3 for the production of a medicament for the prevention and treatment of endothelial dysfunction syndrome.

6. The use of an Na⁺/H⁺ exchange inhibitor as claimed in claims 1 to 3 for the production of a medicament for the prevention and treatment of cardiac hypertrophies and cardiomyopathies.

7. The use of an Na⁺/H⁺ exchange inhibitor as claimed in claims 1 to 3 for the production of a medicament for the prevention and treatment of coronary vascular spasms and myocardial infarcts, which are caused as a result of increased lipid levels and endothelial dysfunction syndrome.

8. The use of an Na⁺/H⁺ exchange inhibitor as claimed in claims 1 to 3 for the production of a medicament for the prevention and treatment of thromboses which are caused by increased lipid levels and endothelial dysfunction syndrome.

9. The use of an Na⁺/H⁺ exchange inhibitor as claimed in claims 1 to 3 for the production of a medicament for the prevention and treatment of high blood pressure which is caused by increased lipid levels and endothelial dysfunction syndrome.

10. The use of an Na⁺/H⁺ exchange inhibitor as claimed in claims 1 to 3 for the production of a medicament for the prevention and treatment of peipheral vascular disorders which are caused by increased lipid levels and endothelial dysfunction syndrome.

11. The use of an Na⁺/H⁺ exchange inhibitor for the production of a medicament as claimed in claims 1 to 10 in combination with a hypotensive medicament.

12. The use of an Na⁺/H⁺ exchange inhibitor for the production of a medicament as claimed in claims 1 to 10 in combination with an angiotensin converting enzyme (ACE) inhibitor.

13. The use of an Na⁺/H⁺ exchange inhibitor for the production of a medicament as claimed in claims 1 to 10 in combination with an angiotensin receptor antagonist.

14. The use of an Na⁺/H⁺ exchange inhibitor for the production of a medicament as claimed in claims 1 to 10 in combination with a blood lipid level-lowering active compound.

15. The use of an Na⁺/H⁺ exchange inhibitor for the production of a medicament as claimed in claims 1 to 10 in combination with an HMG-CoA reductase inhibitor.

16. The use of an Na⁺/H⁺ exchange inhibitor for the production of a medicament as claimed in claims 1 to 10 in combination with lovastatin or pravastatin.
